SEQUENCE LISTING (6 sequences)

SEQ ID NO: 1 - protein, 183 aa, synthetic construct
GLNDIFEAQKIEWHETEYKLVVVGACGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK

SEQ ID NO: 2 - protein, 183 aa, synthetic construct
GLNDIFEAQKIEWHETEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 3 - protein, 183 aa, synthetic construct
GLNDIFEAQKIEWHETEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 4 - protein, 172 aa, synthetic construct
GGGGSHMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQH

SEQ ID NO: 5 - protein, 176 aa, synthetic construct
GGGGMTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLN

SEQ ID NO: 6 - protein, 524 aa, synthetic construct
MGSSHHHHHHSGENLYFQGSSGLNDIFEAQKIEWHESSEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

(19) United States
(12) Patent Application Publication
(10) Pub. No.: US 2026/0200925 A1
(43) Pub. Date: Jul. 16, 2026

(54) SMALL MOLECULE INHIBITORS OF KRAS MUTATED PROTEINS

(71) Applicants: MERCK SHARP & DOHME LLC, Rahway, NJ (US); ASTEX THERAPEUTICS LTD., Cambridge (GB); TAIHO PHARMACEUTICAL CO., LTD., Tokyo (JP)

(72) Inventors: DAVID L. SLOMAN, Newton, MA (US); SYMON GATHIAKA, Waltham, MA (US); SHUHEI KAWAMURA, Cambridge, MA (US); TIMOTHY HENDERSON, Natick, MA (US); ANDREW J. HOOVER, Framingham, MA (US); UMA SWAMINATHAN, Auburndale, MA (US); INDU BHARATHAN, Somerville, MA (US); THOMAS H. GRAHAM, Somerville, MA (US); YONGXIN HAN, Needham, MA (US); PATRICK SCHÖPF, London (GB); YU KOBAYAKAWA, Ibaraki (JP); TSUYOSHI OSHIMA, Ibaraki (JP); TAKAO UNO, Ibaraki (JP)

(21) Appl. No.: 18/713,496

(22) PCT Filed: Nov. 22, 2022
(86) PCT No.: PCT/US2022/080348

§ 371 Date: May 24, 2024

Related U.S. Application Data
(60) Provisional application No. 63/282,878, filed on Nov. 24, 2021.

(51) Int. Cl. C07D 487/04; A61K 31/517; A61K 31/519; A61K 31/55; A61K 31/553; C07D 401/04; C07D 401/14; C07D 417/14; C07D 471/04; C07D 519/00

(52) U.S. Cl. CPC C07D 487/04; A61K 31/517; A61K 31/519; A61K 31/55; A61K 31/553; C07D 401/04; C07D 401/14; C07D 417/14; C07D 471/04; C07D 519/00

(57) ABSTRACT

Compounds of Formula (I) or their pharmaceutically acceptable salts can inhibit the G12C, G12D and/or G12V mutants of Kirsten rat sarcoma (KRAS) protein and are expected to have utility as therapeutic agents, for example, for treating cancer. The disclosure also provides pharmaceutical compositions which comprise compounds of Formula (I) or pharmaceutically acceptable salts thereof. The disclosure also relates to methods for use of the compounds or their pharmaceutically acceptable salts in the therapy and prophylaxis of cancer and for preparing pharmaceuticals for this purpose.

### CROSS-REFERENCE

This application claims priority to U.S. Provisional Patent Application No. 63/282,878 filed Nov. 24, 2021, the contents of which are incorporated herein by reference in their entirety.

### FIELD OF THE INVENTION

The present disclosure relates to small molecule inhibitors of KRAS that inhibit, for example, the G12C mutant, G12D mutant and G12V mutant of Kirsten rat sarcoma (KRAS) protein and relates to a pharmaceutical composition comprising a compound of Formula (I) as well as methods of using such a compound for treatment of diseases, including cancers.

### BACKGROUND

RAS, which is a small monomeric GTP-binding protein having a molecular weight of about 21 kDa, acts as a molecular on/off switch. RAS can bind to GTP by binding to proteins of a guanine nucleotide exchange factor (GEF) (e.g., SOS1), which forces the release of a bound nucleotide, and releases GDP. When RAS binds to GTP, it becomes activated (turned on) and recruits and activates proteins necessary for the propagation of other receptors' signals, such as c-Raf and PI 3-kinase. RAS also possesses enzymatic activity with which it cleaves the terminal phosphate of the GTP nucleotide and converts the nucleotide into GDP. The rate of conversion is usually slow, but can be dramatically sped up by a protein of the GTPase-activating protein (GAP) class, such as RasGAP. When GTP is converted into GDP, RAS is deactivated (turned off).

The commonly known members of the RAS subfamily include HRAS, KRAS, and NRAS. Of these, mutations of KRAS are observed in many malignant tumors: in 86% of pancreatic ductal adenocarcinoma (PDAC), in 41% of colorectal cancers (CRC), and in 32% of lung adenocarcinoma (LUAD; a subtype of non-small-cell lung cancer (NSCLC)). The mutations often occur in the glycine residue at position 12 of KRAS (“G12”); the mutation at G12 dominates 91% (PDAC), 68% (CRC) and 85% (LUAD) of the total KRAS mutations, respectively. The distributions of amino acid substitutions at G12 vary among each tissue type. The most prevalent mutation in LUAD is the mutation into cysteine (“G12C”) (46%), while the predominant mutation in PDAC (45%) and CRC (45%) is the mutation into aspartic acid (“G12D”). The mutation at G12 into valine (“G12V”) is observed in a significant portion of G12 mutations in all of PDAC (35%), CRC (30%) and LUAD (23%). (Nature Reviews Drug Discovery, 19, 533-552, 2020).

Intense efforts in developing KRAS-G12C inhibitors are underway. Several covalent inhibitors which focus on the cysteine residue have been reported, and some of them have been subjected to clinical studies, such as AMG510 (NCT03600883), MRTX849 (NCT03785249) and JNJ-74699157 (NCT04006301). However, the KRAS-G12C mutation only accounts for a fraction of all KRAS mutations and is primarily found in LUAD. To effectively inhibit the other commonly-occurring KRAS mutated proteins, such as KRAS-G12D and KRAS-G12V, different approaches are needed as these mutants lack reactive cysteines in the active site (Nature Reviews Drug Discovery, 19, 533-552, 2020).

### SUMMARY OF THE DISCLOSURE

The present disclosure provides small molecule inhibitors which modulate mutant KRAS, HRAS, and/or NRAS proteins and may be valuable pharmaceutically active compounds for the treatment of cancer. In some embodiments the disclosed compounds selectively inhibit the KRAS-G12C, KRAS-G12D and/or KRAS-G12V proteins. The compounds of Formula (I):

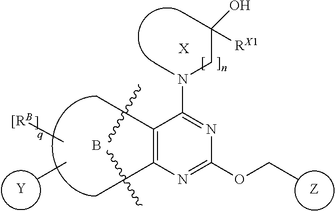

and their pharmaceutically acceptable salts, can modulate the activity of KRAS, HRAS and/or NRAS activity and thereby affect the signaling pathway which regulates cell growth, differentiation, and proliferation associated with oncological disorders. In certain embodiments, the compounds of Formula (I) can inhibit the KRAS-G12C, KRAS-G12D and/or KRAS-G12V proteins. The disclosure furthermore provides processes for preparing compounds of Formula (I), methods for using such compounds to treat oncological disorders, and pharmaceutical compositions which comprise compounds of Formula (I).

### DETAILED DESCRIPTION OF THE INVENTION

### Compounds of the Disclosure

In one embodiment, the present disclosure provides a compound having structural Formula (I), or a pharmaceutically acceptable salt thereof, as shown above, wherein:
the moiety

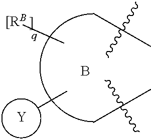

is selected from the group consisting of:

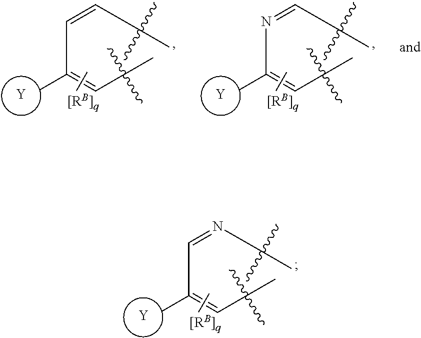

each occurrence of R^{B} is independently selected from the group consisting of halo, cyano, C₁-C₃ alkyl, C₁-C₃ fluoroalkyl, cyclopropyl and C₁-C₄ cyanoalkyl;
Ring X is selected from the group consisting of:
(i) a 5- to 9-membered monocyclic- or fused bicyclic- or bridged bicyclic-heterocycloalkyl, wherein the heterocycloalkyl is saturated and contains 0 to 2 heteroatom groups selected from the group consisting of N, S, S(O), S(O)₂ and O, in addition to the illustrated N atom; and
(ii) an 8- to 10-membered spiroheterocycloalkyl, wherein said spiroheterocycloalkyl is saturated and contains 0 to 2 heteroatom groups selected from the group consisting of N, S, S(O), S(O)₂ and O, in addition to the illustrated N atom;
wherein ring X is unsubstituted or independently substituted by 1 to 3 R^{X2} substituents selected from the group consisting of fluoro, cyano, hydroxy, C₁-C₆ alkyl, C₁-C₆ fluoroalkyl, C₁-C₃ hydroxyalkyl, —N(H)C(O)heteroaryl, wherein heteroaryl is optionally substituted by C₁-C₃ alkyl;
R^{X1} is selected from the group consisting of H, C₁-C₆ alkyl, C₁-C₆ fluoroalkyl, C₁-C₆ hydroxyalkyl and C₁-C₄ cyanoalkyl;
Ring Y is selected from the group consisting of:
(i) a 8- to 10-membered bicyclic ring system, wherein the 8- to 10-membered bicyclic ring system is partially unsaturated or aromatic, and wherein the 8- to 10-membered bicyclic ring system contains 0 to 3 heteroatoms selected from the group consisting of N, S, and O;
(ii) a 13- to 14-membered tricyclic ring system, wherein the 13- to 14-membered tricyclic ring system is partially unsaturated or aromatic, and wherein the 13- to 14-membered tricyclic ring system contains 0 to 3 heteroatoms selected from the group consisting of N, S, and O; and
(iii) phenyl; and
(iv) a 6-membered heteroaryl ring containing 1 to 2 N atoms;
wherein Ring Y is unsubstituted or independently substituted by 1 to 4 RY substituents selected from the group consisting of halo, hydroxy, amino, oxo, C₁-C₃ alkyl, C₁-C₃ alkoxy, C₁-C₃ fluoroalkoxy, C₂-C₃ alkynyl, C₂-C₃ fluoroalkynyl, C₁-C₃ fluoroalkyl, C₁-C₃ cyanoalkyl, C₃-C₆ cycloalkyl, C₃-C₆ cyclofluoroalkyl, C₃-C₆ cycloalkoxy, C₃-C₆ cyclofluoroalkoxy, C₂-C₃ alkenyl, C₂-C₃ fluoroalkenyl and cyano;
Ring Z is selected from the group consisting of:
(i) a 5- to 8-membered monocyclic- or bicyclic-heterocycloalkyl, wherein said heterocycloalkyl is saturated and contains 1 to 3 heteroatoms independently selected from the group consisting of N, S, and O, and wherein said heterocycloalkyl is unsubstituted or substituted with 1-2 substituents R^{ZHC} selected from the group consisting of halo, hydroxy, C₁-C₃ alkyl, C₁-C₃ hydroxyalkyl, —C(H)(OH)CF₂H, —O—CH₂—O—(C₁-C₃ fluoroalkyl), and methylene(C₁-C₃ alkyl)(C₁-C₃ alkyl)carbamate;
(ii)

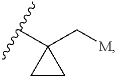

wherein M is selected from the group consisting of hydroxy, C₁-C₃ dialkylamino, and C₁-C₄ alkylamino, and wherein the cyclopropyl group is unsubstituted or independently substituted with up to 2 halo groups;
(iii)

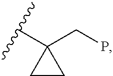

wherein P is 5- to 8-membered monocyclic- or fused bicyclic- or bridged bicyclic-heterocycloalkyl, wherein said heterocycloalkyl is saturated and contains 1 to 2 heteroatoms selected from the group consisting of N and O, wherein said heterocycloalkyl is unsubstituted or substituted with 1 R^{P} substituent selected from the group consisting of halo, hydroxy, C₁-C₃ hydroxyalkyl, C₁-C₃ cyanoalkyl, carbamoyl, C₁-C₃ alkoxy, cyano, and —NHC(O)C₁-C₃alkyl, and wherein the cyclopropyl group is unsubstituted or independently substituted with up to 2 halo groups; and
(iv) a 4- to 8-membered monocyclic- or bicyclic-cycloalkyl, wherein said cycloalkyl is saturated and wherein said cycloalkyl is unsubstituted or independently substituted with 1-3 substituents R^{ZC} selected from the group consisting of halo, hydroxy, C₁-C₃ alkyl, C₁-C₃ fluoroalkyl, C₁-C₃ hydroxyalkyl, C₁-C₃ hydroxyfluoroalkyl, C₃-C₄ cycloalkyl, C₃-C₄ cyclofluoroalkyl, C₃-C₄ hydroxycycloalkyl, and C₃-C₄ hydroxycyclofluoroalkyl;
subscript n is 1 or 2; and
subscript q is 0, 1 or 2.

In another embodiment, the present disclosure provides a compound of Formula (I), or the pharmaceutically acceptable salt thereof, wherein ring X is a 5- to 8-membered monocyclic heterocycloalkyl, wherein the heterocycloalkyl is saturated and contains 0 to 1 heteroatoms selected from the group consisting of N, S, and O, in addition to the illustrated N atom.

In another embodiment, the present disclosure provides a compound of Formula (I), or a pharmaceutically acceptable salt thereof, wherein the group

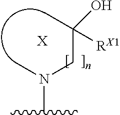

is selected from the group consisting of:

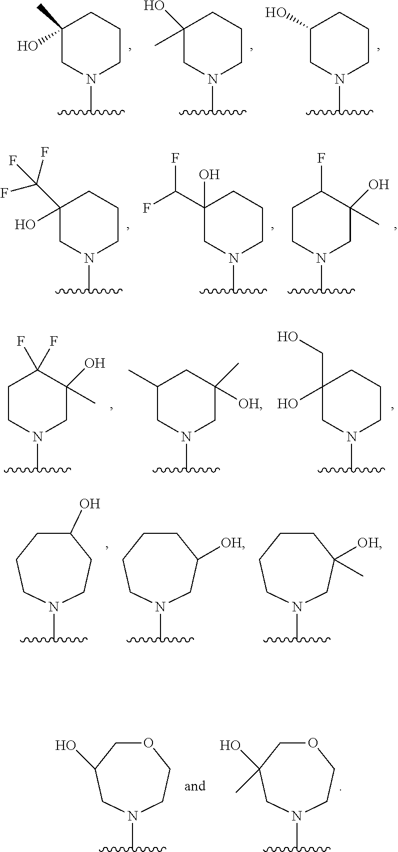

In another embodiment, the present disclosure provides a compound of Formula (I), or a pharmaceutically acceptable salt thereof, wherein the group

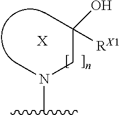

is selected from the group consisting of:

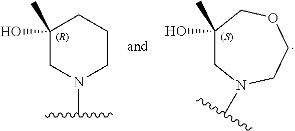

In another embodiment, the present disclosure provides a compound of Formula (I), or a pharmaceutically acceptable salt thereof, wherein the moiety

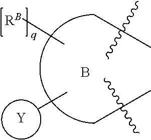

is

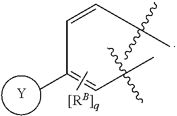

In another embodiment, the present disclosure provides a compound of Formula (I), or a pharmaceutically acceptable salt thereof, wherein ring Y is selected from the group consisting of naphthyl, phenyl, pyridyl, benzoxazolyl, benzopyrazolyl, benzothiazolyl, pyridopyrazolyl and benzothiophenyl.

In another embodiment, the present disclosure provides a compound of Formula (I), or a pharmaceutically acceptable salt thereof, wherein ring Y is selected from the group consisting of naphthyl, phenyl, pyridyl, benzoxazolyl, benzopyrazolyl, and benzothiazolyl.

In another embodiment, the present disclosure provides a compound of Formula (I), or a pharmaceutically acceptable salt thereof, wherein ring Y is selected from the group consisting of:

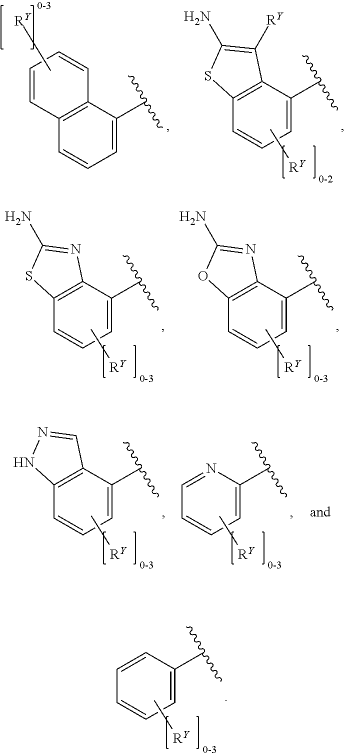

In another embodiment, the present disclosure provides a compound of Formula (I), or a pharmaceutically acceptable salt thereof, wherein ring Y is selected from the group consisting of:

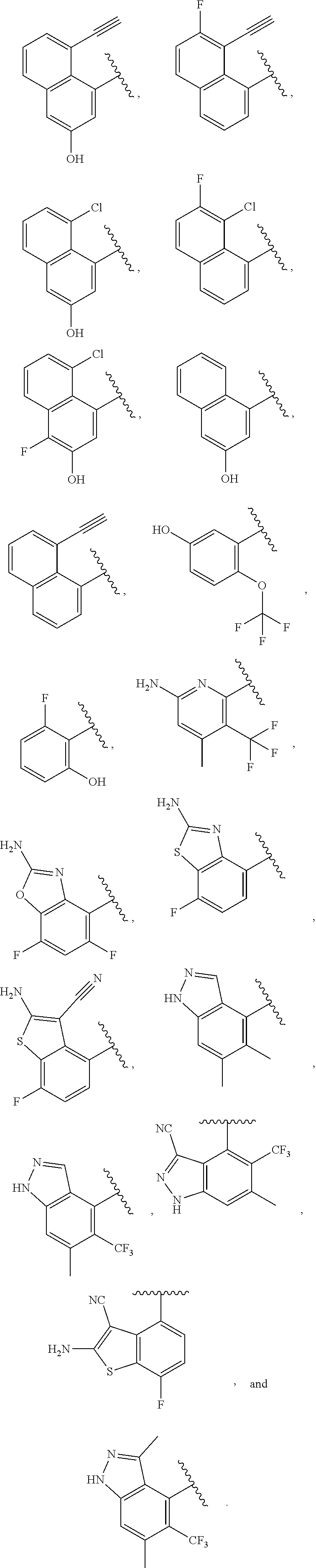

In another embodiment, the present disclosure provides a compound of Formula (I), or a pharmaceutically acceptable salt thereof, wherein ring Y is selected from the group consisting of:

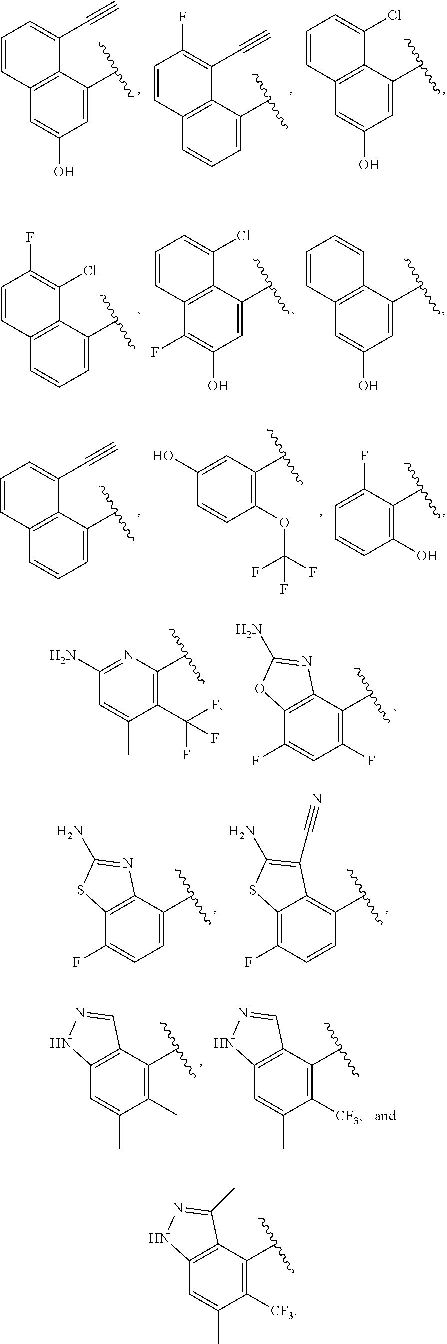

In another embodiment, the present disclosure provides a compound of Formula (I), or a pharmaceutically acceptable salt thereof, wherein Ring Y is unsubstituted or independently substituted by 1 to 4 RY substituents selected from the group consisting of halo, hydroxy, amino, oxo, C₁-C₃ alkyl, C₁-C₃ alkoxy, C₁-C₃ fluoroalkoxy, C₂-C₃ alkynyl, C₂-C₃ fluoroalkynyl, C₁-C₃ fluoroalkyl, C₁-C₃ cyanoalkyl, and cyano.

In another embodiment, the present disclosure provides a compound of Formula (I), or a pharmaceutically acceptable salt thereof, wherein the group

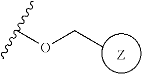

is selected from the group consisting of:

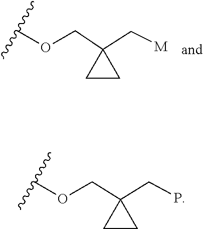

In another embodiment, the present disclosure provides a compound of Formula (I), or a pharmaceutically acceptable salt thereof, wherein the group

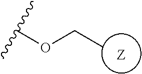

is selected from the group consisting of:

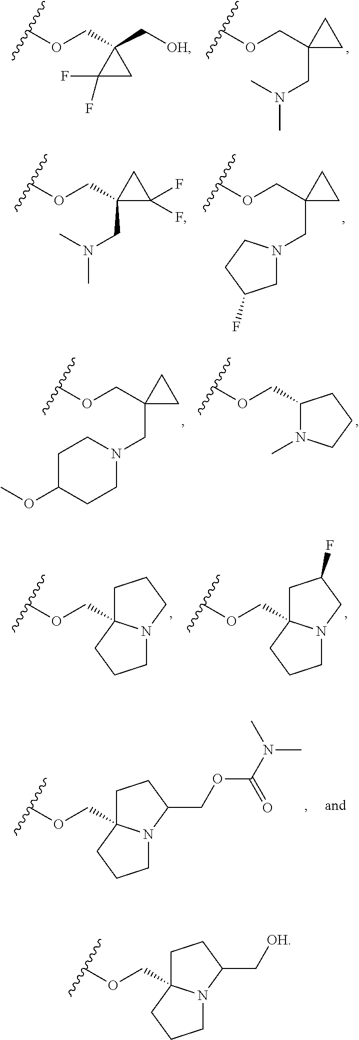

In another embodiment, the present disclosure provides a compound of Formula (I), or a pharmaceutically acceptable salt thereof, wherein Ring Z is selected from the group consisting of:
(i) a 5- to 8-membered monocyclic- or bicyclic-heterocycloalkyl, wherein said heterocycloalkyl is saturated and contains 1 nitrogen heteroatom and wherein said heterocycloalkyl is unsubstituted or substituted with 1-2 substituents R^{ZHC} selected from the group consisting of halo, C₁-C₃ alkyl, C₁-C₃ hydroxyalkyl, —C(H)(OH)CF₂H, —O—CH₂—O—(C₁-C₃ fluoroalkyl), and methylene(C₁-C₃ alkyl)(C₁-C₃ alkyl)carbamate;
(ii)

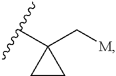

wherein M is selected from the group consisting of hydroxy, C₁-C₃ dialkylamino, and C₁-C₄ alkylamino, and wherein the cyclopropyl group is unsubstituted or substituted with up to 2 halo groups; and
(iii)

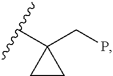

wherein P is 5- to 8-membered monocyclic- or fused bicyclic- or bridged bicyclic-heterocycloalkyl, wherein said heterocycloalkyl is saturated and contains 1 to 2 heteroatoms selected from the group consisting of N and O, wherein said heterocycloalkyl is unsubstituted or substituted with 1 R^{P} substituent selected from the group consisting of halo, hydroxy, C₁-C₃ hydroxyalkyl, C₁-C₃ cyanoalkyl, carbamoyl, C₁-C₃ alkoxy, cyano, and —NHC(O)C₁-C₃alkyl, and wherein the cyclopropyl group is unsubstituted or substituted with up to 2 halo groups.

In certain embodiments, the present disclosure provides a compound of Formula (I), or a pharmaceutically acceptable salt thereof, wherein
ring Y is selected from the group consisting of:

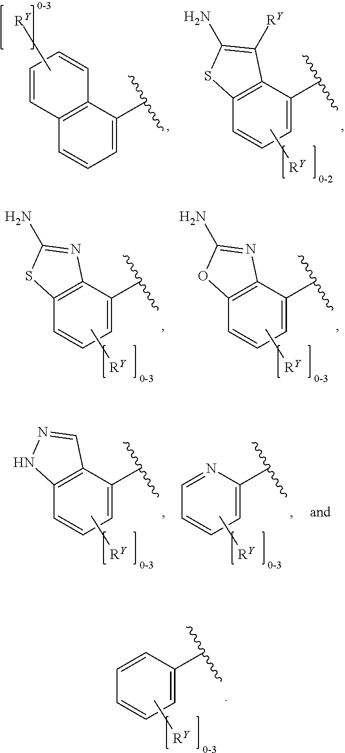

each R^{Y} being independently selected from the group consisting of fluoro, chloro, amino, hydroxy, ethynyl, methyl, trifluoromethyl, and cyano,

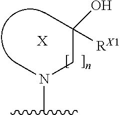

is selected from the group consisting of:

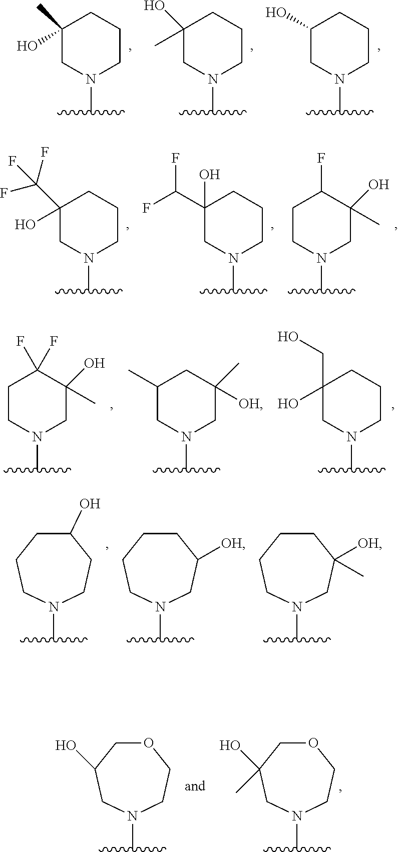

and

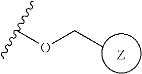

is selected from the group consisting of:

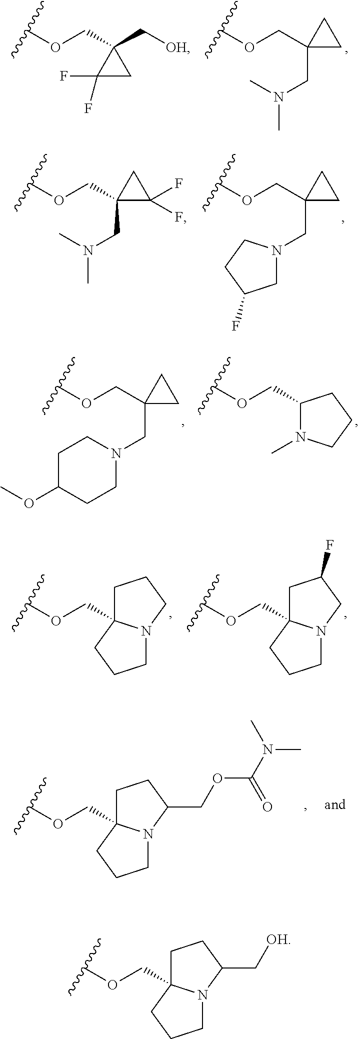

In specific embodiments, the present disclosure provides a compound as described in any one of Examples 1-49 as set forth below, or a pharmaceutically acceptable salt thereof.

The present disclosure includes the pharmaceutically acceptable salts of the compounds defined herein, including the pharmaceutically acceptable salts of all structural formulas, embodiments and classes defined herein.

### Definitions

Unless defined otherwise, all technical and scientific terms used herein have the same meaning as is commonly understood by one of skill in the art to which this invention belongs.

As used throughout this disclosure, “a compound of Formula (I)” is to be understood to include “a compound of Formula (I) or a pharmaceutically acceptable salt thereof”. Likewise “a compound of Formula (I)”, “compound(s) disclosed herein”, “compound(s) described herein”, “compound(s) of the disclosure”, etc., are used interchangeably and include both the compound, as well as a pharmaceutically acceptable salt thereof.

“Alkenyl” means an aliphatic hydrocarbon group containing at least one carbon-carbon double bond and which may be straight or branched. Non-limiting examples include ethenyl, propenyl, and butenyl.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkoxy” and “alkyl-O—” are used interchangeably and refer to an alkyl group linked to oxygen.

“Alkynyl” means an aliphatic hydrocarbon group containing at least one carbon-carbon triple bond and which may be straight or branched. Non-limiting examples include ethynyl, propynyl, and butynyl.

“Aryl” means a monocyclic, bicyclic or tricyclic carbocyclic aromatic ring or ring system containing 5-14 carbon atoms, wherein at least one of the rings is aromatic. Non-limiting examples include phenyl and naphthyl.

“Aminoalkyl” means -alkyl-NH2 group in which the alkyl is as previously defined. The bond to the parent moiety is through a carbon atom of the alkyl component. Non-limiting examples of suitable aminoalkyl groups include aminomethyl and aminoethyl. “Alkylamino” means —NH-alkyl group in which the alkyl is as previously defined. The bond to the parent moiety is through the nitrogen of the amino component.

“Bicyclic ring system” refers to two joined rings. “Tricyclic ring system” refers to three joined rings. The rings may be fused, i.e., share two adjacent atoms, or “spirocyclic”, i.e., share only a single atom, or “bridged”, i.e., share three or more atoms with two bridgehead atoms being connected by a bridge containing at least one atom. Likewise the bicyclic or tricyclic rings may be aryl rings, heterocyclic rings, cycloalkyl rings, etc.

“Carbamoyl” means a H₂N—C(O)— group, which is the univalent group formed by loss of —OH group of carbamic acid. The bond to the parent group is through the carbon atom of the carbonyl component.

“Cyanoalkyl” means an -alkyl-CN group in which the alkyl is as previously defined. The bond to the parent moiety is through a carbon atom of the alkyl component. Non-limiting examples of suitable cyanoalkyl groups include cyanomethyl and 3-cyanopropyl.

“Cycloalkyl” means a saturated cyclic hydrocarbon radical. In particular embodiments, the cycloalkyl group has 3-12 carbon atoms, forming 1-3 carbocyclic rings, wherein cyclic systems having 2-3 rings can be fused. Examples of cycloalkyl include cyclopropyl, cyclobutyl, cyclopentyl, cyclohexyl, cycloheptyl, adamantyl, and the like. “Cyclofluoroalkyl” means a saturated cyclic hydrocarbon radical that is mono- or multiple-fluoro-substituted, e.g., doubly fluoro-substituted cyclopentyl. “Cycloalkoxy” refers to a cycloalkyl group linked through an oxygen to the parent moiety. “Cyclofluoroalkoxy” refers to a cyclofluoroalkyl group linked through an oxygen to the parent moiety.

“Dialkylamino” means an alkylamino as previously defined, wherein the amino atom is substituted by two alkyl substituents, which substitutions can be the same or different, e.g., —N(CH₃)₂ or —N(CH₃)(CH₂CH₃).

“Fluoroalkyl” includes mono-substituted as well as multiple fluoro-substituted alkyl groups, up to perfluoro substituted alkyl. For example, fluoromethyl, 1,1-difluoroethyl, trifluoromethyl or 1,1,1,2,2-pentafluorobutyl are included. “Fluoroalkenyl” includes mono-substituted as well as multiple fluoro-substituted alkenyl groups. “Fluoroalkynyl” includes mono-substituted as well as multiple fluoro-substituted alkynyl groups. “Fluoroalkoxy” includes mono-substituted as well as multiple fluoro-substituted “alkoxy” groups as previously defined.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heteroaryl” refers to aromatic monocyclic, bicyclic and tricyclic ring structures in which one or more atoms in the ring, the heteroatom(s), is an element other than carbon. Heteroatoms are typically O, S, or N atoms. Examples of heteroaryl groups include pyrazolyl, oxadiazolonyl, pyridinyl, pyrimidinyl, pyrrolyl, pyridazinyl, isoxazolyl, thiazolyl, oxazolyl, indolyl, benzoxazolyl, benzothiazolyl, and imidazolyl.

“Heterocycloalkyl” or “heterocyclic ring” or “heterocycle” means a non-aromatic monocyclic, bicyclic, tricyclic or bridged ring system comprising about 3 to about 10 ring atoms, preferably about 5 to about 10 ring atoms, in which one or more of the atoms in the ring system is an element other than carbon, for example, nitrogen, oxygen, phosphorus or sulfur, alone or in combination. There are no adjacent oxygen and/or sulfur atoms present in the ring system. In some embodiments, heterocycloalkyls contain about 5 to about 6 ring atoms. The prefix aza, oxa, phospha or thia before the heterocyclyl root name means that at least a nitrogen, oxygen, phosphorus or sulfur atom respectively is present as a ring atom. In some embodiments, the nitrogen or sulfur atom of the heterocycloalkyl can be optionally oxidized to the corresponding N-oxide, S-oxide or S,S-dioxide. For instance, in some embodiments the heterocycloalkyl can contain N, S, S(O), S(O)₂ and/or O (which are referred to herein as “heteroatom groups”). Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, phosphorinane, phosphinane, 1-oxophosphinan-1-ium and the like. “Spiroheterocycloalkyl” refers to a fused ring system in which the rings share only a single atom and at least one of the rings is a heterocycloalkyl.

“Hydroxyalkyl” means a HO-alkyl- group in which alkyl is as previously defined. The bond to the parent moiety is through a carbon atom of the alkyl group. Preferred hydroxyalkyls contain lower alkyl. Non-limiting examples of suitable hydroxyalkyl groups include hydroxymethyl and 2-hydroxyethyl. “Hydroxyfluoroalkyl” means a HO-fluoroalkyl-group in which fluoroalkyl is as previously defined. “Hydroxycycloalkyl” means a HO-cycloalkyl- group in which cycloalkyl is as previously defined. “Hydroxycyclofluoroalkyl” means a HO-cyclofluoroalkyl- group in which cyclofluoroalkyl is as previously defined.

“Methylene(C₁-C₃ alkyl)(C₁-C₃ alkyl)carbamate” means having the structure of

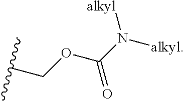

In other words, the carbamate group has alkyl groups, which can be the same or different, as previously defined, attached to the nitrogen atom.

When any variable (e.g., R^{Y}) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{Y}, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, such as R^{X}, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

The compounds of Formula (I) may contain one or more asymmetric centers and can thus occur as racemates and racemic mixtures, single enantiomers, diastereoisomeric mixtures and individual diastereoisomers. Centers of asymmetry that are present in the compounds of Formula (I) can all independently of one another have S configuration or R configuration. The compounds of Formula (I) include all possible enantiomers and diastereomers and mixtures of two or more stereoisomers, for example, mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism, the disclosure includes both the cis form and the trans form as well as mixtures of these forms in all ratios. The present disclosure is meant to comprehend all such stereoisomeric forms of the compounds of Formula (I). Where a structural formula or chemical name specifies a particular configuration at a stereocenter, the enantiomer or stereoisomer of the compound resulting from that specified stereocenter is intended. Where a structural formula of the compounds of Formula (I) indicates a straight line at a chiral center, the structural formula includes both the S and R stereoisomers associated with the chiral center and mixtures thereof.

The compounds of Formula (I) may be separated into their individual diastereoisomers by, for example, fractional crystallization from a suitable solvent, for example, methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Vibrational circular dichroism (VCD) may also be used to determine the absolute stereochemistry. Alternatively, any stereoisomer or isomers of the compounds of Formula (I) may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

The compounds of Formula (I) which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed by the compounds of Formula (I).

Some of the compounds of Formula (I) described herein may exist as atropisomers when the rotational energy barrier around a single bond is sufficiently high to prevent free rotation at a given temperature, thus allowing isolation of individual conformers with distinct properties. The individual atropisomers as well as mixtures thereof are encompassed with compounds of Formula (I) of the present disclosure. When resolved, individual atropisomers can be designated by established conventions such as those specified by the International Union of Pure Applied Chemistry (IUPAC) 2013 Recommendations.

In the compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of Formula (I) and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When a compound of Formula (I) is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When a compound of Formula (I) is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids. If a compound of Formula (I) simultaneously contains acidic and basic groups in the molecule, the disclosure also includes, in addition to the salt forms mentioned, inner salts or betaines (zwitterions). Salts can be obtained from the compounds of Formula (I) by customary methods which are known to the person skilled in the art, for example, by combination with an organic or inorganic acid or base in a solvent or dispersant, or by anion exchange or cation exchange from other salts. The present disclosure also includes all salts of the compounds of Formula (I) which, owing to low physiological compatibility, are not directly suitable for use in pharmaceuticals but which can be used, for example, as intermediates for chemical reactions or for the preparation of pharmaceutically acceptable salts.

Furthermore, the compounds of Formula (I) may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of Formula (I) may form solvates with water (i.e., a hydrate) or common organic solvents such as but not limited to ethyl acetate. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of Formula (I) which results in conversion in vivo to a compound within the scope of this disclosure is also within the scope of this disclosure.

The terms “therapeutically effective (or efficacious) amount” and similar descriptions such as “an amount efficacious for treatment” or “an effective dose” are intended to mean that amount of a compound of Formula (I) that will elicit the biological or medical response of a tissue, a system, animal or human that is being sought by a researcher, veterinarian, medical doctor or other clinician. In a preferred embodiment, the term “therapeutically effective amount” means an amount of a compound of Formula (I) that alleviates at least one clinical symptom in a human patient. The terms “prophylactically effective (or efficacious) amount” and similar descriptions such as “an amount efficacious for prevention” are intended to mean that amount of a compound of Formula (I) that will prevent or reduce the risk of occurrence of the biological or medical event that is sought to be prevented in a tissue, a system, animal or human by a researcher, veterinarian, medical doctor or other clinician.

### Dosages of the Compounds of Formula (I)

The dosage regimen utilizing a compound of Formula (I) is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compounds of Formula (I) is within the skill of the art. For administration to a human in, for example, the curative or prophylactic treatment of the conditions and disorders identified herein, the typical dosages of the compounds of Formula (I) can be about 0.05 mg/kg/day to about 50 mg/kg/day, or at least 0.05 mg/kg, or at least 0.08 mg/kg, or at least 0.1 mg/kg, or at least 0.2 mg/kg, or at least 0.3 mg/kg, or at least 0.4 mg/kg, or at least 0.5 mg/kg, and any amount therebetween, to about 50 mg/kg or less, or about 40 mg/kg or less, or about 30 mg/kg or less, or about 20 mg/kg or less, or about 10 mg/kg or less and any amount therebetween, which can be, for example, about 2.5 mg/day (0.5 mg/kg x 5 kg) to about 5000 mg/day (50 mg/kg x 100 kg). For example, dosages of the compounds can be about 0.1 mg/kg/day to about 50 mg/kg/day, or about 0.05 mg/kg/day to about 10 mg/kg/day, or about 0.05 mg/kg/day to about 5 mg/kg/day, or about 0.05 mg/kg/day to about 3 mg/kg/day, or about 0.07 mg/kg/day to about 3 mg/kg/day, or about 0.09 mg/kg/day to about 3 mg/kg/day, or about 0.05 mg/kg/day to about 0.1 mg/kg/day, or about 0.1 mg/kg/day to about 1 mg/kg/day, or about 1 mg/kg/day to about 10 mg/kg/day, or about 1 mg/kg/day to about 5 mg/kg/day, or about 1 mg/kg/day to about 3 mg/kg/day, or about 3 mg/day to about 500 mg/day, or about 5 mg/day to about 250 mg/day, or about 10 mg/day to about 100 mg/day, or about 3 mg/day to about 10 mg/day, or about 100 mg/day to about 250 mg/day. Such doses may be administered in a single dose or may be divided into multiple doses.

### Pharmaceutical Compositions

The compounds of Formula (I) and their pharmaceutically acceptable salts can be administered to animals, preferably to mammals, and in particular to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of said disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of Formula (I) and their pharmaceutically acceptable salts for use as pharmaceuticals, their use for modulating the activity of mutant KRAS, HRAS and/or NRAS proteins and in particular their use in the therapy and prophylaxis of the below-mentioned diseases or disorders as well as their use for preparing medicaments for these purposes. In certain embodiments, the compounds of Formula (I) and their pharmaceutically acceptable salts inhibit the KRAS-G12C, KRAS-G12D and/or KRAS-G12V proteins.

Furthermore, the present disclosure provides pharmaceutical compositions which comprise as active component an effective dose of at least one compound of Formula (I) and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of at least one compound of Formula (I) and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., cancer, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The amount of active compound of a compound described herein and/or its pharmaceutically acceptable salts in the pharmaceutical composition normally is from 0.01 to 200 mg, or from 0.1 to 200 mg, or from 1 to 200 mg, per dose, but depending on the type of the pharmaceutical composition, it can also be higher. In some embodiments, the amount of active compound of a compound of Formula (I) and/or its pharmaceutically acceptable salts in the pharmaceutical composition is from 0.01 to 10 mg per dose. The pharmaceutical compositions usually comprise 0.5 to 90 percent by weight of at least one compound of Formula (I) and/or its pharmaceutically acceptable salts. The preparation of the pharmaceutical compositions can be carried out in a manner known per se. For this purpose, one or more compounds of Formula (I) and/or their pharmaceutically acceptable salts, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules, it is possible to use, for example, lactose, starch, for example, maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example, of solutions for injection, or of emulsions or syrups are, for example, water, physiologically acceptable sodium chloride solution, alcohols such as ethanol, glycerol, polyols, sucrose, invert sugar, glucose, mannitol, vegetable oils, etc. It is also possible to lyophilize the compounds of Formula (I) and their pharmaceutically acceptable salts and to use the resulting lyophilisates, for example, for preparing preparations for injection or infusion. Suitable carriers for microcapsules, implants or rods are, for example, copolymers of glycolic acid and lactic acid.

Besides the active compounds and carriers, the pharmaceutical compositions can also contain customary additives, for example, fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents and/or antioxidants.

### Methods of Using the Compounds of Formula (I)

The present application provides a method of inhibiting RAS-mediated cell signaling comprising contacting a cell with a compound of Formula (I) or a pharmaceutically acceptable salt thereof. Inhibition of RAS-mediated signal transduction can be assessed and demonstrated by a wide variety of ways known in the art. Non-limiting examples include (a) a decrease in GTPase activity of RAS; (b) a decrease in GTP binding affinity or an increase in GDP binding affinity; (c) an increase in K_{off} of GTP or a decrease in K_{off} of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf. Kits and commercially available assays can be utilized for determining one or more of the above.

The present application also provides methods of using the compounds of Formula (I) (or their pharmaceutically acceptable salts) or pharmaceutical compositions containing such compounds to treat disease conditions, including but not limited to, conditions implicated by mutant KRAS, HRAS and/or NRAS proteins (e.g., cancer), and in some embodiments the KRAS-G12C, KRAS-G12D and/or KRAS-G12V mutants.

In some embodiments, a method for treatment of cancer is provided, the method comprising administering a therapeutically effective amount a compound of Formula (I) (or a pharmaceutically acceptable salt thereof) or any of the foregoing pharmaceutical compositions comprising such a compound to a subject in need of such treatment. In some embodiments, the cancer is mediated by a KRAS, HRAS or NRAS mutation, e.g., the KRAS-G12C, KRAS-G12D and/or KRAS-G12V mutations. In various embodiments, the cancer is pancreatic cancer, colorectal cancer or lung cancer. In some embodiments, the cancer is gall bladder cancer, thyroid cancer, or bile duct cancer.

In some embodiments the present disclosure provides a method of treating a disorder in a subject in need thereof, wherein said method comprises determining if the subject has a KRAS, HRAS or NRAS mutation (e.g., KRAS-G12C, KRAS-G12D and/or KRAS-G12V mutations) and if the subject is determined to have the KRAS, HRAS or NRAS mutation, then administering to the subject a therapeutically effective amount of a compound of Formula (I) or a pharmaceutically acceptable salt thereof.

The disclosed compounds inhibit anchorage-independent cell growth and therefore have the potential to inhibit tumor metastasis. Accordingly, another embodiment of the present disclosure provides a method for inhibiting tumor metastasis, the method comprising administering an effective amount a compound of Formula (I).

KRAS, HRAS or NRAS mutations have also been identified in hematological malignancies (e.g., cancers that affect blood, bone marrow and/or lymph nodes). Accordingly, certain embodiments are directed to administration of the compounds of Formula (I) (e.g., in the form of a pharmaceutical composition) to a subject in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds are useful for treatment of lymphomas such as Hodgkin's lymphoma or non-Hodgkin's lymphoma. In various embodiments, the compounds are useful for treatment of plasma cell malignancies such as multiple myeloma, mantle cell lymphoma, and Waldenstrom's macroglubunemia.

Determining whether a tumor or cancer comprises a KRAS, HRAS or NRAS mutation (e.g., the KRAS-G12C, KRAS-G12D and/or KRAS-G12V mutations) can be undertaken by assessing the nucleotide sequence encoding the KRAS, HRAS or NRAS protein, by assessing the amino acid sequence of the KRAS, HRAS or NRAS protein, or by assessing the characteristics of a putative KRAS, HRAS or NRAS mutant protein. The sequences of wild-type human KRAS, HRAS or NRAS are known in the art.

Methods for detecting a mutation in a KRAS, HRAS or NRAS nucleotide sequence are also known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS, HRAS or NRAS mutations (e.g., the KRAS-G12C, KRAS-G12D and/or KRAS-G12V mutations) by real-time PCR. In real-time PCR, fluorescent probes specific for the KRAS, HRAS or NRAS mutation are used. When a mutation is present, the probe binds and fluorescence is detected. In some embodiments, the KRAS, HRAS or NRAS mutation is identified using a direct sequencing method of specific regions (e.g., exon 2 and/or exon 3) in the KRAS, HRAS or NRAS gene.

Methods for detecting a mutation in a KRAS, HRAS or NRAS protein (e.g., the KRAS-G12C, KRAS-G12D and/or KRAS-G12V mutations) are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing.

A number of tissue samples can be assessed for determining whether a tumor or cancer comprises a KRAS, HRAS or NRAS mutation (e.g., the KRAS-G12C, KRAS-G12D and/or KRAS-G12V mutations). In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

The present application also provides a method of treating a hyperproliferative disorder comprising administering a therapeutically effective amount of a compound of Formula (I), or a pharmaceutically acceptable salt thereof to a subject in need thereof. In some embodiments, said method relates to the treatment of a subject who suffers from a cancer such as acute myeloid leukemia, cancer in adolescents, adrenocortical carcinoma childhood, AIDS-related cancers (e.g., lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer; multiple endocrine neoplasia syndromes, multiple myeloma/plasma cell neoplasm, mycosis fungoides, myelodysplasia syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer; small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some embodiments, the methods for treatment are directed to treating lung cancers, and the methods comprise administering a therapeutically effective amount of the compounds of Formula (I) (or pharmaceutical composition comprising such compounds) to a subject in need thereof. In certain embodiments, the lung cancer is a non-small cell lung carcinoma (NSCLC), for example, adenocarcinoma, squamous-cell lung carcinoma or large-cell lung carcinoma. In some embodiments, the lung cancer is a small cell lung carcinoma. Other lung cancers which the compounds of Formula (I) may provide therapeutic benefit for include, but are not limited to, glandular tumors, carcinoid tumors and undifferentiated carcinomas.

The present disclosure also provides methods of modulating a mutant KRAS, HRAS or NRAS protein activity (e.g., activity resulting from the KRAS-G12C, KRAS-G12D and/or KRAS-G12V mutations) by contacting the protein with an effective amount of a compound of Formula (I). Modulation can be inhibiting or activating protein activity. In some embodiments, the present disclosure provides methods of inhibiting protein activity by contacting the mutant KRAS, HRAS or NRAS protein (e.g., KRAS-G12C, KRAS-G12D and/or KRAS-G12V mutants) with an effective amount of a compound of Formula (I) in solution. In some embodiments, the present disclosure provides methods of inhibiting the mutant KRAS, HRAS or NRAS protein activity by contacting a cell, tissue, or organ that expresses the protein of interest. In some embodiments, the disclosure provides methods of inhibiting protein activity in subjects including, but not limited to, rodents and mammals (e.g., humans) by administering into the subjects an effective amount of a compound of Formula (I).

### Combination Therapies

One or more additional pharmacologically active agents may be administered in combination with a compound of Formula (I) (or a pharmaceutically acceptable salt thereof). An additional active agent (or agents) is intended to mean a pharmaceutically active agent (or agents) that is active in the body, including pro-drugs that convert to pharmaceutically active form after administration, which are different from the compound of Formula (I). The additional active agents also include free-acid, free-base and pharmaceutically acceptable salts of said additional active agents. Generally, any suitable additional active agent or agents, including chemotherapeutic agents or therapeutic antibodies, may be used in any combination with the compound of Formula (I) in a single dosage formulation (e.g., a fixed dose drug combination), or in one or more separate dosage formulations which allows for concurrent or sequential administration of the active agents (co-administration of the separate active agents) to subjects. In addition, the compounds of Formula (I) (or pharmaceutically acceptable salts thereof) can be administered in combination with radiation therapy, hormone therapy, surgery or immunotherapy.

The present application also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal and anti-hormonal agents, targeted therapy agents, and anti-angiogenesis agents, to provide a synergistic or additive therapeutic effect. In another embodiment, such therapy includes radiation treatment to provide a synergistic or additive therapeutic effect.

Examples of additional active agents (i.e., additional anti-cancer agents) include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anti-cancer agents have been categorized within a specific group(s) or subgroup(s) herein, many of these agents can also be listed within one or more other group(s) or subgroup(s), as would be presently understood in the art. It is to be understood that the classification herein of a particular agent into a particular group is not intended to be limiting. Many anti-cancer agents are presently known in the art and can be used in combination with the compounds of the present disclosure.

Further, an agent can be an agonist, antagonist, allosteric modulator, toxin or, more generally, may act to inhibit or stimulate its target (e.g., receptor or enzyme activation or inhibition). For example, suitable for use are one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

In an embodiment, the additional anti-cancer agent is a chemotherapeutic agent, an immunotherapeutic agent, a hormonal agent, an anti-hormonal agent, a targeted therapy agent, or an anti-angiogenesis agent (or angiogenesis inhibitor). In an embodiment, the additional anti-cancer agent is selected from the group consisting of a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, a colony-stimulating factor, an immunomodulator, an immunomodulatory imide (IMiD), an anti-CTLA4 agent, an anti-LAG1 agent, an anti-OX40 agent, a GITR agonist, a CAR-T cell, a BiTE, a signal transduction inhibitor, a growth factor inhibitor, a tyrosine kinase inhibitor, an EGFR inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor, a HIF-2α inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a HER-2 inhibitor, a BRAF-inhibitor, a gene expression modulator, an autophagy inhibitor, an apoptosis inducer, an antiproliferative agent, and a glycolysis inhibitor.

In one embodiment, the additional anti-cancer agent(s) is a chemotherapeutic agent. Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

In one embodiment, the additional anti-cancer agent(s) is a hormonal and/or anti-hormonal agent (i.e., hormone therapy). Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4(5)-imidazoles, EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

In one embodiment, the additional anti-cancer agent(s) is an immunotherapeutic agent (i.e., immunotherapy). Non-limiting examples of immunotherapeutic agents include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, and trastuzumab.

Non-limiting examples of immune checkpoint inhibitors include anti-PD-1 agents or antibodies such as cemiplimab, nivolumab, and pembrolizumab; anti-PD-L1 agents or antibodies such as atezolizumab, avelumab, and durvalumab; anti-CTLA-4 agents or antibodies such as ipilumumab; anti-LAG1 agents; and anti-OX40 agents.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

In one embodiment, the additional anti-cancer agent(s) is a targeted therapy agent (i.e., targeted therapy). Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, MEK inhibitors, ERK inhibitors, PI3K inhibitors, AKT inhibitors, MCL-1 inhibitors, BCL-2 inhibitors, SHP2 inhibitors, HER-2 inhibitors, BRAF-inhibitors, gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR inhibitory agents.

Non-limiting examples of EGFR inhibitory agents include small molecule antagonists of EGFR such as afatinib, brigatinib, erlotinib, gefitinib, lapatinib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1: 1311-1318; Huang, S. M., et al., 1999, Cancer Res. 15:59(8): 1935-40; and Yang, X., et al., 1999, Cancer Res. 59: 1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

In one embodiment, the additional anti-cancer agent(s) is an anti-angiogenic agent (or angiogenesis inhibitor) including, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478; HIF-2α inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. Nos. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAP™.

The additional anti-cancer agent(s) may also be another anti-angiogenic agent including, but not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-111142, GCS-100, GW 2286 (GlaxoSmithKline, UK), IL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and MedImmune, US), MAb VEGF (Xenova, UK), marimastat, maspin (Sosei, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

In embodiments, the additional anti-cancer agent(s) is an additional active agent that disrupts or inhibits RAS-RAF-ERK or PI3K-AKT-TOR signaling pathways or is a PD-1 and/or PD-L1 antagonist. In embodiments, the additional anti-cancer agent(s) is a RAF inhibitor, EGFR inhibitor, MEK inhibitor, ERK inhibitor, PI3K inhibitor, AKT inhibitor, TOR inhibitor, MCL-1 inhibitor, BCL-2 inhibitor, SHP2 inhibitor, proteasome inhibitor, or immune therapy, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and an ERKi as described in WO 2017/068412.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; and ZSTK474.

Non-limiting examples of AKT inhibitors include Akt-1-1 (inhibits Akt1) (Barnett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 3493S-3498S); perifosine, Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1,2-c]pyrido[3,4-e][1,3]oxazin-2-yl)phenyl]-cyclobutanol hydrochloride (WO 2012/137870); afuresertib; capivasertib; MK2206; patasertib, and those disclosed in WO 2011/082270 and WO 2012/177844.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-[3-hydroxy(hydroxymethyl)methylpropanoate]-rapamycin; 40-epi-(tetrazolyl)-rapamycin (also called ABT578); 32-deoxorapamycin; 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and 563845.

Non-limiting examples of SHP2 inhibitors include SHP2 inhibitors described in WO 2019/167000 and WO 2020/022323.

Additional non-limiting examples of anti-cancer agents that are suitable for use include 2-ethylhydrazide, 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflomithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

The present disclosure further provides a method for using the compounds of Formula (I) or pharmaceutical compositions provided herein, in combination with radiation therapy to treat cancer. Techniques for administering radiation therapy are known in the art, and these techniques can be used in the combination therapy described herein. The administration of the compound of Formula (I) in this combination therapy can be determined as described herein.

Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The present disclosure also provides methods for combination therapies in which the additional active agent is known to modulate other pathways, or other components of the same pathway, or even overlapping sets of target enzymes which are used in combination with a compound of Formula (I), or a pharmaceutically acceptable salt thereof. In one embodiment, such therapy includes, but is not limited to, the combination of one or more compounds of Formula (I) with chemotherapeutic agents, immunotherapeutic agents, hormonal therapy agents, therapeutic antibodies, targeted therapy agents, and radiation treatment, to provide a synergistic or additive therapeutic effect.

The compounds of the disclosure can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula (I) and any of the agents described above can be formulated together in the same dosage form and administered simultaneously. Alternatively, a compound of Formula (I) and any of the agents described above can be simultaneously administered, wherein both the agents are present in separate formulations. In another alternative, a compound of Formula (I) can be administered just followed by and any of the agents described above, or vice versa. In some embodiments of the separate administration protocol, a compound of Formula (I) and any of the agents described above are administered a few minutes apart, or a few hours apart, or a few days apart.

As one aspect of the present disclosure contemplates the treatment of the disease/conditions with a combination of pharmaceutically active compounds that may be administered separately, the disclosure further relates to combining separate pharmaceutical compositions in kit form. The kit comprises two separate pharmaceutical compositions: a compound of Formula (I), and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The present disclosure also provides for the compound of Formula (I), or the pharmaceutically acceptable salt thereof, for use in therapy, or use of the compound of Formula (I), or the pharmaceutically acceptable salt thereof, in therapy. The present disclosure also provides for the compound of Formula (I), or the pharmaceutically acceptable salt thereof, for use in treating cancer, or use of a compound of Formula (I), or the pharmaceutically acceptable salt thereof, for treating cancer. The present disclosure also provides for the compound of Formula (I), or the pharmaceutically acceptable salt thereof, for the preparation of a medicament for the treatment of cancer, or use of the compound of Formula (I), or the pharmaceutically acceptable salt thereof, for the preparation of a medicament for the treatment of cancer. The present disclosure also provides for the compound of Formula (I), or the pharmaceutically acceptable salt thereof, and an additional anti-cancer agent, for use in the treatment of cancer, or use of the compound of Formula (I), or the pharmaceutically acceptable salt thereof, and the additional anti-cancer agent for treating cancer. The disclosure also provides the compound of Formula (I), or the pharmaceutically acceptable salt thereof, and an additional anti-cancer agent, for the preparation of a medicament for the treatment of cancer, or use of the compound of Formula (I), or the pharmaceutically acceptable salt thereof, and the additional anti-cancer agent, for the preparation of a medicament for the treatment of cancer. The present disclosure also provides for a pharmaceutical composition comprising the compound of Formula (I), or the pharmaceutically acceptable salt thereof, for use in the treatment of cancer, or use of the pharmaceutical composition comprising the compound of Formula (I), or the pharmaceutically acceptable salt thereof, for treating cancer. The present disclosure also provides for a pharmaceutical composition comprising the compound of Formula (I), or the pharmaceutically acceptable salt thereof, and an additional anti-cancer agent, for use in the treatment of cancer, or use of the pharmaceutical composition comprising the compound of Formula (I), or the pharmaceutically acceptable salt thereof, and the additional anti-cancer agent, for treating cancer.

### Methods of Preparing the Compounds of the Disclosure

The compounds described herein can be prepared according to the procedures of the following schemes and examples, using appropriate materials and are further exemplified by the following specific examples. The compounds illustrated in the examples are not, however, to be construed as forming the only genus that is considered as the disclosure. The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. These examples are provided for the purpose of further illustration only and are not intended to be limitations on the disclosure. Any intermediates described below may be referred to herein by their number preceded by “Int-.”

Throughout the synthetic schemes and examples, abbreviations and acronyms may be used with the following meanings unless otherwise indicated:
anhydr.=Anhydrous; aq.=aqueous; atm=atmosphere; Bodipy-GDP=mixture of ((2R,3S,4R,5R)-5-(2-amino-6-oxo-1,6-dihydro-9H-purin-9-yl)-3-(((2-(3-(5,5-difluoro-7,9-dimethyl-5H-4l4,5l4-d]pyrrolo[1,2-c:2′,1′-f][1,3,2]diazaborinin-3-yl)propanamido)ethyl)carbamoyl)oxy)-4-hydroxytetrahydrofuran-2-yl)methyl hydrogen diphosphate and ((2R,3R,4R,5R)-5-(2-amino-6-oxo-1,6-dihydro-9H-purin-9-yl)-4-(((2-(3-(5,5-difluoro-7,9-dimethyl-5H-4l4,5l4-d]pyrrolo[1,2-c:2′,1′-f][1,3,2]diazaborinin-3-yl)propanamido)ethyl)carbamoyl)oxy)-3-hydroxytetrahydrofuran-2-yl)methyl hydrogen diphosphate (Invitrogen™, catalog number G22360); Boc₂O=Di-tert-butyl dicarbonate; BOP=benzotriazol-1-yloxytris(dimethylamino)phosphonium hexafluorophosphate; B₂pin₂=Bis(pinacolato)diboron; br s=broad singlet; Bu=butyl; t-Bu=tert-butyl; cataCxium® C=trans-Di(μ-acetato)bis[o-(di-o-tolylphosphino)benzyl]dipalladium(II); CDCl₃=deuterated chloroform; Cbz=carbobenzyl; CDI=1,1′-carbonyldiimidazole; CELITE=diatomaceous earth; CF₃=trifluoromethyl; cGMP=cyclic guanosine monophosphate; CH₃NO₂=nitromethane; conc.=concentrated; DAST=(Diethylamino)sulfur trifluoride; DBU=1,8-diazabicyclo[5.4.0]undec-7-ene; DCM=dichloromethane; DCE=dichloroethane; DHP=3,4-dihydropyran; DIAD=Diisopropyl azodicarboxylate; DIEA/DIPEA=N,N-Diisopropylethylamine; DMAP=dimethylaminopyridine; DME=dimethoxyethane; DMEA=N,N-Dimethylethanamine; DMF=N,N-dimethylformamide; DMP=Dess-Martin periodinane; DMS=dimethylsulfide; DMSO=dimethylsulfoxide; DPPF or dppf=1,1′-bis(diphenylphosphino)ferrocene; EDTA=ethylenediaminetetraacetic acid; equiv, eq.=equivalent(s); Et=ethyl; Et₃N=triethylamine; EtOAc=ethyl acetate; EtOH=ethanol; Grubbs Catalyst=(1,3-Bis(2,4,6-trimethylphenyl)-2-imidazolidinylidene)dichloro(phenylmethylene)(tricyclohexylphosphine)ruthenium; GTP=guanosine triphosphate; h=hour; HATU=1-[bis(dimethylamino)methylene]-1H-1,2,3-triazolo[4,5-b]pyridinium 3-oxid hexafluorophosphate; HEPES=4-(2-hydroxyethyl)-1-piperazineethanesulfonic acid; HMDS=hexamethydisilazane; HPLC=High pressure liquid chromatography; Int=intermediate; iPr=isopropyl; IP=inflection points; i-PrOH=Isopropanol; KHMDS=Potassium bis(trimethylsilyl)amide; LAH=lithium aluminum hydride; LCMS, LC/MS=liquid chromatography-mass spectrometry; min, min.=minute; LDA=lithium diisopropylamide; M=Molar; m-CPBA or mCPBA=3-chlorobenzoperoxoic acid; Me=methyl; MeCN, ACN=acetonitrile; MeOH=methanol; mp, m.p.=melting point; mpk=milligrams per kilogram; MsCl=Methanesulfonyl chloride; MOM=methoxymethyl; MTBE=methyl tert-butyl ether; N=Normal; NaOMe=sodium methoxide; NaHMDS=sodium hexamethyldisilazide; NBS=N-bromosuccinimide; NCS=N-chlorosuccinimide; NMO=4-Methylmorpholine N-oxide; NMR=nuclear magnetic resonance; N.D.=not determined; PDA=photodiode array; Pet. ether=petroleum ether; Pd—C=palladium on carbon; Pd₂(dba)₃=tris(dibenzylideneacetone)dipalladium (0); Ph=phenyl; Pr=propyl; psi=pounds per square inch gauge; POCl₃=phosphorus(V) oxide chloride; PPTS=pyridinium p-toluenesulfonate; PTLC, prep TLC=preparative thin layer chromatography; pTsOH=p-toluenesulfonic acid; rac=racemic; RT=retention time; RP-HPLC=reverse phase HPLC; rt=room temperature; sat.=saturated; SFC=supercritical fluid chromatography; SOS=Son of Sevenless; TBAF=tetra-n-butylammonium fluoride; TBSCl=tert-butyldimethylsilyl chloride; TBDPS-Cl=tert-Butyl(chloro)diphenylsilane; TEA=triethylamine; TEMPO=2,2,6,6-Tetramethylpiperidine 1-oxyl; Tf2O=triflic anhydride; TFA=trifluoroacetic acid; TFAA=trifluoroacetic anhydride; THP=tetrahydropyran; TLC=thin layer chromatography; THF=tetrahydrofuran; TMEDA=N,N,N′,N′-Tetramethylethylenediamine; TMS=trimethylsilyl; TWEEN=polyoxyethylene (20) sorbitan monolaurate; VCD=vibrational circular dichroism; v, v/v=volume, volume to volume; w, w/w=weight, weight to weight, XPhos=2-dicyclohexylphosphino-2′,4′,6′-triisopropylbiphenyl; μm=micrometer.

### EXAMPLES

Concentration refers to the removal of the volatile components at reduced pressure (e.g., by rotary evaporation) unless otherwise noted. All temperatures are in degrees Celsius unless otherwise noted. Mass spectra (MS) were measured by electrospray ion-mass spectroscopy (ESI) in positive ion detection mode and m/z refers to the [M+H]⁺ ion unless otherwise noted. ¹H NMR spectra were recorded at 400-600 MHz at ambient temperature unless otherwise noted. Protons reported as 0.5H are due to rotameric signals. RP-HPLC refers to reverse-phase HPLC on C18-functionalized preparative or semi-preparative columns with gradient elution using acetonitrile and water modified with trifluoroacetic acid or ammonium hydroxide as eluents and fractions were lyophilized or concentrated by rotary evaporation unless otherwise noted. Purification by column chromatography on silica gel was accomplished using a flash chromatography system (e.g., ISCO® or Biotage®) and commercial pre-packed silica gel columns with elution using the stated solvent systems. Compounds described herein were synthesized as the racemates unless otherwise noted in the experimental procedures and compound tables. Certain products/intermediates in the examples include indication of “Peak 1” and/or “Peak 2”, which refer to the order of elution of the indicated product/intermediate from the chromatography column (e.g., an SFC column) used to isolate the compound under the specified conditions. Thus, for example, Peak 1 refers to the first eluting compound, e.g., first eluting stereoisomer, under the specified conditions.

SFC Columns used in the chiral resolution of stereoisomers are summarized in the following Table:

| | SFC Column |
|---|---|
| SFC Column | Abbreviation |
| Daicel Chiralpak IC (21 mm × 250 mm, 5 um) | Column A |
| AD-H (21 mm × 250 mm, 5 um) | Column B |
| Phenomenex, LUX-2 (21 mm × 250 mm, 5 um) | Column C |
| Phenomenex, Cellulose-2 (25 mm × 250 mm, 10 um) | Column D |
| Daicel Chiralpak AS (50 mm × 250 mm, 10 um) | Column E |
| Regis, (R, R) Whelk-O1 (50 mm × 250 mm, 10 um) | Column F |
| Daicel Chiralpak AD (30 mm × 250 mm, 10 um) | Column G |
| Daicel Chiralpak OJ-H (21 mm × 250 mm, 5 um) | Column H |
| Phenomenex, LUX-4 (21 mm × 250 mm, 5 um) | Column I |
| Daicel Chiralpak AS (50 mm × 250 mm, 10 um) | Column J |
| Daicel Chiralpak IG (30 mm × 250 mm, 10 um) | Column K |
| Chiral Technologies, IB-N (21 mm × 250 mm, 5 um) | Column L |
| Daicel Chiralpak IA (21 mm × 250 mm, 5 um) | Column M |
| ES Industries, CCAF4 (21 mm × 250 mm, 5 um) | Column N |
| Phenomenex, Cellulose-2 (30 mm × 250 mm, 5 um) | Column O |
| Daicel Chiralpak IG-3 (4.6 mm × 100 mm, 3 um) | Column P |
| CCA (21 mm × 250 mm, 5 um) | Column Q |
| CCO (21 mm × 250 mm, 5 um) | Column R |
| CCC (21 mm × 250 mm, 5 um) | Column S |
| Daicel Chiralpak IC (30 mm × 250 mm, 10 um) | Column T |
| Chiral SFC OD-H (21 mm × 250 mm × 5 μm) | Column U |
| SJ (21 mm × 250 mm × 5 μm) | Column V |
| Phenomenex-Cellulose-2 (30 mm × 250 mm, 10 um) | Column W |

### Intermediate Syntheses

### Intermediate 1: 2-methyl-8-azabicyclo[3.2.1]octan-2-ol (Int-1)

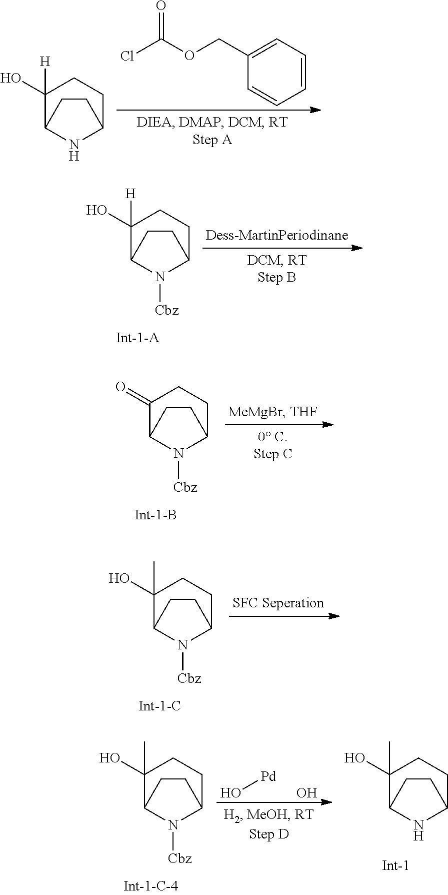

### Step A: benzyl 2-hydroxy-8-azabicyclo[3.2.1]octane-8-carboxylate (Int-1-A)

To a solution of 8-azabicyclo[3.2.1]octan-2-ol, HCl (600 mg, 3.67 mmol) in DCM (18 ml) was added N-ethyl-N-isopropylpropan-2-amine (948 mg, 7.33 mmol) followed by N,N-dimethylpyridin-4-amine (45 mg, 0.37 mmol). To this reaction mixture was added benzyl carbonochloridate (1.00 g, 5.87 mmol) dropwise. The reaction mixture was stirred at ambient temperature for 5 hrs. The mixture was diluted with DCM, washed with saturated sodium bicarbonate solution, saturated brine solution and concentrated under reduced pressure. The racemic mixture was purified by flash silica gel chromatography (eluting with methanol in dichloromethane, 0-25% gradient) to give the title compound. MS (ESI): m/z (M+H)⁺ 262.

### Step B: benzyl 2-oxo-8-azabicyclo[3.2.1]octane-8-carboxylate (Int-1-B)

To a solution of benzyl 2-hydroxy-8-azabicyclo[3.2.1]octane-8-carboxylate (Int-1-A) (800 mg, 3.06 mmol) in DCM (28 mL) was added Dess-Martin Periodinane (1.17 g, 2.76 mmol). The reaction mixture was stirred at ambient conditions for 5 h. The mixture was diluted with DCM, washed with saturated sodium bicarbonate solution, saturated brine solution and concentrated under reduced pressure. Product was purified by flash silica gel chromatography (eluting with ethyl acetate in hexane, 0-45% gradient) to give the title compound. MS (ESI): m/z (M+H)⁺ 260.

### Step C: benzyl 2-hydroxy-2-methyl-8-azabicyclo[3.2.1]octane-8-carboxylate (Int-1-C)

To a solution of benzyl 2-oxo-8-azabicyclo[3.2.1]octane-8-carboxylate (Int-1-B) (540 mg, 2.1 mmol) in THF (19 ml) under nitrogen was added methylmagnesium bromide (1.4 ml, 4.2 mmol, 3M in Et₂O) at 0° C. The mixture was slowly brought to ambient temperature and stirred for 45 mins. The reaction mixture was quenched with saturated ammonium chloride solution and extracted with ethyl acetate (2×10 mL). The combined extracts were washed with saturated brine solution and concentrated under reduced pressure. The product was purified by flash silica gel column chromatography (eluting with ethyl acetate in hexane, 0-40% gradient) to give the title compound as a mixture of stereoisomers. MS (ESI): m/z (M+H)⁺ 276. The mixture of stereoisomers was resolved by SFC (Column A; 15% MeOH with 0.1% NH₄OH). Fractions were concentrated under reduced pressure to give the title compounds Int-1-C-1, Int-1-C-2, Int-1-C-3, and Int-1-C-4 as isolated stereoisomers numbered by their order of elution.

### Step D: 2-methyl-8-azabicyclo[3.2.1]octan-2-ol (Int-1)

An 8 ml vial was charged with palladium hydroxide on carbon (19.18 mg, 0.068 mmol) and to this was added benzyl (1R,5S)-2-hydroxy-2-methyl-8-azabicyclo[3.2.1]octane-8-carboxylate (Int-1-C-4) in MeOH (3 ml) under nitrogen. The flask was evacuated and backfilled with nitrogen (3×). This was then evacuated and backfilled with a hydrogen balloon. Stirred at ambient temperature for 1.5 h, the reaction mixture was evacuated and backfilled with nitrogen. The reaction was filtered and washed with methanol. The filtrate was concentrated to give the title compound (Int-1). MS (ESI): m/z (M+H)⁺ 142.

### Intermediate 2: 3-(difluoromethyl)piperidin-3-ol (Int-2)

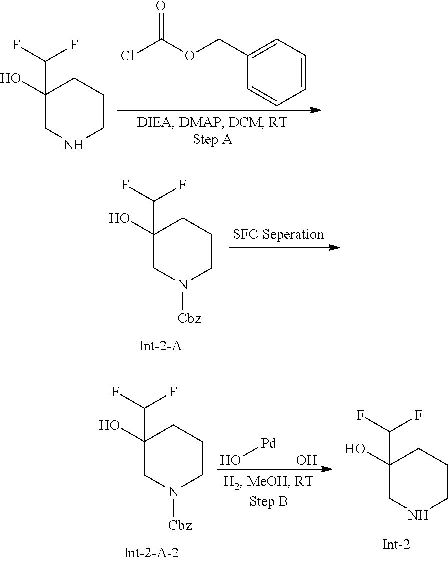

### Step A: benzyl 3-(difluoromethyl)-3-hydroxypiperidine-1-carboxylate (Int-2-A)

To a solution of 3-(difluoromethyl)piperidin-3-ol, HCl (1.5 g, 8.0 mmol) in DCM (30 ml) were added N-ethyl-N-isopropylpropan-2-amine (2.85 ml, 16.0 mmol) and N,N-diisopropylethylamine (0.098 g, 0.80 mmol). To this mixture was added benzyl carbonochloridate (2.28 ml, 16.0 mmol) dropwise. The mixture was stirred at ambient temperature overnight. The mixture was diluted with DCM, washed with saturated sodium bicarbonate solution, saturated brine solution and concentrated under reduced pressure. The racemic mixture was purified by flash silica gel chromatography (eluting with EtOAc in hexane, 0-25% gradient). The product was resolved by Chiral SFC Column B; 25% MeOH with 0.1% NH₄OH to give the title compound as isolated stereoisomers (Int-2-A-1, peak 1) MS (ESI): m/z (M+H)⁺ 286 and (Int-2-A-2, peak 2) MS (ESI): m/z (M+H)⁺ 286.

### Step B: 3-(difluoromethyl)piperidin-3-ol (Int-2)

An 8 ml vial was charged with palladium hydroxide on carbon (39.4 mg, 0.140 mmol) and to this was added benzyl 3-(difluoromethyl)-3-hydroxypiperidine-1-carboxylate (Int-2-A-2) (100 mg, 0.351 mmol) in MeOH (3 ml) under nitrogen. The flask was evacuated and backfilled with nitrogen (3×). This was then evacuated and backfilled with a hydrogen balloon. Stirred at ambient temperature for 3 h, the reaction mixture was evacuated and backfilled with nitrogen. The reaction was filtered and concentrated to give the title compound. ¹H NMR (499 MHz, DMSO-d₆) δ 5.79 (t, J=55.9 Hz, 1H), 5.07 (s, 1H), 2.73 (dd, J=12.8, 3.9 Hz, 1H), 2.64-2.54 (m, 2H), 2.44 (dd, J=15.8, 6.7 Hz, 1H), 1.59 (d, J=9.7 Hz, 1H), 1.57-1.50 (m, 2H), 1.38 (dd, J=11.6, 4.2 Hz, 1H).

### Intermediate 3: 4,4-difluoro-3-methylpiperidin-3-ol (Int-3)

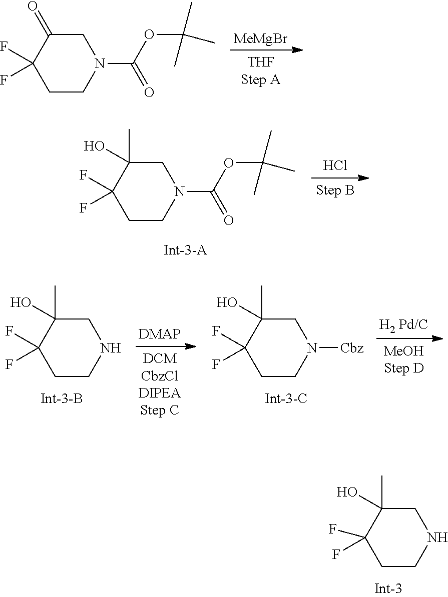

### Step A: tert-butyl 4,4-difluoro-3-hydroxy-3-methylpiperidine-1-carboxylate (Int-3-A)

Tert-butyl 4,4-difluoro-3-oxopiperidine-1-carboxylate (200 mg, 0.921 mmol), in THF (2367 μl) at 0° C. was treated with methylmagnesium bromide (1630 μl, 4.89 mmol, 3M in diethyl ether). The reaction mixture was allowed to warm to and stir at room temperature for 4 h, and the reaction mixture was carried forward to the next step.

### Step B: 4,4-difluoro-3-methylpiperidin-3-ol (Int-3-B)

HCl in dioxane (1183 μl, 4.73 mmol, 4M) was added (dropwise) to the reaction mixture from Step A at 0° C. The reaction mixture was allowed to warm to and stir at room temperature overnight. Sodium bicarbonate (sat. aq.) was used to neutralize. Product was extracted with DCM (3×20 mL) and concentrated under reduced pressure. The crude residue was used in the next step without purification.

### Step C: benzyl 4,4-difluoro-3-hydroxy-3-methylpiperidine-1-carboxylate (Int-3-C)

The crude mixture of 4,4-difluoro-3-methylpiperidin-3-ol obtained from Step B was dissolved in methylene chloride (2.4 mL) and treated with Hunig's Base (2000 μl, 11.45 mmol) and DMAP (11.57 mg, 0.095 mmol), followed by benzyl chloroformate (270 μl, 1.893 mmol) at 0° C. The reaction mixture was allowed to warm to and stir at room temperature for 2 h. The reaction mixture was concentrated in vacuo and re-dissolved in DMSO (3 mL). The product was isolated directly from the solution by Prep-HPLC (C18, 0-100% MeCN/water with 0.10% TFA modifier). Product containing fractions were concentrated under reduced pressure to give the title compound as a mixture of epimers. LCMS (ESI): m/z (M+H)⁺ 286.

### Step D: 4,4-difluoro-3-methylpiperidin-3-ol (Int-3)

Benzyl 4,4-difluoro-3-hydroxy-3-methylpiperidine-1-carboxylate (11 mg, 0.039 mmol) (Int-3-C) in MeOH (1000 μL) was added to a sealed vial of palladium on carbon (14 mg, 0.013 mmol, 10% wt) under argon. Hydrogen was introduced via balloon and an outlet needle added to allow hydrogen to effervesce below solvent level for 1 min. The hydrogen inlet was raised above solvent level and the outlet removed. The reaction was stirred for 1 h under hydrogen. The reaction mixture was filtered and concentrated under reduced pressure to give the title compound. LCMS (ESI): m/z (M+H)⁺ 152.

The compounds in the table below were synthesized using a similar procedure as described in the synthesis of Int-3 by making the appropriate substitutions for starting material, intermediates, and/or reagents optionally resolving stereoisomers after completion of the indicated step using the indicated conditions and carrying forward the indicated peak(s). Appropriate substitutions are available commercially, synthesized as described in the literature, synthesized using methods available to those skilled in the art, or synthesized as described herein.

| | | | | [M + H]⁺ |
|---|---|---|---|---|
| Int | Structure | Compound Name | Resolution | Found |
| Int-4 | 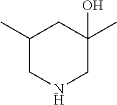 | 3,5-dimethylpiperidin-3- ol | Step C; SFC Column C, 25% MeOH with 0.1% NH₄OH; Overlapping Peaks 2/3 (of 4 total) were carried forward | 130 |
| Int-5 | 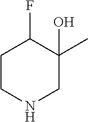 | 4-fluoro-3- methylpiperidin-3-ol | NA | 134 |
| Int-6 | 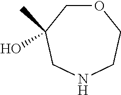 | (S)-6-methyl-1,4- oxazepan-6-ol | Step C; Column D; w/ 0.1% NH₄OH 25% MeOH; Peak 2 (of 2) | 132 |
| Int-7 | 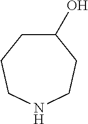 | azepan-4-ol | Step C; Column D; 25% MeOH with 0.1% NH₄OH; Peak 1 (of 2) | 116 |

### Intermediate 8: ((2R,7aS)-2-fluorotetrahydro-1H-pyrrolizin-7a(5H)-yl)methanol (Int-8)

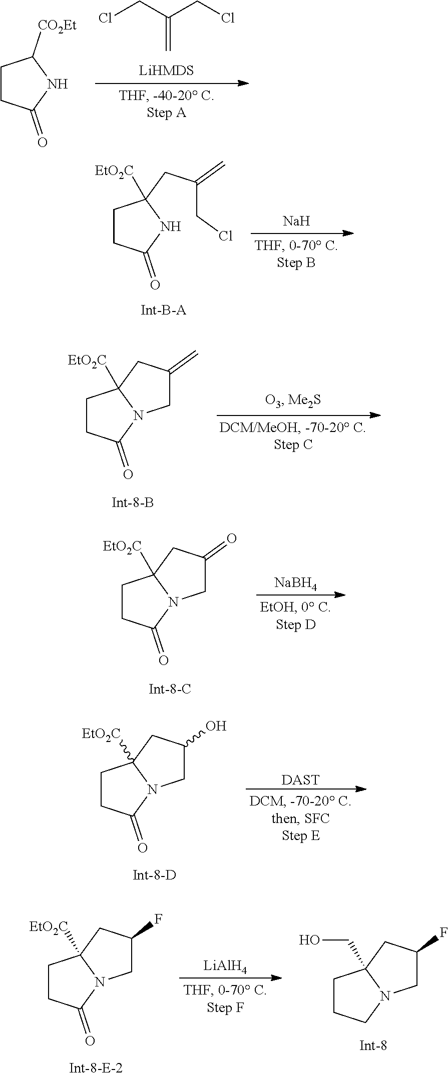

### Step A: ethyl 2-(2-(chloromethyl)allyl)-5-oxopyrrolidine-2-carbonylate (Int-8-A)

LiHMDS (1.00 M, 2.55 L) was added dropwise to a solution of ethyl 5-oxopyrrolidine-2-carboxylate (200. g, 1.27 mol) and 3-chloro-2-(chloromethyl)prop-1-ene (255 g, 2.04 mol, 236 mL) in THF (2.00 L) at −40° C. under N₂. The mixture was stirred at 20° C. for 20 h. The reaction mixture was poured into sat. NH₄Cl solution (1.00 L), and the pH of the mixture was adjusted to 6-7 with 1 N HCL. The biphasic solution was extracted with EtOAc (500 mL×3). The organic layers were combined, washed with brine (600 mL), and concentrated under reduced pressure to give a crude residue. The crude material was purified by silica gel column chromatography (eluent: petroleum ether:ethyl acetate=50:1 to 1:1 gradient) to yield ethyl 2-(2-(chloromethyl)allyl)-5-oxopyrrolidine-2-carboxylate (Int-8-A). ¹H NMR (400 MHz, CDCl₃) δ 5.06 (br d, J=17 Hz, 2H), 4.14-4.38 (m, 3H), 3.73 (br d, J=16 Hz, 1H), 3.06 (br d, J=16 Hz, 1H), 2.70-2.85 (m, 1H), 2.53-2.66 (m, 1H), 2.36-2.50 (m, 2H), 2.09-2.21 (m, 1H), 1.23-1.31 (m, 3H).

### Step B: ethyl 2-methylene-5-oxotetrahydro-1H-pyrrolizine-7a(5H)-carboxylate (Int-8-B)

A solution of ethyl 2-(2-(chloromethyl)allyl)-5-oxopyrrolidine-2-carboxylate (Int-8-A) (500. g, 2.03 mol) in THF (500 mL) was added dropwise to a mixture of sodium hydride (97.7 g, 2.44 mol, 60.0% wt %) in THF (3.00 L) at 0° C. under nitrogen. The reaction mixture was stirred at 70° C. for 12 h under nitrogen. The reaction mixture was cooled and poured into sat. ammonium chloride solution (2.00 L) and stirred at 5° C. for 1 h. The biphasic mixture was extracted with EtOAc (600 mL×3). The combined organic layers were washed with brine (500 mL×2), dried over anhydrous Na2SO4, filtered, and concentrated under reduced pressure. The crude residue was purified by silica gel column chromatography (eluent: Petroleum ether:Ethyl acetate=50:1 to 1:1) to yield ethyl 2-methylene-5-oxotetrahydro-1H-pyrrolizine-7a(5H)-carboxylate (Int-8-B). ¹H NMR (400 MHz, CDCl₃) δ 4.96-5.13 (m, 2H), 4.27 (br d, J=16 Hz, 1H), 4.19 (q, J=7 Hz, 2H), 3.71 (br d, J=16 Hz, 1H), 3.04 (d, J=16 Hz, 1H), 2.69-2.83 (m, 1H), 2.59 (ddd, J=2, 9, 13 Hz, 1H), 2.40-2.52 (m, 2H), 1.96-2.22 (m, 1H), 1.26 (t, J=7 Hz, 3H).

### Step C: ethyl 2,5-dioxotetrahydro-1H-pyrrolizine-7a(5H)-carboxylate (Int-8-C)

Ozone (239 mmol) (0.5-1 m³/h) was bubbled into a solution of ethyl 2-methylene-5-oxotetrahydro-1H-pyrrolizine-7a(5H)-carboxylate (Int-8-B) (160 g, 765 mmol in DCM (1.60 L) and MeOH (160 mL) at −70° C. for 9 h. Nitrogen was bubbled through the reaction mixture to purge excess ozone. Then, dimethyl sulfide (76.0 g, 1.22 mol) was added to the mixture at −70° C. The reaction mixture was stirred at 20° C. for 14 h. The reaction mixture was concentrated under reduced pressure to give a residue. The crude residue was purified by silica gel column chromatography (eluent: Petroleum ether:Ethyl acetate=50:1 to 1:1) to yield ethyl 2,5-dioxotetrahydro-1H-pyrrolizine-7a(5H)-carboxylate (Int-8-C). ¹H NMR (400 MHz, CDCl₃) δ 4.22 (q, J=7 Hz, 2H), 4.07-4.12 (m, 1H), 3.54 (dd, J=1, 18 Hz, 1H), 2.92-3.03 (m, 2H), 2.74-2.88 (m, 1H), 2.42-2.51 (m, 2H), 2.12-2.23 (m, 1H), 1.27 (t, J=7 Hz, 3H).

### Step D: ethyl 2-hydroxy-5-oxotetrahydro-1H-pyrrolizine-7a(5H)-carboxylate (Int-8-D)

To a solution of ethyl 2,5-dioxotetrahydro-1H-pyrrolizine-7a(5H)-carboxylate (Int-8-C) (200 g, 947 mmol) in EtOH (2.00 L) at 0° C. under N₂ was added NaBH₄ (10.8 g, 284 mmol). The reaction mixture was stirred at 0° C. for 10 min. The reaction mixture was quenched by addition of sat. NH₄Cl (50.0 mL) at 5° C., and the mixture was stirred at 5° C. for 0.5 h. The reaction mixture was concentrated under reduced pressure. The crude residue was purified by silica gel column chromatography (eluent: Petroleum ether:Ethyl acetate=50:1 to 1:1) to yield ethyl 2-hydroxy-5-oxotetrahydro-1H-pyrrolizine-7a(5H)-carboxylate (Int-8-D). ¹H NMR (400 MHz, CDCl₃) δ 4.54-4.70 (m, 1H), 4.16-4.31 (m, 2H), 3.93 (dd, J=6, 13 Hz, 1H), 3.09 (d, J=13 Hz, 1H), 2.75-2.90 (m, 1H), 2.39-2.63 (m, 4H), 2.01-2.13 (m, 1H), 1.83 (dd, J=6, 14 Hz, 1H), 1.29 (t, J=7 Hz, 3H).

### Step E: ethyl (2R,7aS)-2-fluoro-5-oxotetrahydro-1H-pyrrolizine-7a(5H)-carboxylate (Int-8-E-2)

To a solution of ethyl 2-hydroxy-5-oxotetrahydro-1H-pyrrolizine-7a(5H)-carboxylate (Int-8-D) (100 g, 468 mmol) in DCM (1L) was added DAST (113 g, 703 mmol, 93 mL) dropwise at −70° C. under N₂. The reaction mixture was warmed to 20° C. and stirred for 16 h. The reaction was quenched by the addition of EtOH (50.0 mL) at 10° C., and then diluted with water (300 mL) and extracted with DCM (200 mL×2). The combined organic layers were washed with brine (200 mL), dried over anhydrous Na₂SO₄, filtered, and concentrated under reduced pressure. The crude product was combined from six identical reactions and purified by silica gel column chromatography (Petroleum ether:Ethyl Acetate=50:1 to 1:1). This material was further purified by prep-HPLC (C18, 0-100% MeCN/water with 0.05% HCl). The racemic mixture was resolved using chiral SFC (Column E; EtOH with 0.1% NH₄OH) to yield ethyl (2R,7aS)-2-fluoro-5-oxotetrahydro-1H-pyrrolizine-7a(5H)-carboxylate (Int-8-E-2, Peak 2). ¹H NMR (400 MHz, CDCl₃): δ 5.16-5.43 (m, 1H), 4.14-4.27 (m, 3H), 3.06-3.26 (m, 1H), 2.57-2.85 (m, 3H), 2.38-2.50 (m, 1H), 2.07-2.30 (m, 2H), 1.28 (t, J=7 Hz, 3H).

### Step F: ((2R,7aS)-2-fluorotetrahydro-1H-pyrrolizin-7a(5H)-yl)methanol (Int-8)

A solution of ethyl (2R,7aS)-2-fluoro-5-oxotetrahydro-1H-pyrrolizine-7a(5H)-carboxylate (Int-8-E-2) (82.0 g, 381 mmol) in THF (300 mL) was added to the mixture of LAH (21.7 g, 571 mmol) in THF (520 mL) at 0° C. under nitrogen. The reaction mixture was warmed to 70° C. and stirred for 3 h. The reaction mixture was cooled to 0° C. and quenched by the addition of Na₂SO₄·10H₂O at 0° C. under nitrogen. The reaction mixture was stirred at 20° C. for 0.5 h and then filtered. The filter cake was washed with EtOAc (600 mL×5), and the filtrate was dried over anhydrous Mg₂SO₄. The mixture was filtered and the filtrate concentrated under reduced pressure to give a residue. The crude residue was purified by silica gel column chromatography (SiO₂, DCM:Methanol=100:1 to 10:1) to give the title compound. ¹H NMR (400 MHz, CDCl₃): δ 5.06-5.34 (m, 1H), 3.25 (s, 2H), 3.08-3.23 (m, 3H), 2.85-3.08 (m, 2H), 2.00-2.12 (m, 2H), 1.74-1.93 (m, 4H).

### Intermediate 9: ((3S,7aS)-3-(((tert-butyldiphenylsilyl)oxy)methyl)tetrahydro-1H-pyrrolizin-7a(5H)-yl)methanol (Int-9)

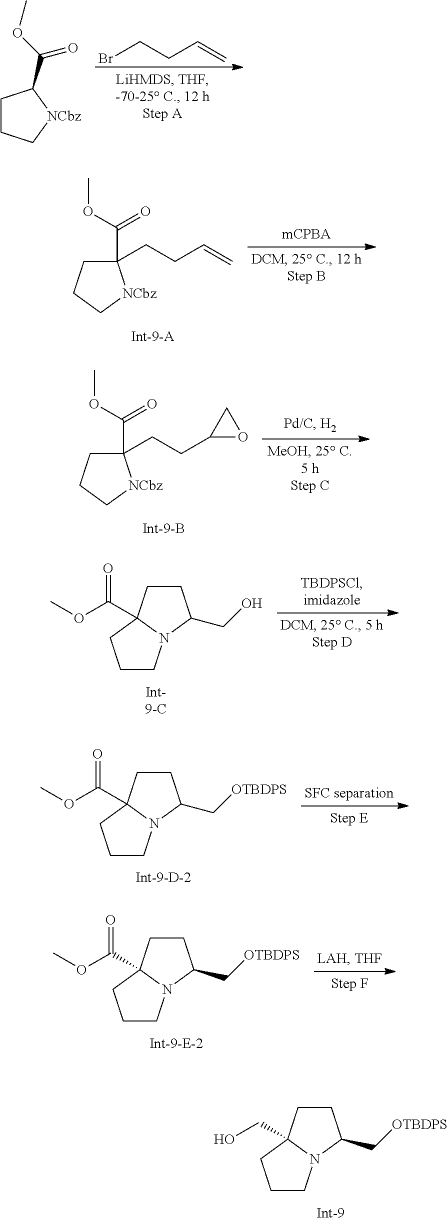

### Step A: 1-benzyl 2-methyl 2-(but-3-en-1-yl)pyrrolidine-1,2-dicarboxylate (Int-9-A)

To a stirred solution of 1-benzyl 2-methyl (S)-pyrrolidine-1,2-dicarboxylate (38 g, 144 mmol) in THF (200 mL) was added LiHMDS (216 mL, 216 mmol) at −78° C. under nitrogen, and the reaction was stirred at −78° C. for 1 h under N₂ atmosphere. Then 4-bromobut-1-ene (39.0 g, 289 mmol) was added at −78° C., and the reaction was stirred at 25° C. for 2 h under nitrogen atmosphere. The mixture was quenched with aq. sat. NH₄Cl (300 mL) and extracted with EtOAc (200 mL×3), and the organic layers were washed with NaCl (aq. sat., 200 mL) dried over Na₂SO₄ and concentrated under vacuum. The residue was purified by flash silica gel chromatography (eluting with ethyl acetate in hexane, 0-30% gradient) to give the title compound. MS (ESI) [M+H]⁺ m/z 318.

### Step B: 1-benzyl 2-methyl 2-(2-(oxiran-2-yl)ethyl)pyrrolidine-1,2-dicarboxylate (Int-9-B)

To a solution of 1-benzyl 2-methyl 2-(but-3-en-1-yl)pyrrolidine-1,2-dicarboxylate (Int-9-A) (27 g, 85 mmol) in DCM (500 mL) was added m-CPBA (45.9 g, 213 mmol) (80%), and the resulting mixture was stirred at 20° C. for 2 h. The reaction was filtered, and the filtrate was washed with Na₂SO₃ (aq. sat., 100 mL) and brine (aq. sat., 100 mL). The organic layer was dried over Na₂SO₄ and filtered, and the filtrate was purified by flash silica gel chromatography (eluting with ethyl acetate in hexanes, 0-20% gradient) to give the title compound. MS (ESI) m/z 334.

### Step C: methyl 3-(hydroxymethyl)tetrahydro-1H-pyrrolizine-7a(5H)-carboxylate (Int-9-C)

To a solution of 1-benzyl 2-methyl 2-(2-(oxiran-2-yl)ethyl)pyrrolidine-1,2-dicarboxylate (Int-9-B) (24 g, 72.0 mmol) in MeOH (500 mL) was added Pd—C (3.83 g, 3.60 mmol, 10% wt), the resulting mixture was stirred at 20° C. for 3 h. The reaction was filtered and washed with MeOH (200 mL), and the filtrate was concentrated under reduced pressure to afford the title compound, which was used directly in the next step. MS (ESI) [M+H]⁺: m/z 200.

### Step D: trans-methyl 3-(((tert-butyldiphenylsilyl)oxy)methyl)tetrahydro-1H-pyrrolizine-7a(5H)-carboxylate (Int-9-D-2)

To a solution of methyl 3-(hydroxymethyl)tetrahydro-1H-pyrrolizine-7a(5H)-carboxylate (Int-9-C) (14 g, 70.3 mmol) in DCM (30 mL) was added imidazole (9.57 g, 141 mmol) and TBDPS-Cl (21.7 mL, 84 mmol), and the resulting mixture was stirred at 20° C. for 1 h. The reaction was filtered and the filtrate was concentrated under vacuum. The residue was purified by flash silica gel chromatography (eluting with ethyl acetate in hexanes) to give the title compound (Int-9-D-2, the second eluting isomer, trans). MS (ESI) [M+H]⁺: m/z 438.

### Step E: methyl (3S,7aS)-3-(((tert-butyldiphenylsilyl)oxy)methyl)tetrahydro-1H-pyrrolizine-7a(5H)-carboxylate (Int-9-E-2)

The mixture of trans-methyl 3-(((tert-butyldiphenylsilyl)oxy)methyl)tetrahydro-1H-pyrrolizine-7a(5H)-carboxylate (Int-9-D-2) (11.5 g, 26.3 mmol) was separated by preparative SFC (Column F; 20% EtOH with 0.1% NH₄OH) to afford the title compound (Int-9-E-2, the second eluting isomer) MS (ESI) [M+H]⁺: m/z 438.

### Step F: ((3S,7aS)-3-(((tert-butyldiphenylsilyl)oxy)methyl)tetrahydro-1H-pyrrolizin-7a(5H)-yl)methanol (Int-9)

To a solution of methyl (3S,7aS)-3-(((tert-butyldiphenylsilyl)oxy)methyl)tetrahydro-1H-pyrrolizine-7a(5H)-carboxylate (4.2 g, 9.6 mmol, Int-9-E-2) in THF (60 mL) was added LiAlH₄ (0.546 g, 14.39 mmol) in portions at 0° C. The reaction mixture was stirred at 0° C. for 1 h. The reaction mixture was quenched with Na₂SO₄·10H₂O (10 g) and diluted with EtOAc (50 mL). The reaction mixture was filtered. The filtrate was concentrated under reduced pressure to give the residue which was purified by flash silica gel chromatography (eluting with Methanol in DCM, 10% isocratic) to give ((3S,7aS)-3-(((tert-butyldiphenylsilyl)oxy)methyl)tetrahydro-1H-pyrrolizin-7a(5H)-yl)methanol (Int-9). MS (ESI) [M+H]⁺ m/z: 410.

### Intermediate 10: (R)-(1-((dimethylamino)methyl)-2,2-difluorocyclopropyl)methanol hydrochloride (Int-10)

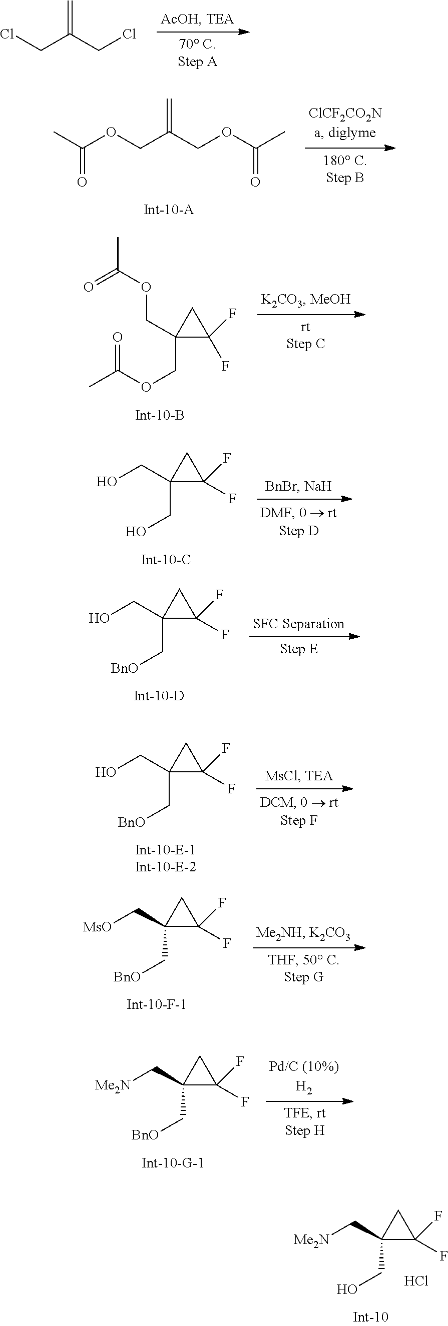

### Step A: 2-[(acetyloxy)methyl]prop-2-en-1-yl acetate (Int-10-A)

A 5 L 4-necked round-bottom flask was charged with 3-chloro-2-(chloromethyl)prop-1-ene (600. g, 4.80 mol), triethylamine (1.46 kg, 14.4 mol), and acetic acid (721 g, 12.0 mol). The resulting solution was stirred overnight at 70° C. The reaction mixture was cooled to room temperature and quenched by the addition of 3 L of water. The resulting solution was extracted with ethyl acetate (3×1 L), and the combined organic layers were washed with brine solution (2×1 L). The organic layers were dried over anhydrous sodium sulfate. The dried solution was filtered, and the filtrate was concentrated. The residue was purified by silica gel column with ethyl acetate/petroleum ether (1:6) to provide 2-[(acetyloxy)methyl]prop-2-en-1-yl acetate (Int-10-A).

### Step B: [1-[(acetyloxy)methyl]-2,2-difluorocyclopropyl]methyl acetate (Int-10-B)

Into a 20-L 4-necked round-bottom flask and maintained with an inert atmosphere of nitrogen was placed a solution of 2-[(acetyloxy)methyl]prop-2-en-1-yl acetate (Int-10-A) (600. g, 3.48 mol) in diglyme (5 L). This was followed by the addition of a solution of ClCF₂CO₂Na (2.65 kg, 17.4 mol) in diglyme (5 L) dropwise with stirring at 180° C. over 5 h.

The resulting solution was stirred for 1 h at 180° C. The reaction mixture was cooled to room temperature and quenched by the addition of H₂O (5 L). The resulting solution was extracted with petroleum ether (4×2 L), and the organic layers were combined. The combined organic layers were washed with water (3×2 L) and dried over anhydrous sodium sulfate. The dried solution was filtered, and the filtrate was concentrated to dryness to afford [1-[(acetyloxy)methyl]-2,2-difluorocyclopropyl]methyl acetate (Int-10-B), which was used directly in the next step without purification.

### Step C: [2,2-difluoro-1-(hydroxymethyl)cyclopropyl]methanol (Int-10-C)

Into a 20-L 4-necked round-bottom flask were placed [1-[(acetyloxy)methyl]-2,2-difluorocyclopropyl]methyl acetate (Int-10-B) (800 g, 3.60 mol), MeOH (10 L) and K₂CO₃ (995 g, 7.20 mol). The resulting solution was stirred overnight at room temperature. The solids were filtered out. The filtrate was concentrated. The resulting mixture was then diluted by the addition of water (2 L). The resulting solution was extracted with ethyl acetate (5×1 L). The organic layers were combined and dried over anhydrous sodium sulfate. The dried solution was filtered, and the filtrate was concentrated under vacuum. The residue was purified by silica gel column chromatography with ethyl acetate/petroleum ether (1:1) to afford [2,2-difluoro-1-(hydroxymethyl)cyclopropyl]methanol (Int-10-C). ¹H NMR (300 MHz, DMSO-d₆) δ 1.30 (t, J=9 Hz, 2H), 3.52 (m, 4H), 4.79 (t, J=5.6 Hz, 2H).

### Step D: (1-((benzyloxy)methyl)-2,2-difluorocyclopropyl)methanol (Int-10-D)

A 500 mL single neck round bottom flask fitted with a pour-through nitrogen adapter was purged with nitrogen and then charged with sodium hydride (4.52 g, 113 mmol) and N,N-dimethylformamide (100 mL). The suspension was cooled to 0° C. Solid [2,2-difluoro-1-(hydroxymethyl)cyclopropyl]methanol (Int-10-C) (12.0 g, 87 mmol) was added portion wise. The mixture was stirred while warming to rt for 1 h. The resultant reaction mixture was cooled to 0° C. and treated with a solution of benzyl bromide (10.3 mL, 87 mmol) in N,N-dimethylformamide (10 mL). The mixture was stirred at rt for 1 h and then treated with saturated aqueous ammonium chloride (10 mL) and water (10 mL). The mixture was partitioned between ethyl acetate (75 mL) and water (75 mL). The organic layer was washed with 1 wt % aqueous LiCl (30 mL×3), dried with anhydrous sodium sulfate, filtered and the filtrate was concentrated. Purification by column chromatography on silica gel (EtOAc in hexanes, 0-40% gradient) afforded (1-((benzyloxy)methyl)-2,2-difluorocyclopropyl)methanol (Int-10-D). 1H NMR (499 MHz, Methanol-d4) δ 7.39-7.32 (m, 4H), 7.32-7.26 (m, 1H), 4.62-4.49 (m, 2H), 3.79-3.64 (m, 3H), 3.60 (dd, J=10.4, 2.1 Hz, 1H), 1.35 (dddd, J=29.1, 12.5, 8.0, 4.5 Hz, 2H).

### Step E: (R)-(1-((benzyloxy)methyl)-2,2-difluorocyclopropyl)methanol (Int-10-E-1) and (S)-(1-((benzyloxy)methyl)-2,2-difluorocyclopropyl)methanol (Int-10-E-2)

Racemic (1-((benzyloxy)methyl)-2,2-difluorocyclopropyl)methanol (Int-10-D) was resolved using SFC chiral chromatography (Column B; 5% MeOH w/0.10% NH₄OH and 5% H₂O) to yield (R)-(1-((benzyloxy)methyl)-2,2-difluorocyclopropyl)methanol (Int-10-E-1, Peak 1) and (S)-(1-((benzyloxy)methyl)-2,2-difluorocyclopropyl)methanol (Int-10-E-2, Peak 2). (Int-10-E-1, Peak 1)¹H NMR (499 MHz, Methanol-d4) δ 7.39-7.26 (m, 5H), 4.58-4.51 (m, 2H), 3.78-3.67 (m, 3H), 3.60 (dd, J=10.4, 2.0 Hz, 1H), 1.35 (dddd, J=28.4, 12.5, 8.0, 4.5 Hz, 2H). (Int-10-E-2 Peak 2)¹H NMR (400 MHz, CDCl₃) δ 7.42-7.29 (m, 5H), 4.63-4.51 (m, 2H), 3.87-3.76 (m, 2H), 3.69 (d, J=2.0 Hz, 2H), 2.40 (t, J=6.1 Hz, 1H), 1.39 (ddd, J=4.5, 8.0, 12.5 Hz, 1H), 1.28 (ddd, J=4.3, 7.9, 12.4 Hz, 1H).

### Step F: (S)-(1-((benzyloxy)methyl)-2,2-difluorocyclopropyl)methyl methanesulfonate (Int-10-F-1)

(R)-(1-((benzyloxy)methyl)-2,2-difluorocyclopropyl)methanol (Int-10-E-1) (3.25 g, 14.24 mmol) in DCM (30 mL) was cooled to 0° C. and treated with triethylamine (7.94 mL, 57.0 mmol) and then a solution of methanesulfonyl chloride (2.22 mL, 28.5 mmol) in DCM (2.2 mL). The reaction mixture was stirred while warming to rt over 3 h. The reaction mixture was purified by column chromatography on silica gel (eluting with ethyl acetate in hexanes, 0-100% gradient) to afford (S)-(1-((benzyloxy)methyl)-2,2-difluorocyclopropyl)methyl methanesulfonate (Int-10-F-1). MS (ESI): m/z (M+Na)+329.

### Step G: (R)-1-(1-((benzyloxy)methyl)-2,2-difluorocyclopropyl)-N,N-dimethylmethanamine (Int-10-G-1)

(S)-(1-((benzyloxy)methyl)-2,2-difluorocyclopropyl)methyl methanesulfonate (Int-10-F-1) (4.10 g, 13.4 mmol) and dimethylamine (2 M in THF) (33.5 mL, 66.9 mmol) were treated with potassium carbonate (3.70 g, 26.8 mmol). The flask was capped and heated at 50° C. for 24 h. The reaction mixture was cooled to rt, diluted with water (20 mL), and extracted with ethyl acetate. The combined organic layers were dried with anhydrous sodium sulfate, filtered, and the filtrate was concentrated. The crude residue was purified by column chromatography on silica gel (eluting with (1:3 EtOH/EtOAc) in hexanes, 0-100% gradient) to afford (R)-1-(1-((benzyloxy)methyl)-2,2-difluorocyclopropyl)-N,N-dimethylmethanamine (Int-10-G-1). MS (ESI): m/z (M+H)⁺ 256.

### Step H: (R)-(1-((dimethylamino)methyl)-2,2-difluorocyclopropyl)methanol hydrochloride (Int-10)

(R)-1-(1-((benzyloxy)methyl)-2,2-difluorocyclopropyl)-N,N-dimethylmethanamine (Int-10-G-1) (2.96 g, 11.6 mmol) and Pd/C (10 wt %, wet support) (0.618 g, 0.580 mmol) in 2,2,2-trifluoroethanol (20.0 mL) were charged in a 100 mL recovery flask and stirred at rt under hydrogen gas (1 atm, balloon) for 20 h. The mixture was filtered through a pad of Celite™, and the pad was rinsed with methanol (3×10 mL). The combined filtrate and washings were treated with 3M HCl in methanol (12 mL, 36.0 mmol) and then concentrated under reduced pressure to afford a clear, colorless viscous syrup. Diethyl ether (10 mL) was added and the mixture agitated to initiate precipitation. The mixture was concentrated in vacuo and then treated with diethyl ether (10 mL) and sonicated for 1 minute. The diethyl ether was decanted. The solid was dried under vacuum to afford the title compound (Int-10). MS (ESI): m/z (M+H)⁺ 166.

### Intermediate 11: 2,4-dichloro-6,8-difluoroquinazoline (Int-11)

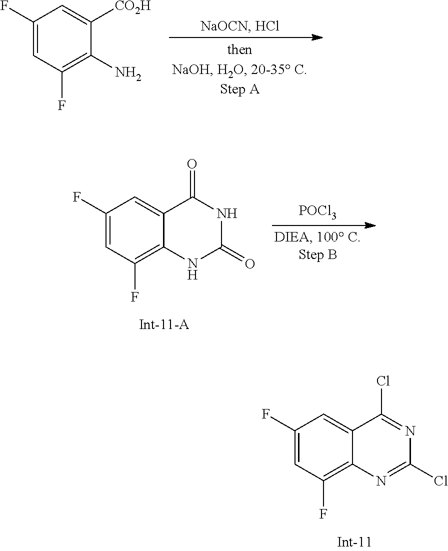

### Step A: 6,8-difluoroquinazoline-2,4(1H,3H)-dione (Int-11-A)

Sodium hydroxide (67.9 g, 1.70 mol) was dissolved in H₂O (2.10 L) in a 5 L three-necked round bottom flask at 20° C. To this mixture was added 2-amino-3,5-difluorobenzoic acid (210 g, 1.21 mol) at 20° C. The resulting mixture was stirred at 35° C. for 1 h. To this mixture was added sodium isocyanate (173 g, 2.67 mol) at 35° C., and the mixture was stirred until the solid dissolved completely. The resulting mixture was stirred at 35° C. for 1 h. To this mixture was added HCl (6 M, 1.01 L, 6.06 mol) dropwise at 30-35° C. for 1 h, and the pH was maintained between 6 and 7. Then NaOH (146 g, 3.64 mol) was added at 25° C. for 1 h. The resulting mixture was stirred at 25° C. for 9 h. To this mixture was added HCl (12 N) dropwise at 20-25° C., and the pH was maintained at ~1 to 2. Acetone (1.2 L) was added into the mixture, and the mixture was stirred at 25° C. for 1 h. The precipitate was removed by filtration, and the filter cake was washed with acetone (500 mL). To the filtrate was added methanol (1.2 L), and the mixture was stirred at 25° C. for 1 h. The product was obtained by collection of the precipitate to afford 6,8-difluoroquinazoline-2,4(1H,3H)-dione (Int-11-A). ¹H NMR (400 MHz DMSO-d₆) δ 11.54 (s, 1H), 11.34 (s, 1H), 7.71-7.72 (m, 1H), 7.46-7.49 (m, 1H).

### Step B: 2,4-dichloro-6,8-difluoroquinazoline (Int-11)

A 5 L three-necked round bottom flask was charged with 6,8-difluoroquinazoline-2,4(1H,3H)-dione (Int-11-A) (166 g, 838 mmol) and POCl₃ (1.03 kg, 6.70 mol) at 20° C. The resulting mixture was cooled to 10° C. and DIPEA (324 g, 2.51 mol) was added. The reaction mixture was stirred at 100° C. for 3 h. The reaction mixture was evaporated under reduced pressure to dryness. The residue was dissolved in EtOAc (1 L) and washed with sat. NaHCO₃ (1 L). The organic layer was washed with brine (300 mL×2), dried with Na₂SO₄ and filtered. The filtrate was concentrated in vacuo to dryness. The residue was triturated with MTBE (300 mL) at 25° C. for 30 min. The mixture was filtered, and the solid was collected to afford 2,4-dichloro-6,8-difluoroquinazoline (Int-11). ¹H NMR (400 MHz DMSO-d₆) δ 8.24-8.29 (m, 1H), 7.97-7.99 (m, 1H).

### Intermediate 12: 4-bromo-6-methyl-1-(tetrahydro-2H-pyran-2-yl)-5-(trifluoromethyl)-1H-indazole (Int-12)

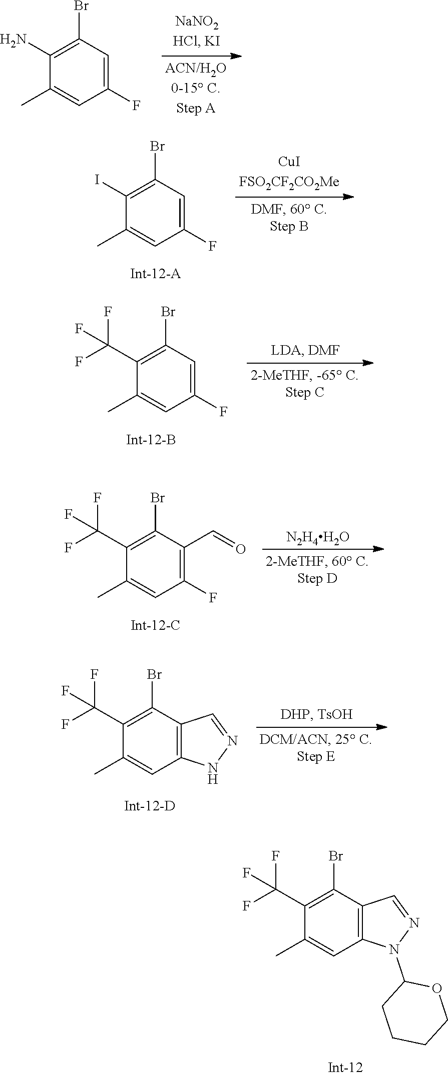

### Step A: 1-bromo-5-fluoro-2-iodo-3-methylbenzene (Int-12-A)

2-Bromo-4-fluoro-6-methylaniline (200. g, 0.983 mol) was dissolved in MeCN (800 mL). The resulting mixture was cooled down to 0° C. Concentrated HCl (12 M, 245 mL) was added into the reaction mixture while maintaining the reaction temperature at 0° C. A solution of NaNO₂ (81.1 g, 1.18 mol eq) in water (400 mL) was added dropwise into the reaction mixture, maintaining the reaction temperature at 0° C. The resulting mixture was stirred for 0.5 h at 0° C. Then a solution of KI (195 g, 1.18 mol) in water (400 mL) was added dropwise into the reaction mixture at 0° C. The resulting mixture was warmed up to room temperature and stirred for 12 h at 20° C. The product mixture was adjusted to pH 8-9 by aq. NaOH, and the aqueous phase was extracted with EtOAc (2.00 L x 2). The organic phase was dried over Na₂SO₄, filtered, and concentrated. The residue was purified by silica gel column chromatography (EtOAc in Hexane, 0-100% gradient) to afford 1-bromo-5-fluoro-2-iodo-3-methylbenzene (Int-12-A). ¹H NMR (400 MHz, CDCl₃) δ 7.27-7.22 (m, 1H), 6.95 (dd, J=2.4, 8.8 Hz, 1H), 2.56 (s, 3H).

### Step B: 1-bromo-5-fluoro-3-methyl-2-(trifluoromethyl)benzene (Int-12-B)

1-Bromo-5-fluoro-2-iodo-3-methylbenzene (Int-12-A) (100 g, 0.317 mol) was dissolved in DMF (1.50 L). To this mixture were added CuI (514 g, 2.70 mol) and methyl 2,2-difluoro-2-(fluorosulfonyl)acetate (518 g, 2.70 mol) at 25° C. The reaction mixture was heated and stirred for 12 h at 60° C. This reaction was repeated in 3 additional batches using the above conditions. The four batches of reactions were combined and quenched with water (24.0 L). The mixture was extracted with petroleum ether (8.00 L×2). The combined organic layers were washed with brine (4.00 L×2) and dried over Na₂SO₄. The dried solution was filtered, and the filtrate was concentrated in vacuo to afford the crude material containing 1-bromo-5-fluoro-3-methyl-2-(trifluoromethyl)benzene (Int-12-B).

### Step C: 2-bromo-6-fluoro-4-methyl-3-(trifluoromethyl)benzaldehyde (Int-12-C)

1-Bromo-5-fluoro-3-methyl-2-(trifluoromethyl)benzene (Int-12-B) (100 g, 0.382 mol) was dissolved in 2-MeTHF (500 mL). The reaction mixture was cooled down to −65° C. A 2 M solution of LDA (213 mL, 426 mmol) was added into the mixture at −65° C. The reaction mixture was stirred for 0.5 h at −65° C. To this mixture was added dropwise DMF (31.2 g, 0.420 mol) at −65° C. The reaction mixture was stirred for 2 h at −65° C. The reaction mixture pH was adjusted to 3-4 by using 1 M HCl, and the aqueous phase was extracted with 2-MeTHF (500 mL×2). The organic phase was dried over Na₂SO₄, filtered, and concentrated to obtain 2-bromo-6-fluoro-4-methyl-3-(trifluoromethyl)benzaldehyde (Int-12-C).

### Step D: 4-bromo-6-methyl-5-(trifluoromethyl)-1H-indazole (Int-12-D)

2-Bromo-6-fluoro-4-methyl-3-(trifluoromethyl)benzaldehyde (Int-12-C) (100 g, 0.351 mol) was dissolved in THF (800 mL). To this mixture was added N₂H₄·H₂O (53.7 g, 1.05 mol) at 25° C. The mixture was heated and stirred for 2 h at 60° C. The product mixture was quenched with water (400 mL) and extracted with EtOAc (200 mL×2). The combined organic layers were washed with brine (200 mL) and dried over Na₂SO₄. The dried solution was filtered, and the filtrate was concentrated in vacuo to give the residue. The residue obtained was triturated with DCM (100 mL) at 15° C. for 2 h. The solid was collected by filtration to afford 4-bromo-6-methyl-5-(trifluoromethyl)-1H-indazole (Int-12-D). ¹H NMR (400 MHz, CDCl₃) δ 10.61-10.20 (m, 1H), 8.20 (d, J=0.8 Hz, 1H), 7.34 (d, J=0.6 Hz, 1H), 2.67-2.63 (m, 3H).

### Step E: 4-bromo-6-methyl-1-(tetrahydro-2H-pyran-2-yl)-5-(trifluoromethyl)-1H-indazole (Int-12)

4-Bromo-6-methyl-5-(trifluoromethyl)-1H-indazole (Int-12-D) (60.0 g, 0.215 mol) was dissolved in DCM (240 mL) and MeCN (240 mL). DHP (21.7 g, 0.258 mol) and TsOH·H₂O (8.18 g, 0.043 mol) were added to the mixture at 20° C. The reaction mixture was stirred for 12 h at 20° C. Water (200 mL) was added to the product mixture. The resulting mixture was extracted with DCM (200 mL×2). The combined organic layers were washed with brine (200 mL) and dried over Na₂SO₄. The dried solution was filtered, and the filtrate was concentrated under reduced pressure. The residue obtained was purified by column chromatography (SiO₂, Petroleum ether:Ethyl acetate=1:0 to 0:1) to afford 4-bromo-6-methyl-1-(tetrahydro-2H-pyran-2-yl)-5-(trifluoromethyl)-1H-indazole (Int-12). ¹H NMR (400 MHz, CDCl₃-d) δ 8.12 (s, 1H), 7.44 (s, 1H), 5.69 (dd, J=3, 9 Hz, 1H), 4.09-3.94 (m, 1H), 3.81-3.69 (m, 1H), 2.69-2.63 (m, 3H), 2.56-2.43 (m, 1H), 2.19-2.14 (m, 1H), 2.12-2.04 (m, 1H), 1.87-1.73 (m, 2H), 1.71-1.63 (m, 1H).

### Intermediate 13: ((2-fluoro-8-(4,4,5,5-tetramethyl-1,3,2-dioxaborolan-2-yl)naphthalen-1-yl)ethynyl)triisopropylsilane (Int-13)

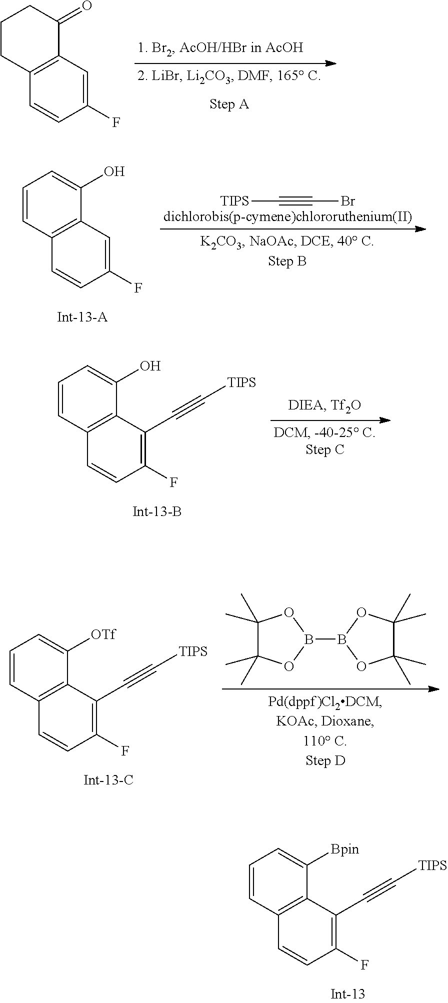

### Step A: 7-fluoronaphthalen-1-ol (Int-13-A)

To a stirred solution of 7-fluoro-3,4-dihydronaphthalen-1(2H)-one (5.00 g, 30.5 mmol) in AcOH (100 mL) and HBr (0.375 mL, 2.279 mmol, 33% in AcOH) was added Br₂ (1.726 mL, 33.5 mmol) in AcOH (3 mL) at 0° C., and the mixture was stirred at 25° C. for 3 h. On completion, the reaction mixture was diluted with DCM (300 mL) and washed with water (30 mL×3). The combined organic phase was dried over anhydrous Na₂SO₄, filtered and concentrated under reduced pressure. The residue was dissolved in DMF (100 mL), and lithium carbonate (3.83 g, 51.8 mmol) and lithium bromide (4.50 g, 51.8 mmol) were added to the solvent. The resulting mixture was stirred at 165° C. for 3 h. The reaction mixture was diluted with EtOAc (300 mL) and washed with brine (aq. sat., 50 mL×3). The combined organic phase was dried over anhydrous Na₂SO₄, filtered and concentrated in vacuo. The residue was purified by flash silica gel chromatography (eluting with ethyl acetate in hexanes, 0-100% gradient) to give 7-fluoronaphthalen-1-ol (Int-13-A). ¹H NMR (400 MHz, CDCl₃) δ 7.86-7.71 (m, 2H), 7.42 (d, J=8.3 Hz, 1H), 7.26-7.22 (m, 2H), 6.82 (d, J=7.6 Hz, 1H).

### Step B: 7-fluoro-8-((triisopropylsilyl)ethynyl)naphthalen-1-ol (Int-13-B)

To a solution of 7-fluoronaphthalen-1-ol (Int-13-A) (7.00 g, 43 mmol) in DCE (70 mL) was added (bromoethynyl)triisopropylsilane (13.5 g, 51.8 mmol), potassium carbonate (5.97 g, 43.2 mmol), sodium acetate (0.708 g, 8.63 mmol) and dichloro(p-cymene)ruthenium(II) dimer (3.97 g, 6.47 mmol) at 25° C. under N₂ atmosphere, and the mixture was stirred at 40° C. for 16 h. The reaction mixture was diluted with DCM (100 mL), and the resulting mixture was filtered. The filtrate was concentrated in vacuo, and the residue was purified by flash silica gel chromatography (eluting with ethyl acetate in hexanes, 0-100% gradient) to give 7-fluoro-8-((triisopropylsilyl)ethynyl)naphthalen-1-ol (Int-13-B). ¹H NMR (400 MHz, CDCl₃) δ 9.07 (s, 1H), 7.76 (dd, J=5.9, 9.0 Hz, 1H), 7.39-7.28 (m, 2H), 7.20 (t, J=8.8 Hz, 1H), 7.07-6.96 (m, 1H), 1.20-1.13 (m, 21H).

### Step C: 7-fluoro-8-((triisopropylsilyl)ethynyl)naphthalen-1-yl trifluoromethanesulfonate (Int-13-C)

To a solution of 7-fluoro-8-((triisopropylsilyl)ethynyl)naphthalen-1-ol (Int-13-B) (10.6 g, 30.9 mmol) in DCM (100 mL) was added DIEA (16.2 mL, 93 mmol) and Tf2O (7.84 mL, 46.4 mmol) at −40° C., and the mixture was stirred at 25° C. for 1 h. The reaction mixture was concentrated in vacuo, and the residue was purified by flash silica gel chromatography (eluting with ethyl acetate in hexanes, 0-100% gradient) to give 7-fluoro-8-((triisopropylsilyl)ethynyl)naphthalen-1-yl trifluoromethanesulfonate (Int-13-C). ¹H NMR (400 MHz, CDCl₃) δ 7.96-7.83 (m, 2H), 7.58 (br d, J=7.6 Hz, 1H), 7.54-7.45 (m, 1H), 7.45-7.35 (m, 1H), 1.31-1.16 (m, 21H).

### Step D: ((2-fluoro-8-(4,4,5,5-tetramethyl-1,3,2-dioxaborolan-2-yl)naphthalen-1-yl)ethynyl)triisopropylsilane (Int-13)

To a solution of 7-fluoro-8-((triisopropylsilyl)ethynyl)naphthalen-1-yl trifluoromethanesulfonate (Int-13-C) (4.6 g, 9.69 mmol) and bis(pinacolato)diboron (3.69 g, 14.5 mmol) in dioxane (50 mL) was added potassium acetate (1.903 g, 19.4 mmol) and PdCl₂(dppf)-CH₂Cl₂ Adduct (0.792 g, 0.969 mmol) at 25° C., and the solution was stirred at 110° C. for 15 hours under N₂ atmosphere. The reaction mixture was diluted with EtOAc (50 mL), and the resulting mixture was filtered. The filtrate was concentrated in vacuo, and the residue was purified by flash silica gel chromatography (eluting with ethyl acetate in hexanes 0-100% gradient) to give ((2-fluoro-8-(4,4,5,5-tetramethyl-1,3,2-dioxaborolan-2-yl)naphthalen-1-yl)ethynyl)triisopropylsilane (Int-13). ¹H NMR (400 MHz, CDCl₃) δ 7.84-7.72 (m, 3H), 7.40 (t, J=7.5 Hz, 1H), 7.26-7.20 (m, 1H), 1.42 (s, 12H), 1.15 (s, 21H).

### Intermediate 14: 2-(8-chloro-7-fluoronaphthalen-1-yl)-4,4,5,5-tetramethyl-1,3,2-dioxaborolane (Int-14)

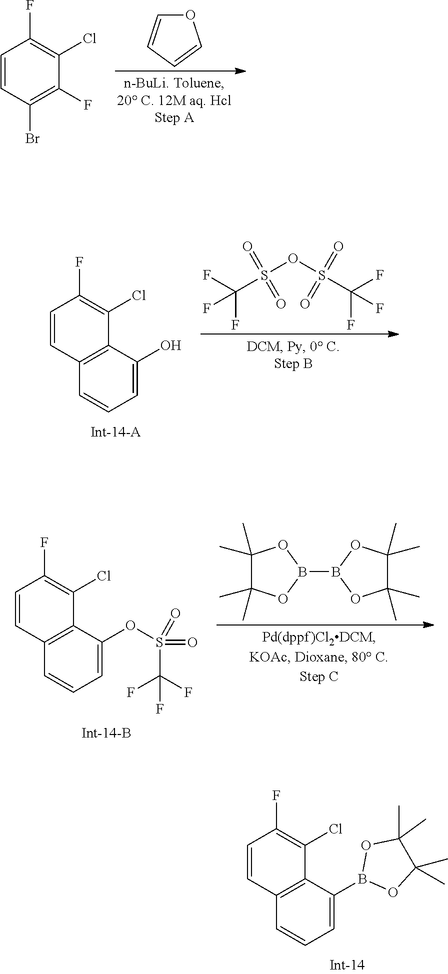

### Step A: 8-chloro-7-fluoronaphthalen-1-ol (Int-14-A)

To a solution of 1-bromo-3-chloro-2,4-difluorobenzene (5.00 g, 22.0 mmol) and furan (3.20 mL, 44.0 mmol) in toluene (100 mL) was added n-BuLi (16.1 mL, 26.4 mmol, 1.64M in hexane) at 20° C. After stirring the mixture at 20° C. to room temperature overnight, the reaction was quenched by the addition of H₂O, filtered and concentrated in vacuo. The residue was dissolved in EtOAc, washed with brine, dried over Na₂SO₄, filtered and concentrated in vacuo. The crude product was purified by flash silica gel chromatography (0-60%, EtOAc gradient in hexane) to afford 5-chloro-6-fluoro-1,4-dihydro-1,4-epoxynaphthalene which was redissolved in EtOH (40 mL). HCl (25.0 mL, 300 mmol, 12M aq.) was added. After stirring the mixture at 80° C. for 3 h, the reaction mixture was cooled to room temperature and concentrated in vacuo. The residue was purified by flash silica gel chromatography (eluting with ethyl acetate in hexane, 0-100% gradient) to afford 8-chloro-7-fluoronaphthalen-1-ol (Int-14-A). ¹H NMR (500 MHz, CDCl₃) δ ppm 7.09 (d, J=7 Hz, 1H) 7.25-7.32 (m, 1H) 7.36-7.44 (m, 2H) 7.75 (dd, J=9, 6 Hz, 1H) 7.92 (s, 1H).

### Step B: 8-chloro-7-fluoronaphthalen-1-yl trifluoromethanesulfonate (Int-14-B)

To a solution of 8-chloro-7-fluoronaphthalen-1-ol (Int-14-A) (2.36 g, 12.0 mmol) in dichloromethane (20 mL) were added pyridine (2.13 mL, 26.4 mmol) and trifluoromethylsulfonic anhydride (2.62 mL, 15.6 mmol) at 0° C. After stirring the mixture at 0° C. for 2 h, the reaction was quenched by the addition of water. The reaction mixture was extracted with EtOAc, washed with brine, dried over Na₂SO₄, filtered and concentrated under reduced pressure. The residue was purified by flash silica gel chromatography (eluting with ethyl acetate in hexane, 0-100% gradient) to afford 8-chloro-7-fluoronaphthalen-1-yl trifluoromethanesulfonate (Int-14-B). ¹H NMR (500 MHz, CDCl₃) δ ppm 7.41-7.48 (m, 1H) 7.49-7.55 (m, 1H) 7.59 (br d, J=8 Hz, 1H) 7.82-7.88 (m, 1H) 7.90 (br d, J=8 Hz, 1H).

### Step C: 2-(8-chloro-7-fluoronaphthalen-1-yl)-4,4,5,5-tetramethyl-1,3,2-dioxaborolane (Int-14)

A mixture of 8-chloro-7-fluoronaphthalen-1-yl trifluoromethanesulfonate (Int-14-B) (200 mg, 0.609 mmol), 4,4,4′,4′,5,5,5′,5′-octamethyl-2,2′-bi(1,3,2-dioxaborolane) (309 mg, 0.122 mmol), KOAc (299 mg, 3.04 mmol) and 1,1′-bis(diphenylphosphino)ferrocenedichloropalladium(II) (49.7 mg, 0.0609 mmol) in DMF (2.0 mL) was stirred at 80° C. for 2 h. The reaction was quenched by the addition of water and the reaction mixture was extracted with EtOAc, washed with brine, dried over Na₂SO₄, filtered and concentrated in vacuo. The residue was purified by flash silica gel chromatography (eluting with ethyl acetate in hexane, 0-100% gradient) to afford 2-(8-chloro-7-fluoronaphthalen-1-yl)-4,4,5,5-tetramethyl-1,3,2-dioxaborolane (Int-14). ¹H NMR (400 MHz, CDCl₃) δ 7.85 (dd, J=8.4, 1.2 Hz, 1H), 7.76 (dd, J=9.2, 5.6 Hz, 1H), 7.70 (d, J=6.8 Hz, 1H), 7.48 (dd, J=8.0, 8.0 Hz, 1H), 7.33 (dd, J=8.8, 8.4 Hz, 1H), 1.45 (s, 12H).

### Intermediate 15: tert-butyl (3-cyano-4-(5,5-dimethyl-1,3,2-dioxaborinan-2-yl)-7-fluorobenzo[b]thiophen-2-yl)carbamate (Int-15)

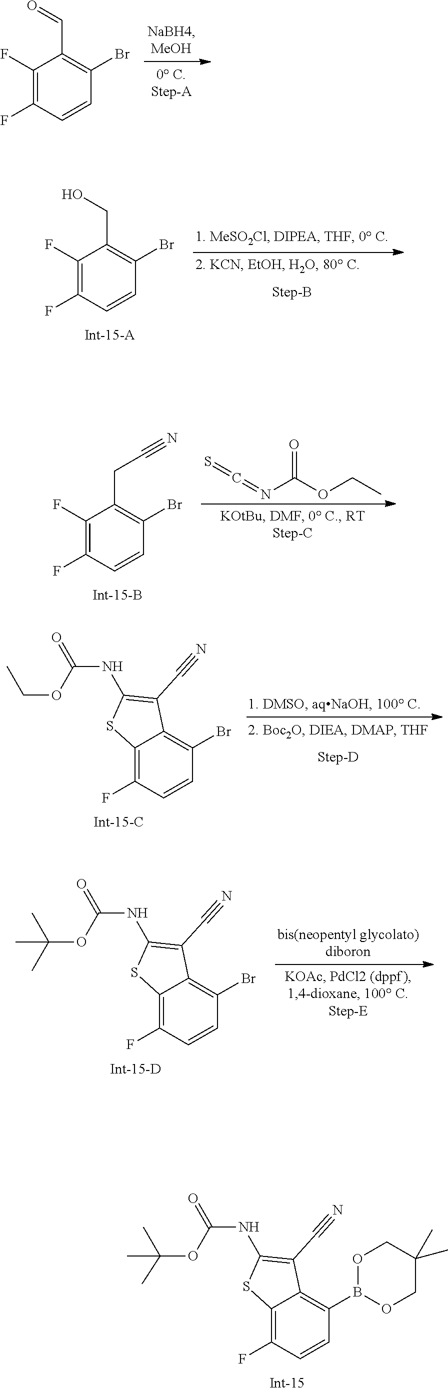

### Step A: (6-bromo-2,3-difluorophenyl)methanol (Int-15-A)

To a solution of 6-bromo-2,3-difluorobenzaldehyde (5.00 g, 22.6 mmol) in MeOH (100 mL) was added sodium borohydride (5.00 g, 22.0 mmol) at 0° C. After stirring the mixture at room temperature for 1 h, the reaction was quenched by the addition of H₂O. The reaction mixture was extracted with EtOAc, washed with brine, dried over Na₂SO₄, filtered and concentrated under reduced pressure to afford (6-bromo-2,3-difluorophenyl)methanol (Int-15-A). ¹H NMR (400 MHz, CDCl₃) δ 7.37-7.31 (m, 1H), 7.09-7.02 (m, 1H), 4.86 (dd, J=6.8, 2.4 Hz, 2H), 2.11 (t, J=6.8 Hz, 1H).

### Step B: 2-(6-bromo-2,3-difluorophenyl)acetonitrile (Int-15-B)

To a solution of (6-bromo-2,3-difluorophenyl)methanol (Int-15-A) (3.00 g, 13.5 mmol) in THF (30 mL) was added N,N-diisopropylethylamine (2.81 mL, 16.1 mmol) and methanesulfonyl chloride (1.15 mL, 14.8 mmol) at 0° C. After stirring the mixture at room temperature for 15 h, the reaction mixture was quenched by the addition of H₂O. The reaction mixture was extracted with EtOAc, washed with brine, dried over Na₂SO₄, filtered and concentrated under reduced pressure to afford benzyl chloride derivative without further purification. To a solution of benzyl chloride derivative in EtOH (30 mL) and H₂O (6 mL) was added potassium cyanide (818 mg, 12.6 mmol). After stirring the mixture at 80° C. for 1 h, the reaction mixture was cooled to room temperature and concentrated under reduced pressure. The reaction mixture was extracted with EtOAc, washed with brine, dried over Na₂SO₄, filtered and concentrated in vacuo. The residue was purified by flash silica gel chromatography (eluting with ethyl acetate in hexanes, 0-100% gradient) to afford 2-(6-bromo-2,3-difluorophenyl)acetonitrile (Int-15-B). ¹H NMR (400 MHz, CDCl₃) δ 7.43-7.38 (m, 1H), 7.17-7.09 (m, 1H), 4.89 (d, J=2.0 Hz, 2H).

### Step C: ethyl (4-bromo-3-cyano-7-fluorobenzo[b]thiophen-2-yl)carbamate (Int-15-C)

To a solution of 2-(6-bromo-2,3-difluorophenyl)acetonitrile (Int-15-B) (2.00 g, 8.62 mmol) in DMF (20 mL) was added potassium tert-butoxide (1.02 g, 9.05 mmol) at 0° C. After stirring the mixture at 0° C. for 10 min, to the reaction mixture was added ethoxycarbonyl isothiocyanate (1.07 mL, 9.05 mmol). The reaction mixture was stirred at room temperature for 1 h, and then heated at 100° C. for 30 min. The mixture was then cooled to 0° C., and H₂O was added slowly with stirring. The resulting precipitate was collected by filtration, rinsed with H₂O and hexane, and dried in vacuo to afford ethyl (4-bromo-3-cyano-7-fluorobenzo[b]thiophen-2-yl)carbamate (Int-15-C) ESI-MS m/z [M−H]-341, 343.

### Step D: tert-butyl (4-bromo-3-cyano-7-fluorobenzo[b]thiophen-2-yl)carbamate (Int-15-D)

To a solution of ethyl (4-bromo-3-cyano-7-fluorobenzo[b]thiophen-2-yl)carbamate (Int-15-C) (2.51 g, 7.31 mmol) in DMSO (10 mL) was added 5.0 M aqueous solution of NaOH (8.00 mL, 40.0 mmol). After stirring the mixture at 100° C. for 13 h, the mixture was then cooled to room temperature, and H₂O was added slowly with stirring. The resulting precipitate was collected by filtration, rinsed with H₂O and hexane, and dried in vacuo. The residue was redissolved in THF (40 mL) and was treated with N,N-diisopropylethylamine (1.54 mL, 8.85 mmol), DMAP (36.0 mg, 0.295 mmol) and di-tert-butyl dicarbonate (1.42 g, 6.49 mmol). After stirring the mixture at room temperature for 13 h, H₂O and EtOAc were added, and then the resulting precipitate was collected by filtration, rinsed with H₂O and hexane, and dried in vacuo to afford tert-butyl (4-bromo-3-cyano-7-fluorobenzo[b]thiophen-2-yl)carbamate. The filtrate layers were extracted with EtOAc, washed with brine, dried over Na₂SO₄, filtered and concentrated in vacuo. The residue was purified by flash silica gel chromatography (0-10%, MeOH gradient in EtOAc) to afford tert-butyl (4-bromo-3-cyano-7-fluorobenzo[b]thiophen-2-yl)carbamate (Int-15-D). ¹H NMR (400 MHz, CDCl₃) δ 7.96 (s, 1H), 7.54-7.49 (m, 1H), 6.94-6.88 (m, 1H), 1.59 (s, 9H).

### Step E: tert-butyl (3-cyano-4-(5,5-dimethyl-1,3,2-dioxaborinan-2-yl)-7-fluorobenzo[b]thiophen-2-yl)carbamate (Int-15)

To a solution of tert-butyl (4-bromo-3-cyano-7-fluorobenzo[b]thiophen-2-yl)carbamate (Int-15-D) (1.00 g, 2.69 mmol) and bis(neopentyl glycolato)diboron (1.83 g, 8.08 mmol) in 1,4-dioxane (15 mL) was added potassium acetate (793 mg, 8.08 mmol). After stirring the mixture at room temperature for 1 h, to the mixture was added dichlorobis(diphenylphosphinophenyl)ether palladium (II) (193 mg, 0.269 mmol), and then the mixture was heated at 100° C. for 2 h. The reaction mixture was cooled to room temperature, and H₂O was added. The reaction mixture was extracted with EtOAc, washed with brine, dried over Na₂SO₄, filtered and concentrated in vacuo. The residue was purified by flash silica gel chromatography (0-30%, EtOAc gradient in hexane) to afford tert-butyl (3-cyano-4-(5,5-dimethyl-1,3,2-dioxaborinan-2-yl)-7-fluorobenzo[b]thiophen-2-yl)carbamate (Int-15). ¹H NMR (400 MHz, DMSO-d₆) δ 11.6 (s, 1H), 7.61-7.56 (m, 1H), 7.22-7.15 (m, 1H), 3.77 (s, 4H), 1.52 (s, 9H), 1.02 (s, 6H).

### Intermediate 16: 4-bromo-5-chloro-1-fluoro-2-(methoxymethoxy)naphthalene (Int-16)

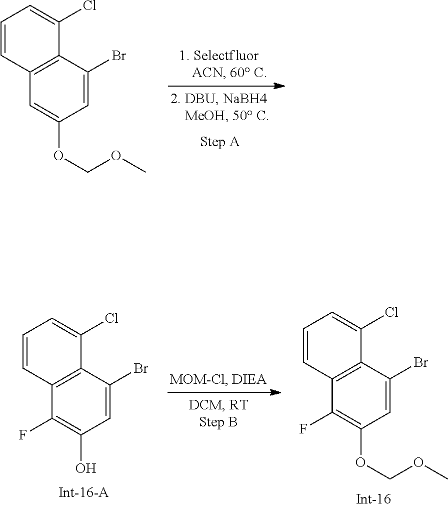 [SMILES: COCOc1cc(Br)c2c(Cl)cccc2c1 ; Oc1cc(Br)c2c(Cl)cccc2c1F ; COCOc1cc(Br)c2c(Cl)cccc2c1F]

### Step A: 4-bromo-5-chloro-1-fluoronaphthalen-2-ol (Int-16-A)

To a solution 1-bromo-8-chloro-3-(methoxymethoxy)naphthalene (200 mg, 0.663 mmol) in MeCN (2.0 mL) was added 1-chloromethyl-4-fluoro-1,4-diazoniabicyclo[2.2.2]octane bis(tetrafluoroborate) (470 mg, 1.33 mmol). After stirring the mixture at 60° C. for 2 h, the reaction was quenched by the addition of saturated aqueous NaHCO₃. The reaction mixture was extracted with EtOAc, washed with brine, dried over Na₂SO₄, filtered and concentrated in vacuo to afford fluorinated compound.

To a solution of fluorinated compound in MeOH (2.2 mL) were added 1,8-diazabicyclo[5.4.0]undec-7-ene (0.428 mL, 3.32 mmol) and sodium tetrahydroborate (125 mg, 3.32 mmol) at 0° C. After stirring the mixture at 50° C. for 30 min, the reaction was quenched by the addition of saturated aqueous NH₄Cl. The reaction mixture was extracted with EtOAc, washed with brine, dried over Na₂SO₄, filtered and concentrated in vacuo. The residue was purified by flash silica gel chromatography (0-20%, EtOAc gradient in hexane) to afford 4-bromo-5-chloro-1-fluoronaphthalen-2-ol (Int-16-A). ¹H NMR (400 MHz, CDCl₃) δ7.95 (ddd, J=8.4, 1.2, 1.2 Hz, 1H), 7.71 (d, J=8.6 Hz, 1H), 7.56 (d, J=7.5 Hz, 1H), 7.39 (dd, J=8.0, 7.9 Hz, 1H), 5.54 (d, J=3.8 Hz, 1H).

### Step B: 4-bromo-5-chloro-1-fluoro-2-(methoxymethoxy)naphthalene (Int-16)

To a solution 4-bromo-5-chloro-1-fluoronaphthalen-2-ol (Int-16-A) (158 mg, 0.573 mmol) in dichloromethane (3.0 mL) were added N,N-diisopropylethylamine (2.96 mL, 17.0 mmol) and chloromethyl methyl ether (0.0871 mL, 1.15 mmol). After stirring the mixture at room temperature for 15 min, the reaction was quenched by the addition of saturated aqueous NaHCO₃. The reaction mixture was extracted with EtOAc, washed with brine, dried over Na₂SO₄, filtered and concentrated in vacuo. The residue was purified by flash silica gel chromatography (0-20%, EtOAc gradient in hexane) to afford 4-bromo-5-chloro-1-fluoro-2-(methoxymethoxy)naphthalene (Int-16). ¹H NMR (400 MHz, CDCl₃) δ 8.05 (ddd, J=8.5, 1.2, 1.2 Hz, 1H), 7.88 (d, J=8.1 Hz, 1H), 7.60 (d, J=7.4 Hz, 1H), 7.40 (dd, J=8.2, 7.8 Hz, 1H), 5.30 (s, 2H), 3.57 (s, 3H).

### Intermediate 17: (R)-1-(7-chloro-2-((1-((dimethylamino)methyl)cyclopropyl)methoxy)-8-fluoropyrido[4,3-d]pyrimidin-4-yl)-3-methylpiperidin-3-ol (Int-17)

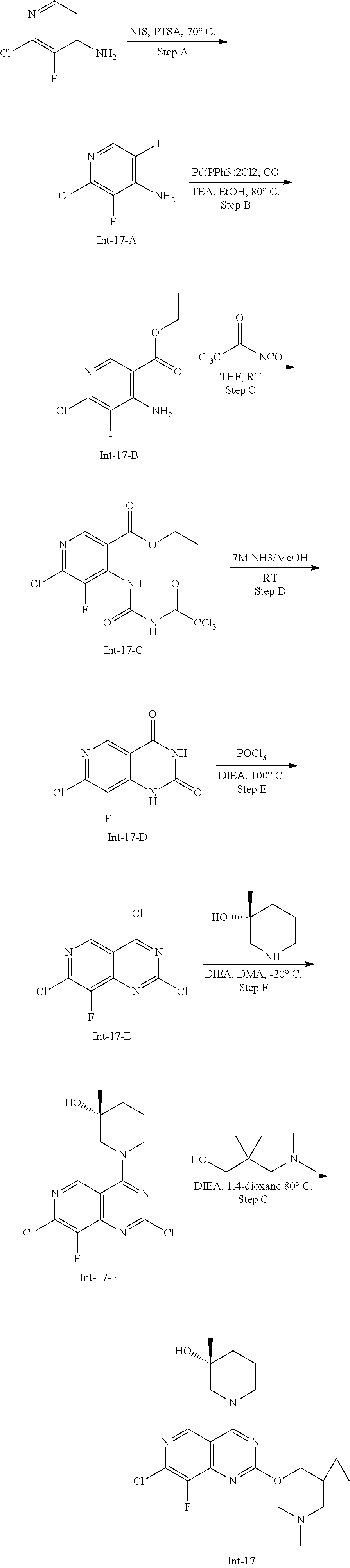

### Step A: 2-chloro-3-fluoro-5-iodopyridin-4-amine (Int-17-A)

To a solution of 2-chloro-3-fluoro-pyridin-4-amine (13.6 g, 92.8 mmol) and N-iodosuccinimide (25.1 g, 111 mmol) in acetonitrile (67 mL) was added p-toluene sulfonic acid monohydrate (883 mg, 4.64 mmol). The mixture was stirred at 70° C. for 17 h. The reaction mixture was cooled to room temperature and diluted with water and ethyl acetate. The organic layer was washed with saturated Na₂CO₃ solution, saturated Na₂SO₃ solution and brine, dried over MgSO₄, filtered and concentrated in vacuo. The residue was dried under reduced pressure for 4 h to afford 2-chloro-3-fluoro-5-iodo-pyridin-4-amine (Int-17-A). ESI-MS m/z [M+H]⁺ 273. ¹H NMR (400 MHz, methanol-d₄) δ 8.08 (d, J=0.8 Hz, 1H).

### Step B: ethyl 4-amino-6-chloro-5-fluoronicotinate (Int-17-B)

To a solution of 2-chloro-3-fluoro-5-iodo-pyridin-4-amine (Int-17-A) (24.6 g, 90.3 mmol) and Pd(PPh₃)₂Cl₂ (6.34 g, 9.03 mmol) in ethanol (492 ml) was added Et₃N (45.4 ml, 326 mmol) under nitrogen atmosphere. The suspension was degassed under reduced pressure and purged with carbon monoxide several times. The mixture was stirred at 80° C. for 15 h under carbon monoxide atmosphere. The reaction mixture was cooled to room temperature and filtered, and the filtrate was concentrated in vacuo to remove 70% of ethanol and the residue was filtered. The filter cake was dried under reduced pressure for 4 h to afford ethyl 4-amino-6-chloro-5-fluoronicotinate (Int-17-B). ESI-MS m/z [M+H]⁺ 219, 221. ¹H NMR (400 MHz, DMSO-d₆) δ 8.38 (s, 1H), 7.59 (br s, 2H), 4.32 (q, 2H, J=7.0 Hz), 1.32 (t, 3H, J=7.1 Hz).

### Step C: ethyl 6-chloro-5-fluoro-4-(3-(2,2,2-trichloroacetyl)ureido)nicotinate (Int-17-C)

To a solution of ethyl 4-amino-6-chloro-5-fluoronicotinate (Int-17-B) (8.44 g, 38.6 mmol) in THF (25 ml) was added 2,2,2-trichloroacetyl isocyanate (6.86 ml, 57.9 mmol) at 25° C. The mixture was stirred at room temperature for 10 min under nitrogen atmosphere. The reaction mixture was concentrated in vacuo. The residue was dried under reduced pressure for 4 h to afford ethyl 6-chloro-5-fluoro-4-(3-(2,2,2-trichloroacetyl)ureido)nicotinate (Int-17-C). ESI-MS m/z [M+H]⁺ 406. ¹H NMR (400 MHz, DMSO-d₆) δ 11.96 (br s, 1H), 10.48 (s, 1H), 8.64 (s, 1H), 4.31 (q, 2H, J=7.1 Hz), 1.29 (t, J=7.1 Hz, 3H).

### Step D: 7-chloro-8-fluoropyrido[4,3-d]pyrimidine-2,4(1H,3H)-dione (Int-17-D)

To a solution of ethyl 6-chloro-5-fluoro-4-(3-(2,2,2-trichloroacetyl)ureido)nicotinate (Int-17-C) (15.7 g, 38.6 mmol) in methanol (157 ml) was added 7M ammonia in methanol (16.5 ml, 116 mmol) at 25° C. The mixture was stirred at 25° C. for 1 h under nitrogen atmosphere. The reaction mixture was concentrated in vacuo. The residue was dried under reduced pressure for 4 h to afford 7-chloro-8-fluoropyrido[4,3-d]pyrimidine-2,4(1H,3H)-dione (Int-17-D). ESI-MS m/z [M+H]⁺ 216. ¹H NMR (400 MHz, DMSO-d₆) δ 8.32 (s, 1H).

### Step E: 2,4,7-trichloro-8-fluoropyrido[4,3-d]pyrimidine (Int-17-E)

To a solution of 7-chloro-8-fluoropyrido[4,3-d]pyrimidine-2,4(1H,3H)-dione (Int-17-D) (6.0 g, 27.8 mmol) in POCl₃ (59.1 ml) was added diisopropylethylamine (38.1 ml, 223 mmol) at 25° C. The mixture was stirred at 100° C. for 1 h under nitrogen atmosphere. The reaction mixture was cooled to room temperature and concentrated in vacuo. The residue was azeotroped with toluene and dried under reduced pressure for 4 h to afford 2,4,7-trichloro-8-fluoropyrido[4,3-d]pyrimidine (Int-17-E). ESI-MS m/z [M+H]⁺ 252.

### Step F: (R)-1-(2,7-dichloro-8-fluoropyrido[4,3-d]pyrimidine-4-yl)-3-methylpiperidin-3-ol (Int-17-F)

To a solution of 2,4,7-trichloro-8-fluoropyrido[4,3-d]pyrimidine (Int-17-E) (3.5 g, 13.9 mmol) in N,N-dimethylacetamide (35 ml) was added (3R)-3-methylpiperidin-3-ol (1.89 g, 12.5 mmol) and diisopropylethylamine (24.1 ml, 139 mmol) at −20° C. The mixture was stirred at −20° C. for 0.5 h under nitrogen atmosphere. The reaction mixture was diluted with water and ethyl acetate. The organic layer was washed with brine, dried over MgSO₄, filtered and concentrated in vacuo. The residue was purified by flash silica chromatography (eluent, 12-100% EtOAc in hexane) to obtain (R)-1-(2,7-dichloro-8-fluoropyrido[4,3-d]pyrimidin-4-yl)-3-methylpiperidin-3-ol (Int-17-F). ESI-MS m/z [M+H]⁺ 331. ¹H NMR (400 MHz, DMSO-d₆) δ 9.19 (s, 1H), 4.74 (br s, 1H), 4.47 (br d, 1H, J=12.5 Hz), 4.12 (d, 1H, J=13.4 Hz), 3.58 (d, J=13.4 Hz, 1H), 3.26-3.29 (m, 1H), 1.90-2.03 (m, 1H), 1.60-1.73 (m, 3H), 1.16 (s, 3H).

### Step G: (R)-1-(7-chloro-2-((1-((dimethylamino)methyl)cyclopropyl)methoxy)-8-fluoropyrido[4,3-d]pyrimidine-4-yl)-3-methylpiperidin-3-ol (Int-17)

To a solution of (R)-1-(2,7-dichloro-8-fluoropyrido[4,3-d]pyrimidin-4-yl)-3-methylpiperidin-3-ol (Int-17-F) (300 mg, 0.91 mmol) and (1-((dimethylamino)methyl)cyclopropyl)methanol (234 mg, 1.81 mmol) in 1,4-dioxane (4.5 ml) was added diisopropylethylamine (473 μl, 2.72 mmol) at room temperature. The mixture was stirred at 80° C. for 6 h under nitrogen atmosphere. The reaction mixture was cooled to room temperature and diluted with water and ethyl acetate. The organic layer was dried over MgSO₄, filtered and concentrated in vacuo. The residue was purified by flash NH-silica chromatography (eluent, 12-100% EtOAc in hexane) and reverse phase HPLC (MeCN/water with 0.1% formic acid) to obtain (R)-1-(7-chloro-2-((1-((dimethylamino)methyl)cyclopropyl)methoxy)-8-fluoropyrido[4,3-d]pyrimidin-4-yl)-3-methylpiperidin-3-ol (Int-17). ESI-MS m/z [M+H]⁺ 424. ¹H NMR (400 MHz, CDCl₃) δ 8.88 (s, 1H), 4.33-4.42 (m, 4H), 3.36-3.43 (m, 1H), 3.25 (d, 1H, J=13.5 Hz), 2.77 (br s, 1H), 2.29-2.38 (m, 2H), 2.25 (s, 6H), 1.97-2.09 (m, 1H), 1.86-1.90 (m, 1H), 1.62-1.75 (m, 2H), 1.33 (s, 3H), 0.64-0.74 (m, 2H), 0.42-0.52 (m, 2H).

### Intermediate 18: (R)-1-(7-bromo-2-((1-((dimethylamino)methyl)cyclopropyl)methoxy)-8-fluoroquinazolin-4-yl)-3-methylpiperidin-3-ol (Int-18)

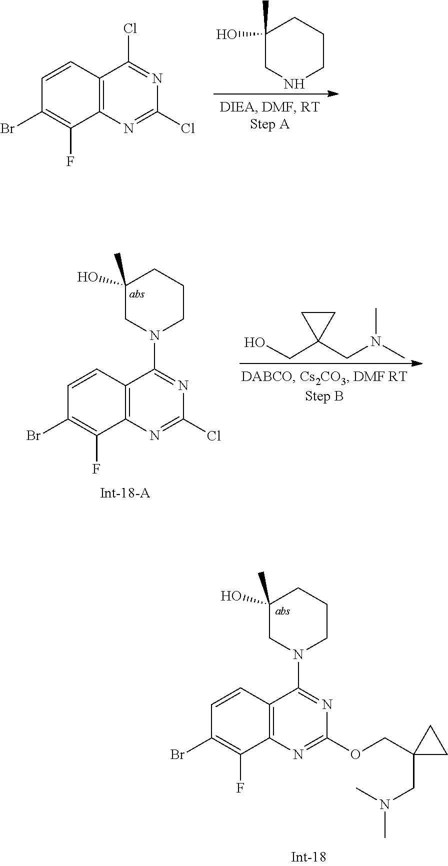

### Step A: (R)-1-(7-bromo-2-chloro-8-fluoroquinazolin-4-yl)-3-methylpiperidin-3-ol (Int-18-A)

To a solution of 7-bromo-2,4-dichloro-8-fluoroquinazoline (200 mg, 0.676 mmol) and (R)-3-methylpiperidin-3-ol hydrochloride (108 mg, 0.676 mmol) in DMF (3.0 mL) was added N,N-diisopropylethylamine (0.471 mL, 2.70 mmol) at room temperature. After stirring the mixture at room temperature for 30 min, the reaction was quenched by the addition of H₂O. The reaction mixture was extracted with EtOAc, washed with brine, dried over Na₂SO₄, filtered and concentrated in vacuo to afford (R)-1-(7-bromo-2-chloro-8-fluoroquinazolin-4-yl)-3-methylpiperidin-3-ol (Int-18-A). ESI-MS m/z [M+H]⁺ 374, 376, 378.

### Step B: (R)-1-(7-bromo-2-((1-((dimethylamino)methyl)cyclopropyl)methoxy)-8-fluoroquinazolin-4-yl)-3-methylpiperidin-3-ol (Int-18)

To a solution of (R)-1-(7-bromo-2-chloro-8-fluoroquinazolin-4-yl)-3-methylpiperidin-3-ol (Int-18-A) (190 mg, 0.507 mmol) and (1-((dimethylamino)methyl)cyclopropyl)methanol (112 mg, 0.867 mmol) in DMF (5.0 mL) were added cesium carbonate (496 mg, 1.52 mmol) and 1,4-diazabicyclo[2.2.2]octane (11.4 mg, 0.101 mmol) at room temperature. After stirring the mixture at room temperature for 13 h, the reaction was quenched by the addition of H₂O. The reaction mixture was extracted with EtOAc, washed with brine, dried over Na₂SO₄, filtered and concentrated in vacuo. The residue was purified by flash NH-silica gel chromatography (20-50%, EtOAc gradient in hexane) to afford (R)-1-(7-bromo-2-((1-((dimethylamino)methyl)cyclopropyl)methoxy)-8-fluoroquinazolin-4-yl)-3-methylpiperidin-3-ol (Int-18). ESI-MS m/z [M+H]⁺ 467, 469.

The compounds in the table below were synthesized using a similar procedure as described in the synthesis of Int-18 by making the appropriate substitutions for starting material, intermediates, and/or reagents. Appropriate substitutions are available commercially, synthesized as described in the literature, synthesized using methods available to those skilled in the art, or synthesized as described herein.

| Int | Structure | Compound Name | [M + H]⁺ Found |
|---|---|---|---|
| Int-19 | 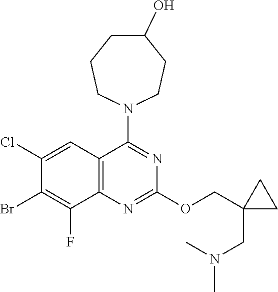 [SMILES: CN(C)CC1(COc2nc(N3CCCC(O)CC3)c3cc(Cl)c(Br)c(F)c3n2)CC1] | 1-(7-bromo-6-chloro-2-((1- ((dimethylamino)methyl)cy- clopropyl)methoxy)-8- fluoroquinazolin-4- yl)azepan-4-ol | 501, 503 |
| Int-20 | 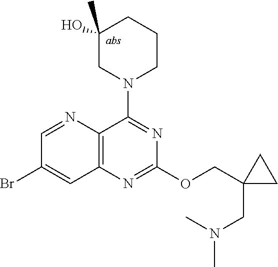 [SMILES: CN(C)CC1(COc2nc(N3CCC[C@@](C)(O)C3)c3ncc(Br)cc3n2)CC1] | (R)-1-(7-bromo-2-((1- ((dimethylamino)methyl)cy- clopropyl)methoxy)pyrido[3,2- d]pyrimidin-4-yl)-3- methylpiperidin-3-ol | 450, 452 |
| Int-21 | 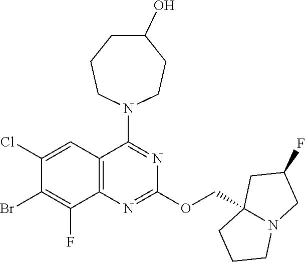 [SMILES: OC1CCCN(c2nc(OC[C@@]34CCCN3C[C@H](F)C4)nc3c(F)c(Br)c(Cl)cc23)CC1] | 1-(7-bromo-6-chloro-8- fluoro-2-(((2R,7aS)-2- fluorotetrahydro-1H- pyrrolizin-7a(5H)- yl)methoxy)quinazolin-4- yl)azepan-4-ol | 531, 533, 535 |

### Intermediate 22: 7-bromo-2,4,6-trichloro-8-fluoroquinazoline (Int-22)

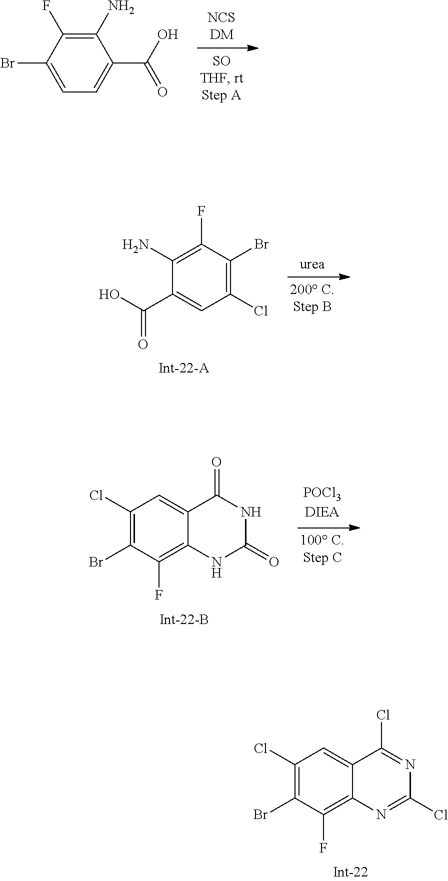 [SMILES: Nc1c(C(=O)O)ccc(Br)c1F ; Nc1c(C(=O)O)cc(Cl)c(Br)c1F ; O=c1[nH]c(=O)c2cc(Cl)c(Br)c(F)c2[nH]1 ; Fc1c(Br)c(Cl)cc2c(Cl)nc(Cl)nc12]

### Step A: 2-amino-4-bromo-5-chloro-3-fluorobenzoic acid (Int-22-A)

Into a 10-L 4-necked round-bottom flask were added 2-amino-4-bromo-3-fluorobenzoic acid (700 g, 2.99 mol), THF (7.0 L) and DMSO (93.5 g, 1.20 mol). This was followed by the addition of NCS (799 g, 5.98 mol) in several batches at 25° C. The resulting solution was stirred overnight at room temperature. The reaction was then quenched by the addition of 7 L of sat. NaCl (aq). The resulting solution was extracted with ethyl acetate (2×2 L), and the organic layer was washed with water (3×1 L) and dried over anhydrous sodium sulfate. The dried solution was filtered, and the filtrate was concentrated. The residue obtained was triturated in petroleum ether:ethyl acetate=10:1 (5 L) for 1 h. The solid was collected by filtration to afford 2-amino-4-bromo-5-chloro-3-fluorobenzoic acid (Int-22-A) which was used directly in the next step without purification.

### Step B: 7-bromo-6-chloro-8-fluoroquinazoline-2,4(1H,3H)-dione (Int-22-B)

A 2-L 4-necked round-bottom flask was charged with 2-amino-4-bromo-5-chloro-3-fluorobenzoic acid (Int-22-A) (100 g, 372 mmol) and urea (112 g, 1.86 mol). The resulting solution was stirred for 2 h at 200° C. The reaction mixture was cooled to 25° C. and the reaction solidified. This reaction was repeated in 6 additional batches using the above conditions. The seven batches of reactions were combined, and the bulk of the solid was ground to afford a crude mixture of 7-bromo-6-chloro-8-fluoroquinazoline-2,4(1H,3H)-dione (Int-22-B) that was used directly in the next step without purification.

### Step C: 7-bromo-2,4,6-trichloro-8-fluoroquinazoline (Int-22)

A 3-L 4-necked round-bottom flask was charged with 7-bromo-6-chloro-8-fluoroquinazoline-2,4(1H,3H)-dione (Int-22-B) (290 g, 0.980 mol) and POCl₃ (1.51 kg, 9.86 mol). This was followed by the addition of DIEA (382 g, 2.96 mol) dropwise with stirring at 25° C. The resulting solution was stirred for 2 h at 100° C. This reaction was repeated in 2 additional batches using the above conditions. The three batches of reactions were combined and concentrated. The residue was purified by silica gel column eluted with petroleum ether:ethyl acetate=10:1. The crude product was slurried in petroleum ether:ethyl acetate=5:1(1 L). The solid was collected by filtration to afford 7-bromo-2,4,6-trichloro-8-fluoroquinazoline (Int-22). ¹H NMR (300 MHz, CDCl₃, ppm): δ 8.227 (s, 1H).

### Intermediate 23: (R)-1-(6,8-difluoro-2-(((2R,7aS)-2-fluorotetrahydro-1H-pyrrolizin-7a(5H)-yl)methoxy)quinazolin-4-yl)-3-methylpiperidin-3-ol (Int-23)

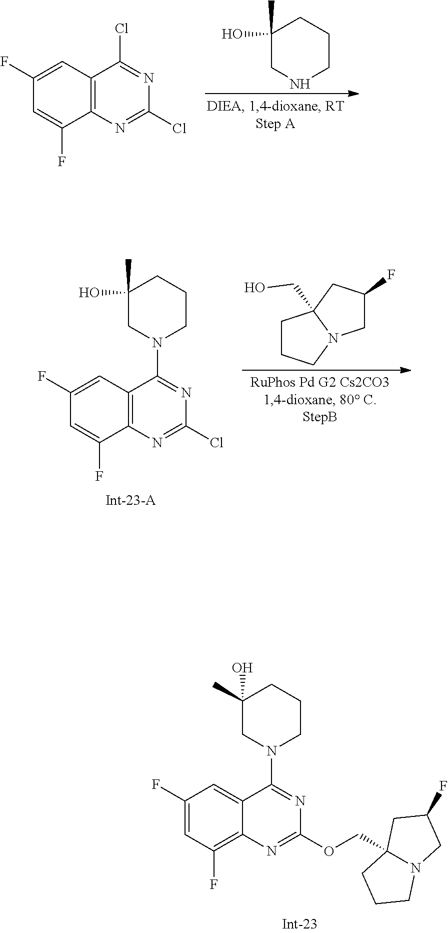

### Step A: (R)-1-(2-chloro-6,8-difluoroquinazolin-4-yl)-3-methylpiperidin-3-ol (Int-23-A)

To a solution of 2,4-dichloro-6,8-difluoroquinazoline (Int-11) (350 mg, 1.49 mmol) in 1,4-dioxane (8.00 ml) was added (R)-3-methylpiperidin-3-ol (226 mg, 1.49 mmol) followed by N-ethyl-N-isopropylpropan-2-amine (1.04 ml, 5.96 mmol). The reaction mixture was stirred at RT for 25 mins. The reaction was then cooled to RT and diluted with DCM and water. The organic layers were shaken, then separated. The organic phase was concentrated. The resulting residue was purified by silica gel column chromatography (0-10% ethyl acetate gradient in hexanes) to afford (R)-1-(2-chloro-6,8-difluoroquinazolin-4-yl)-3-methylpiperidin-3-ol (Int-23-A). MS (ESI): m/z (M+H)⁺ 314.

### Step B: (R)-1-(6,8-difluoro-2-(((2R,7aS)-2-fluorotetrahydro-1H-pyrrolizin-7a(5H)-yl)methoxy)quinazolin-4-yl)-3-methylpiperidin-3-ol (Int-23)

To a solution of (R)-1-(2-chloro-6,8-difluoroquinazolin-4-yl)-3-methylpiperidin-3-ol (Int-23-A) (315 mg, 1.00 mmol) in 1,4-dioxane (6.00 ml) was added ((2R,7aS)-2-fluorotetrahydro-1H-pyrrolizin-7a(5H)-yl)methanol (Int-8) (176 mg, 1.10 mmol), cesium carbonate (818 mg, 2.51 mmol) and RuPhos Pd G2 (78 mg, 0.100 mmol). The reaction mixture was stirred at 80° C. for 3 hrs. It was then cooled to RT, quenched with water and extracted with EtOAc. The combined extracts were washed with saturated brine solution and concentrated. The product was purified by silica gel column chromatography using 100% of EtOAc in hexane to get (R)-1-(6,8-difluoro-2-(((2R,7aS)-2-fluorotetrahydro-1H-pyrrolizin-7a(5H)-yl)methoxy)quinazolin-4-yl)-3-methylpiperidin-3-ol (Int-23). MS (ESI): m/z (M+H)⁺ 437.

The compound in the table below was synthesized using a similar procedure as described in the synthesis of Int-23 by making the appropriate substitutions for starting material, intermediates, and/or reagents. Appropriate substitutions are available commercially, synthesized as described in the literature, synthesized using methods available to those skilled in the art, or synthesized as described herein.

| Int | Structure | Compound Name | [M + H]⁺ Found |
|---|---|---|---|
| Int-24 | 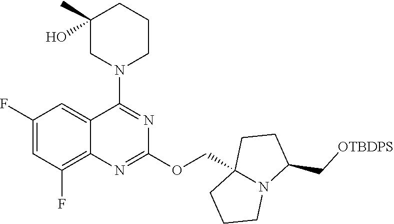 | (R)-1-(2-(((3S,7aS)-3- (((tert- butyldiphenylsilyl)oxy)meth- yl)tetrahydro-1H- pyrrolizin-7a(5H)- yl)methoxy)-6,8- difluoroquinazolin-4-yl)- 3-methylpiperidin-3-ol | 687 |

### Intermediate 25: (R)-1-(7-bromo-2-chloro-6,8-difluoroquinazolin-4-yl)-3-methylpiperidin-3-ol (Int-25)

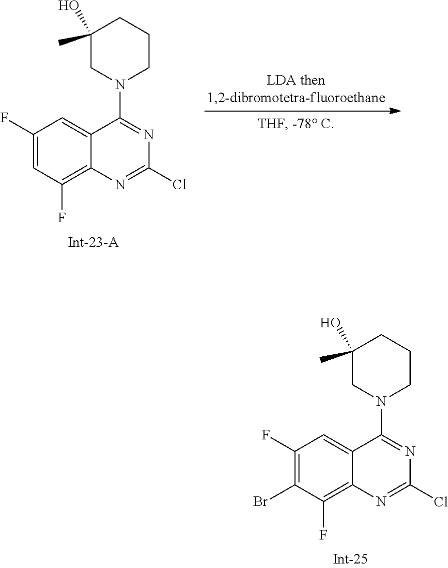 [SMILES: C[C@@]1(O)CCCN(c2nc(Cl)nc3c(F)cc(F)cc23)C1 ; C[C@@]1(O)CCCN(c2nc(Cl)nc3c(F)c(Br)c(F)cc23)C1]

A solution of (R)-1-(2-chloro-6,8-difluoroquinazolin-4-yl)-3-methylpiperidin-3-ol (Int-23-A) (1000 mg, 3.19 mmol) in tetrahydrofuran (15.0 ml) was cooled under nitrogen bubbler to −78° C. and treated with LDA (2 M in THF/heptane/ethylbenzene, 4.8 ml, 9.6 mmol). The mixture was stirred at −78° C. for 30 minutes to afford a red-colored solution. The mixture was treated at −78° C. with a solution of 1,2-dibromotetrafluoroethane (1.14 ml, 9.56 mmol) in tetrahydrofuran (2.00 ml). The mixture was stirred at −78° C. for 30 minutes and then quenched at −78° C. with saturated aqueous ammonium chloride (1 ml). The mixture was warmed to rt, diluted with water (1 ml) and extracted with ethyl acetate. The combined organic layers were dried with anhydrous sodium sulfate, filtered and the filtrate was concentrated. Purification by column chromatography on silica gel (0 to 100% EtOAc gradient in hexanes) afforded the titled compound (Int-25). MS (ESI): m/z (M+H)⁺ 392, 394 ¹H NMR (500 MHz, Acetone-d₆) δ 8.07 (d, J=9.6 Hz, 1H), 4.42 (d, J=13.2 Hz, 1H), 4.19 (d, J=13.5 Hz, 1H), 3.96 (s, 1H), 3.50 (d, J=13.5 Hz, 1H), 3.34-3.24 (m, 1H), 2.25-2.14 (m, 1H), 1.85 (t, J=9.8 Hz, 1H), 1.80-1.70 (m, 2H), 1.28 (s, 3H).

The compound in the table below was synthesized using a similar procedure as described in the synthesis of Int-25 by making the appropriate substitutions for starting material, intermediates, and/or reagents. Appropriate substitutions are available commercially, synthesized as described in the literature, synthesized using methods available to those skilled in the art, or synthesized as described herein.

| | | | [M + H]⁺ |
|---|---|---|---|
| Int | Structure | Compound Name | Found |
| Int-26 | 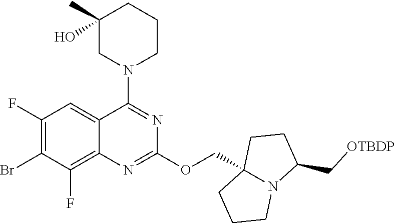 | (R)-1-(7-bromo-2-(((3S,7aS)- 3-(((tert- butyldiphenylsilyl)oxy)methyl) tetrahydro-1H-pyrrolizin- 7a(5H)-yl)methoxy)-6,8- difluoroquinazolin-4-yl)-3- methylpiperidin-3-ol | 765 |

### Intermediate 27: 7-(5,6-dimethyl-1-(tetrahydro-2H-pyran-2-yl)-1H-indazol-4-yl)-6,8-difluoro-2-((1-(((R)-3-fluoropyrrolidin-1-yl)methyl)cyclopropyl)methoxy)quinazolin-4-ol (Int-27)

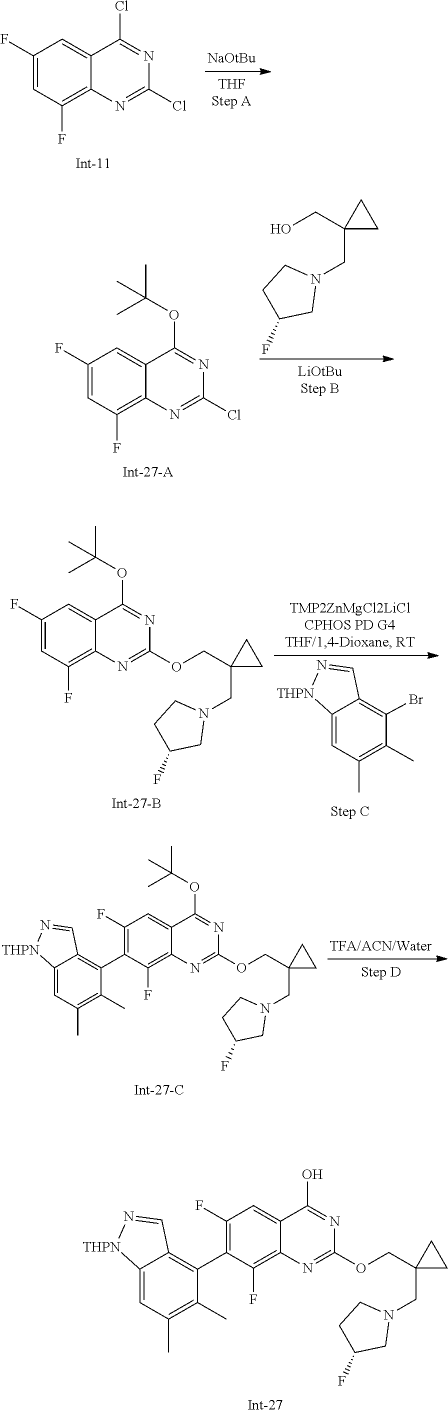

### Step A: 4-(tert-butoxy)-2-chloro-6,8-difluoroquinazoline (Int-27-A)

A solution of 2,4-dichloro-6,8-difluoroquinazoline (Int-11) (2.00 g, 8.51 mmol) in anhydrous THF (42 mL) was cooled in an ice bath under nitrogen. Then sodium tert-butoxide (4.7 mL of 2.0M solution in THF, 9.4 mmol) was added. After stirring for 72 min in the ice bath, additional sodium tert-butoxide (0.63 mL of 2.0M solution in THF, 1.26 mmol) was added. After stirring for an additional 18 min, the mixture was concentrated by rotary evaporation. The residue was extracted with DCM (20 mL), filtered, and concentrated by rotary evaporation. The residue was extracted with DCM (8 mL), filtered, and the filtrate was purified by silica gel column chromatography (90% hexane and 10% DCM, gradient to 100% DCM), to afford 4-(tert-butoxy)-2-chloro-6,8-difluoroquinazoline (Int-27-A). MS (ESI): m/z (M+H−tBu)⁺ 217.1. ¹H NMR (500 MHz, CDCl₃) δ 7.53-7.47 (m, 1H), 7.32 (td, J=9.5, 2.7 Hz, 1H), 1.75 (s, 9H).

### Step B: (R)-4-(tert-butoxy)-6,8-difluoro-2-((1-((3-fluoropyrrolidin-1-yl)methyl)cyclopropyl)methoxy)quinazoline (Int-27-B)

To a suspension of 4-(tert-butoxy)-2-chloro-6,8-difluoroquinazoline (Int-27-A) (14.17 g, 52.0 mmol) in anhydrous 1,4-dioxane (139 mL) was added a solution of (R)-(1-((3-fluoropyrrolidin-1-yl)methyl)cyclopropyl)methanol (6.0 g, 35 mmol) in anhydrous 1,4-dioxane (14 mL), together with further anhydrous 1,4-dioxane (20 mL). To the mixed suspension was added lithium 2-methylpropan-2-olate (38 mL, 1.0M solution in THF, 38 mmol). The mixture was stirred at 100° C. under an atmosphere of nitrogen for 3 h and 42 min, then was allowed to stand at 4° C. for 20.5 h. The mixture was diluted with EtOAc (170 mL), which was washed with sat. aq. NaHCO₃ (170 mL). The separated aqueous layer was extracted twice with EtOAc/MeOH (50 mL/1 mL). The combined organic layers were dried over sodium sulfate, filtered, and concentrated. The residue was purified by silica gel column chromatography (0-10% EtOAc in hexane, with 0.5% triethylamine in both phases), and then again by silica gel column chromatography (1.5% triethylamine in hexane), to afford (R)-4-(tert-butoxy)-6,8-difluoro-2-((1-((3-fluoropyrrolidin-1-yl)methyl)cyclopropyl)methoxy)quinazoline (Int-27-B). MS (ESI): m/z (M+H) 410.2.

### Step C: 4-(tert-butoxy)-7-(5,6-dimethyl-1-(tetrahydro-2H-pyran-2-yl)-1H-indazol-4-yl)-6,8-difluoro-2-((1-(((R)-3-fluoropyrrolidin-1-yl)methyl)cyclopropyl)methoxy)quinazoline (Int-27-C)

To (R)-4-(tert-butoxy)-6,8-difluoro-2-((1-((3-fluoropyrrolidin-1-yl)methyl)cyclopropyl)methoxy)quinazoline (Int-27-B) (1.75 g, 4.27 mmol) was added bis(2,2,6,6-tetramethylpiperidin-1-yl)zinc, complex with MgCl₂ and LiCl (0.13 M solution in THF/toluene, Aldrich cat. no. 748188-100ML, 36 mL, 4.7 mmol). The solution was heated at 40° C. with stirring for 1 h and 21 min, and then was allowed to stir at room temperature for 37 min. Then anhydrous 1,4-dioxane (43 mL) was added, and the mixture was stirred at room temperature for 15 min. Then 4-bromo-5,6-dimethyl-1-(tetrahydro-2H-pyran-2-yl)-1H-indazole (2.65 g, 8.55 mmol) was added, followed by Pd (CPhos) G3 (515 mg, 0.639 mmol). The mixture was heated with stirring under nitrogen at 80° C. for 36 min, then cooled to room temperature. The mixture was diluted with EtOAc (80 mL) and water (40 mL). The mixture was filtered, and the filtered residue was extracted with EtOAc (200 mL). The combined filtrates were concentrated by rotary evaporation, and the residue was suspended in EtOAc (80 mL) and water (40 mL). After mixing, the layers were separated, and the aqueous layer was extracted with EtOAc (30 mL). The combined organic layers were dried over sodium sulfate, filtered, and concentrated. The residue was purified by silica gel column chromatography (30-40% DCM in hexane, with 1.5% triethylamine in both phases), and then by silica gel column chromatography (10-15% DCM in hexane, with 1.5% triethylamine in both phases), and again by silica gel column chromatography (20% DCM in hexane, with 1.5% triethylamine in both phases), to afford 4-(tert-butoxy)-7-(5,6-dimethyl-1-(tetrahydro-2H-pyran-2-yl)-1H-indazol-4-yl)-6,8-difluoro-2-((1-(((R)-3-fluoropyrrolidin-1-yl)methyl)cyclopropyl)methoxy)quinazoline (mixture of atropisomers) which was purified by SFC (Column A, 40% IPA w/0.1% NH4OH), where 4 peaks were resolved. The first two-eluting peaks from SFC purification were combined to afford 4-(tert-butoxy)-7-(5,6-dimethyl-1-(tetrahydro-2H-pyran-2-yl)-1H-indazol-4-yl)-6,8-difluoro-2-((1-(((R)-3-fluoropyrrolidin-1-yl)methyl)cyclopropyl)methoxy)quinazoline (Int-27-C). MS (ESI): m/z (M+H)⁺ 638.3

### Step D: 7-(5,6-dimethyl-1-(tetrahydro-2H-pyran-2-yl)-1H-indazol-4-yl)-6,8-difluoro-2-((1-(((R)-3-fluoropyrrolidin-1-yl)methyl)cyclopropyl)methoxy)quinazolin-4-ol (Int-27)

To 4-(tert-butoxy)-7-(5,6-dimethyl-1-(tetrahydro-2H-pyran-2-yl)-1H-indazol-4-yl)-6,8-difluoro-2-((1-(((R)-3-fluoropyrrolidin-1-yl)methyl)cyclopropyl)methoxy)quinazoline (Int-27-C) (500 mg, 0.784 mmol) was added a solution of MeCN/water/trifluoroacetic acid (4:1:0.05, v/v/v) (19 mL). The solution was stirred at room temperature for 1 h and 20 min; then aq. sodium carbonate (2M solution, 1.53 mL) was added. The mixture was concentrated, and the residue was dissolved in DCM and water. The aqueous layer was extracted with DCM four times, then the combined organic layers were dried over sodium sulfate, filtered and concentrated to afford 7-(5,6-dimethyl-1-(tetrahydro-2H-pyran-2-yl)-1H-indazol-4-yl)-6,8-difluoro-2-((1-(((R)-3-fluoropyrrolidin-1-yl)methyl)cyclopropyl)methoxy)quinazolin-4-ol (Int-27). MS (ESI): m/z (M+H) 582.3.

### Intermediate 28: 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (Int-28)

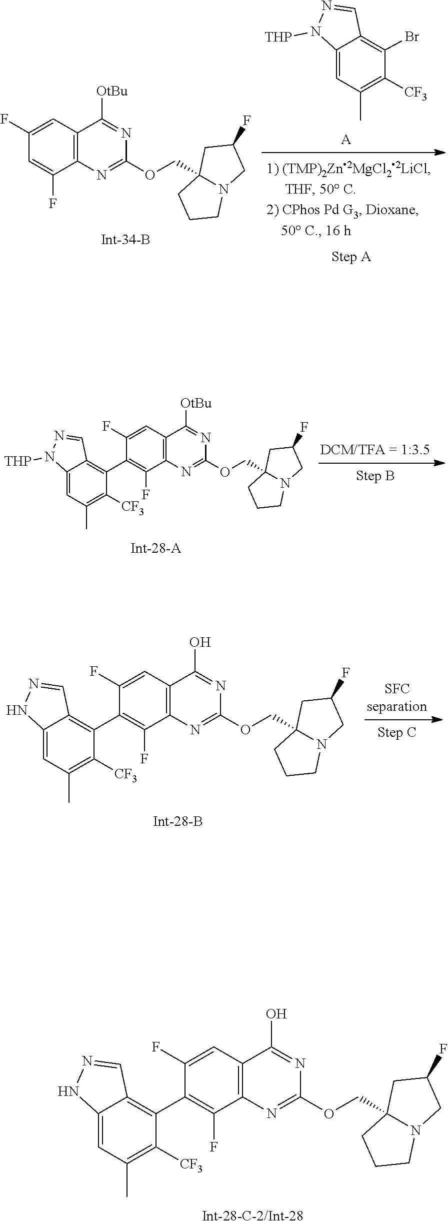

### Step A: 4-(tert-butoxy)-6,8-difluoro-2-(((2R,7aS)-2-fluorotetrahydro-1H-pyrrolizin-7a(5H)-yl)methoxy)-7-(6-methyl-1-(tetrahydro-2H-pyran-2-yl)-5-(trifluoromethyl)-1H-indazol-4-yl)quinazoline (Int-28-A)

To 4-(tert-butoxy)-6,8-difluoro-2-(((2R,7aS)-2-fluorotetrahydro-1H-pyrrolizin-7a(5H)-yl)methoxy)quinazoline (Int-34-B below) (1.8 g, 4.55 mmol) was added (TMP)₂Zn·2MgCl₂·2LiCl (41.4 mL, 13.66 mmol) (0.33 M in THF) at 25° C. under N₂ atmosphere, and the mixture was stirred at 50° C. for 2 h. The reaction mixture was monitored by LCMS-1 that showed the exchange was completed (quenched with 12 (dissolved in THF)). Then to the reaction mixture was added a solution of 4-bromo-6-methyl-1-(tetrahydro-2H-pyran-2-yl)-5-(trifluoromethyl)-1H-indazole (2.480 g, 6.83 mmol) and CPhos Pd G3 (0.367 g, 0.455 mmol) in 1,4-dioxane (30 mL) at 25° C. The reaction was stirred at 50° C. for 40 h. The reaction mixture was diluted with EtOAc (60 mL), and saturated aqueous NaHCO₃ solution (50 mL) was added to the mixture. The mixture was filtered, and the filtrate was dried over Na₂SO₄, then the solvent was evaporated under reduced pressure to give the crude product. The crude product was purified by flash silica gel chromatography (eluting with ethyl acetate in hexanes) to give 4-(tert-butoxy)-6,8-difluoro-2-(((2R,7aS)-2-fluorotetrahydro-1H-pyrrolizin-7a(5H)-yl)methoxy)-7-(6-methyl-1-(tetrahydro-2H-pyran-2-yl)-5-(trifluoromethyl)-1H-indazol-4-yl)quinazoline (Int-28-A). MS (ESI) [M+H]⁺: m/z 678.3.

### Step B: 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (Int-28-B)

To a solution of TFA (9 mL, 117 mmol) in DCM (30 mL) was added 4-(tert-butoxy)-6,8-difluoro-2-(((2R,7aS)-2-fluorotetrahydro-1H-pyrrolizin-7a(5H)-yl)methoxy)-7-(6-methyl-1-(tetrahydro-2H-pyran-2-yl)-5-(trifluoromethyl)-1H-indazol-4-yl)quinazoline (Int-28-A) (4 g, 5.90 mmol) at 25° C. The reaction was stirred at 25° C. for 6 h. The solvent was evaporated under reduced pressure to give the crude which was washed by saturated aqueous NaHCO₃ (3×20 mL). The combined organic phase was dried over anhydrous Na₂SO₄, filtered and concentrated in vacuo to give 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (Int-28-B). MS (ESI) [M+H]⁺: m/z 538.2.

### Step C: 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (Int-28)

The racemic 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (Int-28-B) (3.10 g, 5.77 mmol) was separated by preparative SFC (Column G; 0.1% NH4OH, 60% EtOH) to give 6,8-difluoro-2-(((2R,7aS)-2-fluorotetrahydro-1H-pyrrolizin-7a(5H)-yl)methoxy)-7-(6-methyl-5-(trifluoromethyl)-1H-indazol-4-yl)quinazolin-4-ol (Int-28, the second eluting isomer from SFC). MS (ESI) [M+H]⁺: m/z 538.2. ¹H NMR (400 MHz, MeOD) δ 7.78-7.70 (m, 1H), 7.68 (s, 1H), 7.59 (s, 1H), 5.52-5.25 (m, 1H), 4.50-4.36 (m, 2H), 3.63-3.40 (m, 3H), 3.16 (dt, J=5.9, 9.8 Hz, 1H), 2.70 (br d, J=1.5 Hz, 3H), 2.47-2.28 (m, 2H), 2.25-2.16 (m, 1H), 2.15-2.04 (m, 2H), 2.02-1.90 (m, 1H).

### Intermediate 29: (3R)-1-(2-chloro-7-(8-chloro-3-(methoxymethoxy)naphthalen-1-yl)-6,8-difluoroquinazolin-4-yl)-3-methylpiperidin-3-ol (Int-29-1, Int-29-2)

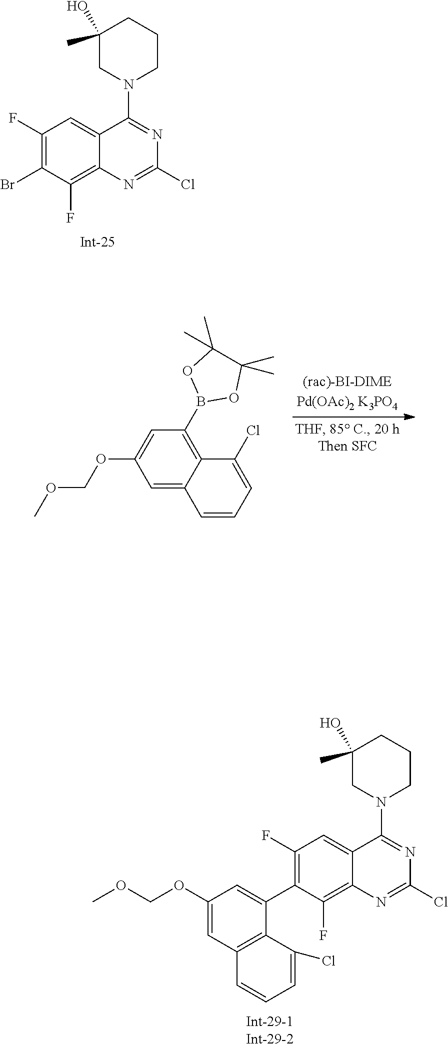

A mixture of (R)-1-(7-bromo-2-chloro-6,8-difluoroquinazolin-4-yl)-3-methylpiperidin-3-ol (Int-25) (775 mg, 1.97 mmol) and 2-(8-chloro-3-(methoxymethoxy)naphthalen-1-yl)-4,4,5,5-tetramethyl-1,3,2-dioxaborolane (826 mg, 2.37 mmol) in tetrahydrofuran (10.0 ml) was treated at rt with potassium phosphate tribasic (1257 mg, 5.92 mmol), (S)—BI-DIME (130 mg, 0.395 mmol), (R)—BI-DIME (130 mg, 0.395 mmol) and palladium(II) acetate (89 mg, 0.395 mmol). The mixture was subsurface sparged with a nitrogen stream for 1 minute. The vial was capped and the mixture was heated at 85° C. for 20 h. The mixture was cooled to rt, diluted with ethyl acetate and filtered through a stratified pad of Florisil over Celite. Pad was rinsed with ethyl acetate, and the combined filtrate was concentrated. The residue was purified by column chromatography on silica gel (eluting with EtOAc in hexanes, 0-100% gradient) and afforded the title compound as a mixture of atropisomers. The atropisomers were resolved by chiral SFC (Column H, 20% MeOH w/0.1% NH4OH) to afford Atropisomer 1 and Atropisomer 2.

### Atropisomer 1 (Int-29-1):

MS (ESI): m/z (M+H)+ 534, 536.

1H NMR (500 MHz, Acetone-d6) δ 8.01-7.97 (m, 1H), 7.95 (d, J=10.1 Hz, 1H), 7.75 (s, 1H), 7.55-7.49 (m, 2H), 7.39 (s, 1H), 5.43 (s, 2H), 4.42 (d, J=13.2 Hz, 1H), 4.23 (d, J=13.4 Hz, 1H), 3.99 (s, 1H), 3.54 (d, J=13.4 Hz, 1H), 3.52 (s, 3H), 3.36 (t, J=11.2 Hz, 1H), 2.29-2.18 (m, 1H), 1.91-1.84 (m, 1H), 1.83-1.74 (m, 2H), 1.30 (s, 3H).

### Atropisomer 2 (Int-29-2):

MS (ESI): m/z (M+H)+ 534, 536.

1H NMR (500 MHz, Acetone-d6) δ 8.02-7.97 (m, 2H), 7.75 (s, 1H), 7.53-7.48 (m, 2H), 7.38 (s, 1H), 5.42 (s, 2H), 4.40 (d, J=13.2 Hz, 1H), 4.23 (d, J=13.4 Hz, 1H), 4.06 (s, 1H), 3.52 (s, 3H), 3.48 (d, J=13.4 Hz, 1H), 3.36 (t, J=11.8 Hz, 1H), 2.27-2.16 (m, 1H), 1.92-1.85 (m, 1H), 1.83-1.74 (m, 2H), 1.32 (s, 3H).

The compound in the table below was synthesized using a similar procedure as described in the synthesis of Int-29 by making the appropriate substitutions for starting material, intermediates, and/or reagents optionally resolving stereoisomers after completion of the indicated step using the indicated conditions and carrying forward the indicated peak. Appropriate substitutions are available commercially, synthesized as described in the literature, synthesized using methods available to those skilled in the art, or synthesized as described herein.

| | | | | [M/2 + H]⁺ |
|---|---|---|---|---|
| Int | Structure | Compound Name | Resolution | Found |
| Int-30 | 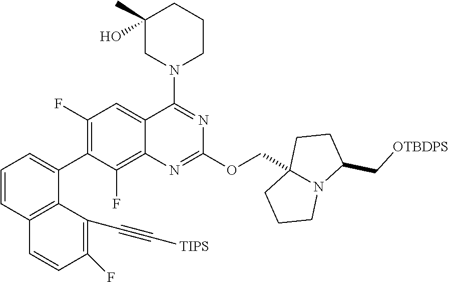 | (3R)-1-(2-(((3R,7aR)-3- (((tert-butyl- diphenylsilyl)oxy)meth- yl)tetrahydro-1H- pyrrolizin-7a(5H)- yl)methoxy)-6,8-difluoro- 7-(7-fluoro-8-((triiso propylsilyl)ethynyl)naph- thalen-1-yl)quinazolin- 4-yl)-3-methylpiperidin-3- ol | Column A. 55% EtOH with 0.1% NH₃H₂O, Peak 1 | 506 |

### Intermediate 31: (3S)-1-(7-(8-chloronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Int-31)

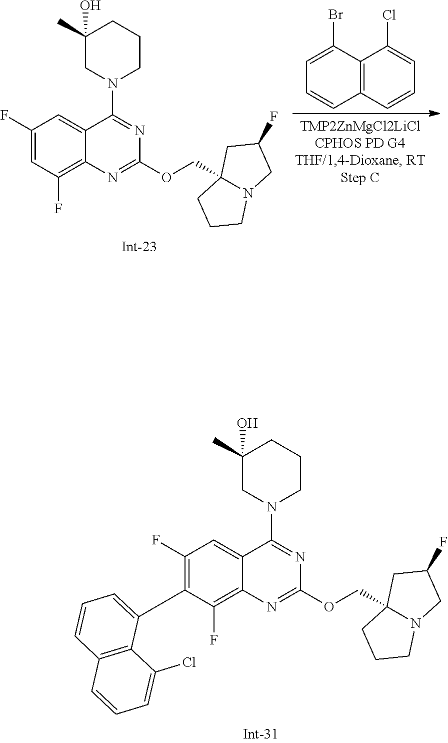

A microwave vial was charged with (R)-1-(6,8-difluoro-2-(((2R,7aS)-2-fluorotetrahydro-TH-pyrrolizin-7a(5H)-yl)methoxy)quinazolin-4-yl)-3-methylpiperidin-3-ol (Int-23) (178 mg, 0.408 mmol). It was then degasified with line vacuum and nitrogen thrice and flushed with argon thrice. To this was added THF (4100 ml) followed by TMP₂ZnMgCl₂LiCl (5.44 ml, 0.816 mmol, in THF/toluene). The solution was purged with argon for 5 mins and stirred at RT for 45 mins. An aliquot was quenched into a mixture of iodine and THF. A mixture of 1-bromo-8-chloronaphthalene (197 mg, 0.816 mmol) and CPHOS PD G4 (50.3 mg, 0.061 mmol) were dissolved in 1,4-dioxane (4100 ml) and sonicated until it turned into a clear solution. This mixture was added to the microwave vial and stirred at RT for 45 mins. The mixture was diluted with ethyl acetate and saturated aqueous ammonium chloride. The organic layer was separated, dried over magnesium sulfate, filtered and concentrated under reduced pressure. The resulting mixture was purified by silica gel column chromatography with 100% of EtOAc in hexane to get (3S)-1-(7-(8-chloronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Int-31). MS (ESI): m/z (M+H)⁺ 597.

The compound in the table below was synthesized using a similar procedure as described in the synthesis of Int-31 by making the appropriate substitutions for starting material, intermediates, and/or reagents. Appropriate substitutions are available commercially, synthesized as described in the literature, synthesized using methods available to those skilled in the art, or synthesized as described herein.

| | | | [M + H]⁺ |
|---|---|---|---|
| Int | Structure | Compound Name | Found |
| Int-32 | 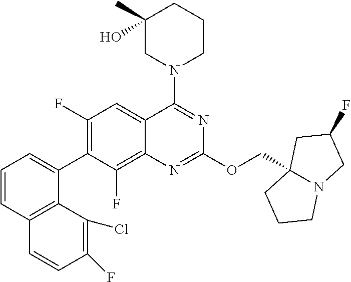 [SMILES: C[C@@]1(O)CCCN(c2nc(OC[C@@]34CCCN3C[C@H](F)C4)nc3c(F)c(-c4cccc5ccc(F)c(Cl)c45)c(F)cc23)C1] | (3R)-1-(7-(8-chloro-7- fluoronaphthalen-1-yl)- 6,8-difluoro-2-(((2R,7aS)- 2-fluorotetrahydro-1H- pyrrolizin-7a(5H)- yl)methoxy)quinazolin-4- yl)-3-methylpiperidin-3-ol | 615 |

### Intermediate 33: 1-(7-(8-chloro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-(hydroxymethyl)piperidin-3-ol (Int-33)

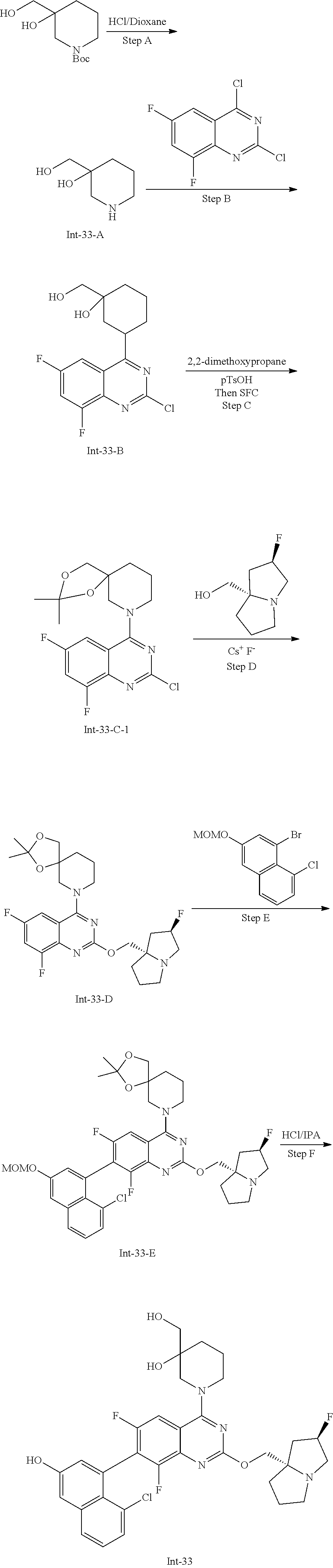

### Step A: (rac)3-(hydroxymethyl)piperidin-3-ol (Int-33-A)

To a solution of (rac)tert-butyl 3-hydroxy-3-(hydroxymethyl)piperidine-1-carboxylate (1.00 g, 4.32 mmol) in dioxane (14 ml) was added HCl (16 ml, 65 mmol, 4M in dioxane) and the reaction stirred at RT for 5 hours. The organic solvent was removed under reduced pressure and to the residue was added ethyl ether and co-evaporated to give the title compound (Int-33-A).

### Step B: (rac)1-(2-chloro-6,8-difluoroquinazolin-4-yl)-3-(hydroxymethyl)piperidin-3-ol (Int-33-B)

To a solution of 2,4-dichloro-6,8-difluoroquinazoline (Int-11) (1.2 g, 5.0 mmol) in dioxane (14 ml) and DIPEA (3.8 ml, 22 mmol) was added Int-33-A, and the resulting mixture was stirred at RT for 2.5 h. The reaction was quenched with cold water, and the resulting mixture was diluted with ethyl acetate. The organic layer was separated, washed with brine, dried over MgSO4, filtered and excess solvent was removed under reduced pressure. The product was purified by column chromatography on silica gel (MeOH in DCM, 0-15% gradient). Product-containing fractions were pooled together, and excess solvent was removed under reduced pressure to give the title compound (Int-33-B). MS (ESI): m/z (M+H)⁺ 330.1.

### Step C: 7-(2-chloro-6,8-difluoroquinazolin-4-yl)-2,2-dimethyl-1,3-dioxa-7-azaspiro[4.5]decane (Int-33-C-1)

To a solution of (rac)1-(2-chloro-6,8-difluoroquinazolin-4-yl)-3-(hydroxymethyl)piperidin-3-ol (Int-33-B) (1.2 g, 3.8 mmol) in DMF (20 ml) and 2,2-dimethoxypropane (0.93 ml, 7.5 mmol) was added pTsOH (716 mg, 3.77 mmol), and the resulting mixture was stirred at RT. The reaction was quenched with cold water, and the resulting mixture was diluted with ethyl acetate. The organic layer was separated, washed with brine, dried over MgSO4, filtered and excess solvent was removed under reduced pressure. The product was purified by column chromatography on silica gel (MeOH in DCM, 0-15% gradient). Fractions containing the desired product were pooled together, excess solvent was removed under reduced pressure, and then dried under vacuum to give a yellow solid. The solid obtained was separated on SFC (Column H, 10% MeOH with 0.1% NH₄OH) to give two enantiomers. Int-33-C-1 (first eluting), 7-(2-chloro-6,8-difluoroquinazolin-4-yl)-2,2-dimethyl-1,3-dioxa-7-azaspiro[4.5]decane was obtained as a yellow solid MS (ESI): m/z (M+H)⁺ 370.1, and Int-33-C-2 (second eluting), 7-(2-chloro-6,8-difluoroquinazolin-4-yl)-2,2-dimethyl-1,3-dioxa-7-azaspiro[4.5]decane was obtained as a yellow foam MS (ESI): m/z (M+H)⁺ 370.1.

### Step D: 7-(6,8-difluoro-2-(((2R,7aS)-2-fluorotetrahydro-1H-pyrrolizin-7a(5H)-yl)methoxy)quinazolin-4-yl)-2,2-dimethyl-1,3-dioxa-7-azaspiro[4.5]decane (Int-33-D)

To a solution of 7-(2-chloro-6,8-difluoroquinazolin-4-yl)-2,2-dimethyl-1,3-dioxa-7-azaspiro[4.5]decane (Int-33-C-1) (200. mg, 0.541 mmol) and ((2R,7aS)-2-fluorotetrahydro-1H-pyrrolizin-7a(5H)-yl)methanol (Int-8) (172 mg, 1.082 mmol) in DMSO (1.00 ml) was added cesium fluoride (329 mg, 2.16 mmol), and the resulting mixture was stirred at 100° C. for 3 hrs. The reaction mixture was cooled to room temperature and diluted with water. This was partitioned into ethyl acetate and water. The aqueous layer was extracted twice with ethyl acetate. The organic layers were combined, washed with brine, dried over MgSO4, filtered and excess solvent was removed under reduced pressure. The product was purified by column chromatography on silica gel (MeOH in DCM, 0-15% gradient). Product-containing fractions were pooled together, excess solvent was removed under reduced pressure, and this was vacuum dried to give the title compound (Int-33-D). MS (ESI): m/z (M+H)⁺ 493.3.

### Step E: 7-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,2-dimethyl-1,3-dioxa-7-azaspiro[4.5]decane (Int-33-E)

To a vial thoroughly purged with N₂, TMP₂Z—MgClLiCl (1.096 mL, 0.164 mmol, in THF/Toluene) was added. To this a solution of 7-(6,8-difluoro-2-(((2R,7aS)-2-fluorotetrahydro-1H-pyrrolizin-7a(5H)-yl)methoxy)quinazolin-4-yl)-2,2-dimethyl-1,3-dioxa-7-azaspiro[4.5]decane (Int-33-D) (123.0 mg, 0.250 mmol) in THF (1 mL) was added, and the reaction mixture was stirred at RT for an hour. A solution of 1-bromo-8-chloro-3-(methoxymethoxy)naphthalene (136 mg, 0.450 mmol) and CPHOS PD G4 (22 mg, 0.027 mmol) in dioxane (1.00 ml) was added to the above reaction mixture and stirred for 2 hours on a preheated oil bath at 100° C. for 2.5 hours. The reaction was cooled in ice and quenched with water. Excess solvent was removed in-vacuo, and the residue was partitioned into ethyl acetate and water. The aqueous layer was extracted twice with ethyl acetate, the organic layers were pooled together, excess solvent was removed under reduced pressure, and the residue was purified on a silica gel column (eluting with MeOH in DCM, 0-15% gradient). Fractions containing the desired product were pooled together, excess solvent was removed under reduced pressure and the residue was vacuum dried to give 7-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,2-dimethyl-1,3-dioxa-7-azaspiro[4.5]decane (Int-33-E). MS (ESI): m/z (M+H)⁺ 713.2.

### Step F: 1-(7-(8-chloro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-(hydroxymethyl)piperidin-3-ol (Int-33)

To a solution of 7-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-2,2-dimethyl-1,3-dioxa-7-azaspiro[4.5]decane (Int-33-E) (158 mg, 0.222 mmol) in isopropanol (1.00 ml) was added HCl (1.1 ml, 4.4 mmol), and the reaction mixture was stirred at 45° C. for 4 hours. Excess solvent was removed under reduced pressure. The residue was dissolved in MeOH and directly purified on a reverse phase HPLC (C18, ACN-Water w/0.5% TFA, 0-100% gradient). Fractions containing the desired product were pooled together, and lyophilized. 1-(7-(8-chloro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-(hydroxymethyl)piperidin-3-ol was obtained as a yellow oil as a mixture of atropisomers (Int-33). MS (ESI): m/z (M+H)⁺ 629.2.

### Intermediate 34: 7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (Int-34)

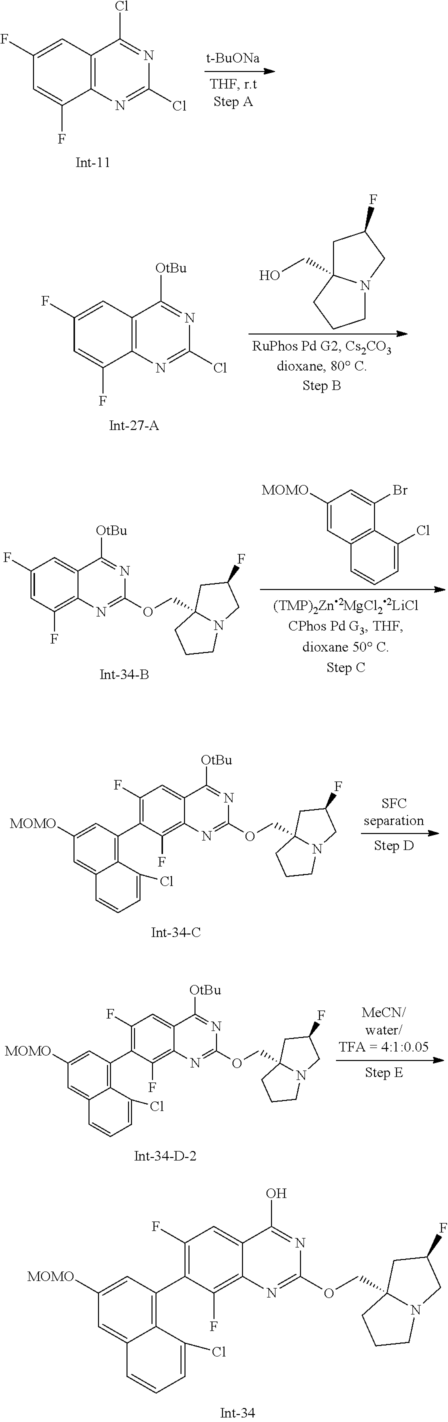

### Step A: 4-(tert-butoxy)-2-chloro-6,8-difluoroquinazoline (Int-27-A)

To a solution of 2,4-dichloro-6,8-difluoroquinazoline (Int-11) (10 g, 42.6 mmol) in THF (100 mL) was added sodium tert-butoxide (21.28 mL, 42.6 mmol, 2.0 M in THF) at 0° C. under N₂ atmosphere. The mixture was stirred at 25° C. for 2 h. The mixture was diluted with EtOAc (200 mL), washed with H₂O (30 mL), dried over Na₂SO₄, filtered, and the solvent was evaporated under reduced pressure to give the crude product. The crude product was purified by flash silica gel chromatography (eluting with ethyl acetate in petroleum ether, 0-100% gradient) to give 4-(tert-butoxy)-2-chloro-6,8-difluoroquinazoline (Int-27-A). ¹H NMR (400 MHz, CDCl₃) δ 7.52-7.44 (m, 1H), 7.35-7.27 (m, 1H), 1.74 (s, 9H).

### Step B: 4-(tert-butoxy)-6,8-difluoro-2-(((2R,7aS)-2-fluorotetrahydro-1H-pyrrolizin-7a(5H)-yl)methoxy)quinazoline (Int-34-B)

To a solution of 4-(tert-butoxy)-2-chloro-6,8-difluoroquinazoline (Int-34-A) (4 g, 14.67 mmol) and ((2R,7aS)-2-fluorotetrahydro-1H-pyrrolizin-7a(5H)-yl)methanol (Int-8) (3.50 g, 22.00 mmol) in dioxane (60 mL) were added Cs₂CO₃ (9.56 g, 29.3 mmol) and RuPhos Pd G2 (1.139 g, 1.467 mmol) in a glove box, and the reaction mixture was stirred at 80° C. for 15 h under N₂ atmosphere. The reaction mixture was quenched with water (20 mL) and extracted with EtOAc (100 mL×3). The combined organic phase was dried over anhydrous Na₂SO₄, filtered and concentrated in vacuo. The residue was purified by flash silica gel chromatography (eluting with EtOAc in hexanes, 0-100% gradient) to give 4-(tert-butoxy)-6,8-difluoro-2-(((2R,7aS)-2-fluorotetrahydro-TH-pyrrolizin-7a(5H)-yl)methoxy)quinazoline (Int-34-B). MS (ESI) [M+H]⁺ m/z 396.

### Step C: 4-(tert-butoxy)-7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazoline (Int-34-C)

To a solution of (TMP)₂Zn·2MgCl₂·2LiCl (90 mL, 29.6 mmol, 0.33 M in THF) was added 4-(tert-butoxy)-6,8-difluoro-2-(((2R,7aS)-2-fluorotetrahydro-1H-pyrrolizin-7a(5H)-yl)methoxy)quinazoline (Int-34-B) (3.9 g, 9.86 mmol) at 25° C. in a glove box, and the mixture was stirred at 50° C. for 3 h. 1-bromo-8-chloro-3-(methoxymethoxy)naphthalene (3.57 g, 11.84 mmol) and CPhos Pd G3 (0.795 g, 0.986 mmol) in THF (24 mL) were added to the reaction solution at 25° C. in the glove box; then the reaction mixture was stirred at 50° C. for 15 h. LCMS showed starting material was consumed and the desired MS was observed. The mixture was diluted with EtOAc (300 mL), and saturated NaHCO₃ solution (30 mL) was added to the mixture. The mixture was filtered and separated. The organic layer was dried over Na₂SO₄, and the solvent was evaporated under reduced pressure to give the crude product. The crude product was purified by flash silica gel chromatography (eluting with ethyl acetate in hexanes, 0-100% gradient) to give 4-(tert-butoxy)-7-(8-chloro-3-(methoxymethoxy)naphthalen-1-yl)-6,8-difluoro-2-(((2R,7aS)-2-fluorotetrahydro-TH-pyrrolizin-7a(5H)-yl)methoxy)quinazoline (Int-34-C). MS (ESI) [M+H]⁺: m/z 616.

### Step D: 4-(tert-butoxy)-7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazoline (Int-34-D-2)

The mixture of 4-(tert-butoxy)-7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazoline (Int-34-C) (3.8 g, 6.17 mmol) was separated by preparative SFC (Column G, 30% EtOH with 0.1% NH₃H₂O) to afford 4-(tert-butoxy)-7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazoline (the second eluting isomer) (Int-34-D-2). MS (ESI) [M+H]⁺: m/z 616.

### Step E: 7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (Int-34)

A mixture of 4-(tert-butoxy)-7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazoline (Int-34-D-2) (1.5 g, 2.435 mmol) in MeCN (16 mL), water (4 mL) and TFA (0.4 mL) was stirred at 25° C. for 1.5 h. The reaction was concentrated to remove MeCN, and the mixture was purified by preparative HPLC (C18, ACN in water with 0.1% TFA) to give 7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol as the TFA salt (Int-34). MS (ESI) [M+H]⁺: m/z 560.

### Intermediate 35: 7-(8-chloro-3-(methoxymethoxy)naphthalen-1-yl)-2-(((R)-2,2-difluoro-1-(hydroxymethyl)cyclopropyl)methoxy)-6,8-difluoroquinazolin-4-ol (Int-35)

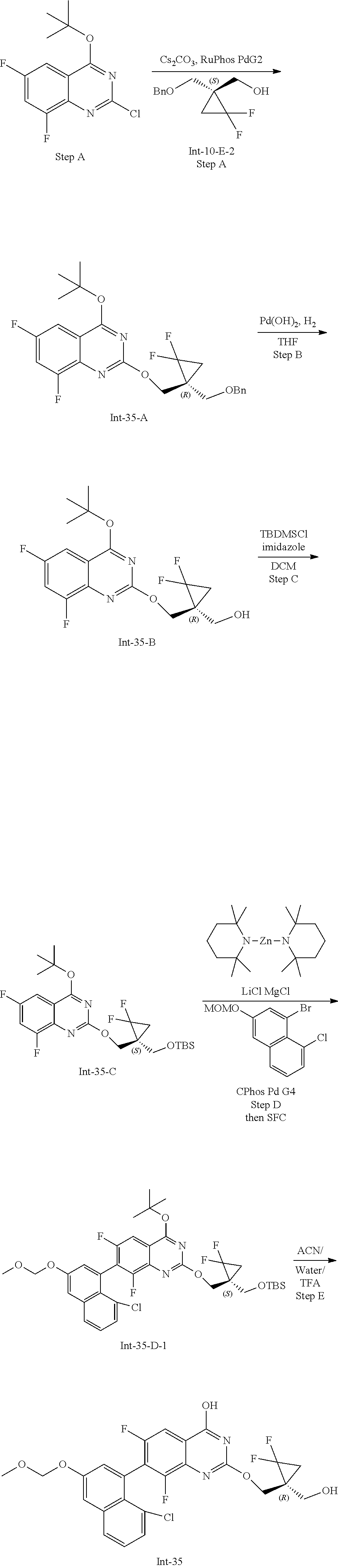

### Step A: (R)-2-((1-((benzyloxy)methyl)-2,2-difluorocyclopropyl)methoxy)-4-(tert-butoxy)-6,8-difluoroquinazoline (Int-35-A)

To a mixture of chloro(2-dicyclohexylphosphino-2′,6′-diisopropoxy-1,1′-biphenyl)[2-(2′-amino-1,1′-biphenyl)]palladium(II) (1.85 g, 2.38 mmol), cesium carbonate (23.3 g, 72 mmol) and 4-(tert-butoxy)-2-chloro-6,8-difluoroquinazoline (Int-27-A) (6.5 g, 24 mmol) was added a solution of (S)-(1-((benzyloxy)methyl)-2,2-difluorocyclopropyl)methanol (8.16 g, 35.8 mmol) in dioxane (100 ml) under argon. The reaction mixture was stirred at 100° C. under argon overnight. The reaction mixture was allowed to cool to room temperature, diluted with EtOAc (300 mL), filtered (Celite) and washed with sodium bicarbonate (sat. aq., 3×100 mL). Organics were retained and concentrated under reduced pressure. The resulting mixture was purified by silica gel column chromatography (ethyl acetate gradient in hexanes) to afford the title compound (Int-35-A). MS (ESI): m/z (M+H-tBu)⁺ 409.

### Step B: (R)-(1-(((4-(tert-butoxy)-6,8-difluoroquinazolin-2-yl)oxy)methyl)-2,2-difluorocyclopropyl)methanol (Int-35-B)

(R)-2-((1-((benzyloxy)methyl)-2,2-difluorocyclopropyl)methoxy)-4-(tert-butoxy)-6,8-difluoroquinazoline (Int-35-A) (4 g, 9 mmol) in THF (43 mL) was added to a flask containing palladium hydroxide (0.61 g, 0.86 mmol) under an inert atmosphere. Hydrogen was allowed to effervesce through the reaction mixture before being sealed with a hydrogen balloon. The reaction mixture was stirred for 6 h at room temperature. Argon was allowed to effervesce through the reaction mixture before the reaction mixture was filtered through celite under argon. The filtrate was concentrated in vacuo. The resulting mixture was purified by silica gel column chromatography (ethyl acetate gradient in hexanes) to afford the title compound. MS (ESI): m/z (M+H-tBu)+ 319. 1H NMR (499 MHz, Methanol-d4) δ 7.55-7.45 (m, 2H), 4.69 (d, J=12 Hz, 1H), 4.60 (d, J=12 Hz, 1H), 3.85 (d, J=12 Hz, 1H), 3.79 (d, J=12 Hz, 1H), 1.79 (s, 9H), 1.68-1.57 (m, 1H), 1.55-1.45 (m, 1H).

### Step C: (S)-4-(tert-butoxy)-2-((1-(((tert-butyldimethylsilyl)oxy)methyl)-2,2-difluorocyclopropyl)methoxy)-6,8-difluoroquinazoline (Int-35-C)

(R)-(1-(((4-(tert-butoxy)-6,8-difluoroquinazolin-2-yl)oxy)methyl)-2,2-difluorocyclopropyl)methanol (Int-35-B) (5.2 g, 14 mmol) was dissolved in dichloromethane (70 ml) and was treated with imidazole (2.8 g, 41 mmol) followed by three equal portions addition of TBDMS-Cl (2.7 g, 18 mmol) at 0° C. The reaction mixture was allowed to warm to and stir at rt for 2 h. The reaction mixture was diluted with DCM (300 mL) and washed with sodium bicarbonate (3×100 mL). Combined organic phase was concentrated in vacuo. The resulting mixture was purified by silica gel column chromatography (ethyl acetate gradient in hexanes). Product-containing fractions were concentrated in vacuo to give the title compound (Int-35-C). MS (ESI): m/z (M+Na)+511. ¹H NMR (Methanol-d4) δ: 7.55-7.42 (m, 2H), 4.62 (s, 2H), 3.98 (d, J=10.8 Hz, 1H), 3.83 (d, J=10.9 Hz, 1H), 1.68-1.59 (m, 1H), 1.52-1.43 (m, 1H), 0.87 (s, 9H), 0.05 (s, 6H).

### Step D: 4-(tert-butoxy)-2-(((S)-1-(((tert-butyldimethylsilyl)oxy)methyl)-2,2-difluorocyclopropyl)methoxy)-7-(8-chloro-3-(methoxymethoxy)naphthalen-1-yl)-6,8-difluoroquinazoline (Int-35-D)

(S)-4-(tert-butoxy)-2-((1-(((tert-butyldimethylsilyl)oxy)methyl)-2,2-difluorocyclopropyl)methoxy)-6,8-difluoroquinazoline (Int-35-C) (19 ml, 5.1 mmol) in THF was added to dis(2,2,6,6-tetramethylpiperidinyl)zinc-LiCl—MgCl₂ in THF/toluene (19 ml, 6.1 mmol). The reaction mixture was stirred for 1 h at room temperature. A separate mixture of CPhos Pd G4 (0.63 g, 0.77 mmol), 1-bromo-8-chloro-3-(methoxymethoxy)naphthalene (3.1 g, 10. mmol) in dioxane (25 ml) was prepared with sonication and gentle heating until homogeneous before adding into the zincate mixture. The reaction mixture was purged with argon before heating to 100° C. for overnight. The reaction mixture was diluted with EtOAc (200 mL) and washed with ammonium chloride (3×100 mL). Combined organics were concentrated in vacuo. The resulting mixture was purified by silica gel column chromatography (ethyl acetate gradient in hexanes). Product-containing fractions were concentrated in vacuo to give the title compound (Int-35-D). Atropisomers were resolved by chiral SFC (Column F, 15% MeOH w/0.1% NH4OH). (Int-35-D-1, Peak 1)—MS (ESI): m/z (M+H-tBu)+ 653. 1H NMR (499 MHz, DMSO-d₆) δ 8.03-7.96 (m, 1H), 7.76 (d, J=2.2 Hz, 1H), 7.63 (d, J=8.5 Hz, 1H), 7.53-7.48 (m, 2H), 7.36 (d, J=2.3 Hz, 1H), 5.40 (s, 2H), 4.64 (d, J=11.3 Hz, 1H), 4.46 (d, J=11.4 Hz, 1H), 3.87 (d, J=10.9 Hz, 1H), 3.77 (d, J=10.9 Hz, 1H), 3.45 (s, 3H), 1.87-1.78 (m, 1H), 1.74 (s, 9H), 1.67-1.55 (m, 1H), 0.79 (s, 9H), −0.01 (s, 6H). (Int-35-D-2, Peak 2—MS (ESI): m/z (M+H-tBu)+ 653. 1H NMR (499 MHz, DMSO-d₆) δ 8.04-7.94 (m, 1H), 7.76 (d, J=2.0 Hz, 1H), 7.63 (d, J=8.5 Hz, 1H), 7.53-7.48 (m, 2H), 7.35 (d, J=2.1 Hz, 1H), 5.40 (s, 2H), 4.57 (d, J=11.5 Hz, 1H), 4.53 (d, J=11.5 Hz, 1H), 3.88 (d, J=10.8 Hz, 1H), 3.75 (d, J=10.9 Hz, 1H), 3.45 (s, 3H), 1.82 (d, J=8.5 Hz, 1H), 1.74 (s, 9H), 1.70-1.58 (m, 1H), 0.79 (s, 9H), −0.01 (s, 3H), −0.01 (s, 3H).

### Step E: 7-(8-chloro-3-(methoxymethoxy)naphthalen-1-yl)-2-(((R)-2,2-difluoro-1-(hydroxvmethyl)cyclopropyl)methoxy)-6,8-difluoroquinazolin-4-ol (Int-35)

4-(tert-butoxy)-2-(((S)-1-(((tert-butyldimethylsilyl)oxy)methyl)-2,2-difluorocyclopropyl)methoxy)-7-(8-chloro-3-(methoxymethoxy)naphthalen-1-yl)-6,8-difluoroquinazoline (Int-35-D-1) (100. mg, 0.141 mmol, Peak 1) was dissolved in acetonitrile (1100 μL), Water (282 μl) and was subsequently treated with TFA (28 μl). The reaction mixture was stirred overnight and then quenched with sodium bicarbonate (sat. aq.). Product was extracted with DCM and concentrated in vacuo to yield the title compound (Int-35). MS (ESI): m/z (M+H)+ 539.

### Intermediate 36: 2—(((R)-2,2-difluoro-1-(hydroxymethyl)cyclopropyl)methoxy)-7-(8-ethynyl-3-(methoxymethoxy)naphthalen-1-yl)-6,8-difluoroquinazolin-4-ol (Int-36)

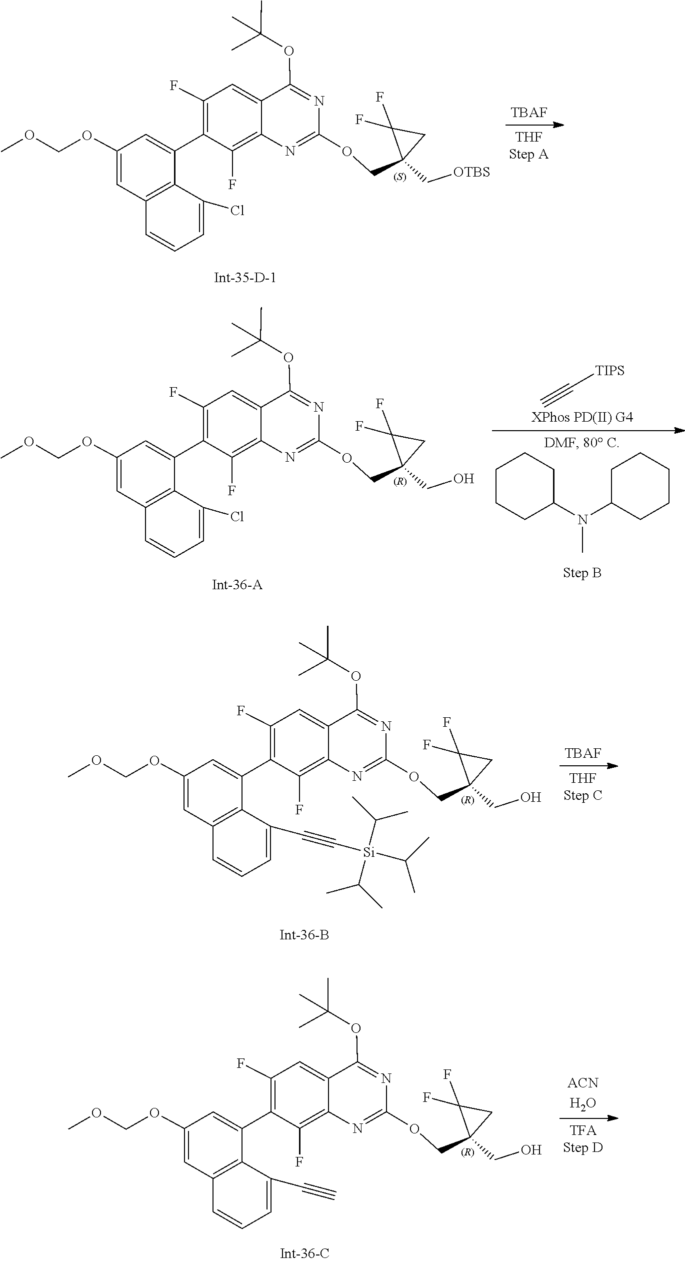

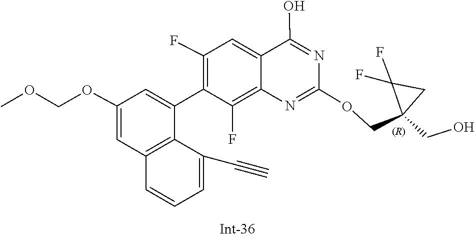

### Step A: ((1R)-1-(((4-(tert-butoxy)-7-(8-chloro-3-(methoxymethoxy)naphthalen-1-yl)-6,8-difluoroquinazolin-2-yl)oxy)methyl)-2,2-difluorocyclopropyl)methanol (Int-36-A)

TBAF (700 μl, 0.700 mmol, 1M in THF) was added to a stirring solution of 4-(tert-butoxy)-2-(((S)-1-(((tert-butyldimethylsilyl)oxy)methyl)-2,2-difluorocyclopropyl)methoxy)-7-(8-chloro-3-(methoxymethoxy)naphthalen-1-yl)-6,8-difluoroquinazoline (Int-35-D-1) (250 mg, 0.352 mmol) in THF (1760 μl) at ambient temperature. The reaction mixture was stirred for 2 h. Ammonium chloride was added (1 mL, sat. aq.) and stirred for 1 h. The product was extracted with EtOAc (3×10 mL) and the combined organics concentrated in vacuo. The product was purified by column chromatography on silica (0-100% EtOAc in Hexanes). Product-containing fractions were concentrated in vacuo to give the title compound (Int-36-A). MS (ESI): m/z (M+H-tBu)+ 539.

### Step B: ((1R)-1-(((4-(tert-butoxy)-6,8-difluoro-7-(3-(methoxymethoxy)-8-((triisopropylsilyl)ethynyl)naphthalen-1-yl)quinazolin-2-yl)oxy)methyl)-2,2-difluorocyclopropyl)methanol (Int-36-B)

((1R)-1-(((4-(tert-butoxy)-7-(8-chloro-3-(methoxymethoxy)naphthalen-1-yl)-6,8-difluoroquinazolin-2-yl)oxy)methyl)-2,2-difluorocyclopropyl)methanol (Int-36-A) (196 mg, 0.329 mmol) was dissolved in DMF (3294 μl). N,N-dicyclohexylmethylamine (350 μl, 1.6 mmol) and (triisopropylsilyl)acetylene (739 μl, 3.29 mmol) were sequentially added. The reaction mixture was degassed with argon and methanesulfonato(2-Dicyclohexylphosphino-2′,4′,6′-Tri-I-Propyl-1,1′-Biphenyl)(2′-Amino-1,1′-Biphenyl-2-yl)Palladium(II) (56 mg, 0.066 mmol) was added. Argon was allowed to effervesce through the reaction mixture before being sealed and stirred at 80° C. overnight. Reaction mixture was diluted with water (6 mL) and extracted with EtOAc (3×10 mL) and organics concentrated in vacuo. The product was purified by silica gel column chromatography (0-100%, EtOAc in Hexanes). Product-containing fractions were concentrated in vacuo to give the title compound (Int-36-B). MS (ESI): m/z (M+H-tBu)+ 685.

### Step C: ((1R)-1-(((4-(tert-butoxy)-7-(8-ethynyl-3-(methoxymethoxy)naphthalen-1-yl)-6,8-difluoroquinazolin-2-yl)oxy)methyl)-2,2-difluorocyclopropyl)methanol (Int-36-C)

TBAF (240 μl, 0.24 mmol, in THF) was added to a solution of ((1R)-1-(((4-(tert-butoxy)-6,8-difluoro-7-(3-(methoxymethoxy)-8-((triisopropylsilyl)ethynyl)naphthalen-1-yl)quinazolin-2-yl)oxy)methyl)-2,2-difluorocyclopropyl)methanol (Int-36-B) (90. mg, 0.12 mmol) in THF (1 ml) at ambient temperature. The reaction mixture was stirred for 1 h then treated with ammonium chloride (sat. aq., 1 mL). Product was extracted with EtOAc (3×1 mL). Organic layers were combined and then concentrated in vacuo. The product was purified by silica gel column chromatography (0-100% EtOAc in hexanes). Product containing fractions were concentrated in vacuo to give the title compound (Int-36-C). MS (ESI): m/z (M+H-tBu)⁺ 529.

### Step D: 2-(((R)-2,2-difluoro-1-(hydroxymethyl)cyclopropyl)methoxy)-7-(8-ethynyl-3-(methoxymethoxy)naphthalen-1-yl)-6,8-difluoroquinazolin-4-ol (Int-36)

TFA (15 μl) was added to a stirring mixture of ((1R)-1-(((4-(tert-butoxy)-7-(8-ethynyl-3-(methoxymethoxy)naphthalen-1-yl)-6,8-difluoroquinazolin-2-yl)oxy)methyl)-2,2-difluorocyclopropyl)methanol (Int-36-C) (45 mg, 0.077 mmol) in acetonitrile (600 μl) and water (154 μl). The reaction mixture was stirred at room temperature for 4 h before being concentrated in vacuo and lyophilized to give the title compound (Int-36). MS (ESI): m/z (M+H)+ 529.

### Intermediate 37: 7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (Int-37)

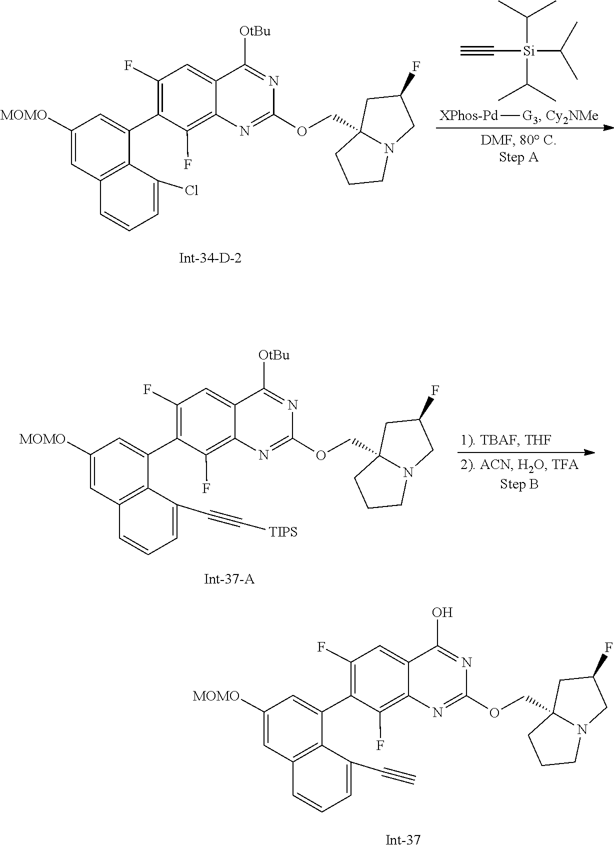

### Step A: 4-(tert-butoxv)-6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazoline (Int-37-A)

To a mixture of 4-(tert-butoxy)-7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazoline (0.50 g, 0.81 mmol) (Int-34-D-2) and DMF (5.4 mL) were added ethynyltriisopropylsilane (1.8 mL, 8.1 mmol), N-cyclohexyl-N-methylcyclohexanamine (0.52 mL, 2.4 mmol) and (2-dicyclohexylphosphino-2′,4′,6′-triisopropyl-1,1′-biphenyl)[2-(2′-amino-1,1′-biphenyl)]palladium(II) methanesulfonate (Xphos Pd G3) (0.34 g, 0.41 mmol) under a nitrogen atmosphere. The mixture was heated to 80° C. for 3 hours. After 3 hours, the mixture was allowed to cool to room temperature, diluted with ethyl acetate and then washed with water (3×8 mL). The organic layer was then dried over magnesium sulfate, filtered and then concentrated under reduced pressure. The resulting mixture was purified by silica gel column chromatography (0-30% ethyl acetate gradient in hexanes) to afford 4-(tert-butoxy)-6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazoline (Int-37-A). MS (ESI): m/z (M+H)⁺ 762.

### Step B: 7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (Int-37)

To a flask containing 4-(tert-butoxy)-6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazoline (Int-37-A) (0.37 g, 0.49 mmol) and THF (9.7 mL) was added TBAF (1.0 M in THF, 1.2 mL, 1.2 mmol). The mixture was allowed to stir at room temperature for 30 minutes. After 30 minutes, the mixture was diluted with ethyl acetate and saturated ammonium chloride. The organic layer was separated, dried over magnesium sulfate, filtered and concentrated under reduced pressure.

The resulting mixture was purified by silica gel column chromatography (0-100% (3:1 ethyl acetate: ethanol) gradient in hexanes) to afford a mixture of 4-(tert-butoxy)-7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazoline and 7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (Int-37). To this mixture was added acetonitrile (3.2 mL), water (0.81 mL) and TFA (0.81 mL). The mixture was allowed to stir for 2 hours at ambient temperature. After 2 hours, the mixture was diluted with ethyl acetate and saturated aqueous sodium bicarbonate. The organic layer was separated, dried over magnesium sulfate, filtered and concentrated under reduced pressure. The resulting mixture was purified by silica gel column chromatography (0-100% (3:1 ethyl acetate:ethanol) gradient in hexanes) to afford 7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (Int-37). MS (ESI): m/z (M+H)⁺ 550. ¹H NMR (499 MHz, DMSO-d₆) δ 8.02 (d, J=8.2 Hz, 1H), 7.70 (d, J=2.0 Hz, 1H), 7.61-7.56 (m, 2H), 7.52 (dd, J=7.7 Hz, 1H), 7.31 (d, J=2.1 Hz, 1H), 5.39 (s, 2H), 5.37-5.22 (m, 1H), 4.16 (d, J=10.3 Hz, 1H), 4.07 (d, J=10.3 Hz, 1H), 3.78 (s, 1H), 3.45 (s, 3H), 3.20-2.97 (m, 4H), 2.87-2.79 (m, 1H), 2.20-1.99 (m, 3H), 1.87-1.76 (m, 3H).

### Intermediate 38: triisopropyl((6-(methoxymethoxy)-8-(4,4,5,5-tetramethyl-1,3,2-dioxaborolan-2-yl)naphthalen-1-yl)ethynyl)silane (Int-38)

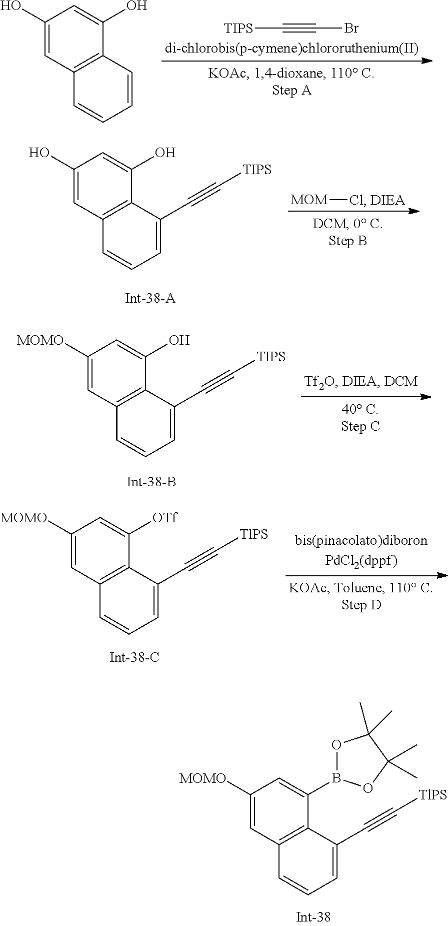

### Step A: 8-((triisopropylsilyl)ethynyl)naphthalene-1,3-diol (Int-38-A)

To a solution of naphthalene-1,3-diol (16.0 g, 100 mmol) in dioxane (160 mL) was added (bromoethynyl)triisopropylsilane (31.3 g, 120 mmol), potassium acetate (19.6 g, 200 mmol) and dichloro(p-cymene)ruthenium(II) dimer (6.12 g, 9.99 mmol) at 25° C. under N₂ atmosphere, and the mixture was stirred at 110° C. for 16 h. The reaction mixture was diluted with DCM (200 mL), and the resulting mixture was filtered. The filtrate was concentrated under vacuum, and the residue was purified by flash silica gel chromatography eluting with ethyl acetate in hexane) to give 8-((triisopropylsilyl)ethynyl)naphthalene-1,3-diol (Int-38-A). ¹H NMR (400 MHz, CDCl₃) δ 9.26 (s, 1H), 7.60 (d, J=8.3 Hz, 1H), 7.44 (d, J=7.1 Hz, 1H), 7.26-7.30 (m, 1H), 6.72 (s, 1H), 6.60 (s, 1H), 4.90 (br s, 1H), 1.14-1.18 (m, 21H).

### Step B: 3-(methoxymethoxy)-8-((triisopropylsilyl)ethynyl)naphthalen-1-ol (Int-38-B)

To a solution of 8-((triisopropylsilyl)ethynyl)naphthalene-1,3-diol (Int-38-A) (13.0 g, 38.2 mmol) in DCM (130 mL) was added DIEA (20 mL, 115 mmol) and MOM-Cl (8.44 g, 7.96 mL, 105 mmol) at 0° C., and the mixture was stirred at 0° C. for 10 min. The reaction mixture was diluted with DCM (200 mL) and washed with brine (30 mL×2). The organic layer was dried over anhydrous Na₂SO₄, filtered and concentrated under vacuum. The residue was purified by flash silica gel chromatography (eluting with ethyl acetate in hexane) to give 3-(methoxymethoxy)-8-((triisopropylsilyl)ethynyl)naphthalen-1-ol (Int-38-B). ¹H NMR (400 MHz, MeOD) δ 7.63-7.71 (m, 1H), 7.44 (dd, J=1.0, 7.1 Hz, 1H), 7.24-7.34 (m, 1H), 6.93 (d, J=2.4 Hz, 1H), 6.62 (d, J=2.2 Hz, 1H), 5.23 (s, 2H), 3.46 (s, 3H), 1.10-1.23 (m, 21H).

### Step C: 3-(methoxymethoxy)-8-((triisopropylsilyl)ethynyl)naphthalen-1-yl trifluoromethanesulfonate (Int-38-C)

To a solution of 3-(methoxymethoxy)-8-((triisopropylsilyl)ethynyl)naphthalen-1-ol (Int-38-B) (9.00 g, 23.40 mmol) in DCM (100 mL) was added DIEA (12.26 mL, 70.2 mmol) and Tf2O (5.9 mL, 35.1 mmol) at −40° C., and the mixture was stirred at −40° C. for 2 min. The reaction mixture was concentrated under vacuum, and the residue was purified by flash silica gel chromatography (eluting with ethyl acetate in hexane) to give 3-(methoxymethoxy)-8-((triisopropylsilyl)ethynyl)naphthalen-1-yl trifluoromethanesulfonate (Int-38-C). ¹H NMR (400 MHz, CHLOROFORM-d) 6=7.71 (t, J=7.6 Hz, 2H), 7.45-7.38 (m, 2H), 7.29 (d, J=2.0 Hz, 1H), 5.27 (s, 2H), 3.54-3.46 (m, 3H), 1.18-1.10 (m, 21H).

### Step D: triisopropyl((6-(methoxymethoxy)-8-(4,4,5,5-tetramethyl-1,3,2-dioxaborolan-2-yl)naphthalen-1-yl)ethynyl)silane (Int-38)

To a solution of 3-(methoxymethoxy)-8-((triisopropylsilyl)ethynyl)naphthalen-1-yl trifluoromethanesulfonate (Int-38-C) (9.00 g, 17.42 mmol) and bis(pinacolato)diboron (8.85 g, 34.8 mmol) in toluene (180 mL) was added potassium acetate (5.98 g, 61.0 mmol) and PdCl₂(dppf) (1.275 g, 1.742 mmol) at 20° C., and the solution was stirred at 110° C. for 15 h under N₂ atmosphere. The reaction mixture was diluted with EtOAc (500 mL), and the resulting mixture was filtered. The filtrate was concentrated under vacuum, and the residue was purified by flash silica gel chromatography (eluting with EtOAc in hexane) to give triisopropyl((6-(methoxymethoxy)-8-(4,4,5,5-tetramethyl-1,3,2-dioxaborolan-2-yl)naphthalen-1-yl)ethynyl)silane (Int-38). ¹H NMR (400 MHz, CDCl₃) δ 7.69 (dd, J=2.8, 7.7 Hz, 2H), 7.47 (d, J=2.4 Hz, 1H), 7.31-7.40 (m, 2H), 5.27-5.31 (m, 2H), 3.47-3.52 (m, 3H), 1.44 (s, 12H), 1.12-1.21 (m, 21H).

### Intermediate 39: 6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-ol (Int-39)

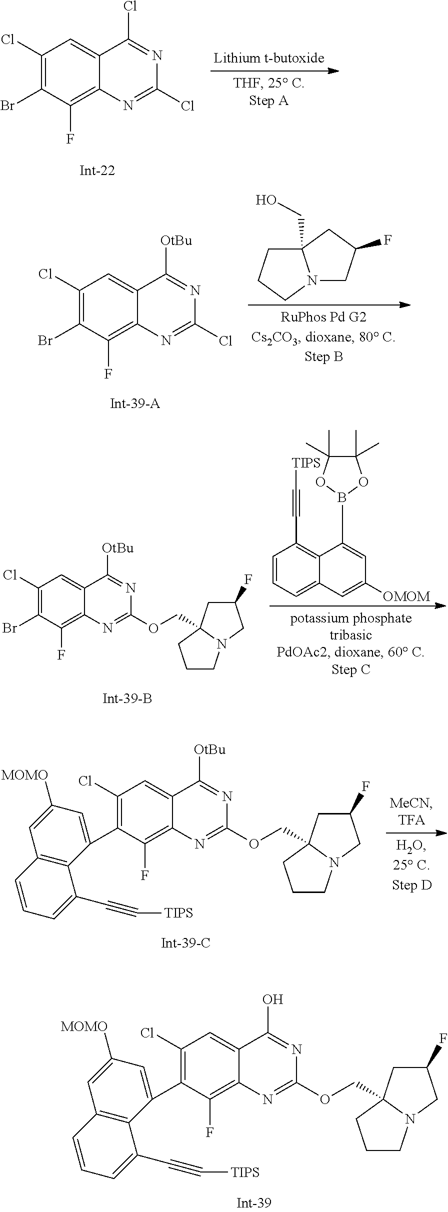

### Step A: 7-bromo-4-(tert-butoxy)-2,6-dichloro-8-fluoroquinazoline (Int-39-A)

To a solution of 7-bromo-2,4,6-trichloro-8-fluoroquinazoline (Int-22) (5 g, 15.13 mmol) in THF (50 mL) was added lithium 2-methylpropan-2-olate (7 mL, 15.40 mmol, 2.2 M in THF) at 25° C. under N₂ atmosphere. The mixture was stirred at 25° C. for 1 h. LCMS showed starting material was consumed and desired peak was formed. TLC (SiO₂; petroleum ether:ethyl acetate=20:1) showed starting material was consumed and new spots were observed. The mixture was quenched with H₂O (20 mL), extracted with EtOAc (2×60 mL), dried over Na₂SO₄, filtered, and the solvent was evaporated under reduced pressure to give the crude product. The crude product was purified by flash silica gel chromatography (basified with TEA, eluting with EtOAc in Hexane) to give 7-bromo-4-(tert-butoxy)-2,6-dichloro-8-fluoroquinazoline (Int-39-A). MS (ESI): m/z (M+H−tBu)⁺ 311.3, 313.3. ¹H NMR (400 MHz, CDCl₃) δ 7.98 (d, J=1.7 Hz, 1H), 1.77 (s, 9H).

### Step B: 7-bromo-4-(tert-butoxy)-6-chloro-8-fluoro-2-(((2R,7aS)-2-fluorotetrahydro-1H-pyrrolizin-7a(5H)-yl)methoxy)quinazoline (Int-39-B)

To a solution of 7-bromo-4-(tert-butoxy)-2,6-dichloro-8-fluoroquinazoline (Int-39-A) (4 g, 10.87 mmol) and ((2R,7aS)-2-fluorotetrahydro-1H-pyrrolizin-7a(5H)-yl)methanol (Int-8) (2.60 g, 16.30 mmol) in dioxane (60 mL) was added Cs₂CO₃ (7.08 g, 21.74 mmol) and RuPhos Pd G2 (0.844 g, 1.087 mmol). The solution was stirred at 80° C. for 15 hours under N₂ atmosphere. LCMS showed the starting material was consumed and desired MS was found. The reaction mixture was quenched with water (10 mL), and the resulting mixture was extracted with EtOAc (150 mL×3). The combined organic phase was dried over anhydrous Na₂SO₄, filtered and concentrated under vacuum. The residue was purified by flash silica gel chromatography (eluting with EtOAc in hexane) to give 7-bromo-4-(tert-butoxy)-6-chloro-8-fluoro-2-(((2R,7aS)-2-fluorotetrahydro-1H-pyrrolizin-7a(5H)-yl)methoxy)quinazoline (Int-39-B). MS (ESI): n/z (M+H)⁺ 489.8, 491.8.

### Step C: 4-(tert-butoxy)-6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazoline (Int-39-C)

To a solution of 7-bromo-4-(tert-butoxy)-6-chloro-8-fluoro-2-(((2R,7aS)-2-fluorotetrahydro-1H-pyrrolizin-7a(5H)-yl)methoxy)quinazoline (Int-39-B) (2 g, 4.08 mmol) and triisopropyl((6-(methoxymethoxy)-8-(4,4,5,5-tetramethyl-1,3,2-dioxaborolan-2-yl)naphthalen-1-yl)ethynyl)silane (Int-38) (3.02 g, 6.11 mmol) in dioxane (40 mL) was added potassium phosphate tribasic (2.60 g, 12.23 mmol), rRac-BI-DIME (0.404 g, 1.223 mmol) and Pd(OAc)2 (0.137 g, 0.611 mmol) under N₂, and the solution was stirred at 60° C. for 15 h under N₂ atmosphere. The reaction mixture was diluted with EtOAc (100 mL) and washed with water (10 mL) and brine (10 mL). The organic phase was dried over anhydrous Na₂SO₄, filtered and concentrated under vacuum. The residue was purified by flash silica gel chromatography (eluting with EtOAc in hexane) to give 4-(tert-butoxy)-6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazoline (Int-39-C). MS (ESI): n/z (M+H)⁺ 778.4.

### Step D: 6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-ol (12) (Int-39)

A mixture of 4-(tert-butoxy)-6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazoline (Int-39-C) (250 mg, 0.225 mmol) in MeCN (4 mL), water (1 mL) and TFA (0.25 mL) was stirred at 25° C. for 2 h. LCMS showed the starting material was consumed and desired MS was formed. The reaction mixture was concentrated under vacuum at 25° C. The residue was purified by preparative HPLC (Column: Boston Green ODS, ACN/water with 0.1% TFA) to give 6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-ol (Int-39). MS (ESI): n/z (M+H)⁺ 722.3.

### Intermediate 40: 7-(2-(bis(2,4-dimethoxybenzyl)amino)-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-ol (Int-40)

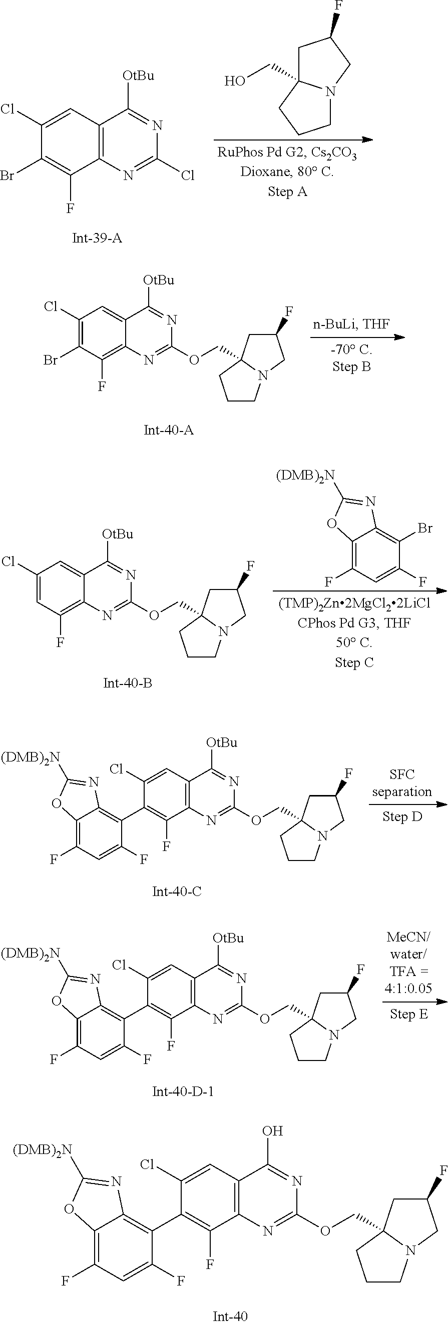

### Step A: 7-bromo-4-(tert-butoxy)-6-chloro-8-fluoro-2-(((2R,7aS)-2-fluorotetrahydro-1H-pyrrolizin-7a(5H)-yl)methoxy)quinazoline (Int-40-A)

To a solution of 7-bromo-4-(tert-butoxy)-2,6-dichloro-8-fluoroquinazoline (Int-39-A) (10 g, 27.2 mmol) and ((2R,7aS)-2-fluorotetrahydro-1H-pyrrolizin-7a(5H)-yl)methanol (Int-8) (6.49 g, 40.8 mmol) in dioxane (150 mL) was added Cs₂CO₃ (17.71 g, 54.3 mmol) and RuPhos Pd G2 (2.111 g, 2.72 mmol). The solution was stirred at 80° C. for 15 h under N₂ atmosphere. The reaction mixture was quenched with water (50 mL), and the resulting mixture was extracted with EtOAc (300 mL×3). The combined organic phase was dried over anhydrous Na₂SO₄, filtered and concentrated in vacuo. The residue was purified by flash silica gel chromatography (eluting with ethyl acetate in hexanes) to give 7-bromo-4-(tert-butoxy)-6-chloro-8-fluoro-2-(((2R,7aS)-2-fluorotetrahydro-1H-pyrrolizin-7a(5H)-yl)methoxy)quinazoline (Int-40-A). MS (ESI) [M+H]⁺ m/z: 490.

### Step B: 4-(tert-butoxy)-6-chloro-8-fluoro-2-(((2R,7aS)-2-fluorotetrahydro-1H-pyrrolizin-7a(5H)-yl)methoxy)quinazoline (Int-40-B)

To a stirred solution of 7-bromo-4-(tert-butoxy)-6-chloro-8-fluoro-2-(((2R,7aS)-2-fluorotetrahydro-1H-pyrrolizin-7a(5H)-yl)methoxy)quinazoline (Int-40-A) (8.7 g, 18 mmol) in THF (100 mL) was added nBuLi (10.64 mL, 26.6 mmol) (2.5 M in hexanes) at −78° C., and the resulting mixture was stirred at −78° C. for 2 h. The reaction was quenched with water (10 mL), and the resulting mixture was extracted with EtOAc (200 mL×3). The combined organic phase was dried over anhydrous Na₂SO₄, filtered and concentrated in vacuo. The residue was purified by flash silica gel chromatography (eluting with ethyl acetate in hexane, 0-100% gradient) to give 4-(tert-butoxy)-6-chloro-8-fluoro-2-(((2R,7aS)-2-fluorotetrahydro-1H-pyrrolizin-7a(5H)-yl)methoxy)quinazoline (Int-40-B). MS (ESI) [M+H]⁺ m/z: 412.

### Step C: 4-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(2,4-dimethoxybenzyl)-5,7-difluorobenzo[d]oxazol-2-amine (Int-40-C)

A solution of 4-(tert-butoxy)-6-chloro-8-fluoro-2-(((2R,7aS)-2-fluorotetrahydro-1H-pyrrolizin-7a(5H)-yl)methoxy)quinazoline (Int-40-B) (5.7 g, 13.84 mmol) in (TMP)₂Zn·2MgCl₂·2LiCl (180 mL, 59.5 mmol, 0.33 M in THF) was stirred at 50° C. for 5 h. 4-bromo-N-(2,4-dimethoxybenzyl)-N-(3,5-dimethoxybenzyl)-5,7-difluorobenzo[d]oxazol-2-amine (13.30 g, 24.22 mmol) and CPhos Pd G3 (2.232 g, 2.77 mmol) in THF (24 mL) were added to the reaction solution at 25° C. in a glove box, and then the reaction mixture was stirred at 50° C. for 15 h. The reaction was diluted with EtOAc (500 mL) and saturated NaHCO₃ aqueous solution (150 mL), and the resulting mixture was filtered. The filtrate was concentrated in vacuo, and the residue was purified by flash silica gel chromatography (eluting with EtOAc in hexane, 0-100% gradient) to give 4-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(2,4-dimethoxybenzyl)-5,7-difluorobenzo[d]oxazol-2-amine (Int-40-C). MS (ESI) [M+H]⁺ m/z: 880.

### Step D: 4-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(2,4-dimethoxybenzyl)-5,7-difluorobenzo[d]oxazol-2-amine (Int-40-D-1)

The atropisomeric mixture of 4-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(2,4-dimethoxybenzyl)-5,7-difluorobenzo[d]oxazol-2-amine (Int-40-C) (6.5 g, 7.38 mmol) was separated by preparative SFC (Column H; 45% EtOH with 0.1% NH₃H₂O) to afford 4-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(2,4-dimethoxybenzyl)-5,7-difluorobenzo[d]oxazol-2-amine (Int-40-D-1, the first eluting isomer). MS (ESI) [M+H]⁺ m/z: 880.

### Step E: 7-(2-(bis(2,4-dimethoxybenzyl)amino)-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-ol (Int-40)

A mixture of 4-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(2,4-dimethoxybenzyl)-5,7-difluorobenzo[d]oxazol-2-amine (Int-40-D-1) (2.5 g, 2.8 mmol) in MeCN (20 mL), water (5 mL) and TFA (1 mL) was stirred at 25° C. for 4 h. The reaction mixture was concentrated in vacuo, and the residue was basified with aqueous NH₃·H₂O (1 mL). The mixture was purified by preparative HPLC (C18 ACN/water with (0.04% NH₃H₂O+10 mM NH₄HCO₃)) to give 7-(2-(bis(2,4-dimethoxybenzyl)amino)-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-ol (Int-40). MS (ESI) [M+H]⁺ m/z: 824.

### Intermediate 41: 7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol (Int-41)

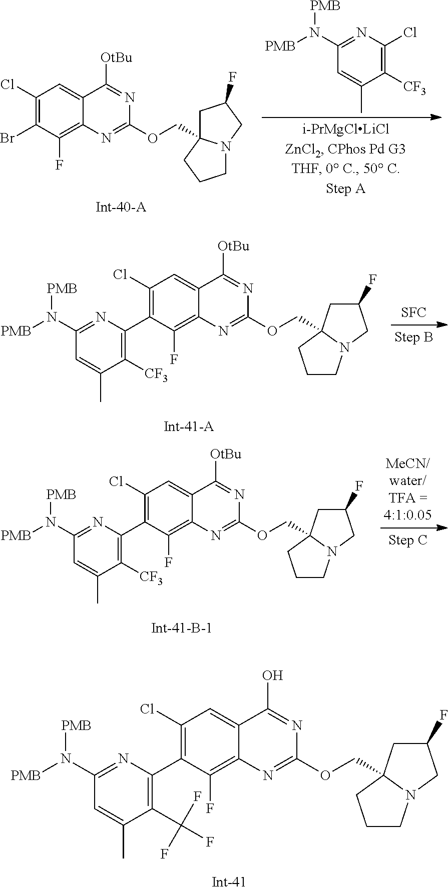

### Step A: 6-(4-(tert-butoxy)-6-chloro-8-fluoro-2-(((2R,7aS)-2-fluorotetrahydro-TH-pyrrolizin-7a(5H)-yl)methoxy)quinazolin-7-yl)-N,N-bis(4-methoxybenzyl)-4-methyl-5-(trifluoromethyl)pyridin-2-amine (Int-41-A)

To a solution of 7-bromo-4-(tert-butoxy)-6-chloro-8-fluoro-2-(((2R,7aS)-2-fluorotetrahydro-1H-pyrrolizin-7a(5H)-yl)methoxy)quinazoline (Int-40-A) (235 mg, 0.479 mmol) in THF (2 mL) was added isopropylmagnesium chloride lithium chloride complex (0.774 mL, 1.006 mmol) (1.3 M in THF) at 0° C. under N₂ atmosphere, and the mixture was stirred at 0° C. for 20 min. Zinc chloride (0.766 mL, 0.766 mmol, 1 M in THF) was added to the reaction solution at 0° C., and the mixture was stirred at 25° C. for 10 min. 6-chloro-N,N-bis(4-methoxybenzyl)-4-methyl-5-(trifluoromethyl)pyridin-2-amine (216 mg, 0.479 mmol) and CPhos Pd G3 (39 mg, 0.048 mmol) were added to the reaction solution at 25° C. in a glove box; then the reaction mixture was stirred at 50° C. for 15 h. The mixture was cooled, quenched with H₂O (1 mL), extracted with EtOAc (2×3 mL), dried over Na₂SO₄, filtered, and the solvent was evaporated under reduced pressure to give the crude product. The crude product was purified by flash silica gel chromatography (basified with TEA, eluting with ethyl acetate in hexane) to give 6-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine (Int-41-A). MS (ESI) [M+H]⁺: m/z 826.

### Step B: 6-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine (Int-41-B-1)

Racemic 6-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine (Int-41-A) (90 mg, 0.109 mmol) was separated by SFC (Column: G; 25% Ethanol with 0.1% NH₃·H₂O) to give 6-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine (Int-41-B-1) (the first eluting isomer from SFC). MS (ESI) [M+H]⁺: m/z 826.

### Step C: 7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol (Int-41)

6-(4-(tert-butoxy)-6-chloro-8-fluoro-2-(((2R,7aS)-2-fluorotetrahydro-1H-pyrrolizin-7a(5H)-yl)methoxy)quinazolin-7-yl)-N,N-bis(4-methoxybenzyl)-4-methyl-5-(trifluoromethyl)pyridin-2-amine (Int-41-B-1) (42 mg, 0.051 mmol, from Step D) was added to a solution of MeCN (4 mL), water (1 mL) and TFA (0.05 mL) at 20° C. The mixture was stirred at 20° C. for 1 h. LCMS showed starting material was consumed and desired peak was formed. The solvent was evaporated under reduced pressure to give the crude product. The crude product was purified by reverse phase preparative HPLC (C18 ACN/water with 0.225% TFA) to give 7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol (Int-41). MS (ESI) [M+H]⁺: m/z 770.

### Example 1: (3R)-1-(6-chloro-7-(8-ethynyl-3-hydroxvnaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (Ex. 1)

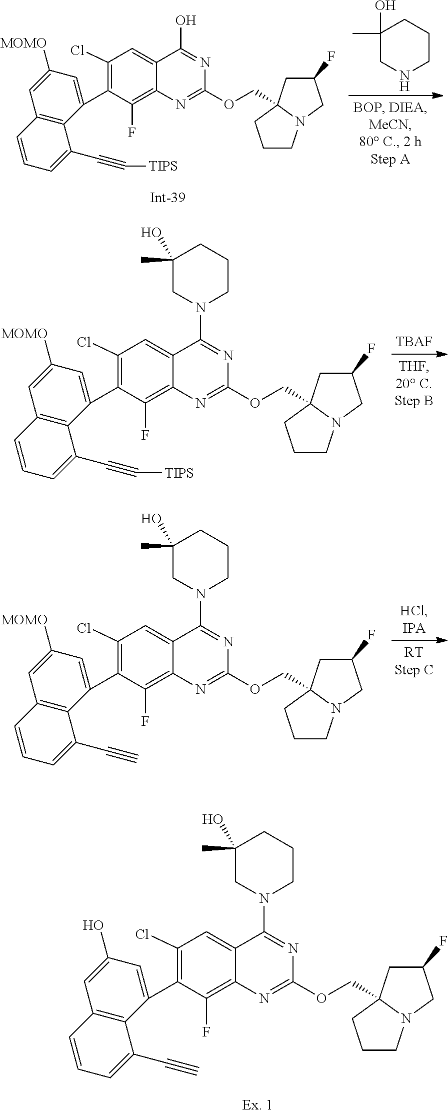

### Step A: (3R)-1-(6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-3-methylpiperidin-3-ol

To a solution of 6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-ol (Int-39) (80 mg, 0.11 mmol) in MeCN (1 mL) was added ((1H-benzo[d][1,2,3]triazol-1-yl)oxy)tris(dimethylamino)phosphonium hexafluorophosphate(V) (74 mg, 0.17 mmol) at 25° C. The mixture was stirred at 25° C. for 30 min. N-ethyl-N-isopropylpropan-2-amine (72 mg, 0.55 mmol) and (R)-3-methylpiperidin-3-ol (19 mg, 0.167 mmol) was added to the reaction solution; then the mixture was stirred at 80° C. for 2 h. The reaction mixture was cooled to room temperature and concentrated under reduced pressure, and the residue was purified by preparative TLC plate (silica, hexanes/EtOAc=0/1) to give (3R)-1-(6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-3-methylpiperidin-3-ol as yellow oil. MS (ESI): m/z (M+H)⁺ 819.4. The mixture of atropisomers (3R)-1-(6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-3-methylpiperidin-3-ol was separated by preparative SFC (Column G; 35% IPA with 0.1% NH₃H₂O) to afford (3R)-1-(6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-3-methylpiperidin-3-ol (Pk1, the first eluting isomer) as yellow solid and (3R)-1-(6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-3-methylpiperidin-3-ol (Pk2, the second eluting isomer) as yellow solid. MS (ESI): m/z (M+H)⁺ 819.4.

### Step B: (3R)-1-(6-chloro-7-(8-ethynyl-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol

To a solution of (3R)-1-(6-chloro-8-fluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphthalen-1-yl)quinazolin-4-yl)-3-methylpiperidin-3-ol (Pk1 from Step A, 38 mg, 0.046 mmol) in THF (0.5 mL) was added TBAF (0.070 mL, 0.070 mmol) (1M in THF) at 25° C. The mixture was stirred at 25° C. for 30 min. The reaction solution was concentrated under reduced pressure, and the residue was purified by preparative TLC plate (Silica, DCM/MeOH=10/1) to give (3R)-1-(6-chloro-7-(8-ethynyl-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol as yellow oil. LCMS (ESI): m/z (M+H)⁺ 663.2.

### Step C: (3R)-1-(6-chloro-7-(8-ethynyl-3-hydroxvnaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (Ex. 1)

To a solution of (3R)-1-(6-chloro-7-(8-ethynyl-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (from Step B, 22 mg, 0.033 mmol) in 2-propanol (0.25 mL) was added HCl/dioxane (8.29 μl, 0.033 mmol) (4M in dioxane) at 25° C. The mixture was stirred at 25° C. for 30 min. LCMS showed starting material was consumed and desired MS was formed. The reaction solution was concentrated under vacuum, and the residue was purified by preparative HPLC (C18, 0-100% ACN/water with 0.1% TFA) to give (3R)-1-(6-chloro-7-(8-ethynyl-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (Ex. 1). LCMS (ESI): n/z (M+H)⁺ 619.2. ¹H NMR (400 MHz, MeOD) δ 8.09-8.22 (m, 1H), 7.84 (d, J=8.2 Hz, 1H), 7.51 (d, J=7.2 Hz, 1H), 7.38-7.44 (m, 1H), 7.35 (d, J=2.4 Hz, 1H), 7.06 (d, J=2.5 Hz, 1H), 5.39-5.69 (m, 1H), 4.56-4.78 (m, 3H), 4.25-4.41 (m, 1H), 3.81-4.06 (m, 3H), 3.57-3.67 (m, 1H), 3.35-3.52 (m, 2H), 3.10-3.18 (m, 1H), 2.56-2.80 (m, 2H), 2.30-2.49 (m, 3H), 2.19 (br d, J=11.1 Hz, 2H), 1.75-1.91 (m, 3H), 1.30 (s, 3H).

### Example 2: (3R)-1-(7-(8-chloro-3-hydroxynaphthalen-1-yl)-2-(((R)-1-((dimethylamino)methyl)-2,2-difluorocyclopropyl)methoxy)-6,8-difluoroquinazolin-4-yl)-3-methylpiperidin-3-ol (Ex. 2)

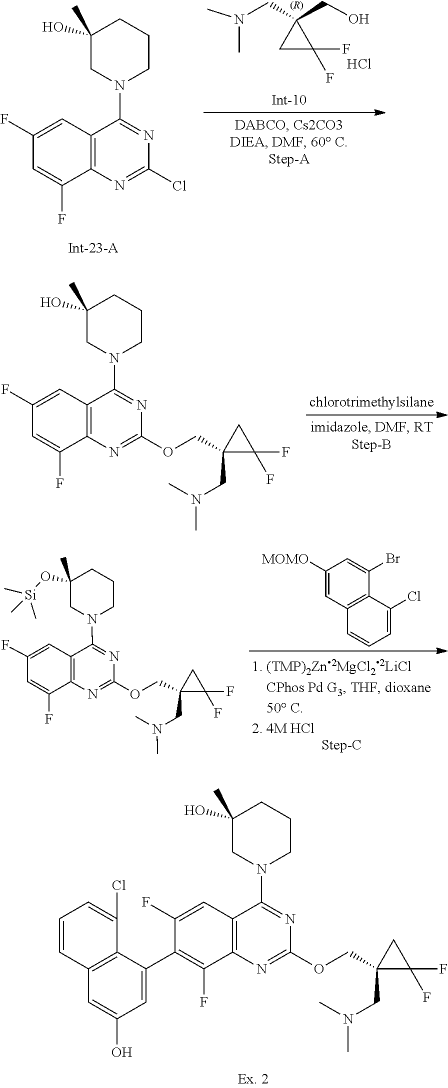

### Step A: (R)-1-(2-(((R)-1-((dimethylamino)methyl)-2,2-difluorocyclopropyl)methoxy)-6,8-difluoroquinazolin-4-yl)-3-methylpiperidin-3-ol

To a solution of (R)-1-(2-chloro-6,8-difluoroquinazolin-4-yl)-3-methylpiperidin-3-ol (Int-23-A) (273 mg, 0.870 mmol) and (R)-(1-((dimethylamino)methyl)-2,2-difluorocyclopropyl)methanol hydrochloride (Int-10) (193 mg, 0.957 mmol) in DMF (9.0 mL) were added cesium carbonate (851 mg, 2.61 mmol), 1,4-diazabicyclo[2.2.2]octane (19.5 mg, 0.174 mmol) and N,N-diisopropylethylamine (0.152 mL, 0.870 mmol) at room temperature. After stirring the mixture at room temperature for 17 h, the reaction mixture was heated to 60° C. for 9 h. The reaction mixture was quenched with H₂O, extracted with EtOAc, washed with brine, dried over Na₂SO₄, filtered and concentrated under reduced pressure. The residue was purified by flash NH-silica gel chromatography (20-50%, EtOAc gradient in hexane) to afford (R)-1-(2-(((R)-1-((dimethylamino)methyl)-2,2-difluorocyclopropyl)methoxy)-6,8-difluoroquinazolin-4-yl)-3-methylpiperidin-3-ol. ESI-MS m/z [M+H]⁺ 443.

### Step B: 1-((R)-1-(((6,8-difluoro-4-((R)-3-methyl-3-((trimethylsilyl)oxy)piperidin-1-yl)quinazolin-2-yl)oxy)methyl)-2,2-difluorocyclopropyl)-N,N-dimethylmethanamine

To a solution of (R)-1-(2-(((R)-1-((dimethylamino)methyl)-2,2-difluorocyclopropyl)methoxy)-6,8-difluoroquinazolin-4-yl)-3-methylpiperidin-3-ol (261 mg, 0.590 mmol) in DMF (6.0 mL) were added imidazole (201 mg, 2.95 mmol) and chlorotrimethylsilane (0.150 mL, 1.18 mmol) at room temperature. After stirring the mixture at room temperature for 15 h, the reaction was quenched by the addition of H₂O. The reaction mixture was extracted with EtOAc, washed with brine, dried over Na₂SO₄, filtered and concentrated in vacuo. The residue was purified by flash silica gel chromatography (0-50%, EtOAc gradient in hexane) to afford 1-((R)-1-(((6,8-difluoro-4-((R)-3-methyl-3-((trimethylsilyl)oxy)piperidin-1-yl)quinazolin-2-yl)oxy)methyl)-2,2-difluorocyclopropyl)-N,N-dimethylmethanamine. ESI-MS m/z [M+H]⁺ 515.

### Step C: (3R)-1-(7-(8-chloro-3-hydroxynaphthalen-1-yl)-2-(((R)-1-((dimethylamino)methyl)-2,2-difluorocyclopropyl)methoxy)-6,8-difluoroquinazolin-4-yl)-3-methylpiperidin-3-ol (Ex. 2)

To a 12 wt. % solution of (TMP)₂Zn·xMgCl2·xLiCl in THF/toluene (4.27 mL, 0.583 mmol) was added 1-((R)-1-(((6,8-difluoro-4-((R)-3-methyl-3-((trimethylsilyl)oxy)piperidin-1-yl)quinazolin-2-yl)oxy)methyl)-2,2-difluorocyclopropyl)-N,N-dimethylmethanamine (100 mg, 0.194 mmol). After stirring the mixture at 50° C. for 1 h, to the reaction mixture was added a solution of 1-bromo-8-chloro-3-(methoxymethoxy)naphthalene (70.3 mg, 0.233 mmol) and CPhos Pd G3 (15.7 mg, 0.0194 mmol) in 1,4-dioxane (1.0 mL). After stirring the mixture at 50° C. for 18 h, the reaction was quenched by the addition of saturated aqueous NaHCO₃ and EtOAc, and the mixture was filtered and concentrated under reduced pressure. The residue was purified by flash silica gel chromatography (0-100%, EtOAc gradient in hexane) to afford coupling compound.

To a solution of the coupling compound in dichloromethane (4.0 mL) was added 4.0 M 1,4-dioxane solution of HCl (2.0 mL). After stirring the mixture at room temperature for 30 min, the reaction mixture was concentrated under reduced pressure. The residue was purified by reverse phase HPLC (C18, 0-100% MeCN/water with 0.1% formic acid) to afford (3R)-1-(7-(8-chloro-3-hydroxynaphthalen-1-yl)-2-(((R)-1-((dimethylamino)methyl)-2,2-difluorocyclopropyl)methoxy)-6,8-difluoroquinazolin-4-yl)-3-methylpiperidin-3-ol (Ex. 2). ESI-MS m/z [M+H]⁺ 619, 621. ¹H NMR (400 MHz, DMSO-d₆) δ 10.31 (s, 0.5H), 10.30 (s, 0.5H), 7.88-7.82 (m, 1.5H), 7.76 (d, J=9.3 Hz, 0.5H), 7.44-7.34 (m, 3H), 7.13-7.09 (m, 1H), 4.79 (s, 0.5H), 4.65 (s, 0.5H), 4.45 (d, J=11.0 Hz, 1H), 4.38 (d, J=11.2 Hz, 1H), 4.13-3.99 (in, TH), 3.85 (d, J=13.2 Hz, TH), 3.46-3.33 (in, TH), 3.30-3.19 (in, TH), 2.65-2.57 (m, 1H), 2.38-2.29 (m, 1H), 2.15 (s, 6H), 2.09-1.94 (m, 1H), 1.84-1.54 (m, 4H), 1.51-1.42 (m, 1H), 1.17 (s, 1.5H), 1.14 (s, 1.5H).

The compounds in the table below were synthesized using a similar procedure as described in the synthesis of Ex. 2 by making the appropriate substitutions for starting material, intermediates, and reagents. Appropriate substitutions are available commercially, synthesized as described in the literature, synthesized using methods available to those skilled in the art, or synthesized as described herein.

| | | | [M + H]⁺ |
|---|---|---|---|
| Ex | Structure | Compound Name | Found |
| Ex. 3 | 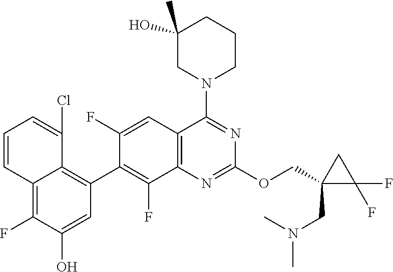 [SMILES: CN(C)C[C@@]1(COc2nc(N3CCC[C@@](C)(O)C3)c3cc(F)c(-c4cc(O)c(F)c5cccc(Cl)c45)c(F)c3n2)CC1(F)F] | (3R)-1-(7-(8-chloro-4-fluoro-3- hydroxynaphthalen-1-yl)-2- (((R)-1- ((dimethylamino)methyl)-2,2- difluorocyclopropyl)methoxy)- 6,8-difluoroquinazolin-4-yl)-3- methylpiperidin-3-ol | 637, 639 |
| Ex. 4 | 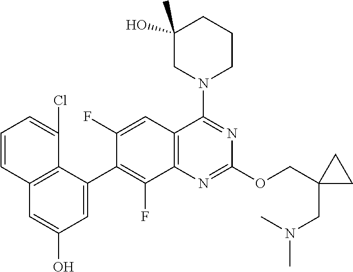 [SMILES: CN(C)CC1(COc2nc(N3CCC[C@@](C)(O)C3)c3cc(F)c(-c4cc(O)cc5cccc(Cl)c45)c(F)c3n2)CC1] | (3R)-1-(7-(8-chloro-3- hydroxynaphthalen-1-yl)-2-((1- ((dimethylamino)methyl)cyclopro- pyl)methoxy)-6,8- difluoroquinazolin-4-yl)-3- methylpiperidin-3-ol | 583, 585 |

### Example 5: (3R)-1-(7-(8-chloro-3-hydroxnagphthalen-1-yl)-6,8-difluoro-2-((1-((4-methoxypiperidin-1-yl)methyl)cyclopropyl)methoxy)quinazolin-4-yl)-3-methylpiperidin-3-ol (Ex. 5)

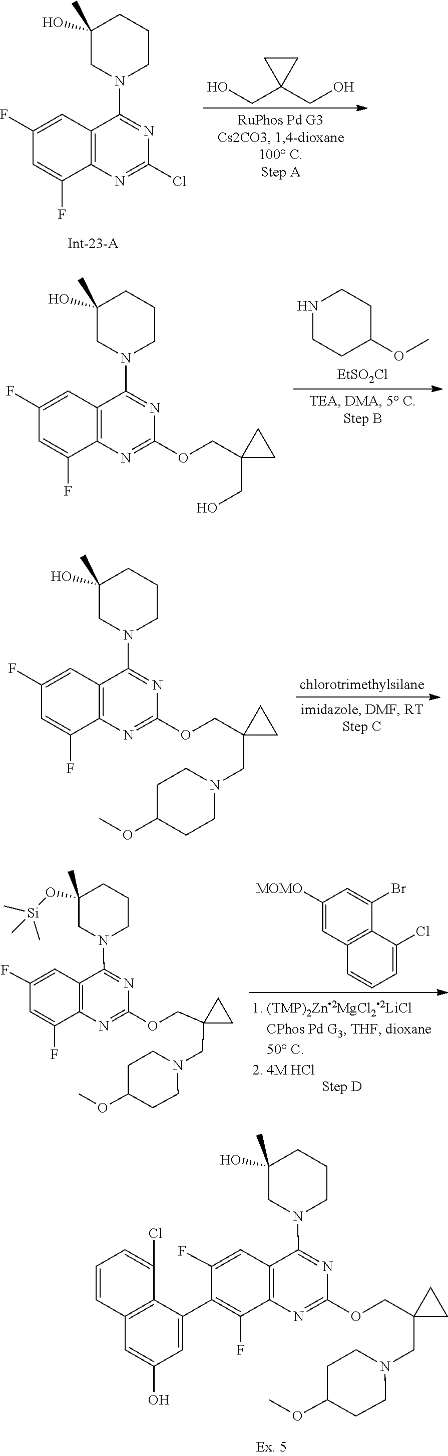

### Step A: (R)-1-(6,8-difluoro-2-((1-(hydroxvmethyl)cyclopropyl)methoxy)quinazolin-4-yl)-3-methylpiperidin-3-ol

To a solution of (R)-1-(2-chloro-6,8-difluoroquinazolin-4-yl)-3-methylpiperidin-3-ol (Int-23-A) (300 mg, 0.956 mmol), cyclopropane-1,1-diyldimethanol (195 mg, 1.91 mmol) and RuPhos Pd G3 (24.0 mg, 0.0287 mmol) in 1,4-dioxane (5.0 mL) was added cesium carbonate (935 mg, 2.87 mmol) at room temperature. After stirring the mixture at 100° C. for 2 h, the reaction mixture was cooled to room temperature, quenched with H₂O, extracted with EtOAc, washed with brine, dried over Na₂SO₄, filtered and concentrated under reduced pressure. The residue was purified by flash silica gel chromatography (50-100%, EtOAc gradient in hexane) to afford (R)-1-(6,8-difluoro-2-((1-(hydroxymethyl)cyclopropyl)methoxy)quinazolin-4-yl)-3-methylpiperidin-3-ol. ESI-MS m z [M+H]⁺ 380.

### Step B: (R)-1-(6,8-difluoro-2-((1-((4-methoxypiperidin-1-yl)methyl)cyclopropyl)methoxy)quinazolin-4-yl)-3-methylpiperidin-3-ol

To a solution of (R)-1-(6,8-difluoro-2-((1-(hydroxymethyl)cyclopropyl)methoxy)quinazolin-4-yl)-3-methylpiperidin-3-ol (150 mg, 0.395 mmol) in N,N-dimethylacetamide (2.0 mL) were added ethane sulfonyl chloride (0.0719 mL, 0.791 mmol) and triethylamine (0.278 mL, 1.98 mmol) at 5° C. After stirring the mixture at 5° C. for 30 min, the reaction mixture was treated with 4-methoxypiperidine (0.245 mL, 1.98 mmol). After stirring the mixture at 75° C. for 2 h, the reaction mixture was cooled to room temperature, quenched with H₂O, extracted with EtOAc, washed with brine, dried over Na₂SO₄, filtered and concentrated under reduced pressure. The residue was purified by flash NH-silica gel chromatography (20-100%, EtOAc gradient in hexane) to afford (R)-1-(6,8-difluoro-2-((1-((4-methoxypiperidin-1-yl)methyl)cyclopropyl)methoxy)quinazolin-4-yl)-3-methylpiperidin-3-ol. ESI-MS m z [M+H]⁺ 477.

### Step C: (R)-6,8-difluoro-2-((1-((4-methoxypiperidin-1-yl)methyl)cyclopropyl)methoxy)-4-(3-methyl-3-((trimethylsilyl)oxy)piperidin-1-yl)quinazoline

To a solution of (R)-1-(6,8-difluoro-2-((1-((4-methoxypiperidin-1-yl)methyl)cyclopropyl)methoxy)quinazolin-4-yl)-3-methylpiperidin-3-ol (161 mg, 0.338 mmol) in DMF (3.4 mL) were added imidazole (115 mg, 1.69 mmol) and chlorotrimethylsilane (0.129 mL, 1.01 mmol) at room temperature. After stirring the mixture at room temperature for 2 h, the reaction was quenched by the addition of water. The reaction mixture was extracted with EtOAc, washed with brine, dried over Na₂SO₄, filtered and concentrated under reduced pressure. The residue was purified by flash NH-silica gel chromatography (0-30%, EtOAc gradient in hexane) to afford (R)-6,8-difluoro-2-((1-((4-methoxypiperidin-1-yl)methyl)cyclopropyl)methoxy)-4-(3-methyl-3-((trimethylsilyl)oxy)piperidin-1-yl)quinazoline. ESI-MS m/z [M+H]⁺ 549.

### Step D: (3R)-1-(7-(8-chloro-3-hydroxvnaphthalen-1-yl)-6,8-difluoro-2-((1-((4-methoxypiperidin-1-yl)methyl)cyclopropyl)methoxy)quinazolin-4-yl)-3-methylpiperidin-3-ol (Ex. 5)

To a 12 wt. % solution of (TMP)₂Zn·xMgCl₂·xLiCl in THF/toluene (4.33 mL, 0.590 mmol) was added (R)-6,8-difluoro-2-((1-((4-methoxypiperidin-1-yl)methyl)cyclopropyl)methoxy)-4-(3-methyl-3-((trimethylsilyl)oxy)piperidin-1-yl)quinazoline (108 mg, 0.197 mmol). After stirring the mixture at 50° C. for 1.5 h, to the reaction mixture was added a solution of 1-bromo-8-chloro-3-(methoxymethoxy)naphthalene (71.2 mg, 0.236 mmol) and CPhos Pd G3 (15.9 mg, 0.0197 mmol) in 1,4-dioxane (1.0 mL). After stirring the mixture at 50° C. for 15 h, the reaction was quenched by the addition of saturated aqueous NaHCO₃ and EtOAc, and the mixture was filtered and concentrated in vacuo. The residue was purified by flash silica gel chromatography (20-100%, EtOAc gradient in hexane). Intermediate containing fractions were concentrated in vacuo. The residue was redissolved in dichloromethane (2.0 mL) and treated with 4.0 M 1,4-dioxane solution of HCl (1.0 mL). After stirring the mixture at room temperature for 30 min, the reaction mixture was concentrated in vacuo. The residue was purified by reverse phase HPLC (MeCN/water with 0.1% formic acid) to afford (3R)-1-(7-(8-chloro-3-hydroxynaphthalen-1-yl)-6,8-difluoro-2-((1-((4-methoxypiperidin-1-yl)methyl)cyclopropyl)methoxy)quinazolin-4-yl)-3-methylpiperidin-3-ol (Ex. 5). ESI-MS m/z [M+H]⁺ 653, 655. ¹H NMR (400 MHz, DMSO-d₆) δ 10.31 (s, 0.5H), 10.30 (s, 0.5H), 7.87-7.79 (m, 1.5H), 7.74 (d, J=10.1 Hz, 0.5H), 7.44-7.35 (m, 3H), 7.12-7.09 (m, 1H), 4.79 (s, 0.5H), 4.65 (s, 0.5H), 4.26-4.18 (m, 2H), 4.09-3.95 (m, 1H), 3.80 (d, J=13.0 Hz, 1H), 3.40 (d, J=12.7 Hz, 0.5H), 3.31-3.20 (m, 1.5H), 3.18 (s, 3H), 3.14-3.05 (m, 1H), 2.79-2.65 (m, 2H), 2.34-2.20 (m, 2H), 2.10-1.94 (m, 3H), 1.82-1.55 (m, 5H), 1.41-1.28 (m, 2H), 1.16 (s, 1.5H), 1.14 (s, 1.5H), 0.61 (brs, 2H), 0.37 (brs, 2H).

The compound in the table below was synthesized using a similar procedure as described in the synthesis of Ex. 5 by making the appropriate substitutions for starting material, intermediates, and reagents. Appropriate substitutions are available commercially, synthesized as described in the literature, synthesized using methods available to those skilled in the art, or synthesized as described herein.

| | | | [M + H]⁺ |
|---|---|---|---|
| Ex | Structure | Compound Name | Found |
| Ex. 6 | 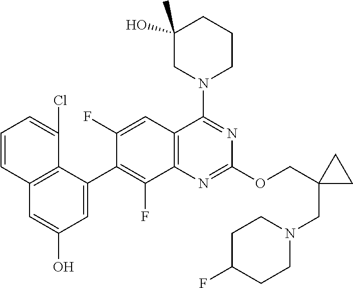 [SMILES: C[C@@]1(O)CCCN(c2nc(OCC3(CN4CCC(F)CC4)CC3)nc3c(F)c(-c4cc(O)cc5cccc(Cl)c45)c(F)cc23)C1] | (3R)-1-(7-(8-chloro-3- hydroxynaphthalen-1-yl)-6,8- difluoro-2-((1-((4-fluoropiperidin- 1- yl)methyl)cyclopropyl)methoxy)quin- azolin-4-yl)-3-methylpiperidin-3- ol | 641, 643 |

### Example 7: (3R)-1-(7-(8-ethynyl-3-hydroxvnaphthalen-1-yl)-6,8-difluoro-2-((1-((4-methoxypiperidin-1-yl)methyl)cyclopropyl)methoxy)quinazolin-4-yl)-3-methylpiperidin-3-ol (Ex. 7)

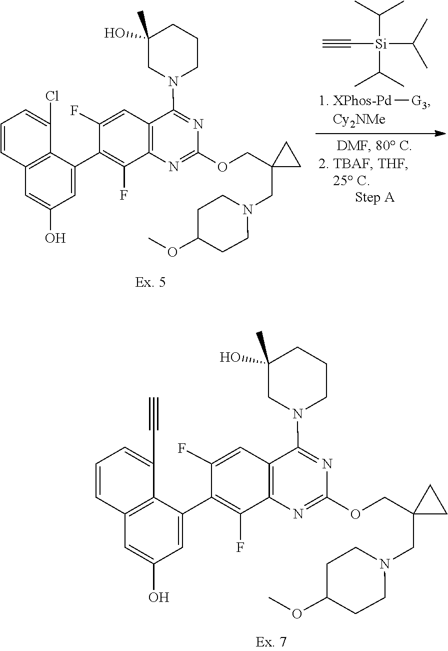

To a solution of (3R)-1-(7-(8-chloro-3-hydroxynaphthalen-1-yl)-6,8-difluoro-2-((1-((4-methoxypiperidin-1-yl)methyl)cyclopropyl)methoxy)quinazolin-4-yl)-3-methylpiperidin-3-ol (Ex. 5) (25.6 mg, 0.0392 mmol), triisopropylsilylacetylene (87.2 μL, 0.392 mmol) and XPhos Pd G3 (16.6 mg, 0.0196 mmol) in DMF (0.4 mL) was added N-cyclohexyl-N-methylcyclohexanamine (0.0420 mL, 0.196 mmol). The vessel was evacuated and backfilled with nitrogen, and the mixture was stirred at 100° C. for 12 h. The mixture was diluted with EtOAc, the diluted mixture was washed with saturated aqueous NaHCO₃, and the mixture was concentrated under reduced pressure. The residue was purified by reverse phase HPLC (MeCN/water with 0.1% formic acid) to give the coupling product (peak 1, first elution). To a solution of the coupling product (peak 1, first elution) in THF (1 mL) was added tetrabutylammonium fluoride (0.100 mL, 0.100 mmol, 1M in tetrahydrofuran), and the mixture was stirred at room temperature for 3 h. The mixture was diluted with ethyl acetate, the diluted mixture was washed with water, and the mixture was concentrated under reduced pressure. The residue was purified by flash NH₂-silica gel chromatography (0-10%, MeOH gradient in EtOAc) to afford (3R)-1-(7-(8-ethynyl-3-hydroxynaphthalen-1-yl)-6,8-difluoro-2-((1-((4-methoxypiperidin-1-yl)methyl)cyclopropyl)methoxy)quinazolin-4-yl)-3-methylpiperidin-3-ol (Ex. 7). ESI-MS m/z [M+H]⁺ 643. ¹H NMR (400 MHz, DMSO-d₆) δ 10.17 (s, 1H), 7.89 (dd, J=8.2, 1.4 Hz, 1H), 7.65 (d, J=10.1 Hz, 1H), 7.49-7.39 (m, 2H), 7.35 (d, J=2.6 Hz, 1H), 7.10 (d, J=2.6 Hz, 1H), 4.67 (s, 1H), 4.24 (d, J=10.8 Hz, 1H), 4.17 (d, J=10.8 Hz, 1H), 4.05 (d, J=13.5 Hz, 1H), 3.84 (d, J=13.1 Hz, 1H), 3.57 (s, 1H), 3.38-3.32 (m, 1H), 3.26-3.20 (m, 1H), 3.18 (s, 3H), 3.15-3.06 (m, 1H), 2.79-2.66 (m, 2H), 2.34-2.21 (m, 2H), 2.11-1.95 (m, 3H), 1.82-1.73 (m, 2H), 1.73-1.55 (m, 3H), 1.42-1.29 (m, 2H), 1.14 (s, 3H), 0.61 (brs, 2H), 0.37 (brs, 2H).

### Example 8: 1-(2-((1-((dimethylamino)methyl)cyclopropyl)methoxy)-6,8-difluoro-7-(3-hydroxynaphthalen-1-yl)quinazolin-4-yl)-3-methylpiperidin-3-ol (Ex. 8)

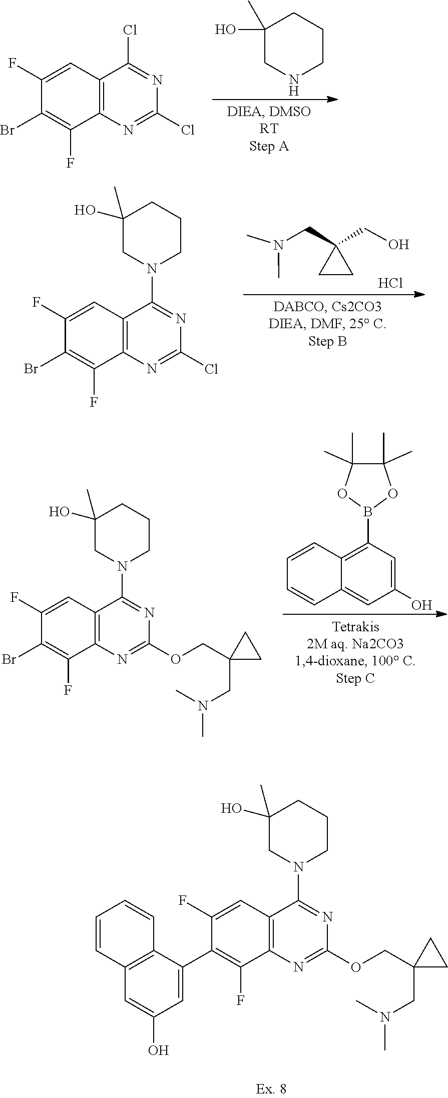

### Step A: 1-(7-bromo-2-chloro-6,8-difluoroquinazolin-4-yl)-3-methylpiperidin-3-ol

To a solution of 7-bromo-2,4-dichloro-6,8-difluoroquinazoline (500 mg, 1.59 mmol) and 3-methylpiperidin-3-ol hydrochloride (266 mg, 1.75 mmol) in DMSO (3 mL) was added N,N-diisopropylethylamine (1.10 mL, 6.37 mmol) at room temperature. After stirring the mixture at room temperature for 30 min, the reaction was quenched by the addition of water. The reaction mixture was extracted with EtOAc, washed with brine, dried over Na₂SO₄, filtered and concentrated in vacuo. The residue was purified by flash silica gel chromatography (20-50%, EtOAc gradient in hexane) to afford 1-(7-bromo-2-chloro-6,8-difluoroquinazolin-4-yl)-3-methylpiperidin-3-ol. ESI-MS m/z [M+H]⁺ 392, 394, 396.

### Step B: 1-(7-bromo-2-((1-((dimethylamino)methyl)cyclopropyl)methoxy)-6,8-difluoroquinazolin-4-yl)-3-methylpiperidin-3-ol

To a solution of 1-(7-bromo-2-chloro-6,8-difluoroquinazolin-4-yl)-3-methylpiperidin-3-ol (560 mg, 1.43 mmol) and (1-((dimethylamino)methyl)cyclopropyl)methanol (203 mg, 1.57 mmol) in DMF (14 mL) were added cesium carbonate (929 mg, 2.85 mmol) and 1,4-diazabicyclo[2.2.2]octane (32.0 mg, 0.285 mmol) at room temperature. After stirring the mixture at room temperature for 63 h, the reaction was quenched by the addition of water. The reaction mixture was extracted with EtOAc, washed with brine, dried over Na₂SO₄, filtered and concentrated in vacuo. The residue was purified by flash NH₂-silica gel chromatography (0-20%, MeOH gradient in EtOAc) to afford 1-(7-bromo-2-((1-((dimethylamino)methyl)cyclopropyl)methoxy)-6,8-difluoroquinazolin-4-yl)-3-methylpiperidin-3-ol. ESI-MS m/z [M+H]⁺ 485, 487.

### Step C: 1-(2-((1-((dimethylamino)methyl)cyclopropyl)methoxy)-6,8-difluoro-7-(3-hydroxvnaphthalen-1-yl)quinazolin-4-yl)-3-methylpiperidin-3-ol

To a solution of 1-(7-bromo-2-((1-((dimethylamino)methyl)cyclopropyl)methoxy)-6,8-difluoroquinazolin-4-yl)-3-methylpiperidin-3-ol (25.0 mg, 0.0515 mmol), 4-(4,4,5,5-tetramethyl-1,3,2-dioxaborolan-2-yl)naphthalen-2-ol (18.1 mg, 0.0670 mmol) and tetrakis(triphenylphosphine)palladium(0) (6.0 mg, 5.2 μmol) in 1,4-dioxane (0.5 mL) was added 2.0 M aqueous solution of Na₂CO₃ (51.5 μL, 0.103 mmol) at room temperature. After stirring the mixture at 100° C. for 16 h, the reaction was quenched by the addition of H₂O. The reaction mixture was extracted with EtOAc, washed with brine, dried over Na₂SO₄, filtered and concentrated in vacuo. The residue was purified by reverse phase HPLC (0-100%, MeCN/water with 0.1% formic acid) to afford 1-(2-((1-((dimethylamino)methyl)cyclopropyl)methoxy)-6,8-difluoro-7-(3-hydroxynaphthalen-1-yl)quinazolin-4-yl)-3-methylpiperidin-3-ol (Ex. 8). ESI-MS m/z [M+H]⁺ 549. ¹H NMR (400 MHz, DMSO-d₆) δ 10.02-10.00 (m, 1H), 7.89 (d, J=10.3 Hz, 1H), 7.81 (d, J=8.3 Hz, 1H), 7.48-7.41 (m, 1H), 7.35-7.21 (m, 3H), 7.16-7.12 (m, 1H), 4.69-4.68 (m, 1H), 4.24-4.16 (m, 2H), 4.14-4.04 (m, 1H), 3.91-3.82 (m, 1H), 3.42-3.33 (m, 1H), 3.29-3.20 (m, 1H), 2.22 (brs, 2H), 2.15 (s, 6H), 2.09-1.95 (m, 1H), 1.76-1.56 (m, 3H), 1.16-1.14 (m, 3H), 0.62 (brs, 2H), 0.39 (brs, 2H).

The compound in the table below was synthesized using a similar procedure as described in the synthesis of Ex. 8 by making the appropriate substitutions for starting material, intermediates, and/or reagents. Appropriate substitutions are available commercially, synthesized as described in the literature, synthesized using methods available to those skilled in the art, or synthesized as described herein.

| Ex | Structure | Compound Name | [M + H]⁺ Found |
|---|---|---|---|
| Ex. 9 | 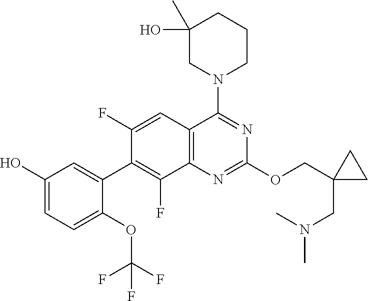 [SMILES: CN(C)CC1(COc2nc(N3CCCC(C)(O)C3)c3cc(F)c(-c4cc(O)ccc4OC(F)(F)F)c(F)c3n2)CC1] | 1-(2-((1- ((dimethylamino)methyl) cyclopropyl)methoxy)- 6,8-difluoro-7-(5- hydroxy-2- (trifluoromethoxy)phenyl) quinazolin-4-yl)-3- methylpiperidin-3-ol | 583 |

### Example 10: (3R)-1-(2-(((R)-1-((dimethylamino)methyl)-2,2-difluorocyclopropyl)methoxy)-7-(8-ethynyl-3-hydroxvnaphthalen-1-yl)-6,8-difluoroquinazolin-4-yl)-3-methylpiperidin-3-ol (Ex. 10)

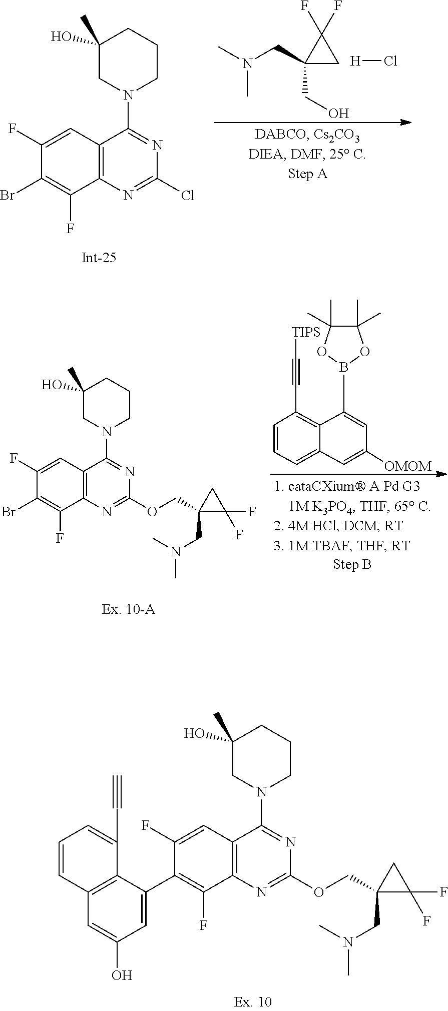

### Step A: (R)-1-(7-bromo-2-(((R)-1-((dimethylamino)methyl)-2,2-difluorocyclopropyl)methoxy)-6,8-difluoroquinazolin-4-yl)-3-methylpiperidin-3-ol

To a solution of (R)-1-(7-bromo-2-chloro-6,8-difluoroquinazolin-4-yl)-3-methylpiperidin-3-ol (Int-25) (300 mg, 0.764 mmol) and (R)-(1-((dimethylamino)methyl)-2,2-difluorocyclopropyl)methanol hydrochloride (Int-10) (169 mg, 0.840 mmol) in DMF (7.5 mL) were added cesium carbonate (747 mg, 2.29 mmol), 1,4-diazabicyclo[2.2.2]octane (17.1 mg, 0.153 mmol) and N,N-diisopropylethylamine (0.399 mL, 2.29 mmol) at room temperature. After stirring the mixture at room temperature for 87 h, the reaction was quenched by the addition of H₂O. The reaction mixture was extracted with EtOAc, washed with brine, dried over Na₂SO₄, filtered and concentrated in vacuo. The residue was purified by flash silica gel chromatography (20-100%, EtOAc gradient in hexane) to afford (R)-1-(7-bromo-2-(((R)-1-((dimethylamino)methyl)-2,2-difluorocyclopropyl)methoxy)-6,8-difluoroquinazolin-4-yl)-3-methylpiperidin-3-ol (Ex. 10-A). ESI-MS m/z [M+H]⁺ 521, 523. ¹H NMR (400 MHz, CDCl₃) δ 8.02 (dd, J=9.8, 2.0 Hz, 1H), 4.66 (s, 1H), 4.23 (d, J=13.0 Hz, 1H), 3.93 (d, J=13.3 Hz, 1H), 3.42 (d, J=13.4 Hz, 1H), 3.24-3.13 (m, 1H), 2.05-1.89 (m, 1H), 1.72-1.54 (m, 3H), 1.11 (s, 3H).

### Step B: (3R)-1-(2-(((R)-1-((dimethylamino)methyl)-2,2-difluorocyclopropyl)methoxy)-7-(8-ethynyl-3-hydroxvnaphthalen-1-yl)-6,8-difluoroquinazolin-4-yl)-3-methylpiperidin-3-ol

To a solution of (R)-1-(7-bromo-2-(((R)-1-((dimethylamino)methyl)-2,2-difluorocyclopropyl)methoxy)-6,8-difluoroquinazolin-4-yl)-3-methylpiperidin-3-ol (182 mg, 0.349 mmol), triisopropyl((6-(methoxymethoxy)-8-(4,4,5,5-tetramethyl-1,3,2-dioxaborolan-2-yl)naphthalen-1-yl)ethynyl)silane (Int-38) (259 mg, 0.524 mmol) and cataCXium® A Pd G3 (50.8 mg, 0.0698 mmol) in THF (3.5 mL) was added 1.0 M aqueous solution of K₃PO₄ (1.05 mL, 1.05 mmol) at room temperature. After stirring the mixture at 65° C. for 16 h, the reaction was quenched by the addition of H₂O. The reaction mixture was extracted with EtOAc, washed with brine, dried over Na₂SO₄, filtered and concentrated in vacuo. The residue was purified by flash NH₂-silica gel chromatography (20-50%, EtOAc gradient in hexane) to afford coupling product. To a solution of the coupling product in dichloromethane (4.0 mL) was added 4.0 M 1,4-dioxane solution of HCl (2.0 mL). After stirring the mixture at room temperature for 1 h, the reaction mixture was concentrated in vacuo. The residue was purified by reverse phase HPLC (MeCN/water with 0.10% formic acid) to afford naphthol product (peak 1, first elution). To a solution of the naphthol product (peak 1, first elution) in THF (3.5 mL) was added tetrabutylammonium fluoride (0.350 mL, 0.350 mmol, 1M in tetrahydrofuran), and the mixture was stirred at room temperature for 15 h. The mixture was diluted with ethyl acetate, the diluted mixture was washed with water, and the mixture was concentrated under reduced pressure. The residue was purified by flash NH-silica gel chromatography (0-10%, MeOH gradient in EtOAc) to afford (3R)-1-(2-(((R)-1-((dimethylamino)methyl)-2,2-difluorocyclopropyl)methoxy)-7-(8-ethynyl-3-hydroxynaphthalen-1-yl)-6,8-difluoroquinazolin-4-yl)-3-methylpiperidin-3-ol (Ex. 10). ESI-MS m/z [M+H]⁺ 609. ¹H NMR (400 MHz, DMSO-d₆) δ 10.18 (s, 1H), 7.89 (dd, J=8.2, 1.4 Hz, 1H), 7.68 (d, J=9.7 Hz, 1H), 7.50-7.39 (m, 2H), 7.37 (d, J=2.6 Hz, 1H), 7.11 (d, J=2.6 Hz, 1H), 4.68 (s, 1H), 4.43 (d, J=11.2 Hz, 1H), 4.38 (d, J=10.9 Hz, 1H), 4.08 (d, J=13.6 Hz, 1H), 3.87 (d, J=13.3 Hz, 1H), 3.58 (s, 1H), 3.39-3.33 (m, 1H), 3.27-3.17 (m, 1H), 2.60 (d, J=12.0 Hz, 1H), 2.35 (d, J=12.8 Hz, 1H), 2.15 (s, 6H), 2.09-1.96 (m, 1H), 1.82-1.56 (m, 4H), 1.52-1.42 (m, 1H), 1.14 (s, 3H).

The compounds in the table below were synthesized using a similar procedure as described in the synthesis of Ex. 10 by making the appropriate substitutions for starting material, intermediates, and/or reagents, optionally resolving stereoisomers after completion of the indicated step using the indicated conditions and carrying forward the indicated peak. Appropriate substitutions are available commercially, synthesized as described in the literature, synthesized using methods available to those skilled in the art, or synthesized as described herein.

| | | | | [M + H]⁺ |
|---|---|---|---|---|
| Ex | Structure | Compound Name | resolution | Found |
| Ex. 11 | 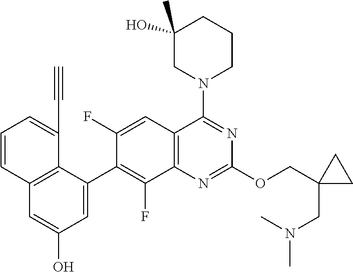 [SMILES: C#Cc1cccc2cc(O)cc(-c3c(F)cc4c(N5CCC[C@@](C)(O)C5)nc(OCC5(CN(C)C)CC5)nc4c3F)c12] | (3R)-1-(2-((1- ((dimethylamino)methyl)cy- clopropyl)methoxy)-7-(8- ethynyl-3- hydroxynaphthalen-1-yl)- 6,8-difluoroquinazolin-4- yl)-3-methylpiperidin-3-ol | Step B-2, Peak 1 (C18, 0-100% ACN/Water, w/0.1% formic acid) | 573 |
| Ex. 12 | 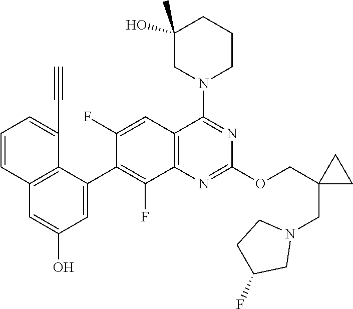 [SMILES: C#Cc1cccc2cc(O)cc(-c3c(F)cc4c(N5CCC[C@@](C)(O)C5)nc(OCC5(CN6CC[C@@H](F)C6)CC5)nc4c3F)c12] | (3R)-1-(7-(8-ethyny1-3- hydroxynaphthalen-1-yl)- 6,8-difluoro-2-((1-(((R)-3- fluoropyrrolidin-1- yl)methyl)cyclopropyl)meth- oxy)quinazolin-4-yl)-3- methylpiperidin-3-ol | N/A | 617 |

### Example 13: (3R)-1-(2-(((R)-1-((dimethylamino)methyl)-2,2-difluorocyclopropyl)methoxy)-6,8-difluoro-7-(3-hydroxvnaphthalen-1-yl)quinazolin-4-yl)-3-methylpiperidin-3-ol (Ex. 13)

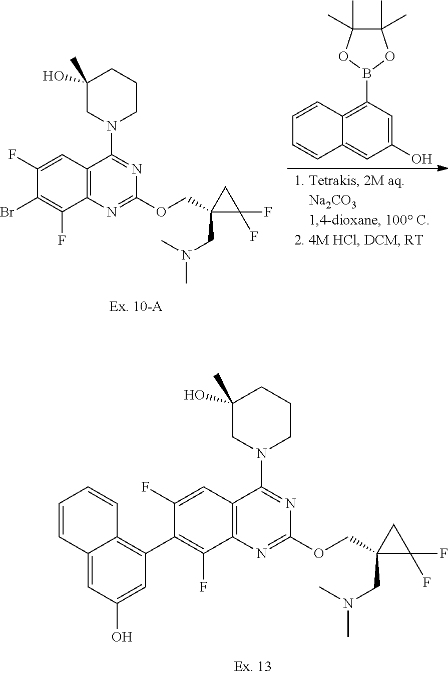 [SMILES: CN(C)C[C@@]1(COc2nc(N3CCC[C@@](C)(O)C3)c3cc(F)c(Br)c(F)c3n2)CC1(F)F ; CC1(C)OB(c2cc(O)cc3ccccc23)OC1(C)C ; CN(C)C[C@@]1(COc2nc(N3CCC[C@@](C)(O)C3)c3cc(F)c(-c4cc(O)cc5ccccc45)c(F)c3n2)CC1(F)F]

To a solution of (R)-1-(7-bromo-2-(((R)-1-((dimethylamino)methyl)-2,2-difluorocyclopropyl)methoxy)-6,8-difluoroquinazolin-4-yl)-3-methylpiperidin-3-ol (Ex. 10-A) (30.0 mg, 0.0575 mmol), 4-(4,4,5,5-tetramethyl-1,3,2-dioxaborolan-2-yl)naphthalen-2-ol (20.2 mg, 0.0748 mmol) and tetrakis(triphenylphosphine)palladium(0) (6.7 mg, 5.8 μmol) in 1,4-dioxane (0.6 mL) was added 2.0 M aqueous solution of Na₂CO₃ (57.5 μL, 0.115 mmol) at room temperature. After stirring the mixture at 100° C. for 14 h, the reaction was quenched by the addition of H₂O. The reaction mixture was extracted with EtOAc, washed with brine, dried over Na₂SO₄, filtered and concentrated in vacuo. The residue was purified by reverse phase HPLC (MeCN/water with 0.1% formic acid) to afford (3R)-1-(2-(((R)-1-((dimethylamino)methyl)-2,2-difluorocyclopropyl)methoxy)-6,8-difluoro-7-(3-hydroxynaphthalen-1-yl)quinazolin-4-yl)-3-methylpiperidin-3-ol (Ex. 13). ESI-MS m z [M+H]⁺ 585. ESI-MS m/z [M+H]⁺ 621, 623. ¹H NMR (400 MHz, DMSO-d₆) δ 10.02-10.00 (m, 1H), 7.91 (d, J=10.3 Hz, 1H), 7.81 (d, J=8.1 Hz, 1H), 7.48-7.41 (m, 1H), 7.36-7.21 (m, 3H), 7.17-7.13 (m, 1H), 4.70-4.68 (m, 1H), 4.45 (d, J=11.2 Hz, 1H), 4.39 (d, J=11.2 Hz, 1H), 4.11 (d, J=13.0 Hz, 1H), 3.95-3.85 (m, 1H), 3.40 (d, J=13.2 Hz, 1H), 3.30-3.20 (m, 1H), 2.61 (d, J=13.2 Hz, 1H), 2.35 (d, J=13.0 Hz, 1H), 2.15 (s, 6H), 2.09-1.95 (m, 1H), 1.84-1.56 (m, 4H), 1.52-1.42 (m, 1H), 1.17-1.16 (m, 3H).

### Example 14: (3R)-1-(7-(8-chloro-7-fluoronaphthalen-1-yl)-2-(((R)-1-((dimethylamino)methyl)-2,2-difluorocyclopropyl)methoxy)-6,8-difluoroquinazolin-4-yl)-3-methylpiperidin-3-ol (Ex. 14)

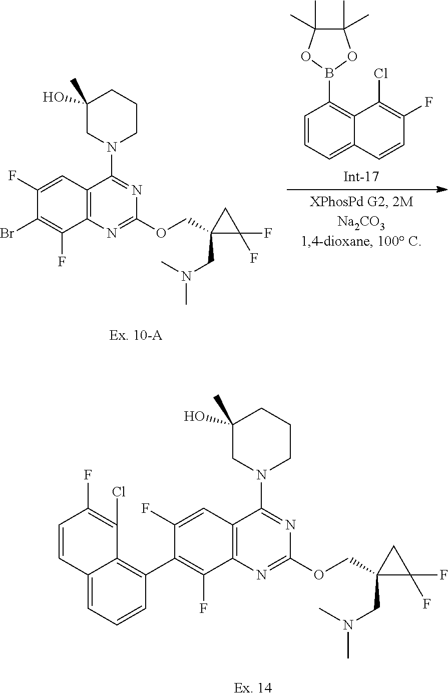 [SMILES: CN(C)C[C@@]1(COc2nc(N3CCC[C@@](C)(O)C3)c3cc(F)c(Br)c(F)c3n2)CC1(F)F ; CC1(C)OB(c2cccc3ccc(F)c(Cl)c23)OC1(C)C ; CN(C)C[C@@]1(COc2nc(N3CCC[C@@](C)(O)C3)c3cc(F)c(-c4cccc5ccc(F)c(Cl)c45)c(F)c3n2)CC1(F)F]

To a solution of (R)-1-(7-bromo-2-(((R)-1-((dimethylamino)methyl)-2,2-difluorocyclopropyl)methoxy)-6,8-difluoroquinazolin-4-yl)-3-methylpiperidin-3-ol (Ex. 10-A) (30.0 mg, 0.0575 mmol), 2-(8-chloro-7-fluoronaphthalen-1-yl)-4,4,5,5-tetramethyl-1,3,2-dioxaborolane (35.3 mg, 0.115 mmol) and XPhos Pd G2 (4.5 mg, 5.8 μmol) in 1,4-dioxane (0.6 mL) was added 2.0 M aqueous solution of Na₂CO₃ (57.5 mL, 0.115 mmol) at room temperature. After stirring the mixture at 100° C. for 6 h, the reaction was quenched by the addition of H₂O. The reaction mixture was extracted with EtOAc, washed with brine, dried over Na₂SO₄, filtered and concentrated in vacuo. The residue was purified by reverse phase HPLC (0-100%, MeCN/water with 0.1% formic acid) to afford (3R)-1-(7-(8-chloro-7-fluoronaphthalen-1-yl)-2-(((R)-1-((dimethylamino)methyl)-2,2-difluorocyclopropyl)methoxy)-6,8-difluoroquinazolin-4-yl)-3-methylpiperidin-3-ol (Ex. 14). ESI-MS m/z [M+H]⁺ 621, 623. ¹H NMR (400 MHz, DMSO-d₆) δ 8.28-8.19 (m, 2H), 7.88 (d, J=9.0 Hz, 0.5H), 7.79 (d, J=9.4 Hz, 0.5H), 7.77-7.65 (m, 3H), 4.79-4.65 (m, 1H), 4.45 (d, J=10.9 Hz, 1H), 4.38 (d, J=11.4 Hz, 1H), 4.15-4.00 (m, 1H), 3.86 (d, J=12.8 Hz, 1H), 3.45-3.33 (m, 1H), 3.30-3.20 (m, 1H), 2.61 (d, J=13.2 Hz, 1H), 2.38-2.30 (m, 1H), 2.15 (s, 6H), 2.09-1.92 (m, 1H), 1.84-1.55 (m, 4H), 1.52-1.42 (m, 1H), 1.16-1.14 (m, 3H).

### Example 15: (3R)-1-(2-(((R)-1-((dimethylamino)methyl)-2,2-difluorocyclopropyl)methoxy)-7-(8-ethynyl-7-fluoronaphthalen-1-yl)-6,8-difluoroquinazolin-4-yl)-3-methylpiperidin-3-ol (Ex. 15)

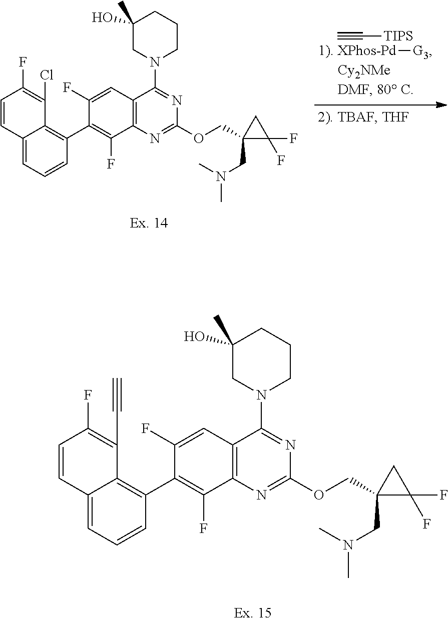

To a solution of (3R)-1-(7-(8-chloro-7-fluoronaphthalen-1-yl)-2-(((R)-1-((dimethylamino)methyl)-2,2-difluorocyclopropyl)methoxy)-6,8-difluoroquinazolin-4-yl)-3-methylpiperidin-3-ol (Ex. 14) (7.2 mg, 12 μmol), triisopropylsilylacetylene (25.8 μL, 0.116 mmol) and XPhos Pd G3 (4.9 mg, 5.8 μmol) in DMF (0.25 mL) was added N-cyclohexyl-N-methylcyclohexanamine (12.4 μL, 58.0 μmol). The vessel was evacuated and backfilled with nitrogen, and the mixture was stirred at 100° C. for 15 h. The mixture was diluted with EtOAc, the diluted mixture was washed with saturated aqueous NaHCO₃, and the mixture was concentrated under reduced pressure to afford coupling product. The residue was purified by flash NH₂-silica gel chromatography (20-50%, EtOAc gradient in hexane) to give the coupling product.

To a solution of the coupling product in THF (0.2 mL) was added tetrabutylammonium fluoride (0.0200 mL, 0.0200 mmol, 1M in tetrahydrofuran), and the mixture was stirred at room temperature for 30 min. The mixture was diluted with ethyl acetate, the diluted mixture was washed with water, and the mixture was concentrated under reduced pressure. The residue was purified by reverse phase HPLC (MeCN/water with 0.1% formic acid) to afford (3R)-1-(2-(((R)-1-((dimethylamino)methyl)-2,2-difluorocyclopropyl)methoxy)-7-(8-ethynyl-7-fluoronaphthalen-1-yl)-6,8-difluoroquinazolin-4-yl)-3-methylpiperidin-3-ol (Ex. 15). ESI-MS m/z [M+H]⁺ 611. ¹H NMR (400 MHz, DMSO-d₆) δ 8.27-8.14 (m, 2H), 7.81-7.58 (m, 4H), 4.72-4.69 (m, 1H), 4.48-4.33 (m, 2H), 4.15-4.00 (m, 2H), 3.95-3.78 (m, 1H), 3.45-3.35 (m, 1H), 3.28-3.15 (m, 1H), 2.65-2.56 (m, 1H), 2.40-2.31 (m, 1H), 2.15 (s, 6H), 2.09-1.95 (m, 1H), 1.84-1.55 (m, 4H), 1.52-1.42 (m, 1H), 1.16-1.14 (m, 3H).

### Example 16: (R)-1-(2-((1-((dimethylamino)methyl)cyclopropyl)methoxy)-7-(8-ethynyl-3-hydroxynaphthalen-1-yl)-8-fluoroquinazolin-4-yl)-3-methylpiperidin-3-ol (Ex. 16)

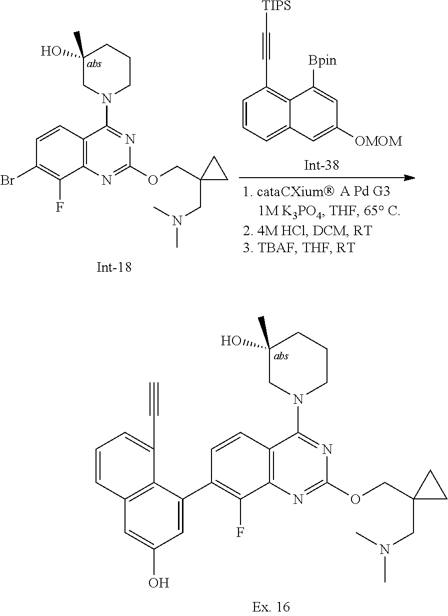

To a solution of (R)-1-(7-bromo-2-((1-((dimethylamino)methyl)cyclopropyl)methoxy)-8-fluoroquinazolin-4-yl)-3-methylpiperidin-3-ol (Int-18) (123 mg, 0.263 mmol), triisopropyl((6-(methoxymethoxy)-8-(4,4,5,5-tetramethyl-1,3,2-dioxaborolan-2-yl)naphthalen-1-yl)ethynyl)silane (Int-38) (156 mg, 0.316 mmol) and cataCXium® A Pd G3 (38.3 mg, 0.0526 mmol) in THF (2.6 mL) was added 1.0 M aqueous solution of K3P04 (0.790 mL, 0.790 mmol) at room temperature. After stirring the mixture at 65° C. for 15 h, the reaction was quenched by the addition of H₂O. The reaction mixture was extracted with EtOAc, washed with brine, dried over Na₂SO₄, filtered and concentrated in vacuo. The residue was purified by flash silica gel chromatography (0-30%, MeOH gradient in EtOAc) to afford the coupled intermediate which was redissolved in DCM (4 mL). The reaction mixture was treated with 4.0 M 1,4-dioxane solution of HCl (2.0 mL). After stirring the mixture at room temperature for 30 min, the reaction mixture was concentrated in vacuo. The residue was purified by reverse phase HPLC (0-100%, MeCN/water with 0.1% formic acid) to afford the naphthol deprotected intermediate which was redissolved in THF (4 mL). The reaction mixture was treated with tetrabutylammonium fluoride (0.800 mL, 0.800 mmol, 1M in tetrahydrofuran), and the mixture was stirred at room temperature for 20 h. The mixture was diluted with ethyl acetate, the diluted mixture was washed with water, and the mixture was concentrated under reduced pressure. The residue was purified by flash NH₂-silica gel chromatography (0-10%, MeOH gradient in EtOAc) to afford (R)-1-(2-((1-((dimethylamino)methyl)cyclopropyl)methoxy)-7-(8-ethynyl-3-hydroxynaphthalen-1-yl)-8-fluoroquinazolin-4-yl)-3-methylpiperidin-3-ol (Ex. 16). ESI-MS m/z [M+H]⁺ 555. ¹H NMR (400 MHz, DMSO-d₆) δ 10.09 (s, 1H), 7.89-7.75 (m, 2H), 7.47-7.37 (m, 2H), 7.29 (d, J=2.5 Hz, 1H), 7.21-7.14 (m, 1H), 7-7.00 (m, 1H), 4.65-4.60 (m, 1H), 4.23-4.14 (m, 2H), 4.10-3.92 (m, 1H), 3.90-3.72 (m, 1H), 3.50-3.36 (m, 2H), 3.29-3.22 (m, 1H), 2.22 (brs, 2H), 2.15 (s, 6H), 2.10-1.94 (m, 1H), 1.74-1.56 (m, 3H), 1.15-1.14 (m, 3H), 0.62 (brs, 2H), 0.38 (brs, 2H).

### Example 17: 1-(7-(2-amino-7-fluorobenzo[d]thiazol-4-yl)-6-chloro-2-((1-((dimethylamino)methyl)cyclopropyl)methoxy)-8-fluoroquinazolin-4-yl)azepan-4-ol (Ex. 17)

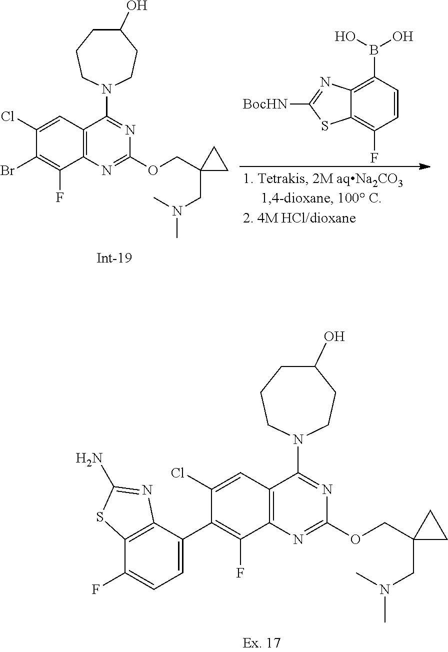 [SMILES: CN(C)CC1(COc2nc(N3CCCC(O)CC3)c3cc(Cl)c(Br)c(F)c3n2)CC1 ; CC(C)(C)OC(=O)Nc1nc2c(B(O)O)ccc(F)c2s1 ; CN(C)CC1(COc2nc(N3CCCC(O)CC3)c3cc(Cl)c(-c4ccc(F)c5sc(N)nc45)c(F)c3n2)CC1]

To a mixture of 1-(7-bromo-6-chloro-2-((1-((dimethylamino)methyl)cyclopropyl)methoxy)-8-fluoroquinazolin-4-yl)azepan-4-ol (Int-19) (25 mg, 0.050 mmol), [2-(tert-butoxycarbonylamino)-7-fluoro-1,3-benzothiazol-4-yl]boronic acid (20 mg, 0.065 mmol), and tetrakis(triphenylphosphine)palladium(0) (5.8 mg, 5.0 μmol) in 1,4-dioxane (2 mL) was added sodium carbonate (0.050 mL, 2M solution in water), and the mixture was stirred at 100° C. for 16 h. The mixture was diluted with ethyl acetate, and the diluted mixture was concentrated under reduced pressure. To the residue was added hydrochloride (3 mL, 4M in 1,4-dioxane), and the mixture was stirred at 50° C. for 1 h. After concentration under reduced pressure, the residue was purified by reverse phase HPLC (MeCN/water with 0.1% formic acid) to give 1-(7-(2-amino-7-fluorobenzo[d]thiazol-4-yl)-6-chloro-2-((1-((dimethylamino)methyl)cyclopropyl)methoxy)-8-fluoroquinazolin-4-yl)azepan-4-ol (Ex. 17). ESI-MS m/z [M+H]⁺ 589, 591. ¹H NMR (400 MHz, DMSO-d₆) δ 7.99-7.90 (m, 3H), 7.26-7.21 (m, 1H), 7.09-7.03 (m, 1H), 4.65-4.63 (m, 1H), 4.25-4.17 (m, 2H), 4.00-3.75 (m, 6H), 2.55 (s, 2H), 2.30-2.05 (m, 7H), 1.94-1.69 (m, 3H), 1.63-1.53 (m, 1H), 0.65-0.59 (m, 2H), 0.43-0.37 (m, 2H).

### Example 18: 2-amino-4-(6-chloro-8-fluoro-2-(((2R,7aS)-2-fluorotetrahydro-1H-pyrrolizin-7a(5H)-yl)methoxy)-4-(4-hydroxyazepan-1-yl)quinazolin-7-yl)-7-fluorobenzo[b]thiophene-3-carbonitrile (Ex. 18)

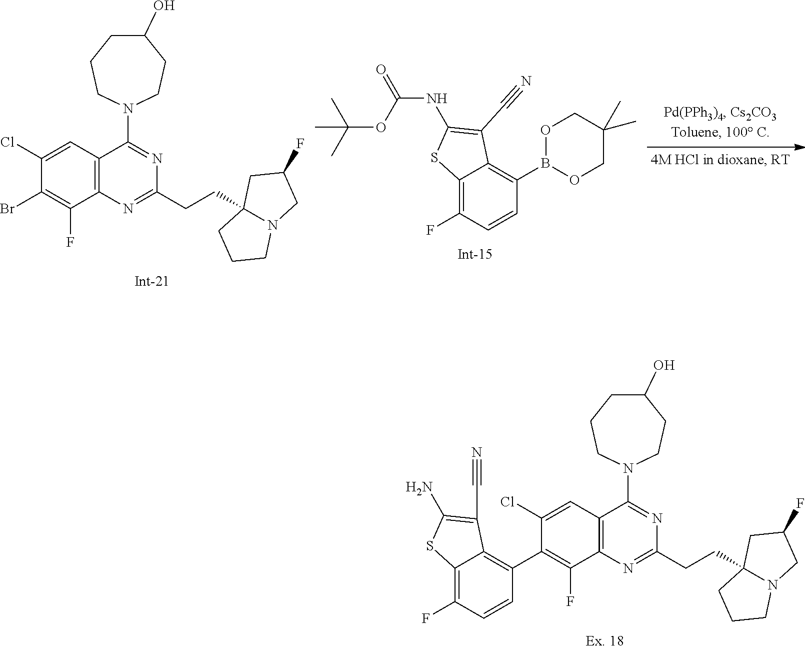

To a solution of 1-(7-bromo-6-chloro-8-fluoro-2-(((2R,7aS)-2-fluorotetrahydro-1H-pyrrolizin-7a(5H)-yl)methoxy)quinazolin-4-yl)azepan-4-ol (Int-21) (30.0 mg, 0.0564 mmol), tert-butyl (3-cyano-4-(5,5-dimethyl-1,3,2-dioxaborinan-2-yl)-7-fluorobenzo[b]thiophen-2-yl)carbamate (Int-15) (34.2 mg, 0.0846 mmol) and dichlorobis(diphenylphosphinophenyl)ether palladium (II) (6.1 mg, 8.5 μmol) in toluene (1.5 mL) was added cesium carbonate (45.9 mg, 0.141 mmol). After stirring the mixture at 100° C. for 13 h, the reaction mixture was cooled to room temperature, quenched with H₂O, extracted with EtOAc, washed with brine, dried over Na₂SO₄, filtered and concentrated in vacuo. The residue was purified by flash silica gel chromatography (0-50%, MeOH gradient in EtOAc) to afford the coupled intermediate which was redissolved in DCM (0.5 mL). The reaction mixture was treated with HCl (0.5 mL, 4.0 M in 1,4-dioxane). After stirring the mixture at room temperature for 6 h, the reaction mixture was concentrated in vacuo. The residue was purified by flash NH-silica gel chromatography (0-10%, MeOH gradient in EtOAc) to afford 2-amino-4-(6-chloro-8-fluoro-2-(((2R,7aS)-2-fluorotetrahydro-1H-pyrrolizin-7a(5H)-yl)methoxy)-4-(4-hydroxyazepan-1-yl)quinazolin-7-yl)-7-fluorobenzo[b]thiophene-3-carbonitrile (Ex. 18). ESI-MS m/z [M+H]⁺ 643, 645. ¹H NMR (400 MHz, DMSO-d₆) δ 8.09 (s, 2H), 7.95 (s, 1H), 7.26-7.21 (m, 1H), 7.17-7.10 (m, 1H), 5.33-5.20 (m, 1H), 4.66-4.61 (m, 1H), 4.12-4.05 (m, 1H), 4.02-3.70 (m, 6H), 3.13-2.96 (m, 3H), 2.87-2.75 (m, 1H), 2.15-1.67 (m, 9H), 1.61-150 (m, 1H), 1.26-1.19 (m, 2H).

### Example 19: (R)-1-(2-((1-((dimethylamino)methyl)cyclopropyl)methoxy)-7-(8-ethynyl-3-hydroxynaphthalen-1-yl)pyrido[3,2-d]pyrimidine-4-yl)-3-methylpiperidin-3-ol (Ex. 19)

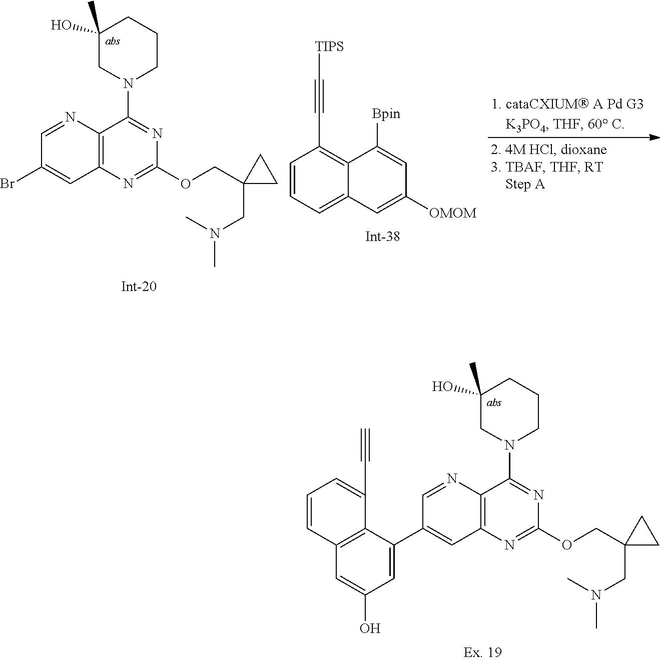

To a solution of (R)-1-(7-bromo-2-((1-((dimethylamino)methyl)cyclopropyl)methoxy)pyrido[3,2-d]pyrimidin-4-yl)-3-methylpiperidin-3-ol (Int-20) (60 mg, 0.13 mmol), triisopropyl((6-(methoxymethoxy)-8-(4,4,5,5-tetramethyl-1,3,2-dioxaborolan-2-yl)naphthalen-1-yl)ethynyl)silane (Int-38) (79 mg, 0.16 mmol) and cataCXium® A Pd G3 (19 mg, 0.027 mmol) in THF (3.0 mL) was added 1.0 M aqueous solution of K₃PO₄ (0.40 mL, 0.40 mmol) at room temperature. After stirring the mixture at 60° C. for 4 h, the reaction was quenched by the addition of H₂O. The reaction mixture was extracted with EtOAc, washed with water, and the mixture was concentrated under reduced pressure. To the residue was added hydrochloride (0.3 mL, 4M in 1,4-dioxane), and the mixture was stirred at room temperature for 1 h. After concentration, tetrabutylammonium fluoride (0.3 mL, 1M in THF) was added to the residue, and the mixture was stirred at room temperature for 1 h. The mixture was diluted with ethyl acetate, the diluted mixture was washed with water, and the mixture was concentrated under reduced pressure. The residue was purified by reverse phase HPLC (MeCN/water with 0.1% formic acid) to give (R)-1-(2-((1-((dimethylamino)methyl)cyclopropyl)methoxy)-7-(8-ethynyl-3-hydroxynaphthalen-1-yl)pyrido[3,2-d]pyrimidin-4-yl)-3-methylpiperidin-3-ol (Ex. 19). ESI-MS m/z [M+H]⁺ 538. ¹H NMR (400 MHz, DMSO-d₆) δ 10.17 (br s, 1H), 8.48 (dd, J=4.4, 2.4 Hz, 1H), 7.91-7.88 (m, 1H), 7.72 (dd, J=12.8, 2 Hz, 1H), 7.51-7.40 (m, 2H), 7.31 (d, J=2.8 Hz, 1H), 7.07 (dd, J=17.4, 2.4 Hz, 1H), 4.57-4.49 (m, 1H) 4.38-4.15 (m, 3H), 4.10-3.90 (m, 1H), 3.74-3.68 (m, 1H), 2.29-2.21 (m, 2H), 2.18 (s, 6H), 2.01-1.86 (m, 2H), 1.71-1.50 (m, 3H), 1.25-1.22 (m, 1H), 1.15-1.07 (m, 3H), 0.65-0.61 (m, 2H), 0.43-0.38 (m, 2H).

### Example 20: (R)-1-(7-(8-chloro-3-hydroxynaphthalen-1-yl)-2-((1-((dimethylamino)methyl)cyclopropyl)methoxy)-8-fluoropyrido[4,3-d]pyrimidin-4-yl)-3-methylpiperidin-3-ol (Ex. 20)

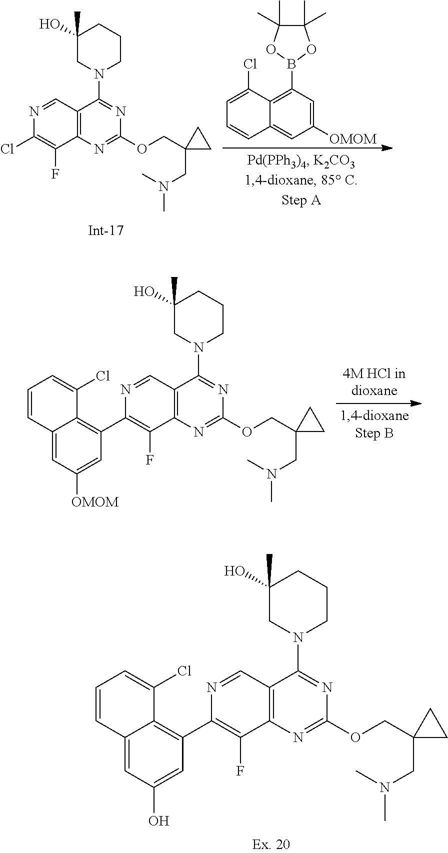

### Step A: (R)-1-(7-(8-chloro-3-(methoxymethoxy)naphthalen-1-yl)-2-((1-((dimethylamino)methyl)cyclopropyl)methoxy)-8-fluoropyrido[4,3-d]pyrimidin-4-yl)-3-methylpiperidin-3-ol

To a solution of (R)-1-(7-chloro-2-((1-((dimethylamino)methyl)cyclopropyl)methoxy)-8-fluoropyrido[4,3-d]pyrimidin-4-yl)-3-methylpiperidin-3-ol (30 mg, 0.071 mmol), 2-(8-chloro-3-(methoxymethoxy)naphthalen-1-yl)-4,4,5,5-tetramethyl-1,3,2-dioxaborolane (30 mg, 0.085 mmol) and potassium carbonate (48.9 mg, 0.354 mmol) in 1,4-dioxane (3.0 ml) and water (0.6 ml) was added tetrakis(triphenylphosphine)palladium(0) (16 mg, 0.014 mmol) at room temperature. The mixture was degassed under reduced pressure and purged with nitrogen several times. The mixture was stirred at 85° C. for 24 h under nitrogen atmosphere. The reaction mixture was cooled to room temperature and diluted with water and ethyl acetate. The organic layer was dried over MgSO₄, filtered and concentrated in vacuo. The residue was purified by flash NH₂-silica chromatography (eluent, 12-100% EtOAc in hexane) and reverse phase HPLC (MeCN/water with 0.1% formic acid) to obtain (R)-1-(7-(8-chloro-3-(methoxymethoxy)naphthalen-1-yl)-2-((1-((dimethylamino)methyl)cyclopropyl)methoxy)-8-fluoropyrido[4,3-d]pyrimidin-4-yl)-3-methylpiperidin-3-ol. ESI-MS m/z [M+H]⁺ 610, 612. ¹H NMR (400 MHz, CDCl₃) δ 9.12-9.22 (m, 1H), 7.32-7.84 (m, 5H), 5.30-5.35 (m, 2H), 4.28-4.45 (m, 4H), 3.44-3.75 (m, 5H), 3.26-3.34 (m, 1H), 3.14 (br s, 1H), 2.31-2.62 (m, 8H), 1.43-2.10 (m, 4H), 1.23-1.36 (m, 3H), 0.72 (br s, 2H), 0.51 (br s, 2H).

### Step B: (R)-1-(7-(8-chloro-3-hydroxynaphthalen-1-yl)-2-((1-((dimethylamino)methyl)cyclopropyl)methoxy)-8-fluoropyrido[4,3-d]pyrimidin-4-yl)-3-methylpiperidin-3-ol (Ex. 20)

To a solution of (R)-1-(7-(8-chloro-3-(methoxymethoxy)naphthalen-1-yl)-2-((1-((dimethylamino)methyl)cyclopropyl)methoxy)-8-fluoropyrido[4,3-d]pyrimidin-4-yl)-3-methylpiperidin-3-ol (7.6 mg, 0.013 mmol) in 1,4-dioxane (1.0 mL) was added 4M HCl in 1,4-dioxane (93.4 μL, 0.374 mmol) at room temperature under nitrogen atmosphere. The mixture was stirred at room temperature for 1 h under nitrogen atmosphere. The reaction mixture was concentrated in vacuo. The residue was purified by reverse phase HPLC (0-100% MeCN/water with 0.1% formic acid) to obtain (R)-1-(7-(8-chloro-3-hydroxynaphthalen-1-yl)-2-((1-((dimethylamino)methyl)cyclopropyl)methoxy)-8-fluoropyrido[4,3-d]pyrimidin-4-yl)-3-methylpiperidin-3-ol (Ex. 20). ESI-MS m/z [M+H]⁺ 566, 568. ¹H NMR (400 MHz, CDCl₃) δ 9.00-9.19 (m, 1H), 7.51-7.76 (m, 1H), 7.18-7.25 (m, 2H), 6.37-7.08 (m, 2H), 3.94-4.49 (m, 5H), 3.15-3.23 (m, 1H), 2.91-3.00 (m, 1H), 2.53-2.66 (m, 2H), 2.42-2.46 (m, 6H), 1.29-1.88 (m, 4H), 1.24-1.26 (m, 3H), 0.73 (br s, 2H), 0.55 (br s, 2H).

### Example 21: (R)-1-(2-((1-((dimethylamino)methyl)cyclopropyl)methoxy)-7-(8-ethynyl-3-hydroxynaphthalen-1-yl)-8-fluoropyrido[4,3-d]pyrimidin-4-yl)-3-methylpiperidin-3-ol (Ex. 21)

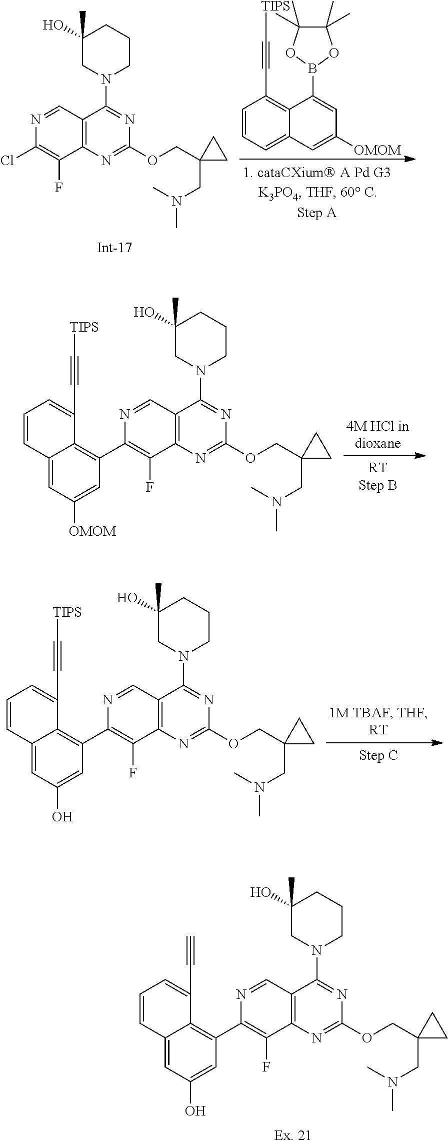

### Step A: (R)-1-(2-((1-((dimethylamino)methyl)cyclopropyl)methoxy)-8-fluoro-7-(3-(methoxymethoxy)-8-((triisopropylsilyl)ethynyl)naphthalen-1-yl)pyrido[4,3-d]pyrimidin-4-yl)-3-methylpiperidin-3-ol

To a solution of (R)-1-(7-chloro-2-((1-((dimethylamino)methyl)cyclopropyl)methoxy)-8-fluoropyrido[4,3-d]pyrimidin-4-yl)-3-methylpiperidin-3-ol (Int-17) (100 mg, 0.236 mmol), triisopropyl((6-(methoxymethoxy)-8-(4,4,5,5-tetramethyl-1,3,2-dioxaborolan-2-yl)naphthalen-1-yl)ethynyl)silane (Int-38) (140 mg, 0.283 mmol) and cataCXium® A Pd G3 (34 mg, 0.047 mmol) in THF (2.0 mL) was added potassium phosphate tribasic (150 mg, 0.708 mmol) at room temperature. The mixture was degassed under reduced pressure and purged with nitrogen several times. The mixture was stirred at 60° C. for 16 h under nitrogen atmosphere. The reaction mixture was cooled to room temperature and diluted with water and ethyl acetate. The organic layer was dried over MgSO₄, filtered and concentrated in vacuo. The residue was purified by flash NH-silica chromatography (eluent, 12-100% EtOAc in hexane) and reverse phase HPLC (C18, 0-100%, MeCN/water with 0.1% formic acid) to obtain (R)-1-(2-((1-((dimethylamino)methyl)cyclopropyl)methoxy)-8-fluoro-7-(3-(methoxymethoxy)-8-((triisopropylsilyl)ethynyl)naphthalen-1-yl)pyrido[4,3-d]pyrimidin-4-yl)-3-methylpiperidin-3-ol. ESI-MS m/z [M+H]⁺ 756. ¹H NMR (400 MHz, CDCl₃) δ 9.15-9.18 (m, 1H), 7.80-7.82 (m, 1H), 7.66-7.70 (m, 1H), 7.51-7.52 (m, 1H), 7.38-7.43 (m, 1H), 7.30-7.33 (m, 1H), 5.29-5.34 (m, 2H), 4.31-4.48 (m, 4H), 2.85-3.75 (m, 6H), 2.27-2.43 (m, 8H), 1.60-2.17 (m, 4H), 1.22-1.35 (m, 3H), 0.85-0.89 (m, 18H), 0.48-0.74 (m, 7H).

### Step B: (R)-1-(2-((1-((dimethylamino)methyl)cyclopropyl)methoxy)-8-fluoro-7-(3-hydroxy-8-((triisopropylsilyl)ethynyl)naphthalen-1-yl)pyrido[4,3-d]pyrimidin-4-yl)-3-methylpiperidin-3-ol

To a solution of (R)-1-(2-((1-((dimethylamino)methyl)cyclopropyl)methoxy)-8-fluoro-7-(3-(methoxymethoxy)-8-((triisopropylsilyl)ethynyl)naphthalen-1-yl)pyrido[4,3-d]pyrimidin-4-yl)-3-methylpiperidin-3-ol (78 mg, 0.10 mmol) in 1,4-dioxane (1.0 mL) was added 4M HCl in 1,4-dioxane (0.129 mL, 0.516 mmol) at room temperature under a nitrogen atmosphere. The mixture was stirred at room temperature for 1 h under a nitrogen atmosphere. The reaction mixture was concentrated in vacuo. The residue was purified by reverse phase HPLC (C18, 0-100%, MeCN/water with 0.1% formic acid) to obtain (R)-1-(2-((1-((dimethylamino)methyl)cyclopropyl)methoxy)-8-fluoro-7-(3-hydroxy-8-((triisopropylsilyl)ethynyl)naphthalen-1-yl)pyrido[4,3-d]pyrimidin-4-yl)-3-methylpiperidin-3-ol. ESI-MS m/z [M+H]⁺ 712. ¹H NMR (400 MHz, CDCl₃) δ 9.12-9.01 (m, 1H), 7.59-7.68 (m, 1H), 7.48-7.53 (m, 1H), 7.48-7.50 (m, 1H), 7.24-7.32 (m, 1H), 6.90-6.97 (m, 1H), 6.14-6.67 (m, 1H), 3.94-4.90 (m, 5H), 2.18-3.22 (m, 10H), 1.21-1.74 (m, 7H), 0.44-0.81 (m, 25H).

### Step C: (R)-1-(2-((1-((dimethylamino)methyl)cyclopropyl)methoxy)-7-(8-ethynyl-3-hydroxvnaphthalen-1-yl)-8-fluoropyrido[4,3-d]pyrimidine-4-yl)-3-methylpiperidin-3-ol (Ex. 21)

To a solution of (R)-1-(2-((1-((dimethylamino)methyl)cyclopropyl)methoxy)-8-fluoro-7-(3-hydroxy-8-((triisopropylsilyl)ethynyl)naphthalen-1-yl)pyrido[4,3-d]pyrimidin-4-yl)-3-methylpiperidin-3-ol (62 mg, 0.087 mmol) in THF (1.0 mL) was added 1M-tetrabutylammonium fluoride in THF (0.26 mL, 0.26 mmol) at room temperature under a nitrogen atmosphere. The mixture was stirred at room temperature for 3 h under a nitrogen atmosphere. The reaction mixture was cooled to room temperature and diluted with water and ethyl acetate. The aqueous layer was extracted with ethyl acetate. The combined organic layer was washed with brine, dried over MgSO₄, filtered and concentrated in vacuo. The residue was purified by reverse phase HPLC (MeCN/water with 0.1% formic acid) to obtain (R)-1-(2-((1-((dimethylamino)methyl)cyclopropyl)methoxy)-7-(8-ethynyl-3-hydroxynaphthalen-1-yl)-8-fluoropyrido[4,3-d]pyrimidin-4-yl)-3-methylpiperidin-3-ol (Ex. 21). ESI-MS m/z [M+H]⁺ 556. ¹H NMR (400 MHz, CDCl₃) δ 8.90-9.07 (m, 1H), 7.58-7.65 (m, 1H), 7.36-7.45 (m, 1H), 7.19-7.30 (m, 1H), 6.56-7.02 (m, 2H), 4.00-4.49 (m, 5H), 3.72 (q, 2H, J=7.0 Hz), 3.01-3.19 (m, 2H), 2.48-2.62 (m, 3H), 2.34-2.39 (m, 6H), 1.37-1.89 (m, 4H), 1.20-1.28 (m, 3H), 0.66-0.74 (m, 2H), 0.50-0.54 (m, 2H).

The compounds in the table below were synthesized using a similar procedure as described in the synthesis of Ex. 21 by making the appropriate substitutions for starting material, intermediates, and/or reagents. Appropriate substitutions are available commercially, synthesized as described in the literature, synthesized using methods available to those skilled in the art, or synthesized as described herein.

| | | | ESI-MS |
|---|---|---|---|
| Example | Structure | Name | [M + H]⁺ |
| Ex. 22 | 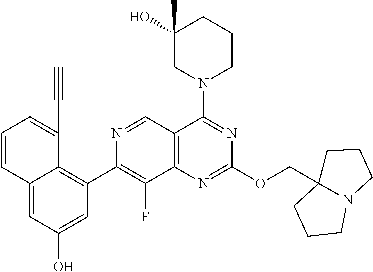 [SMILES: C#Cc1cccc2cc(O)cc(-c3ncc4c(N5CCC[C@@](C)(O)C5)nc(OCC56CCCN5CCC6)nc4c3F)c12] | (R)-1-(7-(8-ethyny1- 3- hydroxynaphthalen- 1-yl)-8-fluoro-2- ((tetrahydro-1H- pyrrolizin-7a(5H)- yl)methoxy)pyrido[4,3- d]pyrimidin-4- yl)-3- methylpiperidin-3-ol | 568 |
| Ex. 23 | 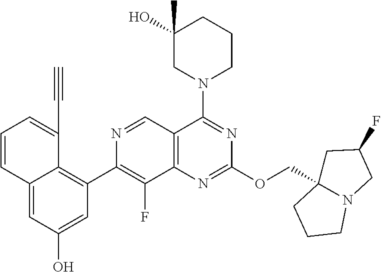 [SMILES: C#Cc1cccc2cc(O)cc(-c3ncc4c(N5CCC[C@@](C)(O)C5)nc(OC[C@@]56CCCN5C[C@H](F)C6)nc4c3F)c12] | (R)-1-(7-(8-ethynyl- 3- hydroxynaphthalen- 1-yl)-8-fluoro-2- (((2R,7aS)-2- fluorotetrahydro- 1H-pyrrolizin- 7a(5H)- yl)methoxy)pyrido[4,3- d]pyrimidin-4- yl)-3- methylpiperidin-3-ol | 586 |
| Ex. 24 | 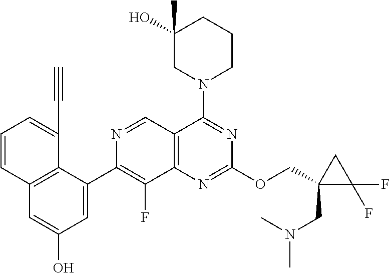 [SMILES: C#Cc1cccc2cc(O)cc(-c3ncc4c(N5CCC[C@@](C)(O)C5)nc(OC[C@]5(CN(C)C)CC5(F)F)nc4c3F)c12] | (R)-1-(2-(((R)-1- ((dimethylamino)meth- yl)-2,2- difluorocyclopropyl) methoxy)-7-(8- ethynyl-3- hydroxynaphthalen- 1-yl)-8- fluoropyrido[4,3- d]pyrimidin-4-yl)-3- methylpiperidin-3-ol | 592 |

### Example 25: 1-(7-(2-amino-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-yl)azepan-4-ol (Ex. 25)

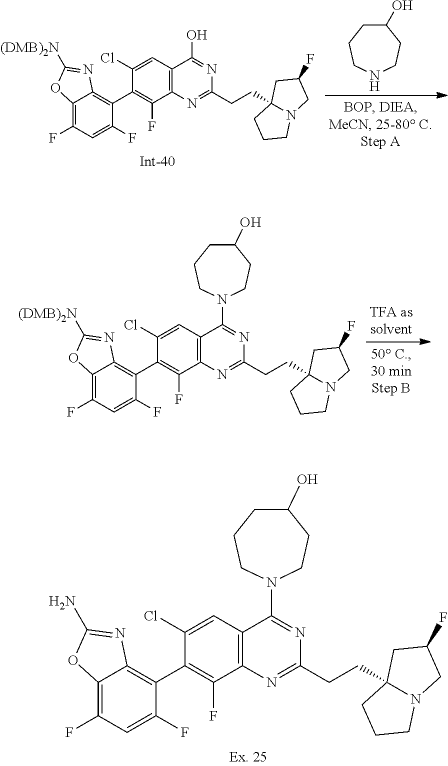

### Step A: 1-(7-(2-(bis(2,4-dimethoxybenzyl)amino)-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-yl)azepan-4-ol

To a solution of 7-(2-(bis(2,4-dimethoxybenzyl)amino)-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-ol (Int-40) (50 mg, 0.061 mmol) in MeCN (1 mL) was added BOP (40.2 mg, 0.091 mmol) at 25° C. The mixture was stirred at 25° C. for 30 min. Azepan-4-ol hydrochloride (13.80 mg, 0.091 mmol) and DIEA (0.053 mL, 0.303 mmol) were added to the reaction solution; then the mixture was stirred at 80° C. for 15 h. LCMS showed starting material was consumed and desired MS was formed. TLC (SiO₂; DCM:MeOH=15:1) showed starting material was consumed and new spots were observed. After cooling to room temperature, the reaction solution was concentrated under reduced pressure, and the residue was purified by preparative TLC plate (SiO₂, DCM/MeOH=15/1) to give 1-(7-(2-(bis(2,4-dimethoxybenzyl)amino)-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-yl)azepan-4-ol. MS (ESI) [M+H]⁺ m/z: 921.

### Step B: 1-(7-(2-amino-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-yl)azepan-4-ol

A solution of 1-(7-(2-(bis(2,4-dimethoxybenzyl)amino)-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-yl)azepan-4-ol (35 mg, 0.038 mmol) in TFA (1 mL) was stirred at 50° C. for 30 min. The reaction mixture was concentrated in vacuo, and the residue was dissolved in MeOH (1 mL). K₂CO₃ (26.2 mg, 0.190 mmol) was added to the mixture, and the mixture was stirred at 25° C. for 3 h. The reaction mixture was filtered, and the filtrate was purified by preparative HPLC (C18, 0-100%, ACN/water with 0.1% TFA), followed by preparative HPLC (C18, ACN/Water with 10 mM NH₄HCO₃) to give 1-(7-(2-amino-5,7-difluorobenzo[d]oxazol-4-yl)-6-chloro-8-fluoro-2-(((2R,7aS)-2-fluorotetrahydro-1H-pyrrolizin-7a(5H)-yl)methoxy)quinazolin-4-yl)azepan-4-ol (Ex. 25). MS (ESI) [M+H]⁺ m/z: 621 ¹H NMR (400 MHz, MeOD) δ 8.04 (s, 1H), 6.85 (t, J=10.3 Hz, 1H), 5.38-5.18 (m, 1H), 4.32-4.24 (m, 1H), 4.23-4.16 (m, 1H), 4.14-3.84 (m, 5H), 3.28-3.09 (m, 3H), 3.05-2.94 (m, 1H), 2.38-2.08 (m, 5H), 2.06-1.79 (m, 6H), 1.78-1.61 (m, 1H).

### Example 26: 1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol (Ex. 26)

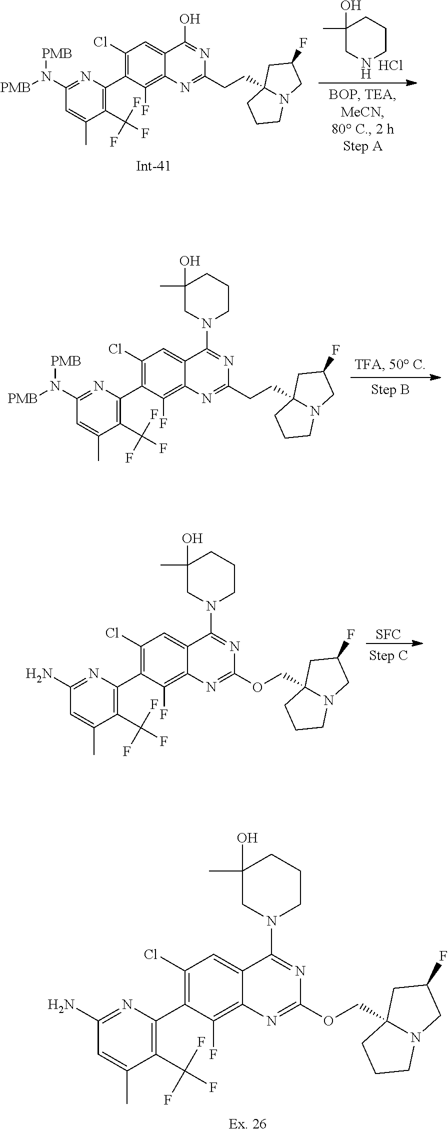

### Step A: 1-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol

To a solution of 7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-ol (Int-41, 100 mg, 0.130 mmol) in MeCN (1 mL) was added BOP (86 mg, 0.195 mmol) at 20° C. The mixture was stirred at 20° C. for 30 min. TEA (0.09 mL, 0.646 mmol) and 3-methylpiperidin-3-ol, HCl (30 mg, 0.198 mmol) were added to the reaction solution; the mixture was stirred at 80° C. for 2 h. LCMS showed starting material was consumed and desired peak was formed. TLC (SiO₂; petroleum ether: ethyl acetate=1:1) showed starting material was consumed and a new spot was observed. The reaction solution was purified by preparative TLC (SiO₂, petroleum ether:ethyl acetate=1:1) to give 1-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol. MS (ESI) [M+H]⁺: m/z 867.

### Step B: 1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol

A solution of 1-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol (113 mg, 0.130 mmol) in TFA (2 mL, 26.0 mmol) was stirred at 50° C. for 15 h. LCMS showed starting material was consumed and desired peak was formed. TLC (SiO₂; DCM:MeOH=10:1) showed starting material was consumed and a new spot was observed. The mixture was cooled, and the solvent was evaporated under reduced pressure to give the crude product. The crude product was purified by preparative TLC (SiO₂, DCM:MeOH=10:1) to give 1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol. MS (ESI) [M+H]⁺: m/z 627.

### Step C: 1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol

The racemic 1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol (80 mg, 0.128 mmol) was separated by SFC (Column D, 40% MeOH w/0.1% NH4OH) to give 1-(7-(6-amino-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol (Ex. 26) (the first eluting isomer from SFC). ¹H NMR (400 MHz, MeOD) δ 8.09 (d, J=1.6 Hz, 1H), 6.61 (s, 1H), 5.54-5.39 (m, 1H), 4.58-4.51 (m, 1H), 4.49-4.42 (m, 1H), 4.30-4.27 (m, 1H), 4.09-4.06 (m, 1H), 3.83-3.69 (m, 1H), 3.66-3.62 (m, 2H), 3.51 (d, J=13.3 Hz, 1H), 3.45-3.32 (m, 2H), 3.30-3.25 (m, 1H), 2.63-2.49 (m, 1H), 2.46-2.42 (m, 3H), 2.35-2.28 (m, 1H), 2.22-2.18 (m, 2H), 2.15-2.01 (m, 2H), 1.88-1.80 (m, 1H), 1.80-1.70 (m, 2H), 1.26 (s, 3H). MS (ESI) [M+H]⁺: m/z 627.

### Example 27: (3R)-1-(7-(8-chloro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)piperidin-3-ol (Ex. 27)

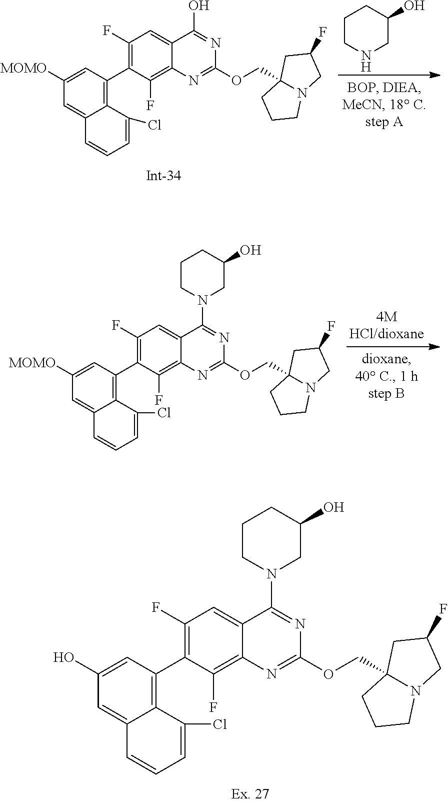

### Step A: (3R)-1-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)piperidin-3-ol

7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (Int-34) (0.0140 g, 0.0250 mmol) was dissolved in acetonitrile (0.250 mL), charged with ((1H-benzo[d][1,2,3]triazol-1-yl)oxy)tris(dimethylamino)phosphonium hexafluorophosphate(V) (0.0170 g, 0.0380 mmol) and N-ethyl-N-isopropylpropan-2-amine (0.0160 g, 0.125 mmol) and allowed to stir at 18° C. for 1 h. The reaction was charged with (R)-piperidin-3-ol (0.00505 g, 0.0500 mmol) and was allowed to stir at 18° C. for 16 h. The mixture was concentrated, and the resulting mixture was extracted by EtOAc (2 mL*2), and washed with H₂O (1 mL). Then the organic layer was combined and concentrated by reduced pressure to provide (3R)-1-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)piperidin-3-ol. MS (ESI) [M+H]⁺ m/z 643.

### Step B: (3R)-1-(7-(8-chloro-3-hydroxvnaphthalen-1-yl)-6,8-difluoro-2-(((2R,7aS)-2-fluorotetrahydro-1H-pyrrolizin-7a(5H)-yl)methoxy)quinazolin-4-yl)piperidin-3-ol (Ex. 27)

(3R)-1-(7-(8-chloro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)piperidin-3-ol (0.0128 g, 0.0200 mmol) was dissolved in dioxane (0.500 mL). HCl/dioxane (0.4 M, 0.50 mL) was added, and the reaction was allowed to shake at 40° C. for 1 h. The mixture was evaporated under vacuum, purified by HPLC (C18, 0-100% ACN/Water w/0.1% TFA) to provide (3R)-1-(7-(8-chloro-3-hydroxynaphihalen-1-yl)-6,8-difluoro-2-(((2R,7aS)-2-fluorotetrahydro-1H-pyrrolizin-7a(511)-yl)methoxy)quinazolin-4-yl)piperidin-3-ol (Ex. 27). MS (ESI) [M+H]⁺: m/z 599.

The compounds in the table below were synthesized using a similar procedure as described in the synthesis of Ex. 27 by making the appropriate substitutions for starting material, intermediates, and/or reagents. Example stereoisomers were optionally 1) resolved after completion of the indicated step using the indicated conditions and carrying forward the indicated peak; 2) synthesized by using the indicated intermediate(s) which were optionally resolved as stated herein; 3) synthesized using commercially available reagents that were optionally chirally pure; or 4) resolved after being synthesized with the indicated resolved intermediate(s). Appropriate substitutions are available commercially, synthesized as described in the literature, synthesized using methods available to those skilled in the art, or synthesized as described herein.

| | | | Intermediate(s)/ | [M + H]⁺ |
|---|---|---|---|---|
| Ex | Structure | Compound Name | Resolution | Found |
| Ex. 28 | 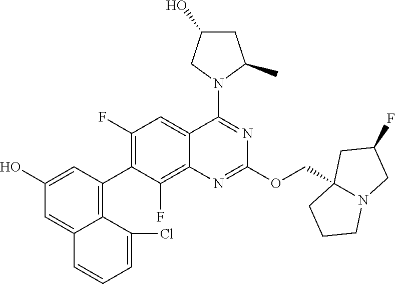 [SMILES: C[C@@H]1C[C@@H](O)CN1c1nc(OC[C@@]23CCCN2C[C@H](F)C3)nc2c(F)c(-c3cc(O)cc4cccc(Cl)c34)c(F)cc12] | (3R,5R)-1-(7-(8- chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)-5- methylpyrrolidin-3-ol | Int-34 | 599 |
| Ex. 29 | 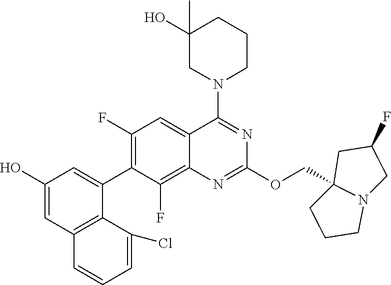 [SMILES: CC1(O)CCCN(c2nc(OC[C@@]34CCCN3C[C@H](F)C4)nc3c(F)c(-c4cc(O)cc5cccc(Cl)c45)c(F)cc23)C1] | 1-(7-(8-chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)-3- methylpiperidin-3-ol | Int-34 | 613 |
| Ex. 30 | 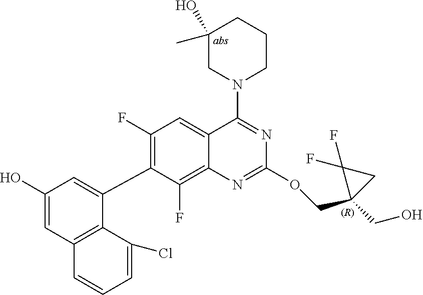 | (3R)-1-(7-(8-chloro-3- hydroxynaphthalen-1- yl)-2-(((R)-2,2- difluoro-1- (hydroxymethyl)cyclo- propyl)methoxy)-6,8- difluoroquinazolin-4- yl)-3-methylpiperidin- 3-ol | Int-35 | 592 |
| Ex. 31 | 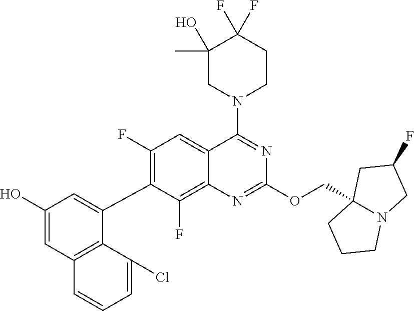 [SMILES: CC1(O)CN(c2nc(OC[C@@]34CCCN3C[C@H](F)C4)nc3c(F)c(-c4cc(O)cc5cccc(Cl)c45)c(F)cc23)CCC1(F)F] | 1-(7-(8-chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)-4,4-difluoro-3- methylpiperidin-3-ol | Int-34/Int-3 | 649 |
| Ex. 32 | 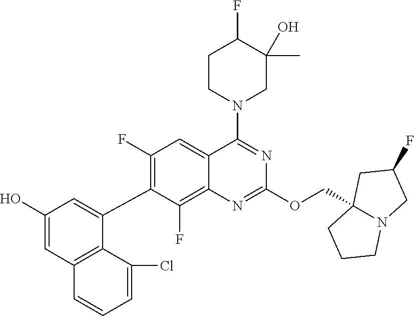 [SMILES: CC1(O)CN(c2nc(OC[C@@]34CCCN3C[C@H](F)C4)nc3c(F)c(-c4cc(O)cc5cccc(Cl)c45)c(F)cc23)CCC1F] | 1-(7-(8-chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)-4-fluoro-3- methylpiperidin-3-ol | Int-34/Int-5 | 631 |
| Ex. 33 | 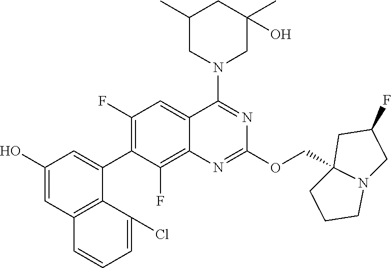 [SMILES: CC1CN(c2nc(OC[C@@]34CCCN3C[C@H](F)C4)nc3c(F)c(-c4cc(O)cc5cccc(Cl)c45)c(F)cc23)CC(C)(O)C1] | 1-(7-(8-chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)-3,5- dimethylpiperidin-3-ol | Int-34/Int-4 Step B (Prep HPLC, C18, 0- 100% ACN/ Water, Peak 1) | 626 |
| Ex. 34 | 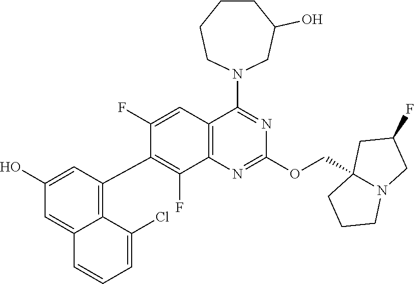 [SMILES: Oc1cc(-c2c(F)cc3c(N4CCCCC(O)C4)nc(OC[C@@]45CCCN4C[C@H](F)C5)nc3c2F)c2c(Cl)cccc2c1] | 1-(7-(8-chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)azepan-3-ol | Int-34 | 613 |
| Ex. 35 | 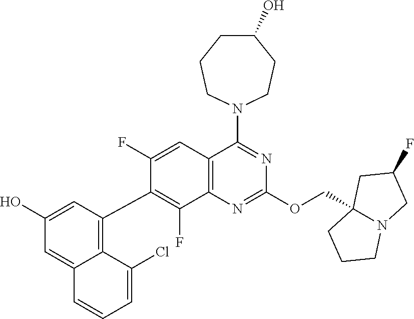 [SMILES: Oc1cc(-c2c(F)cc3c(N4CCC[C@H](O)CC4)nc(OC[C@@]45CCCN4C[C@H](F)C5)nc3c2F)c2c(Cl)cccc2c1] | (4S)-1-(7-(8-chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)azepan-4-ol | Int-34/Int-7 | 613 |
| Ex. 36 | 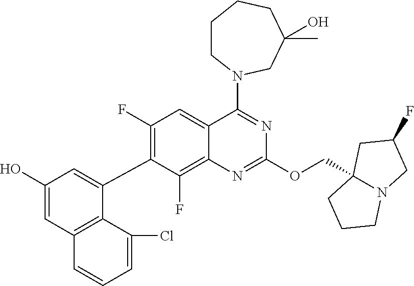 [SMILES: CC1(O)CCCCN(c2nc(OC[C@@]34CCCN3C[C@H](F)C4)nc3c(F)c(-c4cc(O)cc5cccc(Cl)c45)c(F)cc23)C1] | 1-(7-(8-chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)-3- methylazepan-3-ol | Int-34, Step A (SFC, Column R, 30% MeOH w/0.1% NH4OH, Peak 2) | 617 |
| Ex. 37 | 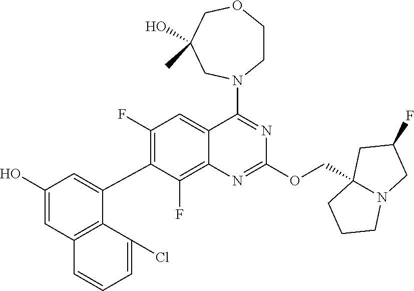 [SMILES: C[C@@]1(O)COCCN(c2nc(OC[C@@]34CCCN3C[C@H](F)C4)nc3c(F)c(-c4cc(O)cc5cccc(Cl)c45)c(F)cc23)C1] | (6S)-4-(7-(8-chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)-6-methyl-1,4- oxazepan-6-ol | Int-34/Int-6 | 629 |
| Ex. 38 | 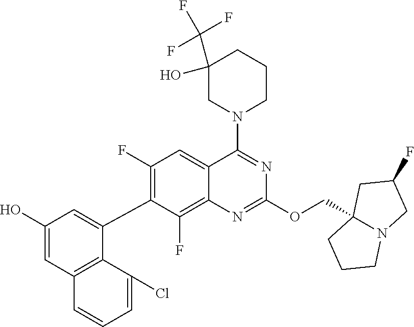 [SMILES: Oc1cc(-c2c(F)cc3c(N4CCCC(O)(C(F)(F)F)C4)nc(OC[C@@]45CCCN4C[C@H](F)C5)nc3c2F)c2c(Cl)cccc2c1] | 1-(7-(8-chloro-3- hydroxynaphthalen-1- yl)-6,8-difluoro-2- (((2R,7aS)-2- fluorotetrahydro-1H- pyrrolizin-7a(5H)- yl)methoxy)quinazolin- 4-yl)-3- (trifluoromethyl)piperi- din-3-ol | Int-34 Step B (SFC, Column Q, 50% IPA w/0.1% NH₄OH, Peak 1) | 667 |
| Ex. 39 | 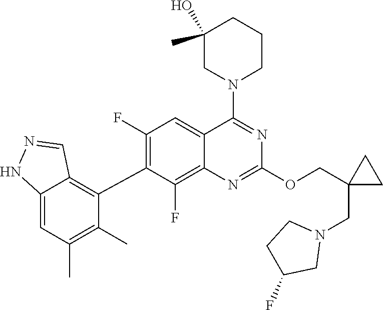 [SMILES: Cc1cc2[nH]ncc2c(-c2c(F)cc3c(N4CCC[C@@](C)(O)C4)nc(OCC4(CN5CC[C@@H](F)C5)CC4)nc3c2F)c1C] | (3R)-1-(7-(5,6- dimethy1-1H-indazol- 4-yl)-6,8-difluoro-2- ((1-(((R)-3- fluoropyrrolidin-1- yl)methyl)cyclopropyl) methoxy)quinazolin- 4-yl)-3- methylpiperidin-3-ol | Int-27 | 595 |
| Ex. 40 | 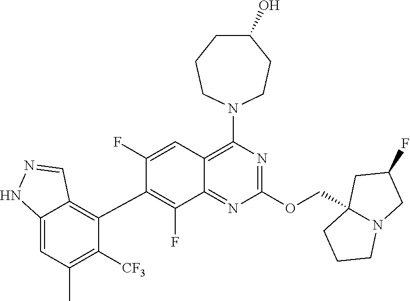 [SMILES: Cc1cc2[nH]ncc2c(-c2c(F)cc3c(N4CCC[C@H](O)CC4)nc(OC[C@@]45CCCN4C[C@H](F)C5)nc3c2F)c1C(F)(F)F] | (4S)-1-(6,8-difluoro-2- (((2R,7aS)-2- fluorotetrahydro-1H- pyrrolizin-7a(5H)- yl)methoxy)-7-(6- methyl-5- (trifluoromethyl)-1H- indazol-4- yl)quinazolin-4- yl)azepan-4-ol | Int-28/Int-7 | 635 |
| Ex. 41 | 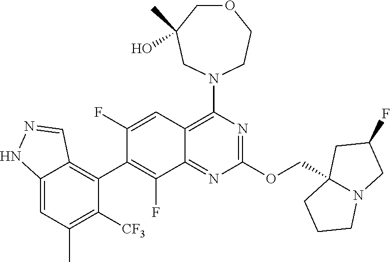 [SMILES: Cc1cc2[nH]ncc2c(-c2c(F)cc3c(N4CCOC[C@@](C)(O)C4)nc(OC[C@@]45CCCN4C[C@H](F)C5)nc3c2F)c1C(F)(F)F] | (6S)-4-(6,8-difluoro-2- (((2R,7aS)-2- fluorotetrahydro-1H- pyrrolizin-7a(5H)- yl)methoxy)-7-(6- methyl-5- (trifluoromethyl)-1H- indazol-4- yl)quinazolin-4-yl)-6- methyl-1,4-oxazepan- 6-ol | Int-28/Int-6 | 651 |

### Example 42: (3R)-1-(7-(8-ethynyl-3-hydroxvnaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Ex. 42)

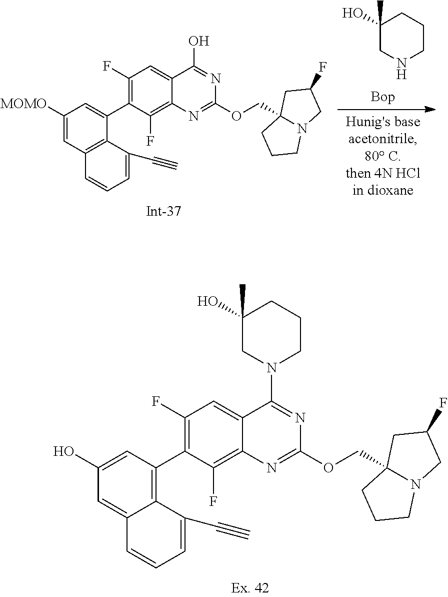

To a flask containing 7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (Int-37) (92 mgs, 0.17 mmol) and acetonitrile (1.0 mL) was added BOP (0.11 g, 0.25 mmol) followed by Hunig's base (0.15 mL, 0.84 mmol) and then (3R)-3-methyl-piperidin-3-ol hydrochloride (25 mg, 0.17 mmol). The mixture was heated to 80° C. for one hour. After 1 hour, the mixture was allowed to cool to room temperature. HCl (4.0 M in dioxane, 0.43 mL, 1.7 mmol) was added, and the mixture was heated to 40° C. for one hour. After one hour, the mixture was allowed to cool to room temperature. DMSO (0.50 mL) was added and the mixture was purified by reverse phase HPLC (MeCN/water with 0.1% TFA modifier) to afford impure product. The impure product was further purified by SFC (Column V; 30% MeOH with 0.1% NH₄OH) to afford (3R)-1-(7-(8-ethynyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Ex. 42). MS (ESI): m/z (M+H)⁺ 603. ¹H NMR (499 MHz, DMSO-d₆) δ 7.90 (d, J=8.2 Hz, 1H), 7.68 (d, J=9.8 Hz, 1H), 7.49 (d, J=7.1 Hz, 1H), 7.46-7.41 (m, 1H), 7.37 (d, J=2.5 Hz, 1H), 7.12 (d, J=2.5 Hz, 1H), 5.29 (d, J=53.9 Hz, 1H), 4.70 (s, 1H), 4.13-4.05 (m, 2H), 4.02-3.96 (m, 1H), 3.90-3.82 (m, 1H), 3.58 (s, 1H), 3.39-3.36 (m, 1H), 3.27-3.21 (m, 1H), 3.14-3.08 (m, 2H), 3.08-3.00 (m, 2H), 2.87-2.80 (m, 1H), 2.16-2.11 (m, 1H), 2.11-2.00 (m, 3H), 1.88-1.76 (m, 3H), 1.72-1.61 (m, 3H), 1.16 (s, 3H).

The compounds in the table below were synthesized using a similar procedure as described in the synthesis of Ex. 42 by making the appropriate substitutions for starting material, intermediates, and/or reagents. Appropriate substitutions are available commercially, synthesized as described in the literature, synthesized using methods available to those skilled in the art, or synthesized as described herein.

| | | | Inter- | [M + H]⁺ |
|---|---|---|---|---|
| Ex | Structure | Compound Name | mediate(s) | Found |
| Ex. 43 | 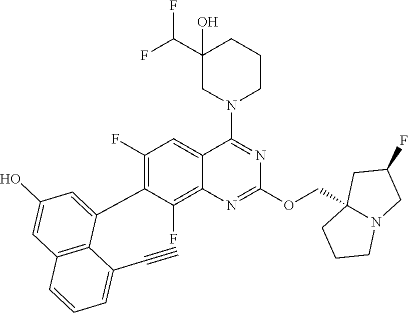 [SMILES: C#Cc1cccc2cc(O)cc(-c3c(F)cc4c(N5CCCC(O)(C(F)F)C5)nc(OC[C@@]56CCCN5C[C@H](F)C6)nc4c3F)c12] | 3-(difluoromethyl)-1-(7-(8- ethynyl-3- hydroxynaphthalen-1-yl)- 6,8-difluoro-2-(((2R,7aS)- 2-fluorotetrahydro-1H- pyrrolizin-7a(5H)- yl)methoxy)quinazolin-4- yl)piperidin-3-ol | Int-37/ Int-2 | 639 |
| Ex. 44 | 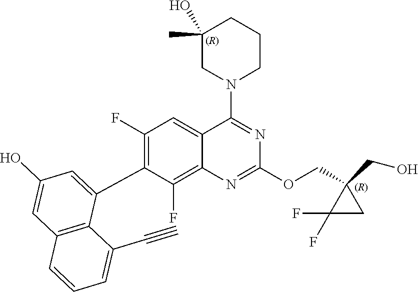 | (3R)-1-(2-(((R)-2,2- difluoro-1-(hydroxy- methyl)cyclopropyl) methoxy)-7-(8-ethynyl-3- hydroxynaphthalen-1-yl)- 6,8-difluoroquinazolin-4- yl)-3-methylpiperidin-3-ol | Int-36 | 582 |

### Example 45: ((3S,7aS)-7a-(((7-(8-ethynyl-7-fluoronaphthalen-1-yl)-6,8-difluoro-4-((R)-3-hydroxy-3-methylpiperidin-1-yl)quinazolin-2-yl)oxy)methyl)hexahydro-1H-pyrrolizin-3-yl)methyl dimethylcarbamate (Ex. 45)

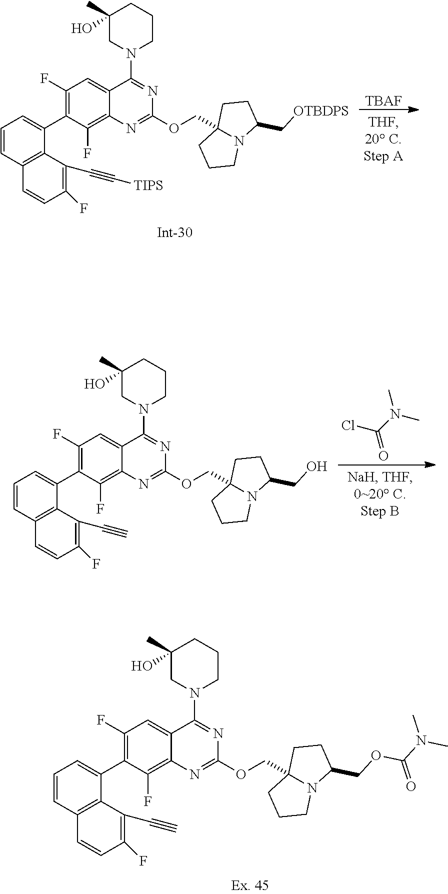

### Step A: (3R)-1-(7-(8-ethynyl-7-fluoronaphthalen-1-yl)-6,8-difluoro-2-(((3S,7aS)-3-(hydroxymethyl)tetrahydro-1H-pyrrolizin-7a(5H)-yl)methoxy)quinazolin-4-yl)-3-methylpiperidin-3-ol

To a solution of (3R)-1-(2-(((3S,7aS)-3-(((tert-butyldiphenylsilyl)oxy)methyl)tetrahydro-1H-pyrrolizin-7a(5H)-yl)methoxy)-6,8-difluoro-7-(7-fluoro-8-((triisopropylsilyl)ethynyl)naphthalen-1-yl)quinazolin-4-yl)-3-methylpiperidin-3-ol (Int-30) (105 mg, 0.104 mmol) in THF (0.7 mL) was added TBAF (0.311 mL, 0.311 mmol). The mixture was stirred at 20° C. for 1 h. LCMS showed major peak with desired product mass was observed. The mixture was concentrated, and the residue was purified by prep-TLC (CH₂Cl₂/NH₃ (7M in MeOH)=100:3) to give (3R)-1-(7-(8-ethynyl-7-fluoronaphthalen-1-yl)-6,8-difluoro-2-(((3S,7aS)-3-(hydroxymethyl)tetrahydro-1H-pyrrolizin-7a(5H)-yl)methoxy)quinazolin-4-yl)-3-methylpiperidin-3-ol. MS (ESI) [M+H]⁺ m/z 617.

### Step B: ((3S,7aS)-7a-(((7-(8-ethynyl-7-fluoronaphthalen-1-yl)-6,8-difluoro-4-((R)-3-hydroxy-3-methylpiperidin-1-yl)quinazolin-2-yl)oxy)methyl)hexahydro-1H-pyrrolizin-3-yl)methyl dimethylcarbamate (Ex. 45)

To a solution of (3R)-1-(7-(8-ethynyl-7-fluoronaphthalen-1-yl)-6,8-difluoro-2-(((3S,7aS)-3-(hydroxymethyl)tetrahydro-1H-pyrrolizin-7a(5H)-yl)methoxy)quinazolin-4-yl)-3-methylpiperidin-3-ol (40 mg, 0.065 mmol) in THF (0.5 mL) was added sodium hydride (6.23 mg, 0.259 mmol) at 0° C. The mixture was stirred at 0° C. for 0.5 h. Then dimethylcarbamic chloride (10.46 mg, 0.097 mmol) in THF (0.5 mL) was added. The mixture was stirred at 20° C. for 15 h under nitrogen. LCMS showed major peak with desired product mass was observed. The mixture was quenched with H₂O (3 mL) and extracted with EtOAc (3 mL*3), the organic layers were dried over Na₂SO₄, filtered and concentrated, and the residue was purified by prep-HPLC (C18, 0-100% ACN/water w/0.225% formic acid) to give ((3S,7aS)-7a-(((7-(8-ethynyl-7-fluoronaphthalen-1-yl)-6,8-difluoro-4-((R)-3-hydroxy-3-methylpiperidin-1-yl)quinazolin-2-yl)oxy)methyl)hexahydro-1H-pyrrolizin-3-yl)methyl dimethylcarbamate (Ex. 45). MS (ESI) [M+H]⁺ m/z 688. ¹H NMR (400 MHz, MeOD) δ 8.06-8.16 (m, 2H), 7.72 (d, J=9.6 Hz, 1H), 7.61-7.69 (m, 1H), 7.58 (d, J=7.2 Hz, 1H), 7.45 (t, J=8.8 Hz, 1H), 4.54-4.65 (m, 2H), 4.46-4.53 (m, 1H), 4.28-4.45 (m, 3H), 4.00-4.20 (m, 2H), 3.45-3.52 (m, 2H), 3.34-3.45 (m, 2H), 2.86 (d, J=17.2 Hz, 6H), 2.38 (d, J=6.4 Hz, 1H), 1.95-2.24 (m, 8H), 1.71-1.88 (m, 3H), 1.26 (s, 3H).

### Example 46: (3S)-1-(7-(8-ethynylnaphthalene-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol Ex. 46)

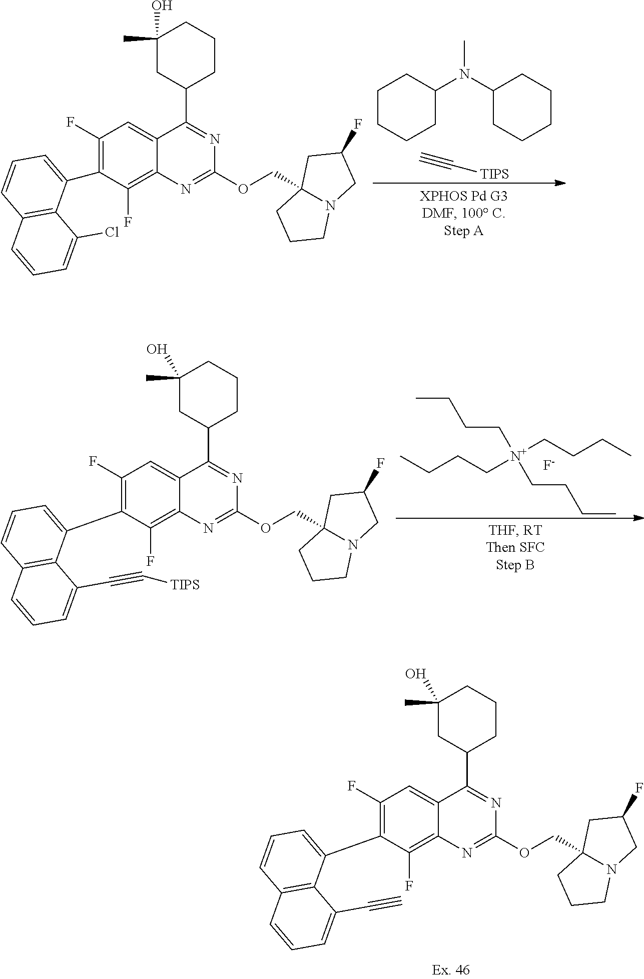

### Step A: (3S)-1-(6,8-difluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazolin-4-yl)-3-methylpiperidin-3-ol

To a solution of (3S)-1-(7-(8-chloronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Int-31) (100 mg, 0.167 mmol) in DMF (2000 μL) was added N-cyclohexyl-N-methylcyclohexanamine (359 μL, 1.675 mmol), ethynyltriisopropylsilane (751 μL, 3.35 mmol) and XPHOS Pd G3 (128 mg, 0.151 mmol). The reaction mixture was degasified with nitrogen and vacuum thrice and heated at 100° C. overnight. The reaction mixture was cooled to RT, diluted with EtOAc and quenched with sat. NH₄Cl solution. The organic layers were separated and washed with brine solution and concentrated to afford (3S)-1-(6,8-difluoro-2-(((2R,7aS)-2-fluorotetrahydro-1H-pyrrolizin-7a(5H)-yl)methoxy)-7-(8-((triisopropylsilyl)ethynyl)naphthalen-1-yl)quinazolin-4-yl)-3-methylpiperidin-3-ol. MS (ESI): m/z [M+H]⁺ 743

### Step B: (3S)-1-(7-(8-ethynylnaphthalene-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Ex. 46)

The crude mixture from step A was taken in THF (2000 μL) and added tetrabutylammonium fluoride (335 μL, 0.335 mmol) was added. The mixture was stirred for 30 mins, diluted with EtOAc, washed with sat. NaHCO₃ solution twice, brine solution and concentrated. The product was purified using silica gel chromatography with 0-25% of methanol in DCM to get a racemic mixture which was separated by Chiral SFC (Column U, 30% MeOH with 0.1% NH₄OH) to get (3S)-1-(7-(8-ethynylnaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Ex. 46). MS (ESI): m/z [M+H]⁺ 587. ¹H NMR (499 MHz, DMSO-d₆) δ 8.17 (dd, J=12.6, 8.3 Hz, 2H), 7.78-7.68 (m, 3H), 7.60 (p, J=8.0 Hz, 2H), 5.28 (d, J=53.9 Hz, 1H), 4.71 (s, 1H), 4.03 (dd, J=48.4, 10.3 Hz, 4H), 3.85 (t, J=13.1 Hz, 1H), 3.66 (s, 1H), 3.14-2.97 (m, 4H), 2.84 (t, J=8.2 Hz, 1H), 2.23-1.91 (m, 5H), 1.91-1.49 (m, 7H), 1.16 (s, 3H).

The compounds in the table below were synthesized using a similar procedure as described in the synthesis of Ex. 46 by making the appropriate substitutions for starting material, intermediates, and/or reagents optionally resolving stereoisomers after completion of the indicated step using the indicated conditions and carrying forward the indicated peak.

Appropriate substitutions are available commercially, synthesized as described in the literature, synthesized using methods available to those skilled in the art, or synthesized as described herein.

| | | | | [M + H]⁺ |
|---|---|---|---|---|
| Ex | Structure | Compound Name | Resolution | Found |
| Ex. 47 | 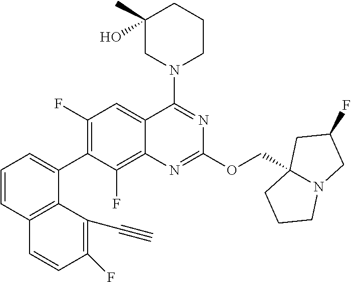 [SMILES: C#Cc1c(F)ccc2cccc(-c3c(F)cc4c(N5CCC[C@@](C)(O)C5)nc(OC[C@@]56CCCN5C[C@H](F)C6)nc4c3F)c12] | (3R)-1-(7-(8-ethynyl-7- fluoronaphthalen-1-yl)-6,8- difluoro-2-(((2R,7aS)-2- fluorotetrahydro-1H- pyrrolizin-7a(5H)- yl)methoxy)quinazolin-4- yl)-3-methylpiperidin-3-ol | STEP B SFC (Column A, EtOH with 0.1% NH4OH) | 605 |
| Ex. 48 | 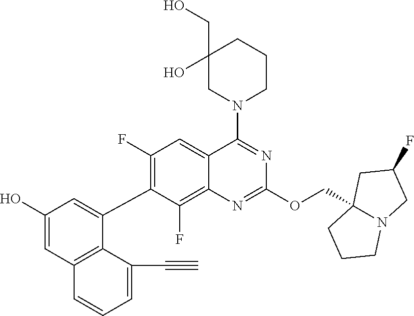 [SMILES: C#Cc1cccc2cc(O)cc(-c3c(F)cc4c(N5CCCC(O)(CO)C5)nc(OC[C@@]56CCCN5C[C@H](F)C6)nc4c3F)c12] | 1-(7-(8-ethynyl-3-hy- droxynaphthalen-1-yl)-6,8- difluoro-2-(((2R,7aS)-2- fluorotetrahydro-1H- pyrrolizin-7a(5H)-yl) methoxy)quinazolin-4-yl)- 3-(hydroxymethyl)piperidin- 3-ol | Step B Prep HPLC (C18, 0-100% ACN/Water w/0.1% TFA) | 618 |

### Example 49: (3R)-1-(7-(8-chloro-3-hydroxynaphthalen-1-yl)-6,8-difluoro-2-(((S)-1-methylpyrrolidin-2-yl)methoxy)quinazolin-4-yl)-3-methylpiperidin-3-ol trifluoracetate salt (Ex. 49)

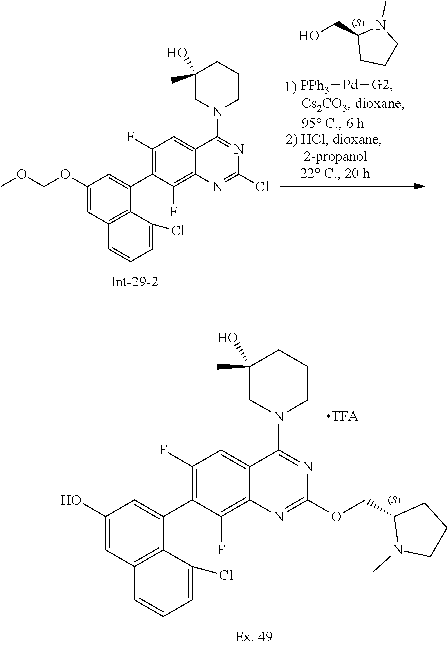

A mixture of (3R)-1-(2-chloro-7-(8-chloro-3-(methoxymethoxy)naphthalen-1-yl)-6,8-difluoroquinazolin-4-yl)-3-methylpiperidin-3-ol (Int-29-2) (30 mg, 0.056 mmol), N-methyl-L-prolinol (19.4 mg, 0.17 mmol) and cesium carbonate (58.5 mg, 0.18 mmol) in dioxane (0.5 ml) in a 2 dram vial was subsurface sparged with nitrogen for 2 minutes and treated with chloro(triphenylphosphine) [2-(2′-amino-1,1′-biphenyl)]palladium(II) (PPh₃-Pd-G2, 6.43 mg, 0.011 mmol). The vial was capped and heated at 95° C. for 6 h, cooled to rt and diluted with ethyl acetate (3 ml), and filtered through a pad of Florisil over Celite. The pad was rinsed with ethyl acetate (2×10 ml), and the combined filtrate was concentrated. The residue was dissolved in 2-propanol (2 ml) and treated at rt with 4 M HCl dioxane (0.5 ml). The mixture was stirred at rt for 20 h, concentrated, and the residue was purified by preparative HPLC (C18, 0-100% ACN/Water w/0.1% TFA) to afford the title compound (Ex. 49). MS (ESI): m/z [M+H]⁺ 569. 1H NMR (500 MHz, DMSO-d₆) δ 10.38 (s, 1H), 10.04 (bs, 1H), 7.92 (d, J=10.3 Hz, 1H), 7.87 (d, J=8.1 Hz, 1H), 7.48-7.35 (m, 3H), 7.14 (s, 1H), 4.71 (dd, J=12.3, 3.3 Hz, 1H), 4.61 (dd, J=12.4, 6.7 Hz, 1H), 4.11 (d, J=13.0 Hz, 1H), 3.92 (d, J=13.1 Hz, 1H), 3.87-3.79 (m, 1H), 3.69-3.54 (m, 2H), 3.33-3.24 (m, 2H), 3.22-3.06 (m, 1H), 2.96 (d, J=4.7 Hz, 3H), 2.34-2.21 (m, 1H), 2.13-1.83 (m, 4H), 1.82-1.57 (m, 3H), 1.19 (s, 3H).

### Assays

### Procedure for SOS-Catalyzed Guanine Nucleotide Exchange (GNE) Assay for KRAS-G12C/D/V, HRAS, and NRAS Recombinant Proteins.

Recombinant KRAS-G12C (SEQ ID NO: 1), which includes an additional triple mutation (C51S/C80L/C118S) in this assay, KRAS-G12D (SEQ ID NO: 2), KRAS-G12V (SEQ ID NO: 3), HRAS (SEQ ID NO: 4), and NRAS (SEQ ID NO: 5) proteins are used in this assay. Specifically, the SOS-catalyzed nucleotide exchange assay utilizes a preformed time-resolved fluorescence resonance energy transfer (TR-FRET) complex containing a specific biotinylated RAS protein (KRAS-G12C/V/D, HRAS, NRAS; described above) with BODIPY-GDP, and Terbium-streptavidin. Test compounds are preincubated with this complex for 60 minutes. Subsequently, recombinant human SOS protein (SEQ ID NO: 6) and unlabeled GTP are added to initiate the exchange reaction. Small molecule inhibitors stabilize the BODIPY-GDP complex whereas the untreated protein rapidly exchanges BODIPY-GDP for unlabeled GTP resulting in reduced TR-FRET signal, allowing for the measurement of inhibitory activity of the test compounds.

To assemble the preformed TR-FRET complexes, each biotinylated RAS protein is diluted to 2 μM in an EDTA Buffer (20 mM HEPES, 50 mM sodium chloride, 10 mM EDTA, and 0.01% Tween) and incubated at room temperature for one hour. This mixture is then further diluted to 90 nM in an Assay Buffer (20 mM HEPES, 150 mM sodium chloride, 10 mM magnesium chloride, and 0.005% Tween) containing 15 nM of Terbium-Streptavidin (Invitrogen, catalog #PV3577) and 900 nM of BODIPY-GDP (Invitrogen, catalog #G22360) and incubated at room temperature for six hours. It should be noted that this preformed TR-FRET complex for each of the RAS proteins was made ahead of time, aliquoted and stored at −80° C. until the day of the experiment.

Each test compound (10 mM stock in DMSO) is diluted in DMSO to make a 10-point, 3-fold dilution in a 384-well low dead volume microplate (Labcyte, catalog #LP-0200). Once titrations are made, 10 nL of the diluted compounds is acoustically dispensed into a 384-well assay plate (Corning, catalog #3820) using an Echo 550 (Labcyte). Each well of the assay plate subsequently receives 3 μL of a specific 3× RAS preformed TR-FRET complex and 3 μL of Assay Buffer and is incubated at room temperature for 60 minutes (preincubation time). Each well then receives 3 μL of 3×recombinant human SOS protein and GTP (Sigma, G8877) in Assay Buffer and is incubated at room temperature for 60 minutes, or 90 minutes for KRAS-G12V. The final reaction in each well of 9 μL consists of 3 mM GTP, and the specific RAS and SOS proteins in the following concentrations: KRAS-G12C/SOS=3 nM/40 nM, KRAS-G12D/SOS=1 nM/40 nM, KRAS-G12V/SOS=1.25 nM/160 nM, HRAS/SOS=1.25 nM/40 nM, and NRAS/SOS=1.25 nM/40 nM.

The TR-FRET signal is measured on an Envision (PerkinElmer) plate reader: Excitation filter=340 nm; emission 1=495 nm; emission 2=520 nm; dichroic mirror=D400/D505; delay time=100 ms. The signal of each well is determined as the ratio of the emission at 520 nm to that at 495 nm. Percent effect of each well is determined after normalization to control wells containing DMSO (no effect) or a saturating concentration of inhibitor (max effect). The apparent effect as a function of compound concentration is fit to a four-parameter logistic equation to calculate IC₅₀ values using Spotfire software. The results of this assay for each RAS type are presented in the table below under heading titles that include “GNE”.

### Procedure for Cellular Phospho-ERK Assay, G12C

NCI-H₃₅₈ cells (ATCC® CRL-5807™), containing homozygous KRAS-G12C activating mutation, were cultured in T150 flask in growth medium (RPMI medium 1640-GlutaMAX™—I (ThermoFisher Scientific 61870) containing 10% fetal bovine serum (ThermoFisher Scientific 10091148)). The cells were harvested in growth medium after TrypLE (ThermoFisher scientific 12604021) digestion and were seeded in a 384-well collagen coated cell culture plate (Corning 356702) at a density of 15,000 cells/well, and incubated at 37° C., 5% CO₂ overnight. The compound dose-response titrations (30 μM final concentration and 1:3 dilutions, 10-point dose response) were prepared and appropriate amounts of test compounds were dispensed in a 384-well intermediate plate using an Echo 550 liquid handler. RPMI medium 1640-GlutaMAX™—I was added to the intermediate plate and the contents of the intermediate plate were then transferred to the 384-well collagen coated cell culture plate, which was incubated at 37° C., 5% CO₂ for 2 hours. After removal of medium from the collagen coated cell culture plate, cells were lysed in lysis buffer from Alpha SureFire® Ultra™ Multiplex p-ERK and total ERK assay kit (PerkinElmer MPSU-PTERK) containing Halt™ Protease and Phosphatase inhibitor cocktail (ThermoFisher Scientific 78446) at room temperature with constant shaking at 300 rpm for 30 minutes. The cell lysates were then transferred to an OptiPlate-384 plate (PerkinElmer 6005620), and the phosphorylation of ERK (p-ERK) and total ERK levels were detected by Alpha SureFire® Ultra™ Multiplex p-ERK kit and total ERK assay kit (PerkinElmer MPSU-PTERK) following the manufacturer's protocol. Assay plates were read on an EnVision Multimode Plate Reader (PerkinElmer), and the ratio of p-ERK vs. total ERK in each well was used as the final readout. Dose response curves were analyzed using a 4-parameter logistic model to calculate IC₅₀ values using Spotfire software. The results of this assay are presented in the table below under the heading titled “G12C H_{358”.}

### Procedure for Cellular Phospho-ERK Assay, G12D

AsPC-1 cells (ATCC® CRL-1682™), containing homozygous KRAS-G12D activating mutation, were cultured in T150 flask in growth medium (RPMI medium 1640-GlutaMAX™—I (ThermoFisher Scientific 61870) containing 10% fetal bovine serum (ThermoFisher Scientific 10091148)). The cells were harvested in growth medium after TrypLE (ThermoFisher scientific 12604021) digestion and were seeded in a 384-well collagen coated cell culture plate (Corning 356702) at a density of 15,000 cells/well, and incubated at 37° C., 5% CO₂ overnight. The compound dose-response titrations (30 μM final concentration and 1:3 dilutions, 10-point dose response) were prepared and appropriate amounts of test compounds were dispensed in a 384-well intermediate plate using an Echo 550 liquid handler. RPMI medium 1640-GlutaMAX™—I was added to the intermediate plate and the contents of the intermediate plate were then transferred to the 384-well collagen coated cell culture plate, which was incubated at 37° C., 5% CO₂ for 2 hours. After removal of medium from the collagen coated cell culture plate, cells were lysed in lysis buffer from Alpha SureFire® Ultra™ Multiplex p-ERK and total ERK assay kit (PerkinElmer MPSU-PTERK) containing Halt™ Protease and Phosphatase inhibitor cocktail (ThermoFisher Scientific 78446) at room temperature with constant shaking at 300 rpm for 30 minutes. The cell lysates were then transferred to an OptiPlate-384 plate (PerkinElmer 6005620), and the phosphorylation of ERK (p-ERK) and total ERK levels were detected by Alpha SureFire® Ultra™ Multiplex p-ERK kit and total ERK assay kit (PerkinElmer MPSU-PTERK) following the manufacturer's protocol. Assay plates were read on a EnVision Multimode Plate Reader (PerkinElmer), and the ratio of p-ERK vs. total ERK in each well was used as the final readout. Dose response curves were analyzed using a 4-parameter logistic model to calculate IC₅₀ values using Spotfire software. The results of this assay are presented in the table below under the heading titled “G12D ASPC-1”.

| | KRAS | KRAS | KRAS | | | | | |
|---|---|---|---|---|---|---|---|---|
| | G12D | G12D | G12C | G12V | G13D | HRAS | NRAS | G12C |
| | GNE | ASPC-1 | GNE | GNE | GNE | GNE | GNE | H358 |
| Ex # | IC₅₀ (nM) | IC₅₀ (nM) | IC₅₀ (nM) | IC₅₀ (nM) | IC₅₀ (nM) | IC₅₀ (nM) | IC₅₀ (nM) | IC₅₀ (nM) |
| Ex-1 | 8 | 70 | 22 | 28 | 21 | >9950 | >9950 | 41 |
| Ex-2 | 7 | | 95 | 25 | 43 | | | |
| Ex-3 | 223 | | 280 | 588 | 616 | | | |
| Ex-4 | 9 | | 143 | 17 | 14 | | | |
| Ex-5 | 11 | 1148 | 63 | 30 | 20 | | | |
| Ex-6 | 92 | 3049 | 104 | 77 | 103 | | | |
| Ex-7 | 1 | 95 | 8 | 2 | 2 | | | |
| Ex-8 | 149 | | 735 | 89 | 105 | >9950 | >9950 | |
| Ex-9 | 315 | 1882 | 297 | 381 | 221 | | | 4208 |
| Ex-10 | 1 | | 7 | 2 | 3 | | | |
| Ex-11 | 1 | 132 | 7 | 2 | 2 | | | |
| Ex-12 | 2 | 86 | 14 | 6 | 9 | | | 41 |
| Ex-13 | 29 | | 52 | 47 | 74 | | | |
| Ex-14 | 1186 | | 678 | 1225 | 5190 | | | |
| Ex-15 | 273 | | 172 | 732 | 1506 | | | |
| Ex-16 | 1 | 352 | 20 | 3 | 3 | | | 123 |
| Ex-17 | 283 | | 195 | 237 | 341 | >9950 | >9950 | |
| Ex-18 | | | | | | | | |
| Ex-19 | 91 | 3345 | 44 | 47 | 129 | | | |
| Ex-20 | 4 | 1184 | 36 | 6 | 6 | | | |
| Ex-21 | 1 | 329 | 5 | 1 | 1 | | | |
| Ex-22 | 1 | 1161 | 6 | 2 | 1 | | | 305 |
| Ex-23 | 1 | 65 | 6 | 1 | 2 | | | 14 |
| Ex-24 | 1 | 20 | 6 | 1 | 1 | | | 2 |
| Ex-25 | 21 | 1405 | 99 | 14 | 20 | | | 611 |
| Ex-26 | 2006 | 3917 | 1184 | 1548 | 711 | | | 7726 |
| Ex-27 | 321 | 3835 | 42 | 54 | 66 | | | |
| Ex-28 | 637 | 1225 | 72 | 354 | 406 | | | 2257 |
| Ex-29 | 15 | 900 | 46 | 17 | 13 | | | >30000 |
| Ex-30 | 2428 | 3151 | 340 | 1033 | 1351 | | | 512 |
| Ex-31 | 984 | 1728 | 196 | 358 | 189 | | | 468 |
| Ex-32 | 59 | 261 | 28 | 40 | 16 | | | 118 |
| Ex-33 | 163 | | 37 | 143 | 86 | | | |
| Ex-34 | 285 | 6299 | 162 | 589 | 441 | | | 3623 |
| Ex-35 | 270 | 4840 | 371 | 153 | 182 | | | 1823 |
| Ex-36 | 398 | 1864 | 175 | 300 | 142 | | | 3583 |
| Ex-37 | 6 | 151 | 12 | 2 | 3 | | | 48 |
| Ex-38 | 857 | 6861 | 1011 | 598 | 777 | | | 2170 |
| Ex-39 | 2709 | | 792 | 2288 | 2663 | | | |
| Ex-40 | 173 | 1328 | 311 | 470 | 931 | | | 981 |
| Ex-41 | 33 | 285 | 22 | 60 | 48 | >11110 | >11110 | 203 |
| Ex-42 | 3 | 91 | 14 | 3 | 4 | >10000 | >10000 | 58 |
| Ex-43 | 13 | 215 | 8 | 10 | 8 | | | 58 |
| Ex-44 | 74 | 142 | 26 | 87 | 93 | | | 59 |
| Ex-45 | 23 | 379 | 24 | 12 | 14 | | | 331 |
| Ex-46 | 986 | 4686 | 431 | 168 | 291 | | | |
| Ex-47 | 239 | 2070 | 39 | 390 | 173 | | | 979 |
| Ex-48 | 13 | 376 | 79 | 5 | 7 | | | 180 |
| Ex-49 | 18 | 690 | 119 | 20 | 27 | | | 427 |

SEQUENCES

SEQ ID NO: 1-Recombinant KRAS G12C
GLNDIFEAQKIEWHETEYKLVVVGACGVGKSALTIQLIQNHFVDEYDPT
IEDSYRKQVVIDGETSLLDILDTAGQEEYSAMRDQYMRTGEGFLLVFAI
NNTKSFEDIHHYREQIKRVKDSEDVPMVLVGNKSDLPSRTVDTKQAQDL
ARSYGIPFIETSAKTRQGVDDAFYTLVREIRKHKEK
SEQ ID NO: 2-Recombinant KRAS G12D
GLNDIFEAQKIEWHETEYKLVVVGADGVGKSALTIQLIQNHFVDEYDPT
IEDSYRKQVVIDGETCLLDILDTAGQEEYSAMRDQYMRTGEGFLCVFAI
NNTKSFEDIHHYREQIKRVKDSEDVPMVLVGNKCDLPSRTVDTKQAQDL
ARSYGIPFIETSAKTRQGVDDAFYTLVREIRKHKEK
SEQ ID NO: 3-Recombinant KRAS G12V
GLNDIFEAQKIEWHETEYKLVVVGAVGVGKSALTIQLIQNHFVDEYDPT
IEDSYRKQVVIDGETCLLDILDTAGQEEYSAMRDQYMRTGEGFLCVFAI
NNTKSFEDIHHYREQIKRVKDSEDVPMVLVGNKCDLPSRTVDTKQAQDL
ARSYGIPFIETSAKTRQGVDDAFYTLVREIRKHKEK
SEQ ID NO: 4-HRAS
GGGGSHMTEYKLVVVGAGGVGKSALTIQLIQNHFVDEYDPTIEDSYRKQ
VVIDGETCLLDILDTAGQEEYSAMRDQYMRTGEGFLCVFAINNTKSFED
IHQYREQIKRVKDSDDVPMVLVGNKCDLAARTVESRQAQDLARSYGIPY
IETSAKTRQGVEDAFYTLVREIRQH
SEQ ID NO: 5-NRAS
GGGGMTEYKLVVVGAGGVGKSALTIQLIQNHFVDEYDPTIEDSYRKQVV
IDGETCLLDILDTAGQEEYSAMRDQYMRTGEGFLCVFAINNSKSFADIN
LYREQIKRVKDSDDVPMVLVGNKCDLPTRTVDTKQAHELAKSYGIPFIE
TSAKTRQGVEDAFYTLVREIRQYRMKKLN
SEQ ID NO: 6-Recombinant Human SOS Protein
MGSSHHHHHHSGENLYFQGSSGLNDIFEAQKIEWHESSEEQMRLPSADV
YRFAEPDSEENIIFEENMQPKAGIPIIKAGTVIKLIERLTYHMYADPNF
VRTFLTTYRSFCKPQELLSLIIERFEIPEPEPTEADRIAIENGDQPLSA
ELKRFRKEYIQPVQLRVLNVCRHWVEHHFYDFERDAYLLQRMEEFIGTV
RGKAMKKWVESITKIIQRKKIARDNGPGHNITFQSSPPTVEWHISRPGH
IETFDLLTLHPIEIARQLTLLESDLYRAVQPSELVGSVWTKEDKEINSP
NLLKMIRHTTNLTLWFEKCIVETENLEERVAVVSRIIEILQVFQELNNF
NGVLEVVSAMNSSPVYRLDHTFEQIPSRQKKILEEAHELSEDHYKKYLA
KLRSINPPCVPFFGIYLTNILKTEEGNPEVLKRHGKELINFSKRRKVAE
ITGEIQQYQNQPYCLRVESDIKRFFENLNPMGNSMEKEFTDYLFNKSLE
IEPRNPKPLPRFPKKYSYPLKSPGVRPSNPRPGT

1. A compound of Formula (I) or a pharmaceutically acceptable salt thereof 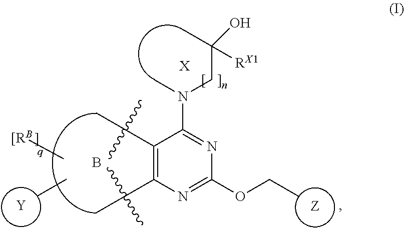
wherein
the moiety 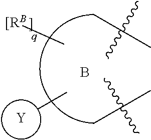
is selected from the group consisting of: 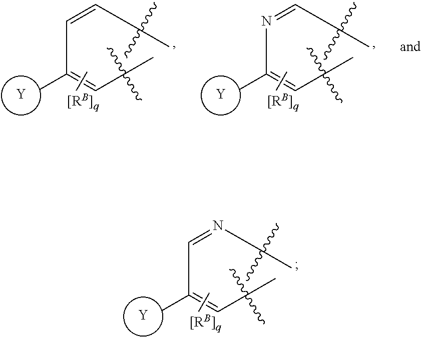
each occurrence of R^{B} is independently selected from the group consisting of halo, cyano, C₁-C₃ alkyl, C₁-C₃ fluoroalkyl, cyclopropyl and C₁-C₄ cyanoalkyl;
Ring X is selected from the group consisting of:
(i) a 5- to 9-membered monocyclic- or fused bicyclic- or bridged bicyclic-heterocycloalkyl, wherein the heterocycloalkyl is saturated and contains 0 to 2 heteroatom groups selected from the group consisting of N, S, S(O), S(O)₂ and O, in addition to the illustrated N atom; and
(ii) an 8- to 10-membered spiroheterocycloalkyl, wherein said spiroheterocycloalkyl is saturated and contains 0 to 2 heteroatom groups selected from the group consisting of N, S, S(O), S(O)₂ and O, in addition to the illustrated N atom;
wherein ring X is unsubstituted or independently substituted by 1 to 3 R^{X2} substituents selected from the group consisting of fluoro, cyano, hydroxy, C₁-C₆ alkyl, C₁-C₆ fluoroalkyl, C₁-C₃ hydroxyalkyl, —N(H)C(O)heteroaryl, wherein heteroaryl is optionally substituted by C₁-C₃ alkyl;
R^{X1} is selected from the group consisting of H, C₁-C₆ alkyl, C₁-C₆ fluoroalkyl, C₁-C₆ hydroxyalkyl and C₁-C₄ cyanoalkyl;
Ring Y is selected from the group consisting of:
(i) a 8- to 10-membered bicyclic ring system, wherein the 8- to 10-membered bicyclic ring system is partially unsaturated or aromatic, and wherein the 8- to 10-membered bicyclic ring system contains 0 to 3 heteroatoms selected from the group consisting of N, S, and O;
(ii) a 13- to 14-membered tricyclic ring system, wherein the 13- to 14-membered tricyclic ring system is partially unsaturated or aromatic, and wherein the 13- to 14-membered tricyclic ring system contains 0 to 3 heteroatoms selected from the group consisting of N, S, and O; and
(iii) phenyl; and
(iv) a 6-membered heteroaryl ring containing 1 to 2 N atoms;
wherein Ring Y is unsubstituted or independently substituted by 1 to 4 R^{Y} substituents selected from the group consisting of halo, hydroxy, amino, oxo, C₁-C₃ alkyl, C₁-C₃ alkoxy, C₁-C₃ fluoroalkoxy, C₂-C₃ alkynyl, C₂-C₃ fluoroalkynyl, C₁-C₃ fluoroalkyl, C₁-C₃ cyanoalkyl, C₃-C₆ cycloalkyl, C₃-C₆ cyclofluoroalkyl, C₃-C₆ cycloalkoxy, C₃-C₆ cyclofluoroalkoxy, C₂-C₃ alkenyl, C₂-C₃ fluoroalkenyl and cyano;
Ring Z is selected from the group consisting of:
(i) a 5- to 8-membered monocyclic- or bicyclic-heterocycloalkyl, wherein said heterocycloalkyl is saturated and contains 1 to 3 heteroatoms independently selected from the group consisting of N, S, and O, and wherein said heterocycloalkyl is unsubstituted or substituted with 1-2 substituents R^{ZHC} selected from the group consisting of halo, hydroxy, C₁-C₃ alkyl, C₁-C₃ hydroxyalkyl, —C(H)(OH)CF₂H, —O—CH₂—O—(C₁-C₃ fluoroalkyl), and methylene(C₁-C₃ alkyl)(C₁-C₃ alkyl)carbamate;
(ii) 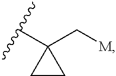
wherein M is selected from the group consisting of hydroxy, C₁-C₃ dialkylamino, and C₁-C₄ alkylamino, and wherein the cyclopropyl group is unsubstituted or independently substituted with up to 2 halo groups;
(iii) 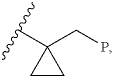
wherein P is 5- to 8-membered monocyclic- or fused bicyclic- or bridged bicyclic-heterocycloalkyl, wherein said heterocycloalkyl is saturated and contains 1 to 2 heteroatoms selected from the group consisting of N and O, wherein said heterocycloalkyl is unsubstituted or substituted with 1 R^{P} substituent selected from the group consisting of halo, hydroxy, C₁-C₃ hydroxyalkyl, C₁-C₃ cyanoalkyl, carbamoyl, C₁-C₃ alkoxy, cyano, and —NHC(O)C₁-C₃alkyl, and wherein the cyclopropyl group is unsubstituted or independently substituted with up to 2 halo groups; and
(iv) a 4- to 8-membered monocyclic- or bicyclic-cycloalkyl, wherein said cycloalkyl is saturated and wherein said cycloalkyl is unsubstituted or independently substituted with 1-3 substituents R^{ZC} selected from the group consisting of halo, hydroxy, C₁-C₃ alkyl, C₁-C₃ fluoroalkyl, C₁-C₃ hydroxyalkyl, C₁-C₃ hydroxyfluoroalkyl, C₃-C₄ cycloalkyl, C₃-C₄ cyclofluoroalkyl, C₃-C₄ hydroxycycloalkyl, and C₃-C₄ hydroxycyclofluoroalkyl;
subscript n is 1 or 2; and
subscript q is 0, 1 or 2.

2. The compound of claim 1 or the pharmaceutically acceptable salt thereof, wherein ring X is a 5- to 8-membered monocyclic heterocycloalkyl, wherein the heterocycloalkyl is saturated and contains 0 to 1 heteroatoms selected from the group consisting of N, S, and O, in addition to the illustrated N atom.

3. The compound of claim 1 or the pharmaceutically acceptable salt thereof, wherein the group 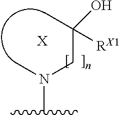
is selected from the group consisting of:

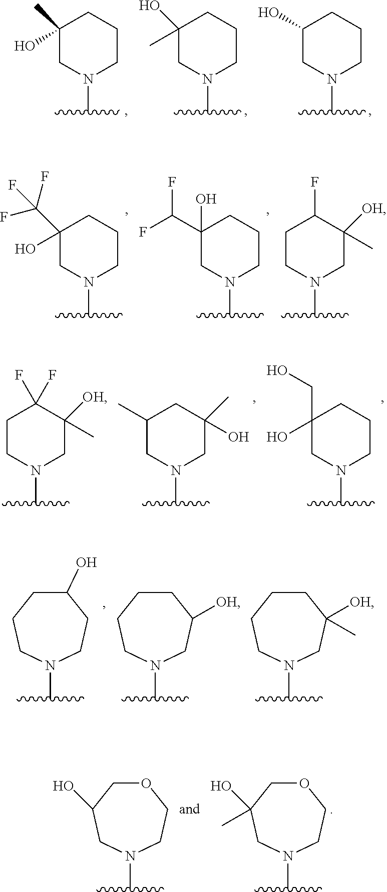

4. The compound of claim 1 or the pharmaceutically acceptable salt thereof, wherein
the group 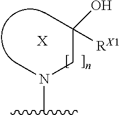
is selected from the group consisting of:

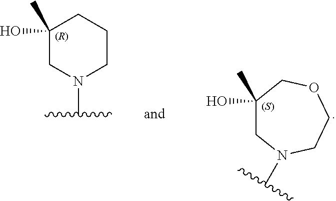

5. The compound of claim 1 or the pharmaceutically acceptable salt thereof, wherein
the moiety 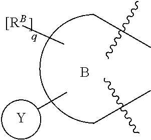
is

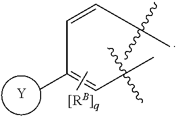

6. The compound of claim 1 or the pharmaceutically acceptable salt thereof, wherein ring Y is selected from the group consisting of naphthyl, phenyl, pyridyl, benzoxazolyl, benzopyrazolyl, benzothiazolyl, pyridopyrazolyl and benzothiophenyl.

7. The compound of claim 1 or the pharmaceutically acceptable salt thereof, wherein ring Y is selected from the group consisting of:

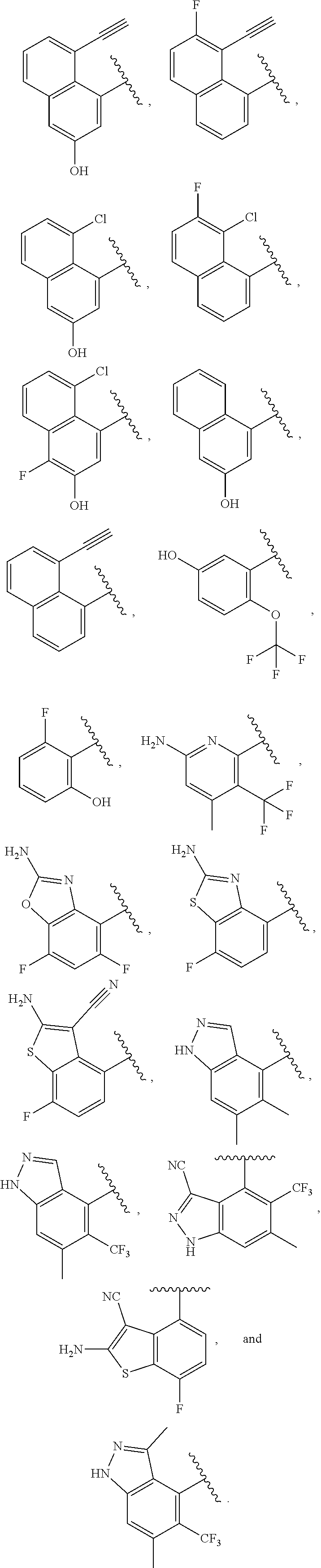

8. The compound of claim 1 or the pharmaceutically acceptable salt thereof, wherein the group 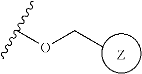
is selected from the group consisting of:

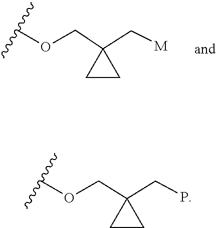

9. The compound of claim 1 or the pharmaceutically acceptable salt thereof, wherein the group 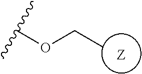
is selected from the group consisting of:

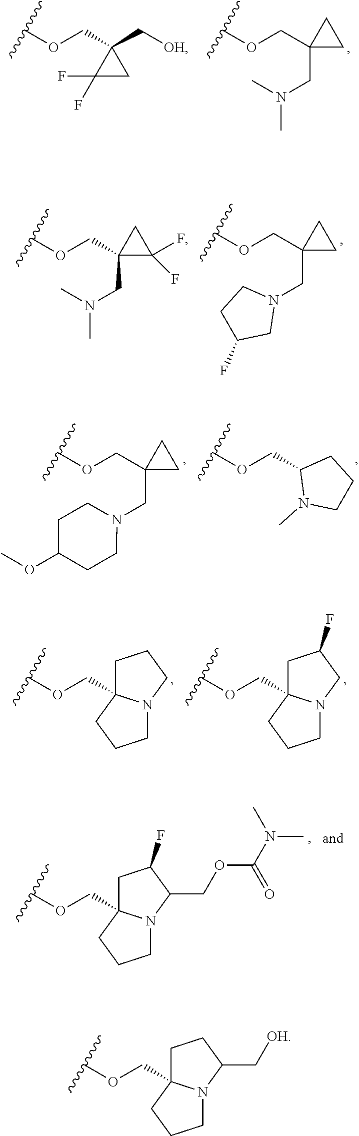

10. The compound of claim 1 selected from Examples 1-49 or the pharmaceutically acceptable salt thereof.

11. A pharmaceutical composition comprising the compound of claim 1 or the pharmaceutically acceptable salt thereof, and a pharmaceutically acceptable carrier.

12. A pharmaceutical composition comprising the compound of claim 1 or the pharmaceutically acceptable salt thereof, an additional anti-cancer agent, and a pharmaceutically acceptable carrier.

13. A method of inhibiting KRAS-G12D protein comprising contacting KRAS-G12D protein with the compound of claim 1, or the pharmaceutically acceptable salt thereof, to inhibit the activity of the KRAS-G12D protein.

14. A method of inhibiting KRAS-G12C protein comprising contacting KRAS-G12C protein with the compound of claim 1, or the pharmaceutically acceptable salt thereof, to inhibit the activity of the KRAS-G12C protein.

15. A method of inhibiting KRAS-G12V protein comprising contacting KRAS-G12V protein with the compound of claim 1, or the pharmaceutically acceptable salt thereof, to inhibit the activity of the KRAS-G12V protein.

16. A method of treating cancer comprising administering a therapeutically effective amount of the compound of claim 1, or the pharmaceutically acceptable salt thereof, to a subject in need of such treatment.

17. The method of claim 16, further comprising administering an additional active agent to the subject.

18.-24. (canceled)
